SEQUENCE LISTING (343 sequences)

SEQ ID NO: 1 - DNA, 403 bp, Cannabis sativa
  source 1..403: mol_type = other DNA
  variation 202: allele = n = t/c
ttgattggagaaaggtcataaaagaaaaatacaccaggaagaaactgaagattcccaaattctgaagtcctaaaatgctcagttacagaaaactgcaatatgcaattggctaaagtcatgctccatgtttcataaatgaaataaaccagatagtaaatatatgcatacgtaaagagataaccacctgattagactgaatagngtgtccagtcacgtctgtgtaaacagtaggtaccacctaaaacataaacagcaacccttgatctgagagctaatagatttcccttatatcgtctagtattaggtatcaatttgtgcgtgtgttttcaaagaatgaatccaaaggacttaccttgataaaatactgatacatcccactaggtgtggcctgcgaccattgcac

SEQ ID NO: 2 - DNA, 403 bp, Cannabis sp.
  source 1..403: mol_type = other DNA
  variation 202: allele = n = t/a
ttggggaaaagagaaaactggtaattttcatcaacaaatctctgaatgtaagagcttgattcggagacttaaagggaagaatgatgaagtgtctgtcgaggcccatctcaaagcggaaagagatctttttgaagtgcttactaaaagggaagtgtttgggaaacaacgctccaagcaactttggctcaaagaaggagatagnaatagtaagtatttccatgtttttgcgagtgctcgtaaacgccaaaatacgattcataagcttcaagattacaatgggagttggcttgattgggagctccgctcatgatcatatggtcgcttctaatcgaagttattttcacttctgcacttcataccgatattcttgatggtattgcctcgctattacttcttgaaatga

SEQ ID NO: 3 - DNA, 403 bp, Cannabis sativa
  source 1..403: mol_type = other DNA
  variation 202: allele = n = t/c
tgtgtgttagatcacaaagaccaaccgtacgagctgttacagatttgcattgaagatcaatgccttatctacaaattagatagcattggccaaaaattcaacccaaaagtgctgaaggatttgcttcacgacagccgcgtgacggtggtcggagtgggtatagagaatgtggcgaggcggttggaggaggatcacggttggntcatgcctaaagtggttgaactgcgagatttggcggcccaggcccaggcccaaaccaaggcccaaaaggcagatattcagggtagtactagcggtcatgataatggaaagaagatcatgacgaagaaggtgatgatgatgaaaagaaactttagccgatttggtatagcgaagctggcgaaagtggttttgggaaaagaga

SEQ ID NO: 4 - DNA, 403 bp, Cannabis sp.
  source 1..403: mol_type = other DNA
  variation 202: allele = n = c/g
tctgccgtgatatttttgaaattttaggttttgtttccgtaaatttgatcgttgtacataatttgttagcttaaagtttatgattttttgctgaacaagcttgggtgatacagctgctgcagcgaatatggatgagtttggtgtattgacggagaggtttggattgaagcctcaaggtaaatcggctccaatggcggcttcnaagagatctgtcaatagtaacgatggccagaattggaattcggggtttgattcgggtgtaaaccccaacttttcatctttttcagacgatcggaacggtttttatcaatccggcaaagataaggtagctcagaacagcggaagcttgaatgattatgatgatatatttggtggacctagtaagcaatcgggtagtcatggt

SEQ ID NO: 5 - DNA, 403 bp, Cannabis sp.
  source 1..403: mol_type = other DNA
  variation 202: allele = n = a/g
atttgttacttcttcgtgtcttgatgccaagatagcgaaaaagtacatagatcattcgttcattttgagattgctagatttgcttgattccgaggatcctagagaaagagactgcttaaagactattcttcataggacctatgggaaatttatggttcataggccatttataaggaagtctataaacaatgtattttaccgntttgtgtttgagactgagaaacataatgggattgctgaggtattggagatctatgggagtatcattagtggatttgcattacctttaaaggaggagcataagatattcttgcggagggttttgattcctctgcataggccaaagtctttgggggtttacttccaacaactctcctactgtgttacacaatttatagagaag

SEQ ID NO: 6 - DNA, 403 bp, Cannabis sp.
  source 1..403: mol_type = other DNA
  variation 202: allele = n = c/t
cccatatgatacatatgaatgtaaaacacttacccgctcttcaggaaaggaacttgagaatccgaatattttaccagaaaatacatttgaagtttttccgttttgagtgcttgaaccaagttgcactctctttggagctttattcttatcacttactcgacccttactttttttattgacagcagtaacgttactctgctgngatgattttgctgcagttgccataatgttgacatcagtcgtgttgatttctggtttttttcctttgcttccgcccaatggtaatggcattccaattccacctcttatgctaccagataaaggattagatggcatgatcggatgaacagcagagactctagcttgatttagactggtcaatcctgtaattgctccttcaaga

SEQ ID NO: 7 - DNA, 403 bp, Cannabis sp.
  source 1..403: mol_type = other DNA
  variation 202: allele = n = c/t
tcgcgattgttattcccacctcgtcttttacttcgaatctcagtgtctcgtagaagtttattagagctgccttcgccgcctacacattagtgtctcgtaaatatcaagtgatgatatgttttattggcttaagcaacacatatgttttaatcaattactcacagcatataagctcattctaggcagaggtaaccagttctcnactgaggcattaactatgactctaccattgctttggtataagtaaggaagagccacaaatgttggataaacatttccccaaaagttaatgtcctgaaaacccaaaaagataaattacaaaaattaagagtgttcacggattggtttagtctagatttttaattttttcaaaataaaccactattacggttttttaaaaatt

SEQ ID NO: 8 - DNA, 403 bp, Cannabis sp.
  source 1..403: mol_type = other DNA
  variation 202: allele = n = c/a
tttccaagtgtatacgaaacttcgcactcttatcaatgttgtggtccattgttatctaaacgtttatcatgttgtgcttttgcagtactattctttggatattacatattttaagtcctctcttgatagccatctactggatctgctatggaacaagtactgggtgaatacactttcttcttcacccctactgggtaatggngattatgttgcaggacaaatttcagacttaggtacctaaattgttatgataccagattttttttttaaatgctcgtctagctttctgtaatgtttatcatggtaataaaatcattaactgtgttgtactttttatcatttatgcagctgaaaagttggagcaagcagagaaccaattgtcacattcccgctttgggccatt

SEQ ID NO: 9 - DNA, 403 bp, Cannabis sp.
  source 1..403: mol_type = other DNA
  variation 202: allele = n = a/g
ttgacttcataacttattagtaactgatgttatttcttcacttccaacatcttgtggttgcttattatgatggtgtggtttctttctgtcctaacaggagagcaagattctgaagtttttaggctttatgtggaatcctttgtcctgggtcatggaagctgctgctataatggctattgccttggcaaatggtggtggaagncctccggattggcaggactttgttggtattattgtcctgctgatcatcaactcaactatcagttttattgaagaaaacaatgctggtaatgcagcagctgctctcatggctggtcttgctccaaagactaaggtaaaacaacagcctcaccatcaccatcagacttgtttggtttttagcttttatggtatttttttttaa

SEQ ID NO: 10 - DNA, 403 bp, Cannabis sp.
  source 1..403: mol_type = other DNA
  variation 202: allele = n = a/g
ctactctctcttctccatttccatctccagctccagttctagttccatctctttctagatactcaactgtatacatatatcatcacaaatttatatcttaaatcaaaaccatgaaactttaatggctatgattcagcttcagaggggtttgtgttcggccacctcaaccacaaccacaaccacagtttctgcttctttctcnaacccttctacttcatcagctctgcaactccgccctttcatggctaaggttcttaatctccatttcattatcttcttcctgctttcttaaatttattatggttttcattattatattaaacttcttctcttcaatacttagctataacaattttttttttcggctgctgtcgtaacactcctctaatgaggaactttacat

SEQ ID NO: 11 - DNA, 403 bp, Cannabis sp.
  source 1..403: mol_type = other DNA
  variation 202: allele = n = a/g
tacaattacatttacaaatcccataaaggcctaaacaaggccatgatgaaacgacgtcgtcgtcgttgatgggtaaaacgcttctcgaagcttgttgtacttttgtatatcaaagctatgaatctccatgagccgtttttgcctggagagaaagcggcgttggaagtagccttggaaagttggctccgtcgaggtacggagnaagaagccgtagcccaaagcgaaatggatcgatgtcacaaagaagcatatgggctccatcacgtcccaactcagctcccaaaaggttagtctcatggccgcaagtgtttgggccagtacgaagcccagcccaatataaagctccccacggaccaattctcttgctttctggtcgatcatcgatttttgtttctccatttcc

SEQ ID NO: 12 - DNA, 403 bp, Cannabis sp.
  source 1..403: mol_type = other DNA
  variation 202: allele = n = a/g
tcttcaaacagtttcctgccataaatgaaaaaatgtttgaaaagagaaaaactatgtttaattttgtatctttaccaaaacaaatgagtaaatagatacctgaacaagtctctcttttttaaaattgctggccaagtgagttctgctaaagcttgtgaaaagacaagcaattcaaacaacttgttatcctcgtggactgganctccccattctttatcgtggaataaagtataaattgggtctgtgattcattacaaaaagaaaaggaaaaaaaagatcgatcgtgtaagcattataaattataattgaaccaccccgacaaggtcaaacgtgccatttttggacaaacataagacatgagccacgaatatctcattacaattatctattcaacaagttacat

SEQ ID NO: 13 - DNA, 403 bp, Cannabis sp.
  source 1..403: mol_type = other DNA
  variation 202: allele = n = c/a
atccaattagtaatccgtgcccttggtaaagagaagtttcagtaacgccaaagatcctcaaagtatatacatcaggtacagagaggaacaaaagagttgtcatttgtcactagaaccagccacatacgaaacaacgactgtgatatcatcaagctttcccccatagtagcggaaaccagcatcttgagcagctgtggaaaanggtgtctgcctgtccttgtcctgtgctcgttgacgcgccaatgctgctattttttgagctgttgcctgaggccctaagccagctcgcatggcatgcactactactgccgttatctcattgttgtacaagttgtcaaagagtccatcagtcccagcaatgatgacatccccgggagctacaggtactgtaaaaacctgaatc

SEQ ID NO: 14 - DNA, 403 bp, Cannabis sp.
  source 1..403: mol_type = other DNA
  variation 202: allele = n = a/g
catccacttcgaaccgtactttgccacacagtaagcgtccgtacaccctttaccgtcgatggtcttgaccggaacaagattctttgcccgaatgataccgagttccactgtgccaacaggaggcttccagagttgcctagccgtggggcggtagtcgctgctcatgtgtgcaacctcatccatcacgtgatatccgccatcnaaacagagccttaggtgaacacgtccgttataagttcttcttttctcatcctgaatattttcaaggctgagccaccgtgacgctacttttctgtcatccactctcctctctacaacagtaagaggaattattgccgttcccaatgtagtttgtcccttttgatgtcgtatctcaagagtgattacaatgtgttcagagaaa

SEQ ID NO: 15 - DNA, 403 bp, Cannabis sp.
  source 1..403: mol_type = other DNA
  variation 202: allele = n = g/c
aaatggagggctcttttagcaggggctatagctggaccttccatgcttcttactggagttaatacgcagcacactagtttggctatttacatacttatgcgagctgcggttttagcgtcgcgttgtgggattaaaagcaaacggtttgggagatattgtaagccactcacatgggctcatggcgacattttccttatgtgtntctcttcttcccaaattctgtacgtatatcttttcaaattggattgttatgttttcctacatctaactttgttttgtttttatttctctcttagtttcgttttgtattctttctaattggggattcgcaaaatgtatttggtttatgggcattgtattgaattttcgagtttggcttgttcccctgtctttatgggcgtgt

SEQ ID NO: 16 - DNA, 403 bp, Cannabis sp.
  source 1..403: mol_type = other DNA
  variation 202: allele = n = t/c
acattgtaaatccaaaaattcaattgcaaccctgtttaagcaagtaaatttcaactgatttaatcaagattttctgagctataaacgcagttcccttattcaaaaagtcaaatttcctcttaacattttaatttgtcataaatccacagtttctgaattgggtccggaagatacaagcaagaacatagtagagatcatattncaatcaagctggctcaaaaaggaagccccaatatgcaaaatcgaccgaatactgaaagttcacaatacccagaaaaccatatccaagttcgaggagtatcgagaagccattaaagccaaggccaccaagctcccaaagaagcaccctcgatgcatcgccgacgggaacgagctcctccgcttccattgcgccaccttcatg

SEQ ID NO: 17 - DNA, 403 bp, Cannabis sp.
  source 1..403: mol_type = other DNA
  variation 202: allele = n = c/t
tatatatacatatatataatcatttctatctttcttcatcatcatcgtcatcagtttctctgtctgccgaaaaatccgtttgttttttctgtaaattgattagatgggaaaggacgactacaacaattacgatgagagagacagagggctcttctctcatcttggtagtgcatactattcccagcagcaccactctggatcntaccctccgcccccgccgtctgcatatccaccgccgcctcatcatcatcaagcctaccctcctccacccggctacccatctgctggataccctcctccgtcgcctcacgccgcatacccaccgccctatactcctcccggtggataccctccttccgcttatcctcatcctcctcctcctgctccatatccatatcctgca

SEQ ID NO: 18 - DNA, 403 bp, Cannabis sp.
  source 1..403: mol_type = other DNA
  variation 202: allele = n = g/a
tttcaccattcctgtcctcttgctcaatttccactacaaccatcctccctccattccgatcataataacacccacatcctcattcgtcgtaaagcagcctcatccaacggtcgacatttcacagctcgctcttctagcccggactcaatccctccttctccttctcctcctccttcttcctccattactcaacaggtcattntcagctcagcgttgaccatcgcattcgccgttgccaaccgcgttctttataagctagctctcgttcccatgaaagaatatcctttctttttagctcagttggccacttttgggtgagtaagtagtgttcattaatttataaagaaaataaaaaataaaaatttcggactcaatgatgatgattaataaataaaatctccct

SEQ ID NO: 19 - DNA, 403 bp, Cannabis sp.
  source 1..403: mol_type = other DNA
  variation 202: allele = n = t/c
aatacgtcaacttgcataacacgataatacaaattgccacctttatgagaggtgatccacaggcagcacgtttctatgtcctttaagggctacaaaattatcattttgattcaatagtgttttcaaagttgaaagacattgatattgtttacccccccatggttttgtgcagttgttggaatttgacgagtcaaaagcctcncaagaagatgaagatgacaaagttagcagcaagaaggaggaatagtttgagaagctcataacctaaaacttccctttttagatgaatacttttttttttttattatcaactttcttctttttcttatttccaaggtcccaaaacggccttagagaacgcattttggctaattcttatgctgctccacaccccatgtaacag

SEQ ID NO: 20 - DNA, 403 bp, Cannabis sp.
  source 1..403: mol_type = other DNA
  variation 202: allele = n = t/c
ctgtctagacctgacatcaccgttgctgtcaactgtctcagccagtttatggccaatccatagacaactcatctccaagcagttcatcatctactacactaccttaaagaaaatcctggtcaagggctcttctattcacctacttcagcctttacactacgcggtttttctgactttgattgggcatcttgccctgttactngatgttcaacaacagggtattgtatcttcctcggagacagtctcatttcttggagagcaaagaagcagtcaataatatccaaaagctctgcagaagtcgagtatagagccttggatgcaacaacaagtaaaatgacatggctgcaatatcttctccatgacttccagataccacagccacatcttgcctttatttactgtg

SEQ ID NO: 21 - DNA, 403 bp, Cannabis sp.
  source 1..403: mol_type = other DNA
  variation 202: allele = n = a/g
gaagacagattttcaatttgcaagctcagagggcggttagtggtggattaaggagatgtcaggaggtggatgtagcatagtatggttcaggagagatctaagggtagaagataatccagctttagcagctggtgttagagcaggtgctgtgattgctgtctttgtctgggcacccgaggaagaaggtcattactatcctggnagggtatcaagatggtggcttaagcaaagtttggctcatcttgatttctctttgcgaagttttggcacttctctcatcaccaaaagatctgctgatagtgtttcttctcttcttgaggttgtcaagtccactggtgccaaacaaatactcttcaaccacttatatggtcagtattttctttcgttatatatttttcacagc

SEQ ID NO: 22 - DNA, 403 bp, Cannabis sp.
  source 1..403: mol_type = other DNA
  variation 202: allele = n = c/g
aacaaaaaaagttatctctagtcaacataagaaagagcaacgacaacagctcttatcttagcataaaaagaacataagccactaccctacttccttgggcgagtgtcctcagactcggccatgccggcagcgaccttggctatatctttggcagtggcatcaggctttttggtagtgtatagtgtgaaatatccaatggtancgatgatgagtaagccaccaatggccatgatggctgggctgtagggaagcttccttctctgatggaggacacccccaggattgtggcctggtggcctctgagacccttctactccagctgcttttatttcctcttgactcattttgtgggtgtctgccattttcctccactcttcgttcttttccattttcctttcctaga

SEQ ID NO: 23 - DNA, 403 bp, Cannabis sp.
  source 1..403: mol_type = other DNA
  variation 202: allele = n = g/a
gaaatacaagccaaaccccataaaatattctcaatcaaataagtagtttcatcttcattgggaggcagcagcagcagcaacagcagaatcgttgggtttgggcaaaggggtattaacttggattctaccatcaatacaggaagagcacttggtttcagcccatgtctccaagtcaaaatttccatagcccatgattctcttncctgagcacaaccgacccaccattactgccattgctcctaagatgagaatagcaactaagacaccaacaacaggaccaacagaagatgaaccatggctcactctctgctgctgcggcgacactactactacactcagtggtggagcctgtgatgacatttcgttaaaaaaagagattagttaacacaagctgaccactaca

SEQ ID NO: 24 - DNA, 403 bp, Cannabis sp.
  source 1..403: mol_type = other DNA
  variation 202: allele = n = c/g
agaaaccttaatcttcaatttaagaccattgttcttattattctgcttcttcgtcagcccatccccctcctgaacaccgaatctccgaataccctccacgtacttcatgtacatgtcgttgtacggaagcttcatgccgccgccacccccaccgtcgtcacgggggttcggagggttcggcgaaacaccaaaacccattccnacctgaccaacgctggcgaataagccaccggggtgataatacccctcttgggaattccattgagaagaccctaaaccgctgacggagtaaaacccatctttcgtctcatcaaatagttgaactcctctcttacccattaatctcaaaaaataaaagacacaaactttcggacacgctctcttttatttgcttcttcttctt

SEQ ID NO: 25 - DNA, 403 bp, Cannabis sp.
  source 1..403: mol_type = other DNA
  variation 202: allele = n = a/t
tgtccaatcaatctcttcccaactcctcctggttccattacaaagaggcgaaaccgtcgtcgttttatgctccccagggctggaactcgtggaaatcatattcggttgccaaagagctggccttttgaccgtacccatttctccaccagacccaacttcagacaactgccaccatcttataagagtcttatcccaaaccaancccaaagccgcaatagctcaccaaacttacataaccaaagttcaccaattcatgacctcttcctcctcggccacttcaaaacttgcaagcttgttaaaaaaactaaagtggatctcaactcaacatgtcaagcagtactccaaaacgacatctttgaagttattcgattctggtttgtcgtataaagggtgtaatgcagaa

SEQ ID NO: 26 - DNA, 403 bp, Cannabis sp.
  source 1..403: mol_type = other DNA
  variation 202: allele = n = a/g
atatcgtgcttaatatataagaacaataaataacatgttattatagctaattaatatagcacttaataaattcttatactataatggtacgtacataatgtgggaagctagcaattaagtgatgatggatgagttattaattacctgtgttgatcattattatgatcattaggaaatcctctaaggtggttgcaggcagagncagagcctctgtaattgggtgtagggaattggtcagattcaattttgaggaggtcctgcacaaggatttcgtagtgtctcttgacttcgtctggagtcttcccacccactgctttggccacattttgccatcggtcaggggtgtccttgtcatacacagctaaagccttttcaaattgcttgttttgcttagacgtccaag

SEQ ID NO: 27 - DNA, 403 bp, Cannabis sp.
  source 1..403: mol_type = other DNA
  variation 202: allele = n = c/t
ggtgaagatacatgatctttctggctcagctgttgccgcagcgttcatgacaactccgtttgtgccttccacaggttgtgattgggttgccaagtccaacccaggagcttggttgattgttcggcctgatgcttgcaggccagagagctggcagccatggggtaaactcgaagcttggcgtgaacgtggtatgagggactcngtttgctgtagatttcgtctcatgtctgaaggtcatcaggaggcaggggagcttctcatgtctgagatttatatcaatgccgaaaggggaggggagtttttcatagacacggataggcaggcgcttgcaacggcagcaactccaatcccaagcccgcaaagcagcggagactttgcaggactcagcccagttgttggaggc

SEQ ID NO: 28 - DNA, 403 bp, Cannabis sp.
  source 1..403: mol_type = other DNA
  variation 202: allele = n = a/g
agccccaacaccacccccaccaccaacaccactgcctccacctatgctcagaccaagaagatcacgcgtcatgggcggactcgcgaacgtcaacgatgggcccatgactagatctgtcaagccagaatttccaccaggctgaagcccaagcccaagcccagctgccaaagtcgacgaaccactttccggcttgatttgagtnctgttccactgcgtgccaagactgtctttaacggaggctgaagatgttgatgttgtcaagccaaaactgcacaacagcgaggaatttgaagccgaagcacccatttgggctgctttttgaagcaatgccgttgcagacattgctggaggctgcagtggtgatgggctgaagttgcggtgctcggcctggtcttgtgtttga

SEQ ID NO: 29 - DNA, 403 bp, Cannabis sp.
  source 1..403: mol_type = other DNA
  variation 202: allele = n = a/t
caagaacttttgcagcgatcgattccgaaggctcttcgtcaaccttgaaatcgaaaacttccatagctagacgaggagaggtgtttgttcccgactccagaaatgaactggctggagtcaaaagtcaactgggaatcgtcaactagtttgcatatggatcggggtgaggagtactttagagagaagaactcgggagtgtgcngcggaaggaggaatttggtggtgtagaatcgtgccctaatttgtactgtactaatccagttatagaagagatcagctgactttacttgtacttgtactttgagaagtaaagtgatgataatgaaacagagcaaatatgatcattatttttttgtgtgtgtatgattagcgtgtttggtaaatcagattttcgtgtttgcat

SEQ ID NO: 30 - DNA, 403 bp, Cannabis sp.
  source 1..403: mol_type = other DNA
  variation 202: allele = n = a/t
agaattttagagacggtggagtttggttgtgtgaaaatgcgccaggcttgcttagcaagcaacgcctggttgaagtgaacaagagatcggaacccaagcccgccttctcttttagacctacaaagttttttccaactttgccaatgaatcttggggcaattgttgtccttgaatccccaccagaaattagccataatggacnccagagagtggcaagtggccacagggagacgaaagcaggccatagtataggtgggaattgcctgaatgacagacttgagaaggatttccttacctcccttagagaagactttatttttccaattatgaaggtgggcccaaactttgtcatgaagatagtgaaaaaaattatttttttcgttcggccaatatattgagggag

SEQ ID NO: 31 - DNA, 403 bp, Cannabis sp.
  source 1..403: mol_type = other DNA
  variation 202: allele = n = g/a
caacccaaagaaagaaaaaaaaaaaatctaacaacgcattatatgagttgtattgctaaaataactcccaatcccaaatccaaaaccatcaatagaagaagaaggagaaggagatgatgataatgaagatgaagatccaattctcaaaaccctaagcaaatccaaacacatactcctcactctgtgaccctcacagtacccnctctgcatcaccatcagaatcttcgcaattccattgctgttcaccaccgcctccttcgtcttcttctcccccaataagcagcacacgctccaaaccaccgccaccgcgtcctctgtcgccgacctcgacaccttcatcattctctccacgattgccaccgcacacctacagtctactccgattgctgatcttccctccgct

SEQ ID NO: 32 - DNA, 403 bp, Cannabis sp.
  source 1..403: mol_type = other DNA
  variation 202: allele = n = a/c
tgaaacttcaaagaatgcatcttggtatccttgaaatgatccggaaaagaatttttttgaaggagccaaaaagtactaatcatttcattggttaccctgtaattggttgagaaccatttcagactcattatctttgtcatcattgtcatcggtattgaaactaccttcctcaaatgctacttcattgtcattgttagcgtcnagtgataagccggcaatcgatgcataccactcacctcctaaatagcctgaatccctcatcaatctaaccaaaacttctttgctttcgagatcattttcttgctcacatagatcgagaaaagcagatacgtagacaggtttgggcctccagttgttcgaaccatcggctgcaaaagcttttttccagtaagtcaaagcctcg

SEQ ID NO: 33 - DNA, 403 bp, Cannabis sp.
  source 1..403: mol_type = other DNA
  variation 202: allele = n = a/g
ggatagaaagaccacgttatcatgagggccacaacttagtagtgtacaaaattcagggaaaaagttaattgttctttagaaaaagcaataatgggagcctaaattttgcaaattagaattcaatttagcatttttctcaccatgagaagattccacgtccaaaatcagatcaagtgcatagtcataatatgggacttgactnctcaaaccacaaaggttgaaatcatcttgaatgtattcatcatcgacttcgcaaaagaactcatttcctcgcaaattgcaaaaccatgaaatccaagaagtgtcatctccatcagagccacttacatcggattcttcactatccgtctcagattcctctgcaaacaacgacacaacacgtcaataagcaagcttagagaat

SEQ ID NO: 34 - DNA, 403 bp, Cannabis sp.
  source 1..403: mol_type = other DNA
  variation 202: allele = n = a/g
tttgggacagataaaactatttgggtaccaatcatgtcaagtccaaaagaacttggttctactagggacccgtcaccaggctttgtccagatgtgccttgaaagtatcaatgtcacaatctggtggaccagaaaatgttaatatgaaaatcaatgacgtcaaggagaaattacagaaagccattcctgactcagttaaggancttgaatgggagaaaggggctgatatactggtgcagcaattgctttctcttggacaaaaggcattcaaatggcttactgttgttttgattgctgtgagctttttatcagatgttatattcactatctctaggaaccaggaattagtcatgccatttggtctcttggctggttgtttattggctgatttctttaaagaaaca

SEQ ID NO: 35 - DNA, 403 bp, Cannabis sp.
  source 1..403: mol_type = other DNA
  variation 202: allele = n = c/t
ttgtccgcctacaaacaaattatgcaatatcagaaccttatcacccactacaacaccatgttctattcaaatcaacatatcttagataaacaaaagctgtagacatggactgacctgagaccagtatgagatccaaatgccgcggatctcatgggtgacacgtgggtcagataacgcatcaatccatcgtttgatgaatcgntcttgcctgaaaaaatcaaggtgtgaaagccaagtcaagattagtgcctgcaataagattgagaaattttgattttactttgcagaaaatagaggaggataagtacctgtctggtgtaaaggatcggtatctctctccaggttgcttgaagttgttctccttctcgataatgcactacactcaccaaataaatacacacat

SEQ ID NO: 36 - DNA, 403 bp, Cannabis sp.
  source 1..403: mol_type = other DNA
  variation 202: allele = n = c/a
aagcaattacagaagcagcagaaggtggtgcaaatgcaggtggatatgatgccccatgatccggtttcttgttcggctgctcaggtaggtttggttgcttttgtaaataccgtttcaaattaacagaaacagcattatcttctggaatgttctgatttcttcgtttacctagttccaaatcaaggcttattccagcatccgnttcgagttcatttaaaccattgccctccaaggactgcttgcgcggaggagggctacaagggcattcggaggtgagtatcttagttccaaaaaccttgatactattactgtccatcactgttcctcctgttatacatgaactctccggaccataagatatgaaccttctgttatcattttgcaagtcccttaatgccacctt

SEQ ID NO: 37 - DNA, 403 bp, Cannabis sp.
  source 1..403: mol_type = other DNA
  variation 202: allele = n = g/c
gagcttcttgttagattcatcaatcgataaattcaacaaagctgttactgcatgctcttgaatctttgaatctggataggataggagctgtactaatggtgggattcctccattgtgggcaatcaaaattctgttctcagggttctctttggagagcaaccgaaccttcttaaccgcctttcgctgcgtttctaaatggctngaagatagtccttcaacaagggacaagatctcctctttgagatcagctgaggagctttcatggtctcctggattatcctttgagggaagtttaaaattgttcttctcactccactgcaagattatgtttttaagagcataatttgacgcaagtgtcagatgaggaagagtttgcctggttttgggacaagtcctgtgatta

SEQ ID NO: 38 - DNA, 403 bp, Cannabis sp.
  source 1..403: mol_type = other DNA
  variation 202: allele = n = g/a
aggaagacaaaagatccaaataaagaaattggaaaacaagagtaacaaacatgtcactttctctaaacggcgctctggtctcttcaagaaagctggcgagctcagccttctttgtgggtccaaggtggcagtcatcgtcttctctcccactggcaaactcttctgcttcggtcatccatctgtggacgacgtcgttcgctcntatctcgattataataaccaaacagtagtagtattacctggtagtaatgataacattactgctactactactactagtactagtacttttgtaaaagctatgactgagttggaagaggccgagaaaaagaagaagcaattggtgagacaagcgagattggcctcgattttagataataatagttgcgcgtggtgggaggaa

SEQ ID NO: 39 - DNA, 403 bp, Cannabis sp.
  source 1..403: mol_type = other DNA
  variation 202: allele = n = g/c
atccccagcagaacaccatcaaagccgtcaatatctcatcacatcagatccatcagtcttccatgcagatctcatccgttgatttcccagctcagggacggcgttgctgagcttcactgctgggtaacccaaccggaatgtcggacctccgtttggctgaccaacgggttgacccgactgagagacctccacgactgtctcnacgatattcttcagcttccacagacccaagaatctctccgccgattacccagctccgtcgtagagaatcttctagaagatttcctccgcttcgtagacgtgtacggtattttccagacctcgattctggtaatcaaggaagaacaatcagcggcgcaagtgggtttacgaaagagagacgagttaaaggtttctctttacg

SEQ ID NO: 40 - DNA, 403 bp, Cannabis sp.
  source 1..403: mol_type = other DNA
  variation 202: allele = n = c/a
tcagtaacagtctaagtctctccattattatgggctgcgccaactcgaagctcaacgatcttccggcggtggccttgtgccgggaccgatgtaaatacctagacgaagctctccgccatagccaagacttcgctgaagctcacgccgcttacctcgactccctcaaggcattgggtcccgcttttgatcgtttcttcagtanaaaagtaccagtcaacaatgaaactttaaaaaagtcttcttctttatctccggcggccgtggcttcttcttccccgctgcataaatcaaactcagcagactcgcaccttcgattcccttccgattcttctgaggacgaggatagtgataatcataataaactcgggcataattgcctcaacgaagaggaatcccaaggctt

SEQ ID NO: 41 - DNA, 403 bp, Cannabis sp.
  source 1..403: mol_type = other DNA
  variation 202: allele = n = c/a
tcatgacctgaacttgtacttttttaatttaaaagtcgttttggaagaagttaggatttgtatattaatctttttcggcttcctatgtttggaccaaaccatctgctgcagggttgtggagagtgttggggaagatgtgaatgaagtagttgaaggagatagagtgatcccaacatttctttcagattgtggggagtgtttngactgcaaatcaaagaagagtaacctctgttcgaatttccccttcaaggtctcaccttggatgcccagatatgagtccaccagattcacagacctcaaaggagaccctttgtaccattttctgtttgtttcaagttttagtgagtatactgtggttgatatctccaatcttacaaaaattagctctgaaatccctccaaac

SEQ ID NO: 42 - DNA, 403 bp, Cannabis sp.
  source 1..403: mol_type = other DNA
  variation 202: allele = n = t/c
cttctctaatatagtatagctatgcttattggataatcctaaagctccattttaattatgctatagctaagttcacttttgccacactgcatatttattgccagaagttgctagtgtccactcccttccagcagggcaccaagagccctccccaatcttcatacaaacattctcaccaattactgcagaatagaggttaggntgagcttccaggattctaatggatgatcggctgtgaatgtcctgacgctttcgaatttcaatctgtggagtagaaataaaacataaagtttcaccaaagcatgaataatttacttggtacactatcaaaacaaaaactagtgtcctaccagcttcacaatttgatcatgaatagagcttccccaatcgtaaaaatggtcat

SEQ ID NO: 43 - DNA, 403 bp, Cannabis sp.
  source 1..403: mol_type = other DNA
  variation 202: allele = n = g/t
gatatctacttgcagatgtacgctaaagatttgtttcagaaagactttctttagaggaagaagaaaatgaagatgaaagagacaagagtgagtgattattaagagaaccaaactcaaaactcaagagattacaaaaaagaaaagaaaagttgtcagtgtaacacgccaaagtaaaaaaagagtgtagagatagagtgtgttngggtgtataattaattactatgaagacgacttctgattatttatcagtatatagatttaaaagttttggcagaaaaaggtaattatgtattgaccgttacatcccacgcgtccaatccctatttcttttgttctcagctttggtcagactaaaatttactccccttctttaattactgtcctatctttggaactagcccat

SEQ ID NO: 44 - DNA, 403 bp, Cannabis sp.
  source 1..403: mol_type = other DNA
  variation 202: allele = n = g/c
ggcagacaacataacccttctatttcattttcctttcttcttgtttttggtctctctctctcttccacatacaatccaagtttcgtgaaagtacacaaataagaaaataacaaagatttataataacgtactagataggtttaatatatagttataatctgatgatgatgcggaaagtgatggcttcatcttcttcttcttnttgtttttattatcgtcacataataataacaacaatagtattgttattttttattaatccatcgtggggtttgataaagttaccgccaaatcagacagttccggcagtgatcgtgttcggagactcgatcatggacacagggaacaacaacgctcttaagactctggtcaagtgtgatttcgctccttatggtgaaaattt

SEQ ID NO: 45 - DNA, 403 bp, Cannabis sp.
  source 1..403: mol_type = other DNA
  variation 202: allele = n = t/c
tttgatattttaggtacggatcggggaaggagaggaagttgttggagatggcgccgactacgattcggaaagcgattggggcggtgaaggaccagactagtattggaatagctaaggtggctagcaacatggcgccggagcttgaggttgcgattgtcaaggcgactagtcacgacgatgatccggctagcgagaagtacanaagagagatcctgaatctcacatcttactctcgaggttacgttcatgcctgtgttacggcgatttcgaagcgtttggggaagacgcgcgactggattgtggcactcaaggctttgatgctgatccatcgtttgcttaatgaaggggatcctttgtttcaggaagagatcttgttcgctaccagaagagggaccaggcttct

SEQ ID NO: 46 - DNA, 403 bp, Cannabis sp.
  source 1..403: mol_type = other DNA
  variation 202: allele = n = c/g
atctcaaatgaattggattgacaatgatgggttatttctcttgtccaacaagtctcaattcgctttcggtttcaccaccactaaccaagatgtcacaaaatttctgctagtaatcgtccacatgggaagccaacgagttatttggacagcgaatatagacaccccagttgccaattccgataagtttgtgttcgatgaaaanggtagagttttcttgcaaaaaggagcaagtgtggtttggtccattgatactggtggcaaaggagcttctgctatggagttgatggattcaggtaatttggttctgtttggagaggacgagaatagtaaaatatgggagagttttgaccatccaaccgatacccttttatggggacaggaatttgttgaagggatgagactt

SEQ ID NO: 47 - DNA, 403 bp, Cannabis sp.
  source 1..403: mol_type = other DNA
  variation 202: allele = n = t/c
agtggatcatctactgaacagaaggttctgttatccatagtaaaatttgttatgagagcactgaatttttcttcgtttctttcattttgttgatgtggcggtgacaaacatttatttaatgggtactctgtcgtatctgatgctgttgacattttttttatgctttcatcagctatctggaaagtctgaggtagctatgccngaggaccttacgtgagtagtgatttgtcctttccaattgttcttatcttgagcttattgatattgataatttgttctttttctctttactataatacaatctttttcttcaaatctttttatagttagtggtctagctatataatctttctttattccccttccctattatggtttggtggcaaatttgacagaatcgatt

SEQ ID NO: 48 - DNA, 403 bp, Cannabis sp.
  source 1..403: mol_type = other DNA
  variation 202: allele = n = c/t
aattaaaaatggcatctttcgcctcagaaactctcactttcatcttcttcatctctctactccatttgggttcatcggggaggattctttccgacgagtctgaccaaacccaacagcctcttccctttcaataccataatggccctcttctgtttggaaaaatctccattaacttaatatggtacggaaacttcaaaccaanccaacgagccattgtctccgacttcattacctccctcacttcatcttctaaaaccaacacagaccaaccatcagtcaacacgtggtggaagaccatcgaaagctaccaataccaccacaagctgagcaactccgtctcgttaggatcccagttcatcgacgagaactactccctggggaaatcgttgaccagtcaacaaat

SEQ ID NO: 49 - DNA, 403 bp, Cannabis sp.
  source 1..403: mol_type = other DNA
  variation 202: allele = n = t/c
gagtattttcaagtttaagatctctatgacatatttgctgccagaagtcatttattaaataattagacaatgacaaagttgataatcaatgcagttctttagagatatgtatgtatatctatcacaaaaggatagttcaaacaaaattcttaccattgaatgacagtaactgactcctgatattagttgttgaaagaaaaancttgcctgattcaatagaacaaaaagatgcttaagtgcttacattcattaagatttcatttggtgtaaaaacataagtttagttagaaatttcctccattacctcatcttcactaaatctaccagcagtgcatattctttcaaagagctctccaccaccagcatactccatcacaatggccagatgagttggagttaaaag

SEQ ID NO: 50 - DNA, 403 bp, Cannabis sp.
  source 1..403: mol_type = other DNA
  variation 202: allele = n = g/c
cttttttattatctgagatttagtaagtgcccatatgtttgtagatcgtgggatttagtcgattagggtactagtttggaggtttttgttttatcaattagttgcttcaagaaactcacttttctttttcgtttttatgctagtagaaatggacttgaactaggatctccaagcaggcgttgtaggaatcactgagagtgtncatgtcgctcttgttgaggaagctccaacggaaggtagccaggctatggttctcgttgtaatgcctcagcaaggggcaactctagaaattgtcgtagaggatgaacctactacagaggaaggaccctccaaaaaggacaatggaaaaaagagggccaagacacccccagccgcacaggcttacaatgactccatttgggag

SEQ ID NO: 51 - DNA, 403 bp, Cannabis sp.
  source 1..403: mol_type = other DNA
  variation 202: allele = n = c/g
gtggttttacataccccagcacgcatctgattgagcccaccattggtatgaactagaaggtaacctcgagacccggcaggggctgatacaacatcaaagataattaatacaattcacatatctgaatagaaaaataataccattatgttccttacgtgtataattaagacttgggtttgcacatggcacgaaatctcgattngatggaggtttccatagcttttccatttccaaaattccactcgcttcatccaactgtcacgtctaaaggagtgaaaatgggaaaaaaatgcatgatttgttcgcattattacaaaagaagggtagttataaatacgaatacccttctcttctttttaatcaaaattttcccttcttattatggaagtctctcctactaaag

SEQ ID NO: 52 - DNA, 403 bp, Cannabis sp.
  source 1..403: mol_type = other DNA
  variation 2: allele = n = c/g
agctcacaacaaagtgaagaatgcggggaccaccttggattccacgtcgacaatattcgacaatgcctactacaaactacttctgcaaggcaagagtattttctcttcagaccaatctctactcaccaccccaccaactaaggccttagtctccaaattcgctgcttctaaacaagacttcgataaagccttcgttgagtcnatgatcaagatgagtagcatccatggtagtggccaagaaattaggctcaactgcaaggtcgttaattaattaattaattagttttttttctttcaatatattattataaatttctccatcaatcgatcgtttaataattgaagaagaggagtgataagatgaaccaaaccaattaagatgcatgtaatgattagtaagttt

SEQ ID NO: 53 - DNA, 403 bp, Cannabis sp.
  source 1..403: mol_type = other DNA
  variation 202: allele = n = t/c
gaaccttgtctgaaaaatagttcacatgtaattagagataggaaaacaaagttccagcagtgaataaagaggaaaattacaaactcaaattgatctcttgaataccataccttctgaccaccaacaggcttcacacaatgcacaccgaagaatttcgtcacaagggaattttcataccgacagacatgttgatgataactanaaagcatccgtaatagcacctgaaataatattacaattaagttattttacctgacccttcaaaaccgcaaattatgctactcaaatggcttttgagacagcataagctccataataaagcaaaagtcaaagaagtaggcaacaaaccttgacttctgatttcttcaccgtctttatcataaacctatcatcttgtgtcaag

SEQ ID NO: 54 - DNA, 403 bp, Cannabis sp.
  source 1..403: mol_type = other DNA
  variation 202: allele = n = t/g
ctataaatatactttcccaaaagggactattcgctttaatctaattgtaatagtacaaatatgtttgtttcactttttttctttattgtagaaaaaagtaccccttttatccgttgaagcattgcaccatttgcatatctttatcttcgtcctagccattgtccatgtcactttctgtgttctcactgttgtgtttggaggngtaaaggtaagtatatcttccaaattcatatgcttctctctcttttatactctactactttgtttttgttcgtttatgagaaatatttctacacaactagatacgtcaatggaaacgttgggaggattctattgcaaatgagagttatgacactgaacaaggtaagtattctcagtaccacttttgacaatattatagaaa

SEQ ID NO: 55 - DNA, 403 bp, Cannabis sp.
  source 1..403: mol_type = other DNA
  variation 202: allele = n = c/a
aatgtcaatctctatcggaacgccgccgtttgatatcttagccattgctgacacaggcagcgatctgacgtggactcagtgcagcccttgcaaaaaatgttacaagcaagtggctcctctcttcaaacccaactcttcaaaaacatacagagatgctacctgtgattcctctgtttgtaagtccgccaccggagctaaaacntcttgctcctccctcgacgattcatgccaatactccgtatcttacggcgaccaatctttctccaacggtaacattgctactgacgttctcaccctctcttccacctctggaagacccgtcaccttccccaatttcatcatcggttgcagccacaatagtgatggtatatatataatattaattatggcgtgataaccttat

SEQ ID NO: 56 - DNA, 403 bp, Cannabis sp.
  source 1..403: mol_type = other DNA
  variation 202: allele = n = g/t
cccttggttgaaccgctgagcaagctctacagttgcacttttgtcaccatcagtaggccagccaatctcaccgatgattatggacaagtttccaaatccatttttctgcagagcccataccagtgtgtcatggtttgcatccaagacattctggtagatttttccattgtcgtttatggcagcggaatagccatcaaagaangcgaaatcaacagggaaattgggatcgttgtggaggctgataaaaggatagatgtttacagtaaaggaacctccattgtcgcttaagaacttaacgatggccaccatgagatcttttatgtctgttctgaagtcaccgtcggaaggtttctcactcgagctgccatatacatcagcgtttaaagggatagtgactttaact

SEQ ID NO: 57 - DNA, 403 bp, Cannabis sp.
  source 1..403: mol_type = other DNA
  variation 202: allele = n = t/c
agtaattttaaaagttctggtttgtttcaggaagaaaattggtgtaataaaagtaggcaatgagagttacagaagagaatcagagtcagatgagttaaatacaaaaaaagcaaatctttcagcttcaaggaaggaaagggttcagctaccaataatcccaaattatgagggtaagaaattccctattggtgaatttttaagncaaccttgtgcaattgaagccctcctcaataccaatgccttaaaaagtttccaatgtctttctcctaacacttacaggtaggctacctttcaacttcaattcaaccctttttctttgtaaaatctaacttagtatatcaaagtagtttaagctagaatataatagaactattttcaccattattagaagaagttgggattg

SEQ ID NO: 58 - DNA, 403 bp, Cannabis sp.
  source 1..403: mol_type = other DNA
  variation 202: allele = n = t/c
gtttgaatataaaagctagcagcaccttcaatgattattatgttctaaccagaaagaatagctacaaactctgaacttttcaagtacaatgtatgtgtatgtttgactaatcaatcttaccaaaatattttgtggcaatgtggaacgaattcctagttgactgctctttccccaagtgtcacggttgagttttcctttcatnaatgattcaggagtgtcatttcctaaaacctcctgagcattggcgtaagcagttgcggtatgctcacttacttgtttcatcaatccaatcgcatcatagctcctctctacagggctcacgtaataccccatatcctgtagaagaagtttccacagaattagcgaaaaaattatttcccatcaaagccagaaagtttaggtg

SEQ ID NO: 59 - DNA, 403 bp, Cannabis sp.
  source 1..403: mol_type = other DNA
  variation 202: allele = n = g/a
taagctttcttacaatgtgctgtccaatgaaagccgcaacagtagccacagcaacaaagtaagcagctgcaaataaatcacatcacgttttagtaaagtaccatattcggttctgctgtaaaggtgttcaacagaacaaatgaaaattagataatgatcaaagaagagtatatgtaaaggacactaaccatagggaactggnaaacgttttagaaggtaatattctacaacagacatagaagaggagaacgtcattgcaaatgtggctgttgcactcgagacctacacagaagtaaccaactaaattataactgattcaattgcataattatcaaataattaactctttaaggttgaatgctattttgcttttactatgctagggaaaaactcaatcccctta

SEQ ID NO: 60 - DNA, 403 bp, Cannabis sp.
  source 1..403: mol_type = other DNA
  variation 202: allele = n = a/c
attcaagaacttgggatgaagatattattcgagacctttttgaggatagagatcagaccctcattttttctattcagctaagtgaaaatgccttggaggatcattggtgctagaagtttgagaacgatggaggttacatggtcaacaatgcgtacagacatcttcaggtgctcaagggagcttggcctgctgcacaaccganaaatctttggcatactctctggagtcttaaagtgcccccaaaggtctgcaattttttgtggagggcagcgtcaggttgtttgcctacttgtgtacaattgcagaagagacatgtaccagtgagtataatctgccctgtttgcaatgtggatgatgagactattttccatgctcttgttgattgtcctgtcgcgagatcgtg

SEQ ID NO: 61 - DNA, 403 bp, Cannabis sp.
  source 1..403: mol_type = other DNA
  variation 202: allele = n = g/a
ccgcacaaaagaaaattatactcataaagttgatgaaaaagaaagggtccaaagcagtataattcatgttaattaagttgtgatattattgttttacattctcatccacacagcgaaagtgtactgataagcagaacttgttatgctccactctgactccagggatggattttgtttcttccaccaaaatagtgtacacctntatgcatgcacatctttataattatttatgtctatatatatataggagagaaaattaaattataaactaattaattaattataattacctcatcaatcattggtgaaaattctgccgcaggttgatacataactgcattcttaatctgagtatcaattaaaaatataaaataaacattaaaattttaatgttaaggtcaac

SEQ ID NO: 62 - DNA, 403 bp, Cannabis sp.
  source 1..403: mol_type = other DNA
  variation 202: allele = n = c/t
accaacaaagagtgcctcagcatcggggctaaaggatattccagcaatctcgccaaatatgtctatctcttgccctttagagtaccctgactgtgtatcgatgatgtgaacaaagtctgcaggttcagccatggccagaaaccggccatcgttagtgaacctaacagctcttattgcccccattcttcccttcaggacggcnaaggactctgacaggttccttatgtcccacaatctgcaggtggtgtcttggttcccggtagccaaaatacgtccatctggatgccaagcagaggcaaaagagtagtccaagtgccctttgaggctcccagtgatctgagaggaaggaaattagaatcaggaacagagaggaatggagaccaaacaaaatcaaggataaagt

SEQ ID NO: 63 - DNA, 403 bp, Cannabis sp.
  source 1..403: mol_type = other DNA
  variation 202: allele = n = c/t
gccaaaaactcgcaacaaagtcaagtttgtgtactcggatgacattaacacgaagaaaataatggaggatcactttgatatggatcaactggagtctgcatttggtggaaatgatactacaggtttcgatattaataaatatgcagagagaatgaaagaggatgacaaaaagatgcctgctttctggactaaaggaaatccnccagctccagcaacctcagaacctgtcccgaacaatgacactccatcttcggactctaccatcaagttagaagatgatgcagctgattccatcgagaagagaaatggatcggaaggggtgctcccagcccccaatcacactatgctcactgttgacagtagcagaaatcctactaaagaggttcagtaattaaggatcctt

SEQ ID NO: 64 - DNA, 403 bp, Cannabis sp.
  source 1..403: mol_type = other DNA
  variation 202: allele = n = t/c
agtttccgcctccagggtagagtaggcagtggtgaattctgatctcatagcccatctacccgtatcaccattaattaccaccgcagcaagaccagctgctccttccttccaagaggcatcagtcatcaagacaatcgaggcgttgctgactctccgacaaggattaaggacagtcgaaccaggaggatctgcagcagaggcngaaatacagaagtccttccattgagcagcaacctgttggaggagcaaagggaggaatactgaacttcctttgtataacaacttgttcctttcagcccaaactgcactaaaaagatagcccatgtagttgatgacagagcttcggtcaggagtgggaaagatggatactaaattctccacatactcctgcattgtctcacca

SEQ ID NO: 65 - DNA, 403 bp, Cannabis sp.
  source 1..403: mol_type = other DNA
  variation 202: allele = n = a/g
tccttgccctgtgggtaatgcctcaatcagatcaatctcccatattataaatgggtatggggaggtcatcattctcaactcggttggtggtatgcaaggtatatgtgaaaacttttggcatatcgcatttctcaacaaattcgtgtgtgtctttgttgattattggctagtaatatccttaacggataatcttcttccatanactctgccaacctacatgatccccacagaagtcttcatgaatttccttgattatttcatttgcttctactggtaacacacatcgtaagagaggcatggaataacgtcttctgtataacttcccctcgaccattatatagcgaggtattgagtataagagtttgtgtgcctcggttttgtctgtaggaagttctccactaac

SEQ ID NO: 66 - DNA, 403 bp, Cannabis sp.
  source 1..403: mol_type = other DNA
  variation 202: allele = n = c/g
attaggaagtctcccaagctgcagaacagcatgttagtgaaattgggaaattctctctccaatacttcggtactacccatcgtcgtctgagttctcagaacattcttccatcgcttcagcagtcataagagcagccacagccttgtgaattgctacatctgctctgtaaaccattctctgagctctttctctcttaagcttngcaatgttaatagcatgttcagcagcgcaagaagcatcacgcaacctgaactcatcaagatctggaccatcaaatttctcaatgatatgcctctgagatctctctaggtggaaatgctgtggactctgccactcagaagaaccaatattccactgatgatgctcatgtcttttttctaacattctacgatcatataaagcc

SEQ ID NO: 67 - DNA, 403 bp, Cannabis sp.
  source 1..403: mol_type = other DNA
  variation 202: allele = n = c/t
tttcccgtttcttaataactatgaatctgcctaaaatttgtctttgattgctcaccgagaatcatcacaaacccgacaagagaagtatagttgtgtaacatgtgaaaagtagaaatagaattgtttgtttagtatgtagaatcgcaaaaggatctctgagagcttacctatcattcacatggactcttctcgtgtcaagaanttctagtaaaggaagcaatggcatctagattttccttcatgaaccattcatgaggatagacagagttagctgattcaagatgaagatgccaaaccatgattctagagcaatcttgctccagtccttccatttggctctcatcttccacaatttgttcaataaatgtgcttacctgaaaatgcaaatcaactagattagtcc

SEQ ID NO: 68 - DNA, 403 bp, Cannabis sp.
  source 1..403: mol_type = other DNA
  variation 202: allele = n = a/t
tgacacaacctgcattgtgattgatatattacctcaagagaagccacctgctccgctgcccccacctaagaagcagggaaaaggagtgtttaagtccatgtttcgtaaaaagtcaactgaatcaacttcttatgttgacaaagagtacatagagccagacgtggtggaggaattatttgaagagggctctgctatgctttcngaaaggtttgtgcttgaaaaagtcactatcaacttaacatgattgccatgagaaagttctctctctctctctctctctctctctctctctctctctctctctctctcttttccttttctttttagtgtcacctttctttctttattcttgttctgttttcgttcttcctgtttaactttttgggggttgacctatgaacag

SEQ ID NO: 69 - DNA, 403 bp, Cannabis sp.
  source 1..403: mol_type = other DNA
  variation 202: allele = n = g/a
gtcgcgtctaagcaaaaactacccacctgatcttttctttcatgggtttcttcacgaaatcaacaaaaacccatctctccaatcttccattttattcatagcatgcgcgcaacgagcccacgcgccaccttgcctgccccttcaacacaaactcgtaagtaaagagttggcctggcgttagcgtcttccatggctattgtcnactcggagcggttgtttcttcttcttctcgtggttgtttcggatatgttggtgctgagtatgggggtttacgtgtccccgctgagctcagagaaatcttacgtgtcagccgttggagacccaggaatgaagagcccaaatgtcagagttggtttagaagcttggaacttctgcaatgaagttggagctgaagctcctggta

SEQ ID NO: 70 - DNA, 403 bp, Cannabis sp.
  source 1..403: mol_type = other DNA
  variation 202: allele = n = c/a
caataattttgacacaaaacacattttctttttgttgttgttttttaggcggagaatcttatgcttgagaggggagaaaacaaagaggtaaaatccaagtagtcgaaatgtttgttgacttgaattctatccctcttgatgatagttactaaatgtgatttgtttacgtatatagtaccttccaattgaaggtttggccgcnttcaataaggcaacggcagaattattgtttggagcagataacccagtaatcaaagaacaaagagtaagcttgaatttcgaatttgagtcaagaaaaattaattcttttcatggtaccttgattatttttgtcatattttctctgctttatatcaggttgcaaccgttcaaggactttcaggaacaggttctctccgtctcg

SEQ ID NO: 71 - DNA, 403 bp, Cannabis sp.
  source 1..403: mol_type = other DNA
  variation 202: allele = n = t/c
atgagataattaagcaatgagcatgcatgttaaatcaattttattaattatacatactgaatttgttgttatcatcgtaatataatagatattaattaatttaaaaataaaattataataataataataatatttacagggatatacacttttactggtgcaagcccatcgaagtgaactaaggcccccaaaatgcgtaganaatattaatcaccaatgcgtagaagcaaccacaggagaggcctcaatcctatacgtggggctctaccttgttgctctggggactggaggaatcaaagcagcactcccatcattgggtgctgaccagttcgatgagagggatccaaaggagtcgagtcaattgtcgagtttcttcaactggttcttgttcagcctcaccatt

SEQ ID NO: 72 - DNA, 403 bp, Cannabis sp.
  source 1..403: mol_type = other DNA
  variation 202: allele = n = a/g
aattaccctaaaaactatatgtttgagaaagaaaaaaataaacaataaggtgtgtgagtgtgacaagacttattcaaatgtgcaggctcttattctagggggcactgatacaacaacagtaacaatgacatgggcattggctctacttgtcaacaaccaagacacgttgagaaaagcacaagaagaattagaccaagtggtngggagagaaagacaagtaaagcaatcggacataaacaagctggtttatctccaagctgttatcaaagaaacaatgcgcttatacccagccgcaccactcgcactcccccaccaatcggttgaagactgtactgtgagtgggtaccacgttccagctggcacacgcctcctcctcaacctttcaaagctacaacgagatcca

SEQ ID NO: 73 - DNA, 403 bp, Cannabis sp.
  source 1..403: mol_type = other DNA
  variation 202: allele = n = c/t
tttattatatgtatcactgtaatgccatgagtttaatatgtaaatttttccaggcattatacatgcttaagaaatttaaacaaggagtccccttattttatcttttttcagtaggatttgtgctggttgccaaagtgagattggttttggacggtatctgaactgtttaaatgcattttggcatccagaatgcttccgttgncgtgcttgccatctacccatttctgattatgaggtatttttaaaccgttataatatgagctgtctaagcaatggtgcattggtataagacttctagtcatttaactggtggttattttcccttttgcagttttctacatctgggaactatccttaccataaaacctgttataaagagaactatcatccgaaatgtgatgtc

SEQ ID NO: 74 - DNA, 403 bp, Cannabis sp.
  source 1..403: mol_type = other DNA
  variation 202: allele = n = t/a
atatatatatagataagaagacagctaatggtttttttttcttcttcttcttcttctttcttcaccccgataagttgtttcgattggttaaaaagataaaggcaaaagtagtgataaaagtttgatgatatgggtaagaagaagaagcagaatcagaaaacaaaggaaatttcagtagccatagctgaagcagcttcatctntaaaaggagaacctcatcatcatcatcatcatcatcatcatcagtcgacgccgagaaagagaggtaggcctcggaaaattagcattgaaaaaagtctacagaaaaaagaagaagaaaaagaaatagtagttggtgatgatgatggtatcattattattagccagagtcaatccaagaaagctaaaatcgatgaacttgaag

SEQ ID NO: 75 - DNA, 403 bp, Cannabis sp.
  source 1..403: mol_type = other DNA
  variation 202: allele = n = a/c
tcctgaattgttgtccactgaaaccgtccggcaactgcatgctaccatcgaaaaagaatgggatgcccttcgaaggtcagcatgtcaaacggctgctggacgagcactgtggaaacatgtcactcatgaccctttggcagccttacttgctggagagacttacctaagaagccttcatgagaagataaagaaagactgtgcnaacaatgcaagtgaaatctctggagttattcttgcagttagaactctctggttcgattcaaaacttgaagcagcccttcattccttacatggcacagaaacacaagttgttctccttggtgcaggtaaatgataaacaccaaattttacatatattaacagatagcataattgtacttttacatatcttaattagtagtac

SEQ ID NO: 76 - DNA, 403 bp, Cannabis sp.
  source 1..403: mol_type = other DNA
  variation 202: allele = n = t/g
atatcataatgcaacattactaattattttgatgactaatttatagatacaagtaactgaaattgatttatttgattctcagaattctagctgtgtaccagtaaactatatggaaaatcaaccggatataaatgaaaatatgagaggggttctgattgattggcttgtagaggtacttaaattgctgctttcctgttgttantgtactcaatgaattcctctttaatggaattctctgaccatttagtgcaaattacaggttcactacaagtttgaattaatggatgagacgttgtatctcatggtcaacttgattgatagattcttagctgttcaatctgtgccaaggaggaaacttcagttggttggggttgctgccctacttctagcctgcaaatacgaa

SEQ ID NO: 77 - DNA, 403 bp, Cannabis sp.
  source 1..403: mol_type = other DNA
  variation 202: allele = n = a/t
tagacgaaaggaaagtagtaggtacaaatttatagaacaaaaaaaaagaacataatgatgcgaatgtatggttataatgttttcaaataactagaaaatacaatactcacttgataagccagtgaaactcttcctccttgtcagaacaaccataatctgaagtccacgcgtggcctgaattttttttttttttggacaaatnattatagcaaacaatcagctgcagtttgaacttatgcacacatttaattagttgtgttaaaaggtgtatatagactaacctatggtaaacttgtggaatcgcagcatgtccattacaccaacatgagctagtgcacagccaaaaagatcaggtctctgtaacaaaatgaaaaaatttgttttgtaatcaaccacgcataag

SEQ ID NO: 78 - DNA, 403 bp, Cannabis sp.
  source 1..403: mol_type = other DNA
  variation 202: allele = n = c/t
caatattgggtgtgatctatctttgcgattgggtcctatgagtgttgagaacaagcaaccccaggctattgcagacgttaatccccgagagggaggcaagttttgtgaccaattaccattattggataaagggtgctccatgtttcctcctagaggcaatgcttgccaggcattaagttcatacaaaggtgtagaagcagcngtgcgaaaacgaaagccatcttttgataatggaatcgacgatcagcagttttgtaatttgaagccaagtcctccttttctccacttaaatggaaaaatgagaaatgctggatcataagttctgattttttcttacttggttaactttgtggtgcaattttgttgtactcccggatgtgttatgcacccaagagctaccaga

SEQ ID NO: 79 - DNA, 403 bp, Cannabis sp.
  source 1..403: mol_type = other DNA
  variation 202: allele = n = a/g
gctacataaagacagaaacaccttttttttttttgaaaactacacaagtaaaaaaaaattgtagaaaaacaaaatccataaaaagaaaaaaaatatatatatttataagaaaaaatacaattttaagtagtgtaaaagatatttataagtaacctgggttttgaaattatgcaatcgcaattaaatgagatatggcagcagngcagattgattgactcaggtatgtcatcttctaaaatttcaataactactgatttcttttaaatgaaaccacacaatccaactcaaataaacaaataaaaaataacatataatgtaatggtcaactactcctacagctatctagaaaatgatacatcaaacatggctgcatctccagatcaagagtggtcatcctcccagc

SEQ ID NO: 80 - DNA, 403 bp, Cannabis sp.
  source 1..403: mol_type = other DNA
  variation 202: allele = n = c/a
cgaatcctcttcttcctcaacgtctcagagcttcacacaatggcgtttcccactaccaaactgtccagttcgtaatatcaatatacgatccgaatccgatttaacaccggaggaaagtgatcagactggtcaccattacggcccgccacctatttctcccacgaacctccaagaagtgttccacgcagctgagctccaactnagcgtcgggtcggattcggatcagttatctgccctacaacttctagagagatctctggttcccaacccgccgaccgatcctgaatgcccgccggagttgatgcgcggtgtagtggggagtttaatttctcaggttggggcgaaacctgcgtcgaagattttgctagctctctgcttggcggagcgaaaccggagagttgcc

SEQ ID NO: 81 - DNA, 403 bp, Cannabis sp.
  source 1..403: mol_type = other DNA
  variation 202: allele = n = g/a
tgatcttcagaagcaggtggccgagatggctggaaatgagaaacagattcttagtattgaagaagaaataagaagattacagaatttggttggtgatgtgctacaagatcctggactagaagatcaaatttcttctggcagtaacattgagtgcttagaaatgttgctgaggaagcttctagaaaattatgcaaaattttcnaccataagacctgtacttggtggtggaattgatgagctgcagactgacgtgatgactgtagaggcagctaagaacttaagcaaaacccatgctggggagtccgatgaagttatcatgaagaaagagcttgaggaagctttgcacgagttgattcttgtgaaggaggagagaggtgtatttgttgagaagcaacaatctttg

SEQ ID NO: 82 - DNA, 403 bp, Cannabis sp.
  source 1..403: mol_type = other DNA
  variation 202: allele = n = g/a
tgtgcaccaaaccaaggaaatggagttgaagaaagacaagtttgtcaggtgaactcctttacttcattatctttcttttacaatcatgcatgcttaactggggtttttcattttctccaattgtagattttaccatgaagaaaacaataaagatagtaacttagagaagccgttaccagtgttcaaagtagcagcagcagancctttgtttgaggcagaaggaggtagaagtagtagtagtagaaacagaatttatattccaaaatttggaaggtttaaagttttcccagaaaatgagaatccatggaggaagaagattcttgaccctggaagtgatattttcctgcaatggaacagagtttttctcttcttttgcttggtagcactttttgttgatccactc

SEQ ID NO: 83 - DNA, 403 bp, Cannabis sp.
  source 1..403: mol_type = other DNA
  variation 202: allele = n = a/t
aaagctcaagaatttccaatcctggcgtggaaactctggtatcctccctacaaaccggttactctcaacgtttaactccacaagcttagtcaacctcacaatggagttgggcaatctcccatcaaactcatttctgtccaagtaaactttcttcaagtaaaccattccttcaaaactatcgtccggtatgtcccctgaaaantcattgtacgacaagtacaaggctctcaaccctcgaagcactttgaattcaggtattccacccccaaagtggttgcctatcacgctgaggctgcgcagagtggttgggagcttggacaatgtgtagacatcgatggtgccacctagtcccatgttttcaagtcttaatccgtaaaggcttccattcacgcatgtcaaccct

SEQ ID NO: 84 - DNA, 403 bp, Cannabis sp.
  source 1..403: mol_type = other DNA
  variation 202: allele = n = a/t
atcaagtaactgcaagttgggaatagaccagagtgaaccaggaagagcaccatcgagtttattgtcactcagaaccacaacccgcagctgagaaaggcttgaaaacaagccctttggcaatggaccctctagaccattagcactaatatcaataaccctcaagctcctcaaacccgtgaattctttaggaaatgacccagtnaaaaaattcttgctacaatttagctcaacaagtcgagaaagtttattcaactgaacaggtatagaagcggtaagactattatcagaagtacttaaaaactcaagtctcgagaggttacctaaacctgatggaatcgacccagaaaagaaatttgaggagaggtcaagagtagtaagatttcgaagtgaggtaaactcgaac

SEQ ID NO: 85 - DNA, 403 bp, Cannabis sp.
  source 1..403: mol_type = other DNA
  variation 202: allele = n = g/a
tgaaagataaaactagcattaaaaaaataaaaattaaaaatattcttatgatatcgaactgcaggcagaatgtctgttggtgaagatcgatatccattgccgtattcaaggggatgttgttcttgagtgtatccacttagacgaagatcttgtacgtgaggagatgatgtttagagttatgttccatacagcatttgtgcgntcaaatattttgttgctaaatcgtgatgaaattgatgttttgtgggatgccaaagaccagtttcccagggactttaaggcagaggtaggtattactattcttctgtggtttctttttgtgaagctgttggagttgaatcatactcatatctgtatcaggtactttttttggatgctgatgccgttgtccctgatatcacca

SEQ ID NO: 86 - DNA, 403 bp, Cannabis sp.
  source 1..403: mol_type = other DNA
  variation 202: allele = n = t/c
ggtacgtgcaataaaatatttaaacttattttcagcacgataaattatttaaaattttataaaatttaacaataaattcaaacaaaatgataaataacagtgtataatattttgatacataattttagaacacactttatttgaaagctgaaaattaattattaatcagtacttaagatagttagagaaccaatccaacatnttggaatgggcctcatttgcagattttgatgctgcttcatcatccaagttgtacctcactgtccaaccatgttcaacctttgggataatctcaacatggctctcaacctataccattgccaaccattcattaatttatgcatacaattaattaatataattaaaacttattcatttatacaattcatatcaattattatca

SEQ ID NO: 87 - DNA, 403 bp, Cannabis sp.
  source 1..403: mol_type = other DNA
  variation 202: allele = n = g/t
aaaatcatcttctgcttcttcttcagctgttagaagacccaaattatcgatctctgatcaacaatctaaaattaacggtggtgatcgcactgttaaatcctctttggactcgatgaccgtcgatggctaccttcgcactgaagcctcagctatcactgctgagtctactcttttagatgcacaaattactctgatcgacccnactccgacaacctccatttctgctgctgcggtggcagtggctcctggagatttgaattccggcagcattggctcatctagtgctcctaagactgttgatgaagtgtggcgggagatcgtgtctggagataggaaggagtgtaaagaggaggagcaggatatggtgatgacgcttgaagactttttacttgccaaaactggg

SEQ ID NO: 88 - DNA, 403 bp, Cannabis sp.
  source 1..403: mol_type = other DNA
  variation 202: allele = n = t/g
gagcacactattgttatgttactattttatgacatttttaatgaatgtgataagtattattcaaatcaattgccatgaatagcttctgaatctccatgccgatgattttcatcgaggttaggattatttgtctgtatttgtggcggggttgcaggtagttgatccatctgagatgtttctacggaagcctgctgttggtttncctgagagcgtcgttgtcttctccattttgaaatctcgacaagtatagaattcccacacatagtaaccccgaatccggtaaatgtagctaacagaacagatagaactccttgcatatgaagctgtagattacatgatagcaacattttaaacagtctatactgaagatcttaattgaaaatagttttcttttgtaaaccaa

SEQ ID NO: 89 - DNA, 403 bp, Cannabis sp.
  source 1..403: mol_type = other DNA
  variation 202: allele = n = t/g
caaagtctagctgtaagatagaactacttctgaattttgattcaacttctgtagttctaactatatcttctaacactggcactgaacgttcagagtggtatagcaaacagagctgtgggagagacccgtatccacaattaacttttacaatataatgacagtagattcctctgtattctctttggggtactctagatttatntgaagaccatattttccttgtttctgaccttattataaatagaaatgaacattaccagcagcagaagtcattgtgtttacatattcactctccagcaagaattgaagaaagataatcggtacaacatctatttgtgtttgtatcaggtttttacataaactaatattgaactttgaaaatctactgacaggtcgagaacta

SEQ ID NO: 90 - DNA, 403 bp, Cannabis sp.
  source 1..403: mol_type = other DNA
  variation 202: allele = n = a/g
gttgtatcgaaacctattcgcccagctttggcaaatctcaagggaaacttttgtttgtagcaaccatttctgttagagaaagcagcttggcaattacactcattcaaacagctttgtttacaagtatccttgtcaaccagtgagaggaattcgtaagtgtccggttcccatctcaaaccgtctaattcagctatagaaaccnccttgtcacagccttctatggttgaatttctgttgcagcctaaagtcttctggtttttatcaatgtaatctaaaccaggaagacaaagacaaacaggttcttgatttataagctgacaatatgaattgatgccacataatccaattggagcacaaagattggtagttgaagaccactcaacaaaccagctgctgttctgaa

SEQ ID NO: 91 - DNA, 22 bp, synthetic construct
  source 1..22: mol_type = other DNA
tggctaaagtcatgctccatgt

SEQ ID NO: 92 - DNA, 23 bp, synthetic construct
  source 1..23: mol_type = other DNA
cacacctagtgggatgtatcagt

SEQ ID NO: 93 - DNA, 20 bp, synthetic construct
  source 1..20: mol_type = other DNA
ctgtcgaggcccatctcaaa

SEQ ID NO: 94 - DNA, 21 bp, synthetic construct
  source 1..21: mol_type = other DNA
agcgaggcaataccatcaaga

SEQ ID NO: 95 - DNA, 20 bp, synthetic construct
  source 1..20: mol_type = other DNA
ggatttgcttcacgacagcc

SEQ ID NO: 96 - DNA, 20 bp, synthetic construct
  source 1..20: mol_type = other DNA
cccaaaaccactttcgccag

SEQ ID NO: 97 - DNA, 20 bp, synthetic construct
  source 1..20: mol_type = other DNA
agcttgggtgatacagctgc

SEQ ID NO: 98 - DNA, 21 bp, synthetic construct
  source 1..21: mol_type = other DNA
acccgattgcttactaggtcc

SEQ ID NO: 99 - DNA, 20 bp, synthetic construct
  source 1..20: mol_type = other DNA
tgcttgattccgaggatcct

SEQ ID NO: 100 - DNA, 21 bp, synthetic construct
  source 1..21: mol_type = other DNA
gactttggcctatgcagagga

SEQ ID NO: 101 - DNA, 20 bp, synthetic construct
  source 1..20: mol_type = other DNA
acacttacccgctcttcagg

SEQ ID NO: 102 - DNA, 21 bp, synthetic construct
  source 1..21: mol_type = other DNA
agaggtggaattggaatgcca

SEQ ID NO: 103 - DNA, 20 bp, synthetic construct
  source 1..20: mol_type = other DNA
tattagagctgccttcgccg

SEQ ID NO: 104 - DNA, 20 bp, synthetic construct
  source 1..20: mol_type = other DNA
aaccaatccgtgaacactct

SEQ ID NO: 105 - DNA, 22 bp, synthetic construct
  source 1..22: mol_type = other DNA
agtcctctcttgatagccatct

SEQ ID NO: 106 - DNA, 20 bp, synthetic construct
  source 1..20: mol_type = other DNA
ccaaagcgggaatgtgacaa

SEQ ID NO: 107 - DNA, 22 bp, synthetic construct
  source 1..22: mol_type = other DNA
tggtgtggtttctttctgtcct

SEQ ID NO: 108 - DNA, 20 bp, synthetic construct
  source 1..20: mol_type = other DNA
tgatggtgatggtgaggctg

SEQ ID NO: 109 - DNA, 20 bp, synthetic construct
  source 1..20: mol_type = other DNA
agcttcagaggggtttgtgt

SEQ ID NO: 110 - DNA, 20 bp, synthetic construct
  source 1..20: mol_type = other DNA
aggagtgttacgacagcagc

SEQ ID NO: 111 - DNA, 20 bp, synthetic construct
  source 1..20: mol_type = other DNA
aggccatgatgaaacgacgt

SEQ ID NO: 112 - DNA, 20 bp, synthetic construct
  source 1..20: mol_type = other DNA
tattgggctgggcttcgtac

SEQ ID NO: 113 - DNA, 20 bp, synthetic construct
  source 1..20: mol_type = other DNA
ttgctggccaagtgagttct

SEQ ID NO: 114 - DNA, 20 bp, synthetic construct
  source 1..20: mol_type = other DNA
ccttgtcggggtggttcaat

SEQ ID NO: 115 - DNA, 20 bp, synthetic construct
  source 1..20: mol_type = other DNA
tccgtgcccttggtaaagag

SEQ ID NO: 116 - DNA, 20 bp, synthetic construct
  source 1..20: mol_type = other DNA
acggcagtagtagtgcatgc

SEQ ID NO: 117 - DNA, 20 bp, synthetic construct
  source 1..20: mol_type = other DNA
tcgaaccgtactttgccaca

SEQ ID NO: 118 - DNA, 20 bp, synthetic construct
  source 1..20: mol_type = other DNA
gaaaagtagcgtcacggtgg

SEQ ID NO: 119 - DNA, 20 bp, synthetic construct
  source 1..20: mol_type = other DNA
ggttttagcgtcgcgttgtg

SEQ ID NO: 120 - DNA, 20 bp, synthetic construct
  source 1..20: mol_type = other DNA
acacgcccataaagacaggg

SEQ ID NO: 121 - DNA, 20 bp, synthetic construct
  source 1..20: mol_type = other DNA
tgggtccggaagatacaagc

SEQ ID NO: 122 - DNA, 20 bp, synthetic construct
  source 1..20: mol_type = other DNA
atgaaggtggcgcaatggaa

SEQ ID NO: 123 - DNA, 20 bp, synthetic construct
  source 1..20: mol_type = other DNA
tcagtttctctgtctgccga

SEQ ID NO: 124 - DNA, 20 bp, synthetic construct
  source 1..20: mol_type = other DNA
ggagtatagggcggtgggta

SEQ ID NO: 125 - DNA, 20 bp, synthetic construct
  source 1..20: mol_type = other DNA
tcctccctccattccgatca

SEQ ID NO: 126 - DNA, 20 bp, synthetic construct
  source 1..20: mol_type = other DNA
actcacccaaaagtggccaa

SEQ ID NO: 127 - DNA, 20 bp, synthetic construct
  source 1..20: mol_type = other DNA
ccacaggcagcacgtttcta

SEQ ID NO: 128 - DNA, 20 bp, synthetic construct
  source 1..20: mol_type = other DNA
ctaaggccgttttgggacct

SEQ ID NO: 129 - DNA, 20 bp, synthetic construct
  source 1..20: mol_type = other DNA
gacctgacatcaccgttgct

SEQ ID NO: 130 - DNA, 20 bp, synthetic construct
  source 1..20: mol_type = other DNA
actcgacttctgcagagctt

SEQ ID NO: 131 - DNA, 20 bp, synthetic construct
  source 1..20: mol_type = other DNA
aggagatgtcaggaggtgga

SEQ ID NO: 132 - DNA, 20 bp, synthetic construct
  source 1..20: mol_type = other DNA
ttgtttggcaccagtggact

SEQ ID NO: 133 - DNA, 20 bp, synthetic construct
  source 1..20: mol_type = other DNA
taccctacttccttgggcga

SEQ ID NO: 134 - DNA, 20 bp, synthetic construct
  source 1..20: mol_type = other DNA
aggaaaatggcagacaccca

SEQ ID NO: 135 - DNA, 20 bp, synthetic construct
  source 1..20: mol_type = other DNA
tcttcattgggaggcagcag

SEQ ID NO: 136 - DNA, 20 bp, synthetic construct
  source 1..20: mol_type = other DNA
aggctccaccactgagtgta

SEQ ID NO: 137 - DNA, 20 bp, synthetic construct
  source 1..20: mol_type = other DNA
tctccgaataccctccacgt

SEQ ID NO: 138 - DNA, 20 bp, synthetic construct
  source 1..20: mol_type = other DNA
gcgtgtccgaaagtttgtgt

SEQ ID NO: 139 - DNA, 20 bp, synthetic construct
  source 1..20: mol_type = other DNA
tcttcccaactcctcctggt

SEQ ID NO: 140 - DNA, 20 bp, synthetic construct
  source 1..20: mol_type = other DNA
tgcaagttttgaagtggccg

SEQ ID NO: 141 - DNA, 20 bp, synthetic construct
  source 1..20: mol_type = other DNA
tctaaggtggttgcaggcag

SEQ ID NO: 142 - DNA, 21 bp, synthetic construct
  source 1..21: mol_type = other DNA
tggacgtctaagcaaaacaag

SEQ ID NO: 143 - DNA, 20 bp, synthetic construct
  source 1..20: mol_type = other DNA
caagtccaacccaggagctt

SEQ ID NO: 144 - DNA, 20 bp, synthetic construct
  source 1..20: mol_type = other DNA
ggctgagtcctgcaaagtct

SEQ ID NO: 145 - DNA, 20 bp, synthetic construct
  source 1..20: mol_type = other DNA
caccactgcctccacctatg

SEQ ID NO: 146 - DNA, 20 bp, synthetic construct
  source 1..20: mol_type = other DNA
caaatgggtgcttcggcttc

SEQ ID NO: 147 - DNA, 20 bp, synthetic construct
  source 1..20: mol_type = other DNA
ttttgcagcgatcgattccg

SEQ ID NO: 148 - DNA, 20 bp, synthetic construct
  source 1..20: mol_type = other DNA
ggcacgattctacaccacca

SEQ ID NO: 149 - DNA, 20 bp, synthetic construct
  source 1..20: mol_type = other DNA
acgcctggttgaagtgaaca

SEQ ID NO: 150 - DNA, 20 bp, synthetic construct
  source 1..20: mol_type = other DNA
acaaagtttgggcccacctt

SEQ ID NO: 151 - DNA, 22 bp, synthetic construct
  source 1..22: mol_type = other DNA
cccaatcccaaatccaaaacca

SEQ ID NO: 152 - DNA, 20 bp, synthetic construct
  source 1..20: mol_type = other DNA
ggtggcaatcgtggagagaa

SEQ ID NO: 153 - DNA, 24 bp, synthetic construct
  source 1..24: mol_type = other DNA
tggttaccctgtaattggttgaga

SEQ ID NO: 154 - DNA, 20 bp, synthetic construct
  source 1..20: mol_type = other DNA
cttttgcagccgatggttcg

SEQ ID NO: 155 - DNA, 22 bp, synthetic construct
  source 1..22: mol_type = other DNA
tgagggccacaacttagtagtg

SEQ ID NO: 156 - DNA, 21 bp, synthetic construct
  source 1..21: mol_type = other DNA
tggctctgatggagatgacac

SEQ ID NO: 157 - DNA, 20 bp, synthetic construct
  source 1..20: mol_type = other DNA
caggctttgtccagatgtgc

SEQ ID NO: 158 - DNA, 20 bp, synthetic construct
  source 1..20: mol_type = other DNA
acaaccagccaagagaccaa

SEQ ID NO: 159 - DNA, 20 bp, synthetic construct
  source 1..20: mol_type = other DNA
tggactgacctgagaccagt

SEQ ID NO: 160 - DNA, 21 bp, synthetic construct
  source 1..21: mol_type = other DNA
gcaacctggagagagataccg

SEQ ID NO: 161 - DNA, 20 bp, synthetic construct
  source 1..20: mol_type = other DNA
atccggtttcttgttcggct

SEQ ID NO: 162 - DNA, 20 bp, synthetic construct
  source 1..20: mol_type = other DNA
atggtccggagagttcatgt

SEQ ID NO: 163 - DNA, 20 bp, synthetic construct
  source 1..20: mol_type = other DNA
ggattcctccattgtgggca

SEQ ID NO: 164 - DNA, 20 bp, synthetic construct
  source 1..20: mol_type = other DNA
acttgtcccaaaaccaggca

SEQ ID NO: 165 - DNA, 20 bp, synthetic construct
  source 1..20: mol_type = other DNA
gcgctctggtctcttcaaga

SEQ ID NO: 166 - DNA, 20 bp, synthetic construct
  source 1..20: mol_type = other DNA
aggccaatctcgcttgtctc

SEQ ID NO: 167 - DNA, 20 bp, synthetic construct
  source 1..20: mol_type = other DNA
gcagaacaccatcaaagccg

SEQ ID NO: 168 - DNA, 20 bp, synthetic construct
  source 1..20: mol_type = other DNA
cgtctacgaagcggaggaaa

SEQ ID NO: 169 - DNA, 20 bp, synthetic construct
  source 1..20: mol_type = other DNA
ctctccgccatagccaagac

SEQ ID NO: 170 - DNA, 20 bp, synthetic construct
  source 1..20: mol_type = other DNA
gccttgggattcctcttcgt

SEQ ID NO: 171 - DNA, 20 bp, synthetic construct
  source 1..20: mol_type = other DNA
ctgcagggttgtggagagtg

SEQ ID NO: 172 - DNA, 21 bp, synthetic construct
  source 1..21: mol_type = other DNA
gtttggagggatttcagagct

SEQ ID NO: 173 - DNA, 21 bp, synthetic construct
  source 1..21: mol_type = other DNA
gttcacttttgccacactgca

SEQ ID NO: 174 - DNA, 20 bp, synthetic construct
  source 1..20: mol_type = other DNA
tgtgaagctggtaggacact

SEQ ID NO: 175 - DNA, 24 bp, synthetic construct
  source 1..24: mol_type = other DNA
gcagatgtacgctaaagatttgtt

SEQ ID NO: 176 - DNA, 20 bp, synthetic construct
  source 1..20: mol_type = other DNA
gcgtgggatgtaacggtcaa

SEQ ID NO: 177 - DNA, 22 bp, synthetic construct
  source 1..22: mol_type = other DNA
tggtctctctctctcttccaca

SEQ ID NO: 178 - DNA, 20 bp, synthetic construct
  source 1..20: mol_type = other DNA
tgatcgagtctccgaacacg

SEQ ID NO: 179 - DNA, 20 bp, synthetic construct
  source 1..20: mol_type = other DNA
cggggaaggagaggaagttg

SEQ ID NO: 180 - DNA, 20 bp, synthetic construct
  source 1..20: mol_type = other DNA
gcatcaaagccttgagtgcc

SEQ ID NO: 181 - DNA, 20 bp, synthetic construct
  source 1..20: mol_type = other DNA
cggtttcaccaccactaacc

SEQ ID NO: 182 - DNA, 20 bp, synthetic construct
  source 1..20: mol_type = other DNA
gggtatcggttggatggtca

SEQ ID NO: 183 - DNA, 20 bp, synthetic construct
  source 1..20: mol_type = other DNA
tgttgatgtggcggtgacaa

SEQ ID NO: 184 - DNA, 22 bp, synthetic construct
  source 1..22: mol_type = other DNA
tgccaccaaaccataataggga

SEQ ID NO: 185 - DNA, 20 bp, synthetic construct
  source 1..20: mol_type = other DNA
tccgacgagtctgaccaaac

SEQ ID NO: 186 - DNA, 20 bp, synthetic construct
  source 1..20: mol_type = other DNA
caacgatttccccagggagt

SEQ ID NO: 187 - DNA, 25 bp, synthetic construct
  source 1..25: mol_type = other DNA
tgacaaagttgataatcaatgcagt

SEQ ID NO: 188 - DNA, 20 bp, synthetic construct
  source 1..20: mol_type = other DNA
tatgctggtggtggagagct

SEQ ID NO: 189 - DNA, 20 bp, synthetic construct
  source 1..20: mol_type = other DNA
ctaggatctccaagcaggcg

SEQ ID NO: 190 - DNA, 20 bp, synthetic construct
  source 1..20: mol_type = other DNA
gtcattgtaagcctgtgcgg

SEQ ID NO: 191 - DNA, 20 bp, synthetic construct
  source 1..20: mol_type = other DNA
tttacataccccagcacgca

SEQ ID NO: 192 - DNA, 20 bp, synthetic construct
  source 1..20: mol_type = other DNA
cgtgacagttggatgaagcg

SEQ ID NO: 193 - DNA, 20 bp, synthetic construct
  source 1..20: mol_type = other DNA
accaccttggattccacgtc

SEQ ID NO: 194 - DNA, 20 bp, synthetic construct
  source 1..20: mol_type = other DNA
cgaccttgcagttgagccta

SEQ ID NO: 195 - DNA, 20 bp, synthetic construct
  source 1..20: mol_type = other DNA
cttctgaccaccaacaggct

SEQ ID NO: 196 - DNA, 22 bp, synthetic construct
  source 1..22: mol_type = other DNA
cagaagtcaaggtttgttgcct

SEQ ID NO: 197 - DNA, 20 bp, synthetic construct
  source 1..20: mol_type = other DNA
ccgttgaagcattgcaccat

SEQ ID NO: 198 - DNA, 20 bp, synthetic construct
  source 1..20: mol_type = other DNA
tcccaacgtttccattgacg

SEQ ID NO: 199 - DNA, 20 bp, synthetic construct
  source 1..20: mol_type = other DNA
atctgacgtggactcagtgc

SEQ ID NO: 200 - DNA, 20 bp, synthetic construct
  source 1..20: mol_type = other DNA
cactattgtggctgcaaccg

SEQ ID NO: 201 - DNA, 20 bp, synthetic construct
  source 1..20: mol_type = other DNA
ccagccaatctcaccgatga

SEQ ID NO: 202 - DNA, 20 bp, synthetic construct
  source 1..20: mol_type = other DNA
gtgagaaaccttccgacggt

SEQ ID NO: 203 - DNA, 23 bp, synthetic construct
  source 1..23: mol_type = other DNA
agttctggtttgtttcaggaaga

SEQ ID NO: 204 - DNA, 20 bp, synthetic construct
  source 1..20: mol_type = other DNA
tgaggagggcttcaattgca

SEQ ID NO: 205 - DNA, 21 bp, synthetic construct
  source 1..21: mol_type = other DNA
agctagcagcaccttcaatga

SEQ ID NO: 206 - DNA, 21 bp, synthetic construct
  source 1..21: mol_type = other DNA
aggagctatgatgcgattgga

SEQ ID NO: 207 - DNA, 20 bp, synthetic construct
  source 1..20: mol_type = other DNA
gtccaatgaaagccgcaaca

SEQ ID NO: 208 - DNA, 20 bp, synthetic construct
  source 1..20: mol_type = other DNA
taggtctcgagtgcaacagc

SEQ ID NO: 209 - DNA, 21 bp, synthetic construct
  source 1..21: mol_type = other DNA
ccttggaggatcattggtgct

SEQ ID NO: 210 - DNA, 20 bp, synthetic construct
  source 1..20: mol_type = other DNA
ccacattgcaaacagggcag

SEQ ID NO: 211 - DNA, 20 bp, synthetic construct
  source 1..20: mol_type = other DNA
aagaaagggtccaaagcagt

SEQ ID NO: 212 - DNA, 20 bp, synthetic construct
  source 1..20: mol_type = other DNA
atgtatcaacctgcggcaga

SEQ ID NO: 213 - DNA, 20 bp, synthetic construct
  source 1..20: mol_type = other DNA
ccaacaaagagtgcctcagc

SEQ ID NO: 214 - DNA, 20 bp, synthetic construct
  source 1..20: mol_type = other DNA
tctgcttggcatccagatgg

SEQ ID NO: 215 - DNA, 20 bp, synthetic construct
  source 1..20: mol_type = other DNA
tggagtctgcatttggtgga

SEQ ID NO: 216 - DNA, 20 bp, synthetic construct
  source 1..20: mol_type = other DNA
gcatagtgtgattgggggct

SEQ ID NO: 217 - DNA, 20 bp, synthetic construct
  source 1..20: mol_type = other DNA
cctccagggtagagtaggca

SEQ ID NO: 218 - DNA, 20 bp, synthetic construct
  source 1..20: mol_type = other DNA
tgcagtttgggctgaaagga

SEQ ID NO: 219 - DNA, 20 bp, synthetic construct
  source 1..20: mol_type = other DNA
aactcggttggtggtatgca

SEQ ID NO: 220 - DNA, 20 bp, synthetic construct
  source 1..20: mol_type = other DNA
accgaggcacacaaactctt

SEQ ID NO: 221 - DNA, 20 bp, synthetic construct
  source 1..20: mol_type = other DNA
cggtactacccatcgtcgtc

SEQ ID NO: 222 - DNA, 20 bp, synthetic construct
  source 1..20: mol_type = other DNA
tggcagagtccacagcattt

SEQ ID NO: 223 - DNA, 21 bp, synthetic construct
  source 1..21: mol_type = other DNA
tgctcaccgagaatcatcaca

SEQ ID NO: 224 - DNA, 21 bp, synthetic construct
  source 1..21: mol_type = other DNA
agagccaaatggaaggactgg

SEQ ID NO: 225 - DNA, 20 bp, synthetic construct
  source 1..20: mol_type = other DNA
atagagccagacgtggtgga

SEQ ID NO: 226 - DNA, 21 bp, synthetic construct
  source 1..21: mol_type = other DNA
ctgttcataggtcaaccccca

SEQ ID NO: 227 - DNA, 20 bp, synthetic construct
  source 1..20: mol_type = other DNA
attcatagcatgcgcgcaac

SEQ ID NO: 228 - DNA, 21 bp, synthetic construct
  source 1..21: mol_type = other DNA
tcagctccaacttcattgcag

SEQ ID NO: 229 - DNA, 20 bp, synthetic construct
  source 1..20: mol_type = other DNA
aggtttggccgccttcaata

SEQ ID NO: 230 - DNA, 20 bp, synthetic construct
  source 1..20: mol_type = other DNA
cgagacggagagaacctgtt

SEQ ID NO: 231 - DNA, 20 bp, synthetic construct
  source 1..20: mol_type = other DNA
tgcaagcccatcgaagtgaa

SEQ ID NO: 232 - DNA, 20 bp, synthetic construct
  source 1..20: mol_type = other DNA
ggtgaggctgaacaagaacc

SEQ ID NO: 233 - DNA, 21 bp, synthetic construct
  source 1..21: mol_type = other DNA
ggctcttattctagggggcac

SEQ ID NO: 234 - DNA, 20 bp, synthetic construct
  source 1..20: mol_type = other DNA
gaaaggttgaggaggaggcg

SEQ ID NO: 235 - DNA, 20 bp, synthetic construct
  source 1..20: mol_type = other DNA
gtgctggttgccaaagtgag

SEQ ID NO: 236 - DNA, 25 bp, synthetic construct
  source 1..25: mol_type = other DNA
acatcacatttcggatgatagttct

SEQ ID NO: 237 - DNA, 22 bp, synthetic construct
  source 1..22: mol_type = other DNA
tcttcttctttcttcaccccga

SEQ ID NO: 238 - DNA, 20 bp, synthetic construct
  source 1..20: mol_type = other DNA
gcctacctctctttctcggc

SEQ ID NO: 239 - DNA, 20 bp, synthetic construct
  source 1..20: mol_type = other DNA
ggcaactgcatgctaccatc

SEQ ID NO: 240 - DNA, 20 bp, synthetic construct
  source 1..20: mol_type = other DNA
acctgcaccaaggagaacaa

SEQ ID NO: 241 - DNA, 21 bp, synthetic construct
  source 1..21: mol_type = other DNA
aggggttctgattgattggct

SEQ ID NO: 242 - DNA, 20 bp, synthetic construct
  source 1..20: mol_type = other DNA
gcaggctagaagtagggcag

SEQ ID NO: 243 - DNA, 20 bp, synthetic construct
  source 1..20: mol_type = other DNA
cacgcgtggcctgaattttt

SEQ ID NO: 244 - DNA, 20 bp, synthetic construct
  source 1..20: mol_type = other DNA
tttggctgtgcactagctca

SEQ ID NO: 245 - DNA, 20 bp, synthetic construct
  source 1..20: mol_type = other DNA
gggaggcaagttttgtgacc

SEQ ID NO: 246 - DNA, 20 bp, synthetic construct
  source 1..20: mol_type = other DNA
tgcataacacatccgggagt

SEQ ID NO: 247 - DNA, 20 bp, synthetic construct
  source 1..20: mol_type = other DNA
atggcagcagagcagattga

SEQ ID NO: 248 - DNA, 20 bp, synthetic construct
  source 1..20: mol_type = other DNA
gctgggaggatgaccactct

SEQ ID NO: 249 - DNA, 20 bp, synthetic construct
  source 1..20: mol_type = other DNA
agagcttcacacaatggcgt

SEQ ID NO: 250 - DNA, 20 bp, synthetic construct
  source 1..20: mol_type = other DNA
taaactccccactacaccgc

SEQ ID NO: 251 - DNA, 21 bp, synthetic construct
  source 1..21: mol_type = other DNA
ccgagatggctggaaatgaga

SEQ ID NO: 252 - DNA, 20 bp, synthetic construct
  source 1..20: mol_type = other DNA
aacttcatcggactccccag

SEQ ID NO: 253 - DNA, 21 bp, synthetic construct
  source 1..21: mol_type = other DNA
tcatgcatgcttaactggggt

SEQ ID NO: 254 - DNA, 20 bp, synthetic construct
  source 1..20: mol_type = other DNA
actctgttccattgcaggaa

SEQ ID NO: 255 - DNA, 20 bp, synthetic construct
  source 1..20: mol_type = other DNA
atcctggcgtggaaactctg

SEQ ID NO: 256 - DNA, 20 bp, synthetic construct
  source 1..20: mol_type = other DNA
ccaagctcccaaccactctg

SEQ ID NO: 257 - DNA, 20 bp, synthetic construct
  source 1..20: mol_type = other DNA
tcactcagaaccacaacccg

SEQ ID NO: 258 - DNA, 20 bp, synthetic construct
  source 1..20: mol_type = other DNA
ctgggtcgattccatcaggt

SEQ ID NO: 259 - DNA, 20 bp, synthetic construct
  source 1..20: mol_type = other DNA
tgccgtattcaaggggatgt

SEQ ID NO: 260 - DNA, 20 bp, synthetic construct
  source 1..20: mol_type = other DNA
atcagggacaacggcatcag

SEQ ID NO: 261 - DNA, 20 bp, synthetic construct
  source 1..20: mol_type = other DNA
tggaatgggcctcatttgca

SEQ ID NO: 262 - DNA, 21 bp, synthetic construct
  source 1..21: mol_type = other DNA
aggttgagagccatgttgaga

SEQ ID NO: 263 - DNA, 20 bp, synthetic construct
  source 1..20: mol_type = other DNA
cggtggtgatcgcactgtta

SEQ ID NO: 264 - DNA, 20 bp, synthetic construct
  source 1..20: mol_type = other DNA
gcgtcatcaccatatcctgc

SEQ ID NO: 265 - DNA, 20 bp, synthetic construct
  source 1..20: mol_type = other DNA
gcttctgaatctccatgccg

SEQ ID NO: 266 - DNA, 21 bp, synthetic construct
  source 1..21: mol_type = other DNA
acagcttcatatgcaaggagt

SEQ ID NO: 267 - DNA, 20 bp, synthetic construct
  source 1..20: mol_type = other DNA
gcaaacagagctgtgggaga

SEQ ID NO: 268 - DNA, 21 bp, synthetic construct
  source 1..21: mol_type = other DNA
agttctcgacctgtcagtaga

SEQ ID NO: 269 - DNA, 20 bp, synthetic construct
  source 1..20: mol_type = other DNA
ttcgtaagtgtccggttccc

SEQ ID NO: 270 - DNA, 20 bp, synthetic construct
  source 1..20: mol_type = other DNA
gcagctggtttgttgagtgg

SEQ ID NO: 271 - DNA, 403 bp, Cannabis sp.
  source 1..403: mol_type = other DNA
  variation 202: allele = n = a/t
ttaatctgaacacactcatttcttcaggagcagattctcaggttaaccttttcaaactttccttgttcaatttcaacatcttaactagcatagttaacataatatacatgcatctcttaattatcttgaggatgtgacatattctagtattccattttctcagattatcgcatggagctctgataattgggaaagacaaaanagcacgttcttgcagactccagctggaaggacaggaatgtcagacacacaagtgcagtttcatcaggaccagacacacttcctcgtcgtacatgagactcagctatctatatacgaagcctcgaaactcgaacgcatattgcaggtagaacaatcaaacattgcttcaaaaacagtcatgaatctaaaaatttattacaat

SEQ ID NO: 272 - DNA, 403 bp, Cannabis sp.
  source 1..403: mol_type = other DNA
  variation 202: allele = n = a/t
ggctacttacggtgagaaagagtggtatttcttttcgccgcgtgatcggaaatacccgaacggttcgagaccgaatcgggcagctggaaccgggtactggaaggcaaccggagcggataaaccgatcgggaatccgaaaccggttgggattaagaaggctctggttttttacgccggaaaagctccgaaaggagagaaaacnaattggattatgcatgagtaccggcttgctgacgtggacagatcagctcgtaaaaaaaacagcttaagggtacattttctaattaaactccatgatattagcaggggtagtttcgtcatttcacagcatgtccaagctgtggtggtagtaaatgggtcccacgtgtgctatctgacacgtggcggcttatagcgctagatt

SEQ ID NO: 273 - DNA, 403 bp, Cannabis sp.
  source 1..403: mol_type = other DNA
  variation 202: allele = n = t/c
ggtagagttaagataattaatttagttatacagaactgcataccgacgtattttatagtgtatacctttcctcgtacgctttagaagttctcctcctctgcaaacaaagttcaatgctgtgaaagctttcgattcatggccttcgcctttttcgtcagccaatgcaaatgtagtacttccacagcccctttgaagccttgtncttagaatagcagctcctcttaaggctgtagtatacacaaagaaaactctccctcataattttattcaaagaagctattgtttaattagttttttacatgctaatctattgttatgagtaaaaataaataggattgtcctctttaaactatgactattgtagtaaataatattttgatgtggcactaacgtgacgttaatc

SEQ ID NO: 274 - DNA, 403 bp, Cannabis sp.
  source 1..403: mol_type = other DNA
  variation 202: allele = n = c/g
caaggagttatctcactagagctacatcactaacaaaattgatttcccactctcataaataaaactaaaagactgtaaccaacacatattattgtactcttgtttagttttggtgagaataaagagatgagaatgaactacatccttattgccacttgatctgcagaacttttcttcacaacacctctagtgcataactcanttaacaactccaaagcctcagttgcaaatccttcataagctagaccttcaatgagaatggtatatgtcacttcagtgggtttacatcctttggataccatatgatccagaaaatcgatggcacgactggtttgcctagtcttacaaagtcccaacatgatagagttgaaggtgattgcattaggctttattccaagtcctt

SEQ ID NO: 275 - DNA, 403 bp, Cannabis sp.
  source 1..403: mol_type = other DNA
  variation 202: allele = n = a/g
atggttcgtacattgttgctatatttattctctttttaacttaaattctttcttgtgaaaactttccgattccggtttgggtcgggtgagtcttgtcttgtgacatgtttgagagttagtatttgtgtttgtgcgtccgattacggttgtttatgatctaatggcgtctagtagtagaactatggaagatttgggacatcantgggctgatctacaagtggaagatgagaatgaagtggggcttttatttgatcaggaggaggtgttggttgatgagtttgatgggaggtggtgtttagtcgggaaattactatctgataggccggctgattttgactctctaaggaatgtaatggcgtctttgtggcgaccggggaaagggatgtacgtcaaagagttggag

SEQ ID NO: 276 - DNA, 403 bp, Cannabis sp.
  source 1..403: mol_type = other DNA
  variation 202: allele = n = g/a
tacaccagaagcaaacttcccaacaagatcatcttcagtcatatcaagaacctcagggctgaagactgagccattgtcataaacagattggacaacaagaccatatgaaaatggcctgatgccaagcttggcaagaagggcagcctcagaggaaccaaccttgtcacccttcttgatgagctcaactggggtgataatttcnacagtacccttgttaatcttagttgggatattaagcacctgttgagcccaacggtgttagtcataaattctaaggaaatacaatgaatgaaaaaatataggttaggtaagtgtgcagacctggaagaaagaagtctgagatggatcgagaccagtgttgccaggaggaacaaccacgtcaatcggggcaatcaaacccaca

SEQ ID NO: 277 - DNA, 403 bp, Cannabis sp.
  source 1..403: mol_type = other DNA
  variation 202: allele = n = t/a
tgtagtttccgagtggatgcatgtgcttttacctatagctatggaaagaggagtttgcataatcacaaacatgggtgcaagtaagattactaaataaaattttatgcaatcaacaaacttttttttttcttaaaagaaatagagtttagataaatgttcttattattgtttctttttctgggcagtggatccatgtggtgcncaggagaaagttgtagaaatagccagcagcctgggactgaacgtatctgtgggtgttgcttatgaggtttttgtcagcaaaacaggtatttgtagtcacttttaggaggatgaattggagatgatacgtttgagattttataaacactaaatgcttcacaacattcaattaaatcaaggattgttttatgatttaatcatg

SEQ ID NO: 278 - DNA, 403 bp, Cannabis sp.
  source 1..403: mol_type = other DNA
  variation 202: allele = n = t/c
gaactcttcttcaaatcatcatattgcttcttctatactactagtactactacttgtacttgtaatgagttttgggcaacaacaagctgaggcaagagctttctttgtgtttggagattctcttgtggacaatggcaataacaattatttagccaccacagcaagagctgactctcctccttatgggattgactaccctacncatagacccactggccgtttctctaatggcctcaacatgcctgaccttatcagtaaatctctcattaattttctctttaattatctaagtatacatatatatgtatgtatctctgatttgtgtatatatctattaacttgaataaataggtgagcaaattgggtcagagcccacattgccatacttgagtccggagctcac

SEQ ID NO: 279 - DNA, 403 bp, Cannabis sp.
  source 1..403: mol_type = other DNA
  variation 202: allele = n = g/t
tagtcaacttggatggccaagagtgtgtatttgtatggagttagaagattatgcattaagtgtattgaacttaaatagctgactggttttcaatctatataatcaacatagaactctcactatcatgcggggatctctgatcactgcttatgatttatactctcagatgttttaatccattattgacttaaatgggtttttngatataggtattcttgaagggcccatccttgtatgcctttaggggcctggctggccggtttgctcctattggagttcatatagcaatgctactaataatggcaggtggaactctaagtgcagctgggagcttcaaaggaacagtcacagtgccgcaggggctgaattttgtggtcggagatgtactaggcccaactgggtt

SEQ ID NO: 280 - DNA, 403 bp, Cannabis sp.
  source 1..403: mol_type = other DNA
  variation 202: allele = n = t/c
aaaaagcacaggaagagattttgagatccatgtttggaccaaaggaaaggagtactctctctctctctctctgaactgtctgtttgagctttggtgagctaaaccaacgaacgagcgagcttcaagctcaacaggccttctttgagtatgtatgtaatgtacccgtcttcaccttcttctcatgaaacgccttatgaaatgncatccaactctactaccactgtcattacccataggatatcaacctctttcccgctctctcacttcctcaacctccacatatcaacgtaaatggaaaccaaagaccactctcatcattaccaacccaactttattactcatggagtcttgctccaccatgctccagatgaagcaaattcaagctcaaatgactctcactggt

SEQ ID NO: 281 - DNA, 403 bp, Cannabis sp.
  source 1..403: mol_type = other DNA
  variation 202: allele = n = a/t
tcatctgtttgtgacattattctttgcttatttttgcttaagaaaacttcgttcgatttattaaattatatgaaagttcggtcctttatgtggaatgtatattttcctttcctgcagacaaatttgcaattgttagccagctgctacttgcggaacagtcaggcatactctgcgtaccatattttgaaaggtacatatgtcntaaaatatatatctttagcttactatggttacggtttactattttttcattttgcttgagtttgattatttttttcctttacactattatctgaaggaacacaattggctcaatcccgctacttgtttgcaatgtcttgctttcgaatggatcttcttaatgaagctgaaacagcattgtgtcctcctaatgaacccagtg

SEQ ID NO: 282 - DNA, 403 bp, Cannabis sp.
  source 1..403: mol_type = other DNA
  variation 202: allele = n = g/a
tcgatctgctaccctagtatgatgagtgtataaaagtacagtatagagattttcatacgtataaataatatcgtggtctgacagtacataatatatttttttatatatatacaaatacaattgagagtacataatatttaacgtatgtccaagttagtttgatcgagccctttcgcagcttcgaagcgcgtgacgcgtgttntgtcaagtcttgctagccgacaaaggtctgctttgtcagtttatgaggactttacttggcagttgccaccttcatctacggactcaataaatcttactttgcacgcgcttcaacgtggtgcgtgaaatcttttcttattttgccaagtggacttagtcaaagctgcctcaccttcgtttatttatttagagaaatttacat

SEQ ID NO: 283 - DNA, 403 bp, Cannabis sp.
  source 1..403: mol_type = other DNA
  variation 202: allele = n = c/g
aaatgaggttaaccccagcactaaacagtctaacttaaaaataagaactacctatcaccatcccctacaaacttacaagtttaattatgccactctagtataaaacgaaaaaggcaatcattgtcatagacaaaacacaaggcatttaaatttaataaagtagcaactgcagcaccaggacaatacaagtttcagtcaatanccaatttacacataaacctatttcaatatctgctgttgtctcaacagaagagcactaagcaccctcaatatttagaacaaacatatatacattatataattacatttatcatccacaaagtaccacaaccaaatagaccctattgaaagttgaaactgtttaccttagcagcaaagtgaaaattagttcagcaaaagagtt

SEQ ID NO: 284 - DNA, 403 bp, Cannabis sp.
  source 1..403: mol_type = other DNA
  variation 202: allele = n = g/a
acactaagggacaccaaaaataataaatttccccttcattattttcagatacaatggggatttagtccccccctcccacacacatatacccaatcacttgagggcatgcagtgccccgtcaatgataattgttcagaagactaatggaataataactgccattcttacccacatctgtataggttgattaaaatgaatttcngcaaggtactccatttctggatctgctcgcatccataacaagggcgagtccatactgtgaaaagaaaagtttgacacacgcatgcaaagacacaagaaaataataattagatcctcctatccaaataaactttaacagagcttaaagaaatgaacctacctggagcgcatatctagaggaagagtaccatctccattgtca

SEQ ID NO: 285 - DNA, 403 bp, Cannabis sp.
  source 1..403: mol_type = other DNA
  variation 202: allele = n = t/c
aaatcaaacagataaacctacaagcatactagtcaaactcctttcagaaacaaccaaccaccatttttgctttataaatatacaatgacccccaatgcacgaaaaaccacctcggtttaggaggaaagaatatcaacaaatagtcaaatagataaagattagaacgatattcatcagaaggtaaggaaacagagctgtgaangcaccgcaccttattctaaaaatcgtggaattgctgcttctatggattctcaatggtggcattcataatccgcagtgatcttgatctttcctgctgcccactgcgtctccaaaaagccctcctccaccaaacttcgaaccctctttgtatgttcgggcaatcttcagcagagttcaagaatcggttaaaccaagcaagcaa

SEQ ID NO: 286 - DNA, 101 bp, Cannabis sp.
  source 1..101: mol_type = other DNA
  variation 51: allele = n = a/g
atgaatcatgagagaatagttctgatgattttgaagatgttcatccagacntgcctaaagatgatcaggacatgcccgccgatggtgctgctagaaagaaa

SEQ ID NO: 287 - DNA, 20 bp, synthetic construct
  source 1..20: mol_type = other DNA
tcccaaaaccactttcgcca

SEQ ID NO: 288 - DNA, 20 bp, synthetic construct
  source 1..20: mol_type = other DNA
tcaggagcagattctcaggt

SEQ ID NO: 289 - DNA, 20 bp, synthetic construct
  source 1..20: mol_type = other DNA
cgaggaagtgtgtctggtcc

SEQ ID NO: 290 - DNA, 20 bp, synthetic construct
  source 1..20: mol_type = other DNA
acgacgaggaagtgtgtctg

SEQ ID NO: 291 - DNA, 20 bp, synthetic construct
  source 1..20: mol_type = other DNA
gtactggaaggcaaccggag

SEQ ID NO: 292 - DNA, 20 bp, synthetic construct
  source 1..20: mol_type = other DNA
tgtcagatagcacacgtggg

SEQ ID NO: 293 - DNA, 20 bp, synthetic construct
  source 1..20: mol_type = other DNA
tataagccgccacgtgtcag

SEQ ID NO: 294 - DNA, 20 bp, synthetic construct
  source 1..20: mol_type = other DNA
cgtaagtgtccggttcccat

SEQ ID NO: 295 - DNA, 20 bp, synthetic construct
  source 1..20: mol_type = other DNA
gccttcgcctttttcgtcag

SEQ ID NO: 296 - DNA, 21 bp, synthetic construct
  source 1..21: mol_type = other DNA
cacgttagtgccacatcaaaa

SEQ ID NO: 297 - DNA, 20 bp, synthetic construct
  source 1..20: mol_type = other DNA
tttcgattcatggccttcgc

SEQ ID NO: 298 - DNA, 21 bp, synthetic construct
  source 1..21: mol_type = other DNA
tgccacttgatctgcagaact

SEQ ID NO: 299 - DNA, 20 bp, synthetic construct
  source 1..20: mol_type = other DNA
aaaccagtcgtgccatcgat

SEQ ID NO: 300 - DNA, 20 bp, synthetic construct
  source 1..20: mol_type = other DNA
aaccagtcgtgccatcgatt

SEQ ID NO: 301 - DNA, 20 bp, synthetic construct
  source 1..20: mol_type = other DNA
tttgggtcgggtgagtcttg

SEQ ID NO: 302 - DNA, 20 bp, synthetic construct
  source 1..20: mol_type = other DNA
cgccacaaagacgccattac

SEQ ID NO: 303 - DNA, 20 bp, synthetic construct
  source 1..20: mol_type = other DNA
gtttgggtcgggtgagtctt

SEQ ID NO: 304 - DNA, 20 bp, synthetic construct
  source 1..20: mol_type = other DNA
tcgccacaaagacgccatta

SEQ ID NO: 305 - DNA, 20 bp, synthetic construct
  source 1..20: mol_type = other DNA
acctcagggctgaagactga

SEQ ID NO: 306 - DNA, 20 bp, synthetic construct
  source 1..20: mol_type = other DNA
tccaggtctgcacacttacc

SEQ ID NO: 307 - DNA, 20 bp, synthetic construct
  source 1..20: mol_type = other DNA
ccaggtctgcacacttacct

SEQ ID NO: 308 - DNA, 20 bp, synthetic construct
  source 1..20: mol_type = other DNA
ttccgagtggatgcatgtgc

SEQ ID NO: 309 - DNA, 20 bp, synthetic construct
  source 1..20: mol_type = other DNA
agcaacacccacagatacgt

SEQ ID NO: 310 - DNA, 20 bp, synthetic construct
  source 1..20: mol_type = other DNA
cgagtggatgcatgtgcttt

SEQ ID NO: 311 - DNA, 20 bp, synthetic construct
  source 1..20: mol_type = other DNA
agctgaggcaagagctttct

SEQ ID NO: 312 - DNA, 20 bp, synthetic construct
  source 1..20: mol_type = other DNA
atggcaatgtgggctctgac

SEQ ID NO: 313 - DNA, 20 bp, synthetic construct
  source 1..20: mol_type = other DNA
gcaacaacaagctgaggcaa

SEQ ID NO: 314 - DNA, 20 bp, synthetic construct
  source 1..20: mol_type = other DNA
ggctctgacccaatttgctc

SEQ ID NO: 315 - DNA, 20 bp, synthetic construct
  source 1..20: mol_type = other DNA
gcggggatctctgatcactg

SEQ ID NO: 316 - DNA, 20 bp, synthetic construct
  source 1..20: mol_type = other DNA
tgggcctagtacatctccga

SEQ ID NO: 317 - DNA, 20 bp, synthetic construct
  source 1..20: mol_type = other DNA
cggggatctctgatcactgc

SEQ ID NO: 318 - DNA, 20 bp, synthetic construct
  source 1..20: mol_type = other DNA
gctaaaccaacgaacgagcg

SEQ ID NO: 319 - DNA, 20 bp, synthetic construct
  source 1..20: mol_type = other DNA
tgcttcatctggagcatggt

SEQ ID NO: 320 - DNA, 20 bp, synthetic construct
  source 1..20: mol_type = other DNA
gagcatggtggagcaagact

SEQ ID NO: 321 - DNA, 20 bp, synthetic construct
  source 1..20: mol_type = other DNA
ttactggtgcaagcccatcg

SEQ ID NO: 322 - DNA, 20 bp, synthetic construct
  source 1..20: mol_type = other DNA
tgcaattgttagccagctgc

SEQ ID NO: 323 - DNA, 22 bp, synthetic construct
  source 1..22: mol_type = other DNA
cactgggttcattaggaggaca

SEQ ID NO: 324 - DNA, 22 bp, synthetic construct
  source 1..22: mol_type = other DNA
actgggttcattaggaggacac

SEQ ID NO: 325 - DNA, 20 bp, synthetic construct
  source 1..20: mol_type = other DNA
gtttgatcgagccctttcgc

SEQ ID NO: 326 - DNA, 20 bp, synthetic construct
  source 1..20: mol_type = other DNA
taaacgaaggtgaggcagct

SEQ ID NO: 327 - DNA, 20 bp, synthetic construct
  source 1..20: mol_type = other DNA
tttgatcgagccctttcgca

SEQ ID NO: 328 - DNA, 20 bp, synthetic construct
  source 1..20: mol_type = other DNA
tgccttggaggatcattggt

SEQ ID NO: 329 - DNA, 20 bp, synthetic construct
  source 1..20: mol_type = other DNA
atccacattgcaaacagggc

SEQ ID NO: 330 - DNA, 20 bp, synthetic construct
  source 1..20: mol_type = other DNA
atgaggttaaccccagcact

SEQ ID NO: 331 - DNA, 21 bp, synthetic construct
  source 1..21: mol_type = other DNA
gggtgcttagtgctcttctgt

SEQ ID NO: 332 - DNA, 21 bp, synthetic construct
  source 1..21: mol_type = other DNA
tgagggtgcttagtgctcttc

SEQ ID NO: 333 - DNA, 20 bp, synthetic construct
  source 1..20: mol_type = other DNA
ccaatcacttgagggcatgc

SEQ ID NO: 334 - DNA, 20 bp, synthetic construct
  source 1..20: mol_type = other DNA
gcgctccaggtaggttcatt

SEQ ID NO: 335 - DNA, 21 bp, synthetic construct
  source 1..21: mol_type = other DNA
ccctcccacacacatataccc

SEQ ID NO: 336 - DNA, 20 bp, synthetic construct
  source 1..20: mol_type = other DNA
aatgacccccaatgcacgaa

SEQ ID NO: 337 - DNA, 20 bp, synthetic construct
  source 1..20: mol_type = other DNA
ctctgctgaagattgcccga

SEQ ID NO: 338 - DNA, 20 bp, synthetic construct
  source 1..20: mol_type = other DNA
atgacccccaatgcacgaaa

SEQ ID NO: 339 - DNA, 22 bp, synthetic construct
  source 1..22: mol_type = other DNA
cctatggtcgaagacgatgaat

SEQ ID NO: 340 - DNA, 22 bp, synthetic construct
  source 1..22: mol_type = other DNA
atttggtcagcattagccattt

SEQ ID NO: 341 - DNA, 21 bp, synthetic construct
  source 1..21: mol_type = other DNA
gaagacgatgaatcatgagag

SEQ ID NO: 342 - DNA, 22 bp, synthetic construct
  source 1..22: mol_type = other DNA
tttatttatcgcgggtgaagat

SEQ ID NO: 343 - DNA, 6294 bp, Cannabis sp.
  source 1..6294: mol_type = other DNA
  variation 1: allele = n = a/g
ntgcctaaagatgatcaggacatgcccgccgatggtgctgctagaaagaaagataatcttgatgagtcagatcctgtggtcacaatggccaagaaattacaaatagctaaaaataaaggtgtcccctaccattttgtaccaagttaaaaattacataaattgagttagaaaacatagccttttccattaaattattctctcggatactattcttatactttccagaaagtcgttaacaaaaatggctaatgctgaccaaatattttcggtaatcttcacccgcgataaataaattaatgtttttccaagtgtgcaccaaacttagaaactcaaaattcccaattaattgtaatggattacatggcatatatttacagtttgtaatatatcgtacggattaccaatattggaagtcagagacatggcttttgttcttttactctaaacggaaaatattgtgtgtatgtgtgtgtgagcttgatgcatgtgatgttacccttttcaacagtgtaaaactctttccttcttcttcttcttcaaactcaacaaattaataaaatgaaaagttaaagtactaactcacgtgtagcaaagttttaacacatattgctcagcatattcaaaataatatatatatatatagaaagccgaaaaattatttaatgtgatgaatatatgttgcacgaaaacgtgaactattttaatatgtactcaatattcactgtacgtacgtgagccatgcatgtgtgattgctctcattaattttgatgaaaaaaaaaaggaaactgatgatatagcacccgtagatgttagaaaataatagtactattaggtaaaactaagaatgactatgacttcacatcagtgacttaagacaaaaaacaaattgatgttgatagctatagcatatactttttttttttttttttttttttatgggagtttaacctataccatagatcttacatcagtgaaattaaggcttttacactgtaggaactcattgacactgttttattgtttaaaatcaaatattatatagttttctaagatgagaaatttagttgtaattataatctttttagggggaaaaaattagcgagtggattttaatttattgaaagaatttttatctttttcagtaataaatcaaaaaatttgatgatgtatacatttaatatgtattattttatttttaaaagaaagaattcttatgtgatattggcatatataggtgggccatgcatagcagcccactcatagttttaatggtcaggttacaaatgaagaaggttaaaacttaagtattagtataatttaatgtcaagttagtcagtagtttgtttatccttaattaatttattgtgtgaatttcgaatctatgatgattgatgaatgttaaaactcaaagtattagtataatttaatgtcaagtttcacctattttatttaatttgtaacagaacatgtaatcaagacttgttactcaattttaccaattaagatgtgatgcaattgagtactgttagagtatagtacaagtgtaagaatattgaacaatttataagagtgtgactaactctactcatcactatagtaggttagatatagcctcataattcttgtgataatattgtattcaatcactaaccaaattgtgtgcacgaaaaaaaatagtagaaagttgcacgagatctctcatagtgcctagatcaaaacaaaataagcgagcaagtgtctcacatgtaaccgctataaatatatatatataaaacaagcaataatatagttccatttattagagtatgagatagaatctcataattattgtcaagcaccataagtgacttattgcagtgctctatttaggattgtatactgctatatataagtaatgtatactacacattacttaatattaatatggttttacacaataataatatttatttaattttagtaaataaggaatataattataaaccaatttataaattgcgtttcaaatggattattcatgtggtatatatactttcataatcacattataattttttaaaaaattaatacaattattatatgcgtgcgtgtataaataataataataatctgaacctctatacgtacttctaattaattgtaattttcttataaagccactttattatttcccatttgtgtcgttatcttatttctttgaactattttaatattaattaaaaaacttatatatttatatatttagttctgttctacctagctaatatatatatacgtagatctctcacaaaccatcctcactactacatatactactactctttctctcttctattcttctacatttctctcacatttatttatatatatacatcgatcttttatttatatatatacatcgatctatttgaaaagaaaaaaatgagtagtttattatctaagaaagcagcatgcaatgctcatggtcaagattcttcctacttcttaggatgggaagaatacgagagaaattcttatgatagaattaccaacccggaagggattatccaaatgggtcttgcagaaaaccaggttaattattatatatatagattatagaaaaatgtattatatatgaattaaaattttcttacaattctttgtttttggacagctttgttaccaccttctagagtcatggcttgctaataatccacatgcacttgggtttaaaagtcaaggtcaatacattttcagaaaacttgctctgttccaagattatcatggactccccgaattcaagaaggtaattatcattagatattctataaaaattaatatattaaattataggaagaatactaaacattattatttttcttttccaaattcaatgtgcatatatataataaaatttggttacagcaggcaatggttgaattcatgtccgaaataagtggaaacaaagtgagatttgagccgcaaagtctagtgctcatcgcaggtgcaacctcagccaacgaggccctcatattttgcctagctgatcccaacgatgccttccttctcccaactccatactaccctgggtacgtaacctaattaattaactttagttacccttattattaatttcattaattattctatcactacttttcactttaattagctaaacgtacgtacgttagtcgtagtaaagtcactaataatgccacagctagctttcactgggtcccacttccttcgtgcatacgtgattcgggttctacatcaatcatcataggatattataatatcttgtttcttattcattttagtatagataaaaattgtttagcctatttttttttaaaagatggtataccttatatgacttagttatttttaaaattatttataaattttttaaaaaaaattaatttttttaaattttaaatattctgaataatttatagtattgtaaattatttaaaattttctaaaaatttacaaataatcttaaataattacgggattatacaaacaaaaattaaactaaaaatctattctaaatgttaaaacagataaagaatttattaaaacactattagtggttttaccaaaaatcttctcaatattttaatatttaaaaacaaatagatatgataaagaagaattgcatatatgtaatgtacttagtttttgcagatttgatagggatcttaagtggcgaactgaagtggtaatagtgcctattcattgcaaaagctcaaatggctttcaaatcacggaagagggattggaacaagcctatgaagatgccacaaaccgtaatctaagagttaaaggtgttttgattaccaacccatctaatccatgtggcaccacaatgacagtggatgagttgaatctcctccttaacttcattgaacttaagaaaatccatctcatcagtgatgaaatctattcaggaacagttttcaacaaaccagactttaaaagtgtcatagaagttctcaacgagagaaacaataacaacaataatagtactaatcaaatcatgattcgggaacaagttcatgtcgtttatagcctttccaaagatctaggactccctggatttcgtgttggagcaatatattcaaatcacaagatggttttagatgcagccacaaaaatgtcgagtttcggtttggtatcttctcaaactcaattcttactctcggttatgttatcagacaaatattttacgaaaacatacataaaagaaaaccaaaagagacttaagagaaggcataaaatgcttgttgggggtcttaagaaagcaggaataagttgtcttaagagtaatgctggattattttgttgggttgatatgagacaccttcttaagtcgaatactttcaatgctgaaattgagctatggaaaaagatcatttacgatgtgaaactcaatatatcacctggttcatcgtgtcattgtaacgaaccaggttggtttagagtttgttttgcaaacatgtccgggagaacattgaagctcgcaataaaaaggttgaaagactttgttgctgatcatgatgagtttaattaccacactaaatatcaccaacctactacgtaataggcttttccacctatcatttcacatccaccaatgctatcagctccccatattgtaagtacagtttatatatatatatatattctcttctcttatatatacgtataaatgaattattacacatgcatatacatcaataaactagtatcatcatcaatccaatcgctcggtagtttttttcaaaaaaaagagaaaaaaacgttaatatgatcattttggaccggtattaattaaaatgcatggtacaacatgttatactgaactcaattttcgataatttttttcttttaatacatatataatcaacttcaaaacaatttctaacacgaacaaatataaaaattgtaacaagtcttgttgtaacacttttatattgggttatttttaaagtttattttaactaaaaatcaattctaggcattactttgtacaaatctagaaaatacaatgtcaaatttattatttaataaactatttaattagtaatttttataaaatacagaatttaaaatagtatttattagattgttaatattattaagaagttatttgtgactaaaaatattattaacatagaaatctgtaattaaatgtgtagttgaggcaaaacttaaaactaaattagtaatgaattgtatctaaatactagcattttaaaaataatatttagttaaaaaattatattataattaaaaatttgttactaaacaaaaactgtttttgcagtgtgattgaatcgtctttgcctctgaattaatggtttttacctagctagcgaaattccccaattacaactaactgttaaaattatgttatatctgtttattttttaacgatttgatggatcagaactgtctaaaaagtaggcagaaagcaaaccaacatatatatgttttttaatggttgttataattgttattaggaacaatttttgtcagctaaaaaaaataatttacggtataaatatctaagtttaattttacgattgtaaataaatatttaaatttaatttttaatcgtaataatacttaatttatatttttaaaatttttgtaagtacctagtctttaactgttaaattattattcaaatgttatttttttattagtatatttaaagtaagttttaaataaaaaatacatatatttaatgacttagaccaattagagatttaacttgacatttaaaggtaaggtatttagaaaaattttaaaattataacttaattgctctaaaaaattaaacttaactattcatgtataaattactctaaaaaaaactttacataagctataagagaaaacaaatcattgctttgtcaaaaacaaaaattaaaaggcactaatagttctaacactacttaaaatgttaaatcactattaataatattaaatgttaagtcatatataatttaatataatatttttttttttttgaaatatggctataaatattaatttattaatcttccttggttctaccatgcataagtactgtatctaattaaagaaaataattattttgtgcccacgtgttgtatgcatatataaattatggaagtttttttttttttttgaaaaaagaaaaattatgtcttggcgcaggtattagtttatgacatctgtaagagagagaggctgagaagtcgaggttaagttgctgtctctgatattttctagggtttgtaatattatatccttaattcata

(19) United States
(12) Patent Application Publication
(10) Pub. No.: US 2025/0277277 A1
(43) Pub. Date: Sep. 4, 2025

(54) QUANTITATIVE TRAIT LOCI ASSOCIATED WITH HERMAPHRODITISM IN CANNABIS

(71) Applicant: Puregene AG, Zeiningen (CH)

(72) Inventors: Claudio CROPANO, Zeiningen (CH); Dániel Árpád CARRERA, Zeiningen (CH); Eduard BRUDERER, Zeiningen (CH); Gavin Mager GEORGE, Zeiningen (CH); Lee-Gavin WILLIAMS, Zeiningen (CH); Leron KATSIR, Zeiningen (CH); Maximilian Moritz VOGT, Zeiningen (CH); Mercedes THIEME, Zeiningen (CH); Michael Eduard RUCKLE, Zeiningen (CH); Michele WYLER, Zeiningen (CH)

(21) Appl. No.: 18/859,505

(22) PCT Filed: Apr. 25, 2023
(86) PCT No.: PCT/IB2023/054220

§ 371 Date: Oct. 23, 2024

(30) Foreign Application Priority Data
Apr. 25, 2022 (GB) 2206005.7

(51) Int. Cl. C12Q 1/6895; A01H 1/00; A01H 1/04; A01H 5/12; A01H 6/28

(52) U.S. Cl. CPC C12Q 1/6895; A01H 1/045; A01H 1/12; A01H 5/12; A01H 6/28; C12Q 2600/13; C12Q 2600/156

(57) ABSTRACT

The invention relates to methods of characterizing a Cannabis spp. plant comprising quantitative trait loci (QTLs) associated with a hermaphroditism trait, and to methods of producing plants having a hermaphroditism trait of interest based on defined allelic states of polymorphisms defining the QTLs or the allelic state of a causal polymorphism provided herein. Also provided are Cannabis spp. plants with the hermaphroditism trait of interest comprising defined allelic states of polymorphisms and plants identified, characterized or produced by the methods described. Further provided are methods of marker assisted selection, genomic selection, marker assisted breeding, and genetic modification, for obtaining plants having a hermaphroditism trait of interest.

### BACKGROUND OF THE INVENTION

The invention relates to methods of characterizing a Cannabis spp. plant comprising quantitative trait loci (QTLs) associated with a hermaphroditism trait, and to methods of producing plants having a hermaphroditism trait of interest based on defined allelic states of polymorphisms defining the QTLs or the allelic state of a causal polymorphism provided herein. Also provided are Cannabis spp. plants with the hermaphroditism trait of interest comprising defined allelic states of polymorphisms and plants identified, characterized or produced by the methods described. The invention further provides for identification of a gene involved in the determination of the hermaphroditism trait. Further provided are methods of marker assisted selection, genomic selection, marker assisted breeding, and genetic modification, for obtaining plants having a hermaphroditism trait of interest.

Modern Cannabis is derived from the cross hybridization of three biotypes; Cannabis sativa L. ssp. indica, Cannabis sativa L. ssp. sativa, and Cannabis sativa L. ssp. ruderalis. Cannabis was divergently bred into two distinct, albeit tentative types, called Hemp and HRT (high-resin-type) Cannabis, respectively, which are used for different purposes. Hemp is primarily used for industrial purposes, for example in feed, food, seed, fiber, and oil production. Conversely, HRT cannabis is largely cultivated and bred for high concentrations of the pharmacological constituents, cannabinoids, derived from resin in the trichomes. Biomass, including the leaf and stem, of cannabis can also be an important source of cannabinoids.

Cannabis is the only species in the plant kingdom to produce phytocannabinoids. Phytocannabinoids are a class of terpenoid acting as antagonists and agonists of mammalian endocannabinoid receptors. The pharmacological action is derived from this ability of phytocannabinoids to disrupt and mimic endocannabinoids. Due to its psychoactive properties, one cannabinoid, delta-9-tetrahydrocannabinol (THC), the decarboxylation product of the plant-produced delta-9-tetrahydrocannabinolic acid (THCA), has received much attention in illegal or unregulated breeding programs, with modern HRT varieties having THC concentrations of 0.5% to 30%.

Cannabis is a dioecious plant, having male and female flowers produced on separate plants. In HRT cannabis production, only female plants are cultivated because unpollinated female flowers are valued for their high cannabinoid content. Male plants are a source of unwanted pollen and are excluded from production environments. Because there are no good methods for selecting female cannabis seedlings based on morphological indicators of gender, molecular markers would be useful for distinguishing male and female plants at the seedling stage.

In Cannabis sex determination is guided largely by sex chromosomes, where male plants have X and Y chromosomes, while female plants have two X chromosomes. Gender in cannabis is labile and can be influenced by a number of factors, including epigenetics, hormonal changes, and environmental stresses, such as changes in daylength. As such, Cannabis plants can produce male flowers in the absence of a Y chromosome. Hermaphroditic inflorescence, either staminate inflorescences produced on female plants or the formation of anthers in pistillate flowers, can be triggered by environmental stresses such as prolonged periods of darkness during the flowering stage, inadequate nutrition, and low temperature. Sex determination is distinct from hermaphroditism as both processes are regulated by differing genetic mechanism, and where hermaphroditism can be influenced by environmental factors.

The emergence of hermaphroditic inflorescence can be a challenge to growers in female cannabis production systems because pollen from male flowers can result in production losses caused by the presence of seeds in flowers and because the hermaphroditic plants are of low or no value in these production systems. In cannabis production, hermaphroditic flowers are common, can be difficult to detect and their emergence can cause significant losses in HRT cannabis production. Additionally, the propensity for hermaphroditism may not be detected for several generations of selection as the genetic and environmental conditions may mask it, leading to an additional complication for breeders of cannabis.

Hermaphroditism in Cannabis can be induced by different chemical applications including silver nitrate (AgNO₃), silver thiosulfate solution (STS), and gibberellic acid (GA₃). There is indication that STS and GA₃ act as ethylene antagonists to promote the production of male flowers on female plants. This is particularly useful in producing all female progeny “feminized seeds” that are generated when male flowers are induced in a female plant in order to produce pollen to fertilize female flowers. Commercially, “feminized” seeds are more expensive to produce than normal dioecious seeds due to demand for all-female seeds and because they require technical expertise to produce.

The changes in phytohormone level that would accompany these stresses may underly the induction of hermaphroditic flower formation through signalling pathways connected to ethylene. However, there is nothing known about the precise signalling pathways or genetic control of hermaphroditism in cannabis. The identification of molecular markers to identify and select for and against cannabis plants that may be predisposed to producing hermaphroditic flowers would be a significant contribution to the cannabis industry in eradicating such flowers in female production systems. In addition, hermaphroditism in hemp may be advantageous in production systems for seed and fibre. Controlling the emergence of hermaphrodite flowers may also be important to developing novel breeding tools.

The present invention relates to markers and putative genes for the control of hermaphroditism in cannabis, with the aim of removing plants with an increased propensity for hermaphroditism from breeding populations.

### SUMMARY OF THE INVENTION

The present invention relates to methods of characterizing a Cannabis spp. plant comprising one or more quantitative trait loci (QTLs) associated with a hermaphroditism trait, and to methods of producing plants having a hermaphroditism trait of interest based on defined allelic states of polymorphisms defining the QTLs or the allelic state of a causal polymorphism provided herein. Also provided are Cannabis spp. plants with the hermaphroditism trait of interest comprising QTLs defined by specific allelic states of polymorphisms as well as plants identified, characterized or produced by the methods described. Also provided are quantitative trait loci that control a hermaphroditic trait in Cannabis spp., wherein the quantitative trait loci are defined by single nucleotide polymorphisms defined herein, including a causal polymorphism for the trait, or genetic markers linked to the QTLs, as well as putative genes that control a hermaphroditism trait in a Cannabis spp. plant. Further provided are methods of marker assisted selection, genomic selection, marker assisted breeding, and genetic modification, for obtaining plants having a hermaphroditism trait of interest.

According to a first aspect of the present invention there is provided for a method for characterizing a Cannabis spp. plant with respect to a hermaphroditism trait, the method comprising the steps of: (i) genotyping at least one plant with respect to a hermaphroditism QTL by detecting (a) one or more polymorphisms associated with the hermaphroditism trait as defined in any one of Tables 1, 3 to 11 and 15; and/or (b) a polymorphism causal for the hermaphroditism trait which is an A/G SNP at position 99811627 on chromosome NC_044370.1 with reference to the cs10 reference genome; and (ii) characterizing the plant as having a hermaphroditism presence QTL or a hermaphroditism absence QTL based on the genotype at the polymorphism. In particular, the polymorphism may be selected from the group consisting of SNP markers “common_491”, “common_512”, “common_517”, “common_518”, “common_525”, “rare_57”, “rare_50”, “common_534”, “common_511”, “GBScompat_common_56”, “common_54”, “common_294”, and combinations thereof, as defined in any one of Tables 3 to 11 and 15. Further, the polymorphism may be the A/G SNP at position 99811627 on chromosome NC_044370.1 with reference to the cs10 reference genome (position 1 of SEQ ID NO:343), wherein the G allele at this position is causal for the hermaphroditism presence trait. In some embodiments, plants comprising a GG genotype at this position have an increased propensity for hermaphroditism, plants having an AA genotype at this position have a decreased propensity for hermaphroditism, and plants that are heterozygous (AG) at this position have an intermediate propensity for hermaphroditism.

In a first embodiment of the method for characterizing a Cannabis spp. plant with respect to a hermaphroditism trait, the genotyping may be performed by PCR-based detection, including using molecular markers, sequencing of PCR products containing the one or more polymorphisms, targeted resequencing, whole genome sequencing, or restriction-based methods, for detecting the one or more polymorphisms. While many suitable genotyping methods are known to those of skill in the art, in one embodiment, the genotyping may be performed using sequencing primers or similar molecular markers, wherein the molecular markers may be selected from the primer pairs as defined in Table 12 or 17 herein, which have been developed by the inventors of the present invention for detecting the polymorphisms provided in Tables 1, 3 to 11 and 15 herein.

According to a second embodiment of the method for characterizing a Cannabis spp. plant with respect to a hermaphroditism trait, the molecular markers may be designed for detecting polymorphisms at regular intervals within the hermaphroditism QTL such that recombination can be excluded.

In a third embodiment of the method for characterizing a Cannabis spp. plant with respect to a hermaphroditism trait, the molecular markers may be designed for detecting polymorphisms at regular intervals within the hermaphroditism QTL such that recombination can be quantified to estimate linkage disequilibrium between a particular polymorphism and a hermaphroditic phenotype, or the absence thereof. For example, molecular markers may be for detecting polymorphisms such that recombination events can be detected to a resolution of 10′000 or 100′000 or 500′000 base pairs within the QTL.

According to a further embodiment of the method for characterizing a Cannabis spp. plant with respect to a hermaphroditism trait, the hermaphroditism QTL may be a quantitative trait locus having a sequence that corresponds to nucleotides 28544332 to 35677966 of NC_044370.1 with reference to the CS10 genome and defined by one or more polymorphisms associated with hermaphroditism as defined in any one of Tables 1, 3 to 11 and 15, or a genetic marker linked to the QTL; a quantitative trait locus having a sequence that corresponds to nucleotides 94129798 to 101726389 of NC_044370.1 with reference to the CS10 genome and defined by one or more polymorphisms associated with hermaphroditism as defined in any one of Tables 1, 3 to 11 and 15, or a genetic marker linked to the QTL; a quantitative trait locus defined by a sequence comprising a single nucleotide polymorphism at position 61687722 of NC_044372.1 with reference to the CS10 genome, or a genetic marker linked to the QTL; a quantitative trait locus defined by a sequence comprising a single nucleotide polymorphism at position 889040 of NC_044370.1 with reference to the CS10 genome, or a genetic marker linked to the QTL; and/or a quantitative trait locus having a sequence that corresponds to nucleotides 70255045 to 79818534 of NC_044370.1 with reference to the CS10 genome and defined by one or more polymorphisms associated with hermaphroditism as defined in any one of Tables 1, 3 to 11 and 15, or a genetic marker linked to the QTL.

According to a second aspect of the present invention there is provided for a method of producing a Cannabis spp. plant having a hermaphroditism trait of interest, the method comprising the steps of: (i) providing a donor parent plant having in its genome a hermaphroditism QTL characterized by (a) one or more polymorphisms associated with the hermaphroditism trait of interest as defined in any one of Tables 1, 3 to 11 and 15; and/or (b) a polymorphism causal for the hermaphroditism trait of interest which is an A/G SNP at position 99811627 on chromosome NC_044370.1 with reference to the cs10 reference genome; (ii) crossing the donor parent plant having the hermaphroditism QTL with at least one recipient parent plant to obtain a progeny population of cannabis plants; (iii) screening the progeny population of cannabis plants for the presence of the hermaphroditism QTL; and (iv) selecting one or more progeny plants having the hermaphroditism QTL, wherein the mature plant displays the hermaphroditism trait of interest.

In a first embodiment of the method of producing a Cannabis spp. plant having a hermaphroditism trait of interest, the method may further comprise: (v) crossing the one or more progeny plants with the donor recipient plant; or (vi) selfing the one or more progeny plants.

According to a second embodiment of the method of producing a Cannabis spp. plant having a hermaphroditism trait of interest, the step of screening may comprise genotyping at least one plant from the progeny population with respect to the hermaphroditism QTL by detecting one or more polymorphisms associated with the hermaphroditism trait of interest as defined in any one of Tables 1, 3 to 11 and 15 and/or for the polymorphism causal for the hermaphroditism trait of interest. In some embodiments, the progeny population of cannabis plants contains a minimum of 100, or 500, or 1000, or 10000 plants.

In a third embodiment of the method of producing a Cannabis spp. plant having a hermaphroditism trait of interest, the method may further comprise a step of genotyping the donor parent plant with respect to the hermaphroditism QTL by detecting one or more polymorphisms associated with the hermaphroditism trait of interest as defined in any one of Tables 1, 3 to 11 and 15 and/or the polymorphism causal for the hermaphroditism trait of interest, prior to step (i).

In another embodiment of the method of producing a Cannabis spp. plant having a hermaphroditism trait of interest, the genotyping may be performed by PCR-based detection using molecular markers, sequencing of PCR products containing the one or more polymorphisms, targeted resequencing, whole genome sequencing, or restriction-based methods, for detecting the one or more polymorphisms.

In some embodiments, the molecular markers may be for detecting polymorphisms at regular intervals within the hermaphroditism QTL such that recombination can be excluded or such that recombination can be quantified to estimate linkage disequilibrium between a particular polymorphism and the hermaphroditic trait of interest. For example, molecular markers may be for detecting polymorphisms such that recombination events can be detected to a resolution of 10′000 or 100′000 or 500′000 base pairs within the QTL. In some embodiments, genome sequencing, or marker-based PCR and resequencing of the QTL may be used for detecting a plurality of polymorphisms defined in any one of Tables 1, 3 to 11 and 15. In some embodiments, the molecular markers may be selected from the primer pairs provided in Table 12 or 17. In particular, the plant may be genotyped for a polymorphism selected from the group consisting of SNP markers “common_491”, “common_512”, “common_517”, “common_518”, “common_525”, “rare_57”, “rare_50”, “common_534”, “common_511”, “GBScompat_common_56”, “common_54”, “common_294”, and combinations thereof, as defined in any one of Tables 3 to 11 and 15. Further, the polymorphism may be the A/G SNP at position 99811627 on chromosome NC_044370.1 with reference to the cs10 reference genome (position 1 of SEQ ID NO:343), wherein the G allele at this position is causal for the hermaphroditism presence trait. In some embodiments, plants comprising a GG genotype at this position have an increased propensity for hermaphroditism, plants having an AA genotype at this position have a decreased propensity for hermaphroditism, and plants that are heterozygous (AG) at this position have an intermediate propensity for hermaphroditism.

In one embodiment, the hermaphroditism QTL is a hermaphroditism absence QTL, and the hermaphroditism trait of interest is a hermaphroditism absence trait.

According to a further embodiment of the method of producing a Cannabis spp. plant having a hermaphroditism trait of interest, the hermaphroditism QTL may be a quantitative trait locus having a sequence that corresponds to nucleotides 28544332 to 35677966 of NC_044370.1 with reference to the CS10 genome and defined by one or more polymorphisms associated with hermaphroditism as defined in any one of Tables 1, 3 to 11 and 15, or a genetic marker linked to the QTL; a quantitative trait locus having a sequence that corresponds to nucleotides 94129798 to 101726389 of NC_044370.1 with reference to the CS10 genome and defined by one or more polymorphisms associated with hermaphroditism as defined in any one of Tables 1, 3 to 11 and 15, or a genetic marker linked to the QTL; a quantitative trait locus defined by a sequence comprising a single nucleotide polymorphism at position 61687722 of NC_044372.1 with reference to the CS10 genome, or a genetic marker linked to the QTL; a quantitative trait locus defined by a sequence comprising a single nucleotide polymorphism at position 889040 of NC_044370.1 with reference to the CS10 genome, or a genetic marker linked to the QTL; and/or a quantitative trait locus having a sequence that corresponds to nucleotides 70255045 to 79818534 of NC_044370.1 with reference to the CS10 genome and defined by one or more polymorphisms associated with hermaphroditism as defined in any one of Tables 1, 3 to 11 and 15, or a genetic marker linked to the QTL.

In a third aspect of the invention there is provided for a method of producing a Cannabis spp. plant comprising a hermaphroditism trait of interest, the method comprising introducing into a Cannabis spp. plant a hermaphroditism QTL: (a) characterized by one or more polymorphisms associated with the hermaphroditism trait of interest as defined in any one of Tables 1, 3 to 11 and 15, wherein said hermaphroditism QTL is associated with the hermaphroditism trait of interest in the plant; and/or (b) comprising a polymorphism causal for the hermaphroditism trait of interest which is an A/G SNP at position 99811627 on chromosome NC_044370.1 with reference to the cs10 reference genome (position 1 of SEQ ID NO:343).

In one embodiment of the method of producing a Cannabis spp. plant comprising a hermaphroditism trait of interest, introducing the hermaphroditism QTL comprises crossing a donor parent plant having the hermaphroditism QTL with a recipient parent plant.

In an alternative embodiment of the method of producing a Cannabis spp. plant comprising a hermaphroditism trait of interest, introducing the hermaphroditism QTL may comprise genetically modifying the Cannabis spp. plant. Several methods of genetic modification are known to those of skill in the art, including targeted mutagenesis, genome editing, and gene transfer. In particular the methods of genetically modifying a plant may be selected from the group consisting of CRISPR-Cas9 targeted gene editing, heterologous gene expression using various expression cassettes; TILLING, and non-targeted chemical mutagenesis using e.g., EMS. Alternatively, a cannabis spp. plant may be transformed with the hermaphroditism QTL or a part thereof, via any of the transformation methods known in the art. In one embodiment, the genetic modification may be by targeted mutagenesis and/or gene editing of guanine to adenine (G>A) at position 99811627 on chromosome NC_044370.1 with reference to the cs10 reference genome (position 1 of SEQ ID NO:343).

In a further embodiment of the method of producing a Cannabis spp. plant comprising a hermaphroditism trait of interest, introducing the hermaphroditism QTL may comprise introducing a hermaphroditism QTL into the Cannabis spp. plant, wherein the hermaphroditism QTL is a quantitative trait locus having a sequence that corresponds to nucleotides 28544332 to 35677966 of NC_044370.1 with reference to the CS10 genome and defined by one or more polymorphisms associated with hermaphroditism as defined in any one of Tables 1, 3 to 11 and 15, or a genetic marker linked to the QTL; a quantitative trait locus having a sequence that corresponds to nucleotides 94129798 to 101726389 of NC_044370.1 with reference to the CS10 genome and defined by one or more polymorphisms associated with hermaphroditism as defined in any one of Tables 1, 3 to 11 and 15, or a genetic marker linked to the QTL; a quantitative trait locus defined by a sequence comprising a single nucleotide polymorphism at position 61687722 of NC_044372.1 with reference to the CS10 genome, or a genetic marker linked to the QTL; a quantitative trait locus defined by a sequence comprising a single nucleotide polymorphism at position 889040 of NC_044370.1 with reference to the CS10 genome, or a genetic marker linked to the QTL; and/or a quantitative trait locus having a sequence that corresponds to nucleotides 70255045 to 79818534 of NC_044370.1 with reference to the CS10 genome and defined by one or more polymorphisms associated with hermaphroditism as defined in any one of Tables 1, 3 to 11 and 15, or a genetic marker linked to the QTL.

In one embodiment of the method of producing a Cannabis spp. plant comprising a hermaphroditism trait of interest, introducing the hermaphroditism QTL may comprise introducing a hermaphroditism absence QTL into the plant, and the hermaphroditism trait of interest may be a hermaphroditism absence trait.

According to a further aspect of the present invention there is provided for a Cannabis spp. plant characterized according to any method of characterizing a Cannabis spp. plant described herein or produced according to any method of producing a Cannabis spp. plant described herein. In some embodiments, the Cannabis spp. plant thus characterized or produced is not exclusively obtained by means of an essentially biological process.

In another aspect of the invention there is provided for a Cannabis spp. plant comprising a hermaphroditism QTL: (a) characterized by one or more polymorphisms associated with the hermaphroditism trait of interest as defined in any one of Tables 1, 3 to 11 and 15, wherein said hermaphroditism QTL is associated with the hermaphroditism trait of interest in the plant; and/or (b) comprising a polymorphism causal for the hermaphroditism trait of interest which is an A/G SNP at position 99811627 on chromosome NC_044370.1 with reference to the cs10 reference genome. In some embodiments, the Cannabis spp. plant comprising a hermaphroditism QTL is not exclusively obtained by means of an essentially biological process.

In one embodiment, the Cannabis spp. plant comprising a hermaphroditism QTL comprises a hermaphroditism absence QTL and displays a hermaphroditism absence trait.

According to further aspect of the present invention there is provided for a quantitative trait locus that controls a hermaphroditic trait in Cannabis spp., wherein the quantitative trait locus is selected from one or more of: a quantitative trait locus having a sequence that corresponds to nucleotides 28544332 to 35677966 of NC_044370.1 with reference to the CS10 genome and defined by one or more polymorphisms associated with hermaphroditism as defined in any one of Tables 1, 3 to 11 and 15, or a genetic marker linked to the QTL; a quantitative trait locus having a sequence that corresponds to nucleotides 94129798 to 101726389 of NC_044370.1 with reference to the CS10 genome and defined by one or more polymorphisms associated with hermaphroditism as defined in any one of Tables 1, 3 to 11 and 15, or a genetic marker linked to the QTL; a quantitative trait locus defined by a sequence comprising a single nucleotide polymorphism at position 61687722 of NC_044372.1 with reference to the CS10 genome, or a genetic marker linked to the QTL; a quantitative trait locus defined by a sequence comprising a single nucleotide polymorphism at position 889040 of NC_044370.1 with reference to the CS10 genome, or a genetic marker linked to the QTL; and/or a quantitative trait locus having a sequence that corresponds to nucleotides 70255045 to 79818534 of NC_044370.1 with reference to the CS10 genome and defined by one or more polymorphisms associated with hermaphroditism as defined in any one of Tables 1, 3 to 11 and 15, or a genetic marker linked to the QTL. The invention further includes a genomic region defined by markers linked to the QTLs defined herein.

In yet a further aspect of the present invention there is provided for an isolated gene that controls a hermaphroditic trait in a Cannabis spp. plant, wherein the gene has the gene identity number LOC115696400 with reference to the CS10 genome and encodes a putative 1-aminocyclopropane-1-carboxylate synthase.

In one embodiment, the isolated gene comprises an A/G SNP at position 99811627 on chromosome NC_044370.1 with reference to the cs10 reference genome (position 1 of SEQ ID NO: 343). In one embodiment, the G allele at this position is causal for the hermaphroditism presence trait. In some embodiments, plants comprising a GG genotype at this position have an increased propensity for hermaphroditism, plants having an AA genotype at this position have a decreased propensity for hermaphroditism, and plants that are heterozygous (AG) at this position have an intermediate propensity for hermaphroditism.

In some embodiments, the gene may be selected from the group consisting of the genes as defined in Table 13 with reference to the CS10 genome.

In one embodiment, the isolated gene has the gene identity number LOC115715793 and encodes a DELLA protein RGL2, as defined in Table 13; or the isolated gene has the gene identity number LOC115702418 and encodes a nodulation-signaling pathway 2 protein, as defined in Table 13; or the isolated gene has the gene identity number LOC115719981 and encodes a FT-interacting protein 7, as defined in Table 13; or the isolated gene has the gene identity number LOC115698538 and encodes a Clavata 1 Receptor like kinase, as defined in Table 13; or the isolated gene has the gene identity number LOC115695629 and encodes a Myb/SANT-like DNA-binding domain, as defined in Table 13; or the isolated gene has the gene identity number LOC115695629 and encodes a Myb/SANT-like DNA-binding domain, as defined in Table 13; or the isolated gene has the gene identity number LOC115700622 and encodes an Ovate-Transcriptional repressor, as defined in Table 13.

### BRIEF DESCRIPTION OF THE FIGURES

Non-limiting embodiments of the invention will now be described by way of example only and with reference to the following figures:

FIG. 1: GWA of hermaphroditic flowering in Cannabis in a mixed F2 population.

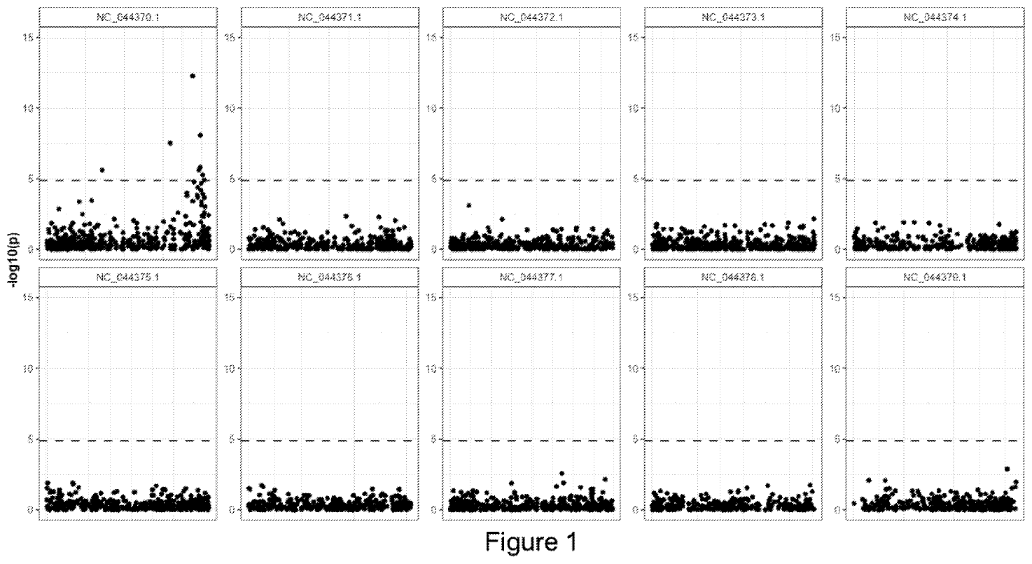

FIG. 2: GWA of hermaphroditic flowering in Cannabis in an expanded mixed F2 population.

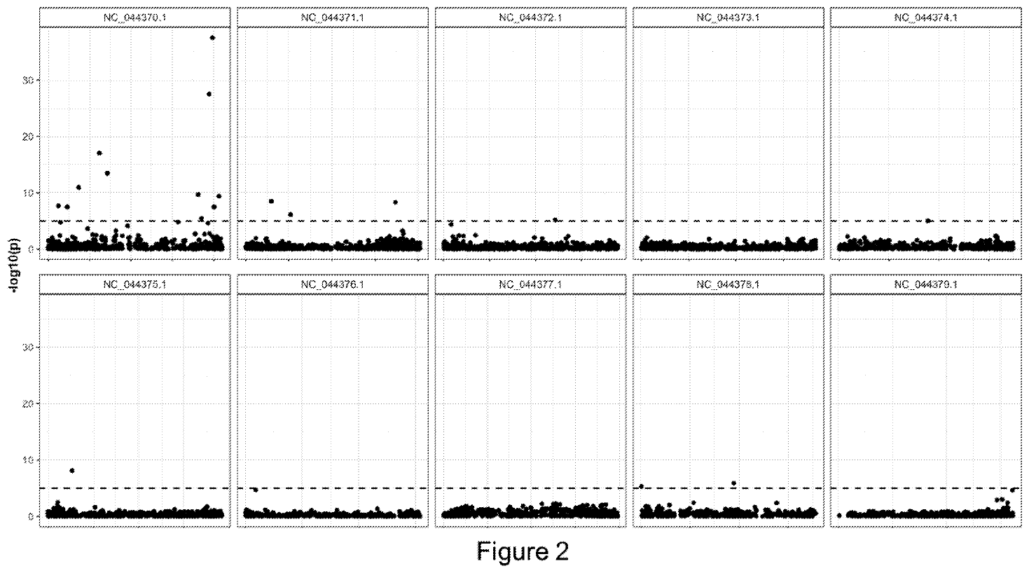

### SEQUENCES

The nucleic acid and amino acid sequences listed herein and in any accompanying sequence listing are shown using standard letter abbreviations for nucleotide bases, and the standard one- or three-letter abbreviations for amino acids. It will be understood by those of skill in the art that only one strand of each nucleic acid sequence is shown, but that the complementary strand is included by any reference to the displayed strand.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims, which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items. It is, however, contemplated as a specific embodiment of the present disclosure that the term “comprising” encompasses the possibility of no further members being present, i.e., for the purpose of such an embodiment “comprising” is to be understood as having the meaning of “consisting of”.

Methods are provided herein for characterizing plants for a hermaphroditism trait and for obtaining plants that do not have a hermaphroditism trait prior to the plant displaying hermaphroditic inflorescence, using a molecular marker detection technique. The inventors of the present invention have further produced cannabis plants that are greatly reduced in hermaphroditic flowering by crossing plants that suppress hermaphroditic flowering to cannabis plants where hermaphroditic flowering occurs. Also demonstrated herein, the inventors were able to use genome wide association (GWA) to identify a QTL linked to hermaphroditism in Cannabis. The inventors were also able to identify single nucleotide polymorphisms (SNPs) associated with the hermaphroditism trait; these SNPs were verified as genetic markers for identifying plants carrying the hermaphroditism trait. The inventors used the methods described herein to identify candidate genes that are causative for the hermaphroditism trait, as well as a causative SNP in one of the candidate genes that regulates the hermaphroditism trait. This finding provides for the improvement of methods for producing plants that do not display hermaphroditism or plants that have a decreased likelihood of producing hermaphroditic inflorescences in Cannabis spp. plants. In addition, this finding provides a method of prescreening a population for the hermaphroditism trait prior to the appearance of the trait.

At least two QTLs for hermaphroditism were identified and confirmed in the mixed populations tested and the 10 F2 populations tested.

Tables 1, 3 to 11 and 15 herein provide several single nucleotide polymorphisms (SNPs) which define the QTLs associated with the hermaphroditic trait and which can be used for characterizing a plant with respect to the hermaphroditism trait. In some embodiments one or more of the identified SNPs can be used to remove the hermaphroditic trait from a recipient plant, containing one or more of the QTLs associated with the trait. For example, the removal of the hermaphroditic phenotype may be performed by crossing a donor parent plant to a recipient parent plant to produce plants containing a haploid genome from both parents. Recombination of these genomes provides F1 progeny where each haploid complement of chromosomes, of the diploid genome, is comprised of genetic material from both parents.

In some embodiments, methods of identifying one or more QTLs that are characterized by a haplotype comprising of a series of polymorphisms in linkage disequilibrium are provided. The QTLs each display limited frequency of recombination within the QTLs. Preferably the polymorphisms are selected from any one of Tables 1, 3 to 11 and 15 herein, representing the hermaphroditism QTLs. Molecular markers may be designed for use in detecting the presence of the polymorphisms and thus the QTLs. Further, the identified QTL polymorphisms and the associated molecular markers may be used in a cannabis breeding program to predict the hermaphroditic trait of plants in a breeding population and can be used to produce cannabis plants that do not display the hermaphroditic trait, or which have a reduced propensity for the trait compared to the plants from which they are derived.

As used herein, reference to a “hermaphroditic inflorescence” or a variety with a “hermaphroditic inflorescence trait”, “hermaphroditic trait” or “hermaphroditism trait” refers to a plant or a variety in which pistillate flowers are accompanied by formation of anthers and/or where pistillate flowers and staminate flowers occur on the same cannabis plant at the time of harvest. The term “pistillate” refers to a flower that bears carpels, also referred to as the gynoecium, the female organs of a flower comprising the stigma, style, and ovary but with no stamens. The term “anthers” as referred to herein are the part of the stamen, the pollen-producing reproductive organ of a flower. The term “staminate” refers to a flower having only functional stamens and lacking functional carpels.

A “hermaphroditism trait of interest” refers to the state of the plant with respect to the hermaphroditism trait and includes the hermaphroditism absence trait and hermaphroditism presence trait.

A “hermaphroditism absence trait” is defined by the absence of hermaphroditism.

A “hermaphroditism presence trait” is defined by the presence of hermaphroditism.

The time of harvest is defined with respect to the maturity of the flower, where approximately greater than 50% of the pistils have turned brown in appearance. Alternatively, the time of harvest can also be determined by initiation of flowering for hemp-type cannabis or by other agronomic criteria common in the art.

It is a particular aim of the present invention to identify and characterize a plant for the hermaphroditism trait of interest early in the plant lifecycle, particularly prior to the plant displaying the hermaphroditism trait, particularly to remove plants having the hermaphroditism trait from the breeding population early on. This can be achieved by genotyping the plant using molecular markers for detecting the QTL associated with the hermaphroditism trait of interest prior to the time of harvest.

As used herein a “quantitative trait locus” or “QTL” is a polymorphic genetic locus with at least two alleles that differentially affect the expression of a continuously varying phenotypic trait when present in a plant or organism which is characterised by a series of polymorphisms in linkage disequilibrium with each other.

As used herein, the term “hermaphroditism QTL” or “hermaphroditism quantitative trait locus” refers to a quantitative trait locus comprising part, or all, of the QTLs characterized by one or more polymorphisms having an allelic state associated with the hermaphroditism trait of interest as described in any one of Tables 1, 3 to 11 and 15, or characterized by combinations of said polymorphisms.

In some cases, it is particularly desirable to obtain a plant that does not display the hermaphroditism trait, or which has a decreased propensity for displaying the hermaphroditism trait. Thus, it is an objective of the invention to provide for cannabis plants having a hermaphroditism absence QTL as described herein. In some cases, this may be achieved by characterizing plants with respect to the hermaphroditism QTL to determine whether they contain a hermaphroditism absence QTL or a hermaphroditism presence QTL.

As used herein, “hermaphroditism absence QTL” or “hermaphroditism absence quantitative trait locus” refers to a quantitative trait locus characterized by one or more polymorphisms having an allelic state associated with the hermaphroditism absence trait, as described in Tables 1, 3 to 11 and 15.

As used herein, “hermaphroditism presence QTL” or “hermaphroditism presence quantitative trait locus” refers to a quantitative trait locus characterized by one or more polymorphisms having an allelic state associated with the hermaphroditism presence trait, as described in Tables 1, 3 to 11 and 15.

As used herein, “haplotypes” refer to patterns or clusters of alleles or single nucleotide polymorphisms that are in linkage disequilibrium and therefore inherited together from a single parent. The term “linkage disequilibrium” refers to a non-random segregation of genetic loci or markers. Markers or genetic loci that show linkage disequilibrium are considered linked.

As used herein, the term “hermaphroditic haplotype” refers to the subset of the polymorphisms contained within the hermaphroditism QTL which exist on a single haploid genome complement of the diploid genome, and which are in linkage disequilibrium with the hermaphroditic trait.

As used herein, the term “donor parent plant” refers to a plant having a hermaphroditic haplotype, or one or more hermaphroditic alleles, associated with the hermaphroditism trait of interest.

As used herein, the term “recipient parent plant” refers to a plant having a hermaphroditic haplotype, or one or more hermaphroditic alleles, not associated with the hermaphroditism trait of interest.

The term “hermaphroditic allele” refers to the haplotype allele within a particular QTL that confers, or contributes to, the hermaphroditic trait of interest, or alternatively, is an allele that allows the identification of plants with the hermaphroditic trait of interest that can be included in a breeding program, particularly to select for the hermaphroditic absence trait (“marker assisted breeding”, “marker assisted selection”, or “genomic selection”).

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same, or genetically identical plant). The term “crossing” refers to the act of fusing gametes via pollination to produce progeny.

The term “GWAS” or “Genome wide association study” or “GWA” or “Genome wide association” as used herein refers to an observational study of a genome-wide set of genetic variants or polymorphisms in different individual plants to determine if any variant or polymorphism is associated with a trait, specifically the hermaphroditic trait of interest.

As used herein a “polymorphism” is a particular type of variance that includes both natural and/or induced multiple or single nucleotide changes, short insertions, or deletions in a target nucleic acid sequence at a particular locus as compared to a related nucleic acid sequence. These variations include, but are not limited to, single nucleotide polymorphisms (SNPs), indel/s, genomic rearrangements, and gene duplications.

As used herein, the term “LOD score” or “logarithm (base 10) of odds” refers to a statistical estimate used in linkage analysis, wherein the score compares the likelihood of obtaining the test data if the two loci are indeed linked, to the likelihood of observing the same data purely by chance. The LOD score is a statistical estimate of whether two genetic loci are physically near enough to each other (or “linked”) on a particular chromosome that they are likely to be inherited together. A LOD score of 3 or higher is generally understood to mean that two genes are located close to each other on the chromosome. In terms of significance, a LOD score of 3 means the odds are 1,000:1 that the two genes are linked and therefore inherited together.

As used herein, the term “quantile-quantile” or “Q-Q” refers to a graphical method for comparing two probability distributions by plotting their quantiles against each other. If the two distributions being compared are similar, the points in the Q-Q plot will approximately lie on the line y=x. If the distributions are linearly related, the points in the Q-Q plot will approximately lie on a line, but not necessarily on the line y=x. Q-Q plots can also be used as a graphical means of estimating parameters in a location-scale family of distributions.

As used herein, a “causal gene” is the specific gene having a genetic variant (the “causal variant”) which is responsible for the association signal at a locus and has a direct biological effect on the hermaphroditic trait. In the context of association studies, the genetic variants which are responsible for the association signal at a locus are referred to as the “causal variants”. Causal variants may comprise one or more “causal polymorphisms” that have a biological effect on the phenotype.

The encompasses both ribonucleotides (RNA) and term “nucleic acid” deoxyribonucleotides (DNA), including cDNA, genomic DNA, isolated DNA and synthetic DNA. The nucleic acid may be double-stranded or single-stranded. Where the nucleic acid is single-stranded, the nucleic acid may be the sense strand or the antisense strand. A “nucleic acid molecule” or “polynucleotide” refers to any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides. By “cDNA” is meant a complementary or copy DNA produced from an RNA template by the action of RNA-dependent DNA polymerase (reverse transcriptase).

In some embodiments, the nucleic acid molecules of the invention may be operably linked to other sequences. By “operably linked” is meant that the nucleic acid molecules, such as those comprising the QTLs of the invention or genes identified herein, and regulatory sequences are connected in such a way as to permit expression of the proteins when the appropriate molecules are bound to the regulatory sequences. Such operably linked sequences may be contained in vectors or expression constructs which can be transformed or transfected into plant cells or plants for expression. A “regulatory sequence” refers to a nucleotide sequence located either upstream, downstream or within a coding sequence. Generally regulatory sequences influence the transcription, RNA processing or stability, or translation of an associated coding sequence. Regulatory sequences include but are not limited to: effector binding sites, enhancers, introns, polyadenylation recognition sequences, promoters, RNA processing sites, stem-loop structures, translation leader sequences and the like.

The term “promoter” refers to a DNA sequence that is capable of controlling the expression of a nucleic acid coding sequence or functional RNA. A promoter may be based entirely on a native gene or it may be comprised of different elements from different promoters found in nature. Different promoters are capable of directing the expression of a gene at different stages of development, or in response to different environmental or physiological conditions. An “inducible promoter” is promoter that is active in response to a specific stimulus. Several such inducible promoters are known in the art, for example, chemical inducible promoters, developmental stage inducible promoters, tissue type specific inducible promoters, hormone inducible promoters, environment responsive inducible promoters.

The term “isolated”, as used herein means having been removed from its natural environment. Specifically, the nucleic acid(s) or gene(s) identified herein may be isolated nucleic acids or gene(s), which have been removed from plant material where they naturally occur.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids and carbohydrates which it is ordinarily associated with in its natural state.

The term “complementary” refers to two nucleic acid molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” or “substantially homologous” sequence is a nucleotide sequence that differs from a reference sequence only by one or more conservative substitutions, or by one or more non-conservative substitutions, deletions, or insertions located at positions of the sequence that do not destroy or substantially alter the activity of the polypeptide encoded by the nucleic acid molecule. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” or “substantially homologous” if they hybridize under high stringency conditions. The “stringency” of a hybridization reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridization generally depends on the ability of denatured DNA to re-anneal when complementary strands are present in an environment below their melting temperature. A typical example of such “stringent” hybridization conditions would be hybridization carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

Nucleotide positions of polymorphisms described herein are provided with reference to the corresponding position on the Cannabis sativa (assembly cs10) representative genome, provided as RefSeq assembly accession: GCF_900626175.2 on NCBI, loaded on 14 Feb. 2019, referred to herein as “cs10 reference genome” or “cs10 genome”.

### Methods of Identifying a QTL or Haplotype Responsible for the Hermaphroditic Phenotype And Molecular Markers Therefor

In some embodiments, methods are provided for identifying a QTL or haplotype responsible for a hermaphroditism trait of interest and for selecting plants having the hermaphroditism trait of interest, thereby to identify the QTL or haplotype responsible for the trait. In some embodiments, the methods may comprise the steps of:
a. Identifying a plant that displays the hermaphroditic presence trait or the hermaphroditic absence trait within a breeding program.
b. Establishing a population by crossing the identified plant to itself (selfing) or a recipient parent plant.
c. Genotyping the resultant F1, or subsequent populations, for example by sequencing methods.
d. Performing association studies, including phenotyping and linkage analysis, to discover QTLs and/or polymorphisms contained within the QTL.
e. Optionally, identifying cannabis paralogs of previously characterized genes that may be involved in conferring the hermaphroditism trait of interest.
f. Developing molecular markers that detect one or more polymorphisms linked to QTLs, alleles within these QTLs, or existing or induced polymorphisms.
g. Validating the molecular markers by determining the linkage disequilibrium between the marker and the hermaphroditic trait.

### Trait Development and Introgression

In some embodiments, methods are provided for marker assisted breeding (MAB) or marker assisted selection (MAS) of plants having the hermaphroditism absence QTL or displaying the hermaphroditic absence trait. The methods may comprise the steps of:
a. Identifying a plant that displays the hermaphroditism trait of interest or which contains a hermaphroditism QTL as defined herein.
b. Establishing a population by crossing the identified plant to itself (selfing) or another recipient parent plant.
c. Genotyping and phenotyping the resultant F1, or subsequent, populations, for example by sequencing methods.
d. Performing association studies, inputting phenotype and genotype information to identify genomic regions enriched with polymorphisms associated with the hermaphroditic trait, to discover QTLs and/or polymorphisms contained within the QTL.
e. Optionally, identifying cannabis paralogs of previously characterized genes that may be involved in the hermaphroditic phenotype.
f. Developing molecular markers that detect one or more polymorphisms linked to QTLs, alleles within these QTLs, or existing or induced polymorphisms.
g. Using the molecular markers when introgressing the QTLs or polymorphisms into new or existing cannabis varieties to select plants where the hermaphroditic haplotype or the hermaphroditism trait is absent.

### Qtls and Marker Assisted Breeding

In some embodiments, during the breeding process, selection of plants displaying the hermaphroditism trait of interest may be based on molecular markers designed to detect polymorphisms linked to genomic regions that control the trait of interest. In some embodiments, QTLs containing such elements are identified using association studies. Knowledge of the mode-of-action is not required for the functional use of these genomic regions in a breeding program. Identification of regions controlling unidentified mechanisms may be useful in obtaining plants with the hermaphroditism absence trait, based on identification of polymorphisms that are either linked to, or found within QTLs that are associated with the hermaphroditic trait of interest using association studies.

### Construction of Breeding Populations

Breeding populations are the offspring of sexual reproduction events between two or more parents. The parent plants (F0) are crossed to create an F1 population each containing a chromosomal complement of each parent. In a subsequent cross (F2), recombination has occurred and allows for mostly independent segregation of traits in the offspring and importantly the reconstitution of recessive phenotypes that existed in only one of the parental lines.

According to some embodiments, QTLs that lead to the hermaphroditism trait of interest identified within synthetic populations of plants capable of revealing dominant, recessive, or complex traits. In one embodiment of the invention, a genetically diverse population of cannabis varieties, that are used to produce the synthetic population are integrate them into a breeding program by unnatural processes. In some embodiments, these processes result in changes in the genomes of the plants. The changes may include, but are not limited to, mutations and rearrangements in the genomic sequences, duplication of the entire genome (polyploidy), or activation of movement of transposable elements which may inactivate, activate or attenuate the activity of genes or genomic elements. According to one embodiment of the invention, the methods employed to integrate the plants into a breeding program include some or all of the following:
a. Growing plants in rich media or soils under artificial lighting;
b. Cloning of plants, often through a multitude of sub-cloning cycles;
c. Introduction of plants into in vitro, sterile growth environments, and subsequent removal to standard growth conditions;
d. Exposure to mutagens such as EMS, colchicine, silver nitrate, ethidium bromide, dinitroanalines, high concentrations mono or poly-chromatic light sources;
e. Growing plants under highly stressful conditions which include restricted space, drought, pathogen, atypical temperatures, and nutrient stresses.

### Hermaphroditism Trait of Interest Association Studies and QTL Identification

In some embodiments, the synthetic populations created are either the offspring of the sexual reproduction or clones of plants in the breeding program such that genetic material of individuals in the synthetic populations is derived from one, or two, or more plants from the breeding program.

In one embodiment, plants identified within the synthetic population as having a trait of interest, such as the hermaphroditic trait of interest, may be used to create a structured population for the identification of the genetic locus responsible for the trait. The structured population may be created by crossing one (selfing) or more plants and recovering the seeds from those plants.

Plants in the structured population may be fully genotyped using genome sequencing to identify genetic markers for use in the association study (AS) database. Association mapping is a powerful technique used to detect quantitative trait loci (QTLs) specifically based on the statistical correlation between the phenotype and the genotype. In this case the trait is the hermaphroditic phenotype. In a population generated by crossing, the amount of linkage disequilibrium (LD) is reduced between genetic marker and the QTL as a function of genetic distance in cannabis varieties with similar genome structures. Simple association mapping is performed by biparental crosses of two closely related lines where one line has a phenotype of interest and the other does not. In some embodiments, advanced population structures may be used, including nested association mapping (NAM) populations or multi-parent advanced generation inter-cross (MAGIC) populations, however it will be appreciated that other population structures can also be effectively used. Biparental, NAM, or MAGIC structured populations can be generated and offspring, at F1 or later generations, may be maintained by clonal propagation for a desired length of time. In some embodiments, QTLs may be identified using the high-density genetic marker database created by genotyping the founder lines and structured population lines. This marker database may be coupled with an extensive phenotypic trait characterization dataset, including, for example, the hermaphroditic phenotype of the plants. Using the association studies described herein, together with accurate phenotyping, this method is able to identify genomic regions, QTLs and even specific genes or polymorphisms responsible for the hermaphroditism trait of interest that are directly introduced into recipient lines. Polygenic phenotypes may also be identified using the methods described herein.

In one embodiment, the structured population is grown to the time of harvest. To characterize the phenotypes of the lines, they are clonally reproduced so the phenotypic data can be collected in feasible replicates.

### Genomic Selection

In some embodiments, during the breeding process, selection of plants by genomic selection (GS) may be conducted. Genomic selection is a method in plant breeding where the genome wide genetic potential of an individual is determined to predict breeding values for those individuals. In some embodiments, the accuracy of genomic selection is affected by the data used in a GS model including size of the training population, relationships between individuals, marker density, use of pedigree information, and inclusion of known QTLs.

In some embodiments, a QTL or a SNP known to be associated with a trait that contributes to selection criteria can improve the accuracy of genomic selection models. In some embodiments, a genomic selection model that incorporates the hermaphroditism phenotype can be improved by the inclusion of the hermaphroditism QTL in the GS model. In some embodiments, the SNPs described in any of Tables 1, 3 to 11 and 15 may be useful in a genomic selection model, for example where genotypes with unknown phenotypes are evaluated using an approach like a random forest algorithm for prediction of the hermaphroditism trait, and particularly in combination, to improve the predictive power of the model.

### Molecular Markers to Detect Polymorphisms

As used herein, the term “marker” or “genetic marker” refers to any sequence comprising a particular polymorphism or haplotype described herein that is capable of detection. For example, a marker may be a binding site for a primer or set of primers that is designed for use in a PCR-based method to amplify and thus detect a polymorphism or haplotype. Alternatively, the marker may introduce a restriction enzyme recognition site, or result in the removal of a restriction enzyme recognition site. Plants can be screened for a particular trait based on the detection of one or more markers confirming the presence of the polymorphism. Marker detection systems that may be used in accordance with the present invention include, but are not limited to polymerase chain reaction (PCR) followed by sequencing, Kompetitive allele specific PCR (KASP), restriction fragment length polymorphisms (RFLPs) analysis, amplified fragment length polymorphisms (AFLPs), cleaved amplified polymorphic sequences (CAPS), or any other markers known in the art.

In some embodiments “molecular markers” refers to any marker detection system and may be PCR primers, such as those described in the examples below. For example, PCR primers may be designed that consist of a reverse primer and two forward primers that are homologous to the part of the genome that contains a polymorphism but differ in the 3′ nucleotide such that the one primer will preferentially bind to sequences containing the polymorphism and the other will bind to sequences lacking it. The three primers are used in single PCR reactions where each reaction contains DNA from a cannabis plant as a template. Fluorophores linked to the forward primers provide, after thermocycling, a different relative fluorescent signal for homozygous and heterozygous alleles containing the polymorphism and for those lacking the polymorphism, respectively.

In some embodiments, allele-specific primers may each harbor a unique tail sequence that corresponds with a universal FRET (fluorescence resonant energy transfer) cassette. For example, the primer specific to the SNP may be labelled with a FAM and the other specific primer with a HEX dye. During the PCR thermal cycling performed with these primers, the allele-specific primer binds to the genomic DNA template and elongates, so attaching the tail sequence to the newly synthesized strand. The complement of the allele-specific tail sequence is then generated during subsequent rounds of PCR, enabling the FRET cassette to bind to the DNA. Alleles are discriminated through the competitive binding of the two allele-specific forward primers. At the end of the PCR reaction a fluorescent plate is read using standard tools which may include RT-PCR devices with the capacity to detect florescent signals and is evaluated with commercial software.

If the genotype at a given polymorphism site is homozygous, one of the two possible fluorescent signals will be generated. If the genotype is heterozygous, a mixed fluorescent signal will be generated. By way of example, genomic DNA extracted from cannabis leaf tissue at seedling stage can be used as a template for PCR amplifications with reaction mixtures containing the three primers. Final fluorescent signals can be detected by a thermocycler and analyzed using standard software for this purpose, which discriminates between individuals that are heterozygotes or homozygotes for either allele.

In some embodiments, molecular markers to one, two or more of the SNPs in the haplotype can be used to identify the presence of the QTL and by association, the hermaphroditism trait of interest.

Further, the QTL may include a number of individual polymorphisms in linkage disequilibrium, which constitute a haplotype and which, with high frequency, can be inherited from a donor parent plant as a unit. Therefore, in some embodiments, molecular markers can be utilized which have been designed to identify numerous polymorphisms which are in linkage disequilibrium with other polymorphisms, any of which can be used to effectively predict the phenotype of the offspring for the hermaphroditism trait of interest.

According to some embodiments, any polymorphism in linkage disequilibrium with one or more of the hermaphroditism QTLs can be used to determine the hermaphroditism haplotype in a breeding population of plants, as long as the polymorphism is unique to the hermaphroditic trait of interest in the donor parent plant when compared to the recipient parent plant.

In some embodiments the desired trait is the hermaphroditism absence trait, and the donor parent plant may be a plant that has been genetically modified or selected to exclude a hermaphroditism absence QTL defined by a polymorphism associated with the hermaphroditic absence trait, for example any, some, or all of the polymorphisms defined in any of Tables 1, 3 to 11 and 15 associated with the trait.

Alternatively, the desired trait may be the hermaphroditism presence trait, and the donor parent plant may be a plant that has been genetically modified or selected to include a hermaphroditism presence QTL defined by a polymorphism associated with the hermaphroditic presence trait, for example any, some, or all of the polymorphisms defined in any of Tables 1, 3 to 11 and 15 associated with the trait.

In some embodiments, donor parent plants, as described above, are used as one of two parents to create breeding populations (F1) through sexual reproduction. Methods for reproduction that are known in the art may be used. The donor parent plant provides the trait of interest to the breeding population. The trait is made to segregate through the population (F2) through at least one additional crossing event of the offspring of the initial cross. This additional crossing event can be either a selfing of one of the offspring or a cross between two individuals, provided that each plant used in the F1 cross contains at least one copy of a desired QTL allele or haplotype.

In some embodiments, the hermaphroditic allele or hermaphroditic haplotype in plants to be used in the F1 cross is determined using the described molecular markers. In some embodiments, the resulting F2 progeny is/are screened for any of the hermaphroditism polymorphisms associated with the hermaphroditism trait of interest described herein.

The plants at any generation can be produced by asexual means like cutting and cloning, or any method that yields a genetically identical offspring.

Production of Cannabis Spp. Plants Having the Hermaphroditism Absence Trait

It is a particular aim of the present invention to provide for the production of Cannabis spp. Plants that do not have the hermaphroditism trait. According, in some embodiments, a Cannabis spp. plant that has the hermaphroditism presence trait may be converted into a plant having a hermaphroditism absence trait according to the methods of the present invention by providing a breeding population where the donor parent plant contains a hermaphroditism absence QTL and the recipient parent plant either displays the hermaphroditism presence trait or contains a hermaphroditism presence QTL.

In some embodiments the hermaphroditism presence trait may be removed from a recipient parent plant by crossing it with a donor parent plant having the hermaphroditism absence QTL. In some embodiments, the donor parent plant does not have a hermaphroditism phenotype and contains a contiguous genomic sequence characterized by one or more of the polymorphisms of Tables 1, 3 to 11 and 15 associated with the hermaphroditism allele or hermaphroditism haplotype conferring the hermaphroditism absence trait.

In some embodiments, the donor parent plant is any Cannabis spp. variety that is cross fertile with the recipient parent plant.

In some embodiments, MAS or MAB may be used in a method of backcrossing plants carrying the hermaphroditism absence trait to a recipient parent plant. For example, an F1 plant from a breeding population can be crossed again to the recipient parent plant. In some embodiments, this method is repeated.

In some embodiments, the resulting plant population is then screened for the hermaphroditism absence trait using MAS with molecular markers to identify progeny plants that contain one or more polymorphism, such as any of those described in Tables 1, 3 to 11 and 15, indicating the presence of an allele of a QTL associated with the hermaphroditism absence phenotype. In another embodiment, the population of cannabis plants may be screened by any analytical methods known in the art to identify plants with desired characteristics, specifically hermaphroditism absence.

Production of Cannabis Spp. Plants Having the Hermaphroditism Presence Trait

In an alternative embodiment, a Cannabis spp. plant that does not have the hermaphroditism trait may be converted into a hermaphroditism plant according to the methods of the present invention by providing a breeding population where the donor parent plant contains a hermaphroditism presence QTL associated with the hermaphroditism presence trait, and recipient parent plant either displays the hermaphroditism absence trait or contains the hermaphroditism absence QTL.

Stable hermaphrodite cannabis plants, in which hermaphroditism occurs irrespective of environmental conditions represent an expansion of the breeding potential and usefulness of cannabis plants in many production systems. Stable or inducible hermaphroditism in Cannabis can be used to facilitate inbreeding, without the need of lengthy and low-throughput chemical induction of male flowers and crossing procedures, in order to produce homozygous inbred lines that can be used as parents to generate hybrid lines that benefit from heterosis. Stable or inducible hermaphroditism may also aid in the generation of double haploid plants, where haploid cells undergo chromosome doubling, leading to plants with high homozygosity, an alternative and accelerated route to conventional inbreeding. The surplus of pollen from hermaphrodite plants versus sex-reversed female plants is an advantage for generating double haploid plants.

Stable or inducible hermaphroditism may also be useful for inbreeding in autoflowering cannabis plants, or other genetic backgrounds where propagation of clones is challenging or where chemical induction of male flowers is not practical or possible. In hemp, where seed yield is a trait of interest, introgression of a stable or inducible hermaphroditic trait into hemp-type has the potential to increase the yield of seed production by maximizing the number of plants that produce seeds, in contrast to the current conditions where both male and female plants are present and 50% of the plants in field are without seed. The generation of hermaphrodite hemp-type cannabis plants may also increase the yield of seed production. Such a system also ensures the synchronisation of flowering of both female and male flowers as compared to conventional dioecious varieties where male plants tend to flower earlier then their female counterparts.

Accordingly, in some embodiments the hermaphroditism presence trait may be introduced into a recipient parent plant by crossing it with a donor parent plant having the hermaphroditism presence QTL. In some embodiments the donor parent plant has a hermaphroditism phenotype and a contiguous genomic sequence characterized by one or more of the polymorphisms of any one of Tables 1, 3 to 11 and 15 associated with the hermaphroditism allele or hermaphroditism haplotype conferring the hermaphroditism presence trait.

In some embodiments, the donor parent plant is any Cannabis spp. variety that is cross fertile with the recipient parent plant.

In some embodiments, MAS or MAB may be used in a method of backcrossing plants carrying the hermaphroditism presence trait to a recipient parent plant. For example, an F1 plant from a breeding population can be crossed again to the recipient parent plant. In some embodiments, this method is repeated.

In some embodiments, the resulting plant population is then screened for the hermaphroditism presence trait using MAS with molecular markers to identify progeny plants that contain one or more polymorphisms, such as those described in any one of Tables 1, 3 to 11 and 15, indicating the presence of an allele of a QTL associated with the hermaphroditism presence phenotype. In another embodiment, the population of cannabis plants may be screened by any analytical methods known in the art to identify plants with desired characteristics, specifically hermaphroditism presence.

### Methods to Genetically Engineer Plants to Achieve the Hermaphroditism Trait of Interest Using Mutagenesis or Gene Editing Techniques

Identifying QTLs, and individual polymorphisms, that correlate with a trait when measured in an F1, F2, or similar, breeding population indicates the presence of one or more causative polymorphisms in close proximity the polymorphism detected by the molecular marker. In some embodiments, the polymorphisms associated with the presence or absence of the hermaphroditism trait are introduced into a plant by other means so that a trait can be introduced into plants that would not otherwise contain associated causative polymorphisms or removed from plants that would otherwise contain associated causative polymorphisms. For example, an A/G SNP at position 99811627 on chromosome NC_044370.1 with reference to the cs10 reference genome (position 1 of SEQ ID NO:343). Further, the polymorphisms detailed in Tables 1, 3 to 11 and 15 are molecular markers that can be used to indicate the presence of a possible causative polymorphism.

The entire QTLs or parts thereof which confer the hermaphroditism trait of interest described herein, or the genes or nucleic acid molecules described herein, may be introduced into the genome of a cannabis plant to obtain plants with a hermaphroditism trait of interest, through a process of genetic modification known in the art, for example, but not limited to, heterologous gene expression using an expression cassette including a sequence encoding the QTL(s) or part thereof, the gene(s), or the nucleic acids. The expression cassettes may contain all or part of the QTL(s) or gene(s), including possible causative polymorphisms.

The trait described herein may be introduced into, or removed from, the genome of a cannabis plant to obtain plants that include or exclude the causative polymorphisms and the potential to display a desired hermaphroditism trait of interest through processes of genetic modification known in the art, for example, but not limited to, CRISPR-Cas9 targeted gene editing, TILLING, non-targeted chemical mutagenesis using e.g., EMS.

The present invention further provides methods for producing a modified Cannabis spp. plant using genome editing or modification techniques. For example, genome editing can be achieved using sequence-specific nucleases (SSNs) the use of which results in chromosomal changes, such as nucleotide deletions, insertions or substitutions at specific genetic loci, particularly those associated with the hermaphroditism trait of interest, more particularly an A/G SNP at position 99811627 on chromosome NC_044370.1 with reference to the cs10 reference genome (position 1 of SEQ ID NO:343). Non limiting examples of SSNs include zinc finger nucleases (ZFNs), TAL effector nucleases (TALENs), meganucleases, and clustered regularly interspaced short palindromic repeats (CRISPR)/CRISPR-associated protein (Cas) system. In some embodiments, non-limiting examples of Cas proteins suitable for use in the methods of the present invention include Csnl, Cpfl, Cas9, Cas 12, Cas 13, Cas 14, CasX, and combinations thereof. In one embodiment, a modified Cannabis spp. plant having a hermaphroditism trait of interest is generated using CRISPR/Cas9 technology, which is based on the Cas9 DNA nuclease guided to a specific DNA target by a single guide RNA (sgRNA). For example, the genome modification may be introduced using guide RNA, e.g., single guide RNA (sgRNA) designed and targeted to introduce a polymorphism associated with the hermaphroditism trait of interest, such as a G>A SNP at position 99811627 on chromosome NC_044370.1 with reference to the cs10 reference genome (position 1 of SEQ ID NO:343).

DNA introduction into the plant cells can be performed using Agrobacterium infiltration, virus-based plasmid delivery of the genome editing molecules and mechanical insertion of DNA (PEG mediated DNA transformation, biolistics, etc.). In some embodiments, the Cas9 protein may be directly inserted together with a gRNA (ribonucleoprotein-RNP's) in order to bypass the need for in vivo transcription and translation of the Cas9+gRNA plasmid in planta to achieve gene editing. In one embodiment, a genome edited plant may be developed and used as a rootstock, so that the Cas protein and gRNA can be transported via the vasculature system to the top of the plant and create the genome editing event in the scion.

According to one embodiment of the present invention, the method of genetically modifying a plant may be achieved by combining the Cas nuclease (e.g., Cas9, Cpf 1) with a predefined guide RNA molecule (gRNA). The gRNA is complementary to a specific DNA sequence targeted for editing in the plant genome and which guides the Cas nuclease to a specific nucleotide sequence. The predefined gene-specific gRNAs may be cloned into the same plasmid as the Cas gene and this plasmid is inserted into plant cells as described above.

In some embodiments, once the gRNA molecule and Cas9 nuclease reach the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which introduce a mutation at the cleavage site.

In one embodiment, a deletion form of the mutation may consist of at least 1 base pair deletion. As a result of this base pair deletion, the gene coding sequence for the putative gene(s) responsible for the hermaphroditism trait of interest, such as the genes described in Table 13, or more particularly a gene having the gene identity number LOC115696400 with reference to the CS10 genome and encoding a putative 1-aminocyclopropane-1-carboxylate synthase (SEQ ID NO: 343), is disrupted and the translation of the encoded protein is compromised by disruption of a start codon, introduction of a premature stop codon or disruption of a functional or structural property of the protein.

In another embodiment, the hermaphroditism trait of interest in Cannabis spp. plants may be introduced by generating gRNA with homology to a specific site of predetermined genes in the Cannabis genome or a QTL defined herein. In one embodiment the gene may be one or more of the genes described in Table 13 herein, or more particularly a gene having the gene identity number LOC115696400 with reference to the CS10 genome and encoding a putative 1-aminocyclopropane-1-carboxylate synthase (SEQ ID NO:343). This gRNA may be sub-cloned into a plasmid containing the Cas9 gene, and the plasmid inserted into the Cannabis plant cells. In this way site specific mutations in the QTL are generated, including the SNPs associated with the hermaphroditism trait of interest described in Tables 1, 3 to 11 and 15, and in particular a causative polymorphism, more particularly the A/G SNP at position 99811627 on chromosome NC_044370.1 with reference to the cs10 reference genome (position 1 of SEQ ID NO:343), thus effectively introducing the hermaphroditism trait of interest into the genome edited plant.

In some embodiments, a modified Cannabis spp. plant exhibiting a hermaphroditism absence trait may be obtained using the targeted genome modification methods described above, wherein the plant comprises a targeted genome modification to introduce one or more polymorphisms associated with the hermaphroditism presence trait defined in Tables 1, 3 to 11 and 15, wherein the modification effects the hermaphroditism presence trait. In a preferred embodiment, the plant comprises a targeted genome modification to introduce a G>A SNP at position 99811627 on chromosome NC_044370.1 with reference to the cs10 reference genome (position 1 of SEQ ID NO:343), to obtain a modified Cannabis spp. plant exhibiting a hermaphroditism absence trait.

In some embodiments, for example where the hermaphroditism trait of interest is a hermaphroditism absence trait, the genetic modification may be introduced using gene silencing, a process by which the expression of a specific gene product is lessened or attenuated. Gene silencing can take place by a variety of pathways, including by RNA interference (RNAi), an RNA dependent gene silencing process. In one embodiment, RNAi may be achieved by the introduction of small RNA molecules, including small interfering RNA (siRNA), microRNA (miRNA) or short hairpin RNA (shRNA), which act in concert with host proteins (e.g., the RNA induced silencing complex, RISC) to degrade messenger RNA (mRNA) in a sequence-dependent fashion. In particular, RNAi may be used to silence one or more of the putative causative genes described in Table 13 herein, or more particularly a gene having the gene identity number LOC115696400 with reference to the CS10 genome and encoding a putative 1-aminocyclopropane-1-carboxylate synthase (SEQ ID NO:343). Such RNAi molecules may be designed based on the sequence of these genes. These molecules can vary in length (generally 18-30 base pairs) and may contain varying degrees of complementarity to their target mRNA in the antisense strand. Some, but not all, RNAi molecules have unpaired overhanging bases on the 5′ or 3′ end of the sense strand and/or the antisense strand. As used herein, the term “RNAi molecule” includes duplexes of two separate strands, as well as single strands that can form hairpin structures comprising a duplex region. The RNAi molecules may be encoded by DNA contained in an expression cassette and incorporated into a vector. The vector may be introduced into a plant cell using Agrobacterium infiltration, virus-based plasmid delivery of the vector containing the expression cassette and/or mechanical insertion of the vector (PEG mediated DNA transformation, biolistics, etc.).

Plants may be screened with the molecular markers as described herein to identify transgenic individuals with the hermaphroditism trait of interest or having a hermaphroditism QTL or polymorphism(s), following the genetic modification.

In some embodiments, Cannabis spp. plants having one or more of the polymorphisms of any one of Tables 1, 3 to 11 and 15 associated with the hermaphroditism QTLs or linked thereto are provided. More particularly, Cannabis spp. plants having a causative A/G SNP at position 99811627 on chromosome NC_044370.1 with reference to the cs10 reference genome (position 1 of SEQ ID NO:343) are provided. The polymorphisms, including the causative polymorphism, may be introduced, for example, by genetic engineering. In some embodiments the one or more polymorphisms associated with the hermaphroditism trait of interest or linked thereto are introduced into the plants by breeding, such as by MAS or MAB, for example as described herein.

The hermaphroditism QTLs described herein, or genes identified herein responsible for effecting the hermaphroditism trait, may be under the control of, or operably linked to, a promoter, for example an inducible promoter. Such QTLs or genes may be operably linked to the inducible promoter so as to induce or suppress the hermaphroditism trait or phenotype in the plant or plant cell.

Accordingly, in a further embodiment, Cannabis spp. plants comprising a hermaphroditism QTL described herein, including a hermaphroditism absence QTL or a hermaphroditism presence QTL, or one or more polymorphisms associated therewith, are provided. In some cases, such plants are provided for with the proviso that the plant is not exclusively obtained by means of an essentially biological process.

The following examples are offered by way of illustration and not by way of limitation.

### Example 1

### Genome-Wide Association Studies (GWAS) of Hermaphroditic Inflorescence in Mixed Population of Cannabis

During outdoor field trials in 2020 it was observed that several populations of cannabis plants were comprised of individuals with hermaphroditic inflorescences. To identify molecular markers for the appearance of hermaphroditism in Cannabis, the study initially focused on the apical inflorescence in a diverse population comprising 3220 individuals.

Through the 2020 field trial, Cannabis sativa genotypes were monitored for the emergence of hermaphroditic flowers, including pistillate flowers that showed the emergence of anthers, as well as the growth of staminate flowers along with pistillate flowers. Individual plants that were scored hermaphroditic were assigned as 1, those that were not coded as hermaphroditic were assigned as 0.

DNA was extracted from about 70 mg of leaf discs from all the plants evaluated using an adapted kit with “sbeadex” magnetic beads by LGC Genomics, which was automated on a KingFisher Flex with 96 Deep-Well Head by Thermo Fisher Scientific.

The extracted DNA served as a template for the subsequent library preparation for sequencing. The library pools were prepared according to the manufacturer's instructions (AgriSeq™ HTS Library Kit-96 sample procedure from Thermo Fisher Scientific). Targeted sequencing of a custom SNP marker panel based on the Cannabis Sativa CS10 reference genome (NCBI GenBank assembly accession number GCA_900626175.2 as updated in April 2020 and accessed in February 2022) was carried out on the Ion Torrent system by Thermo Fisher Scientific. The primers for the SNPs identified are provided in Table 13. The library pool was loaded onto Ion 550 chips with Ion Chef and sequenced with Ion GeneStudio S5 Plus according to the manufacturer's instructions (Ion 550™ Kit from Thermo Fisher Scientific).

From a population of 400 individuals, a genome-wide association study (GWAS) was performed to detect significant associations between genotypic information derived from targeted resequencing of the custom SNP marker panel described above and the appearance of hermaphroditism in the apical inflorescence. Individual plants that were scored hermaphroditic were assigned as 1, those that were not coded as hermaphroditic were assigned as 0.

The genotypic matrix was filtered for SNPs having more than 30% missing values within the population and a minor allele frequency lower than 5%. This resulted in 4815 SNP markers after filtering. The GWAS was performed using GAPIT version 3 (J. Wang & Zhang, 2021) with four statistical models: General Linear Model (GLM), Mixed Linear Model (MLM), FarmCPU and Blink (model=c (“GLM”, “MLM”, “FarmCPU”, “Blink”). A quantile-quantile plot (QQ plot) was used to evaluate the statistical models. The FarmCPU model performed the best by our evaluation and was used for the analysis. SNPs surpassing a LOD (−log 10 (p-value)) value of 5 were considered to have a significant association with trait variation.

SNPs showing a significant association with hermaphroditic inflorescence, with an LOD value greater than 5, were found on chromosome NC_044370.1 and NC_044372.1 with reference to the Cannabis Sativa CS10 genome and are listed in Table 1 below. The homozygous allele of the SNPs in Table 1 that are associated with hermaphroditic inflorescence are listed along with their position and reference sequence. Only one SNP was found associated with hermaphroditic inflorescence on NC_044372.1 defining a QTL there, while four SNP markers were found associated with hermaphroditic inflorescence on NC_044370.1. These comprise three distinct QTLs associated with hermaphroditism defined by the SNPs in Table 1. On Chromosome NC_044370.1 we found three QTLs, the first is defined by the SNP “common_10” at position 889040, the second by SNPs “rare_30” and “GBScompat_common_54” from position 28544332-32763867, and a third at position 99761993 defined by the SNP “common_524”. For all the SNPs identified in Table 1 there is an indication that the allele state that indicates the least likelihood of hermaphroditic inflorescences may be useful in screening and/or breeding plants that have a highly decreased likelihood of producing hermaphroditic inflorescence in environmentally controlled and outdoor environments. Because, hermaphroditism is in part a stress induced trait, conducting this trial in an outdoor exposed environment likely facilitated the identification of the relevant markers.

TABLE 1
SNPs associated with hermaphroditic inflorescence in Cannabis a field trial on Chromosomes NC_044372.1
and NC_044370.1. The presence of the hermaphroditic inflorescence is predicted by the occurrence of the
indicative allele (marked with *). The positions of the SNPs are provided with reference to the CS10 reference genome
as described herein. “Homo_1” denotes the average phenotypic value associated with homozygous allele
1 based on a score from 0-1, as described above, where 1 indicates a plant with hermaphroditic inflorescence and
0 indicates one without, “Homo_2” denotes the average phenotypic value associated with homozygous
allele 2 based on a score from 0-1, as described above, where 1 indicates a plant with hermaphroditic inflorescence
and 0 indicates one without and “Hetero” denotes the average phenotypic value associated with the
heterozygous allele state based on a score from 0-1, as described above, where 1 indicates a plant with hermaphroditic
inflorescence and 0 indicates one without. BP refers to the nucleotide position of the SNP.

| SNP Marker | Chromosome | BP | LOD | Allele1 | Allele2 | Homo_1 | Hetero | Homo_2 |
|---|---|---|---|---|---|---|---|---|
| common_1527 | NC_044372.1 | 61687722 | 9.57142 | A | G* | 0.143 | 0.219 | 0.405 |
| common_524 | NC_044370.1 | 99761993 | 7.58089 | A* | G* | 0.25 | 0.416 | 0.124 |
| rare_30 | NC_044370.1 | 28544332 | 5.42871 | A* | C* | 0.308 | 0.364 | 0.05 |
| common_10 | NC_044370.1 | 889040 | 5.17368 | A* | C* | 0.09 | 0.426 | 0.423 |
| GBScompat_common_54 | NC_044370.1 | 32763867 | 4.99593 | A* | C | 0.309 | 0.259 | 0 |

### Example 2

### Genome-Wide Association Studies (GWAS) of Hermaphroditic Inflorescence in Mixed F2 Population in Cannabis

To better understand the segregation of the hermaphroditic trait and to confirm the QTLs identified in Example 1, several targeted crosses were made between genotypes displaying low rates of hermaphroditic inflorescence, those with intermediate degrees of hermaphroditic inflorescence, and those with higher rates of inflorescence. The progeny of these crosses were selfed to generate 10 F2 populations (Table 2).

TABLE 2
Genotype identification and populations used. “GPA” refers to the identity of the
Grandparent Pollen Acceptor and “GPD” refers to the identity of the Grandparent Pollen Donor.

| | Total # | F2 Percentage | | GPA Percent | | GPD Percent | # of SNP Markers | Significant |
|---|---|---|---|---|---|---|---|---|
| GID of F2 | of Plants | Hermaphroditic | GPA | Hermaphroditic | GPD | Hermaphroditic | After Filtering | GWA |
| 21 002 003 0000 | 146 | 27.40 | 20 000 027 0000 | 24.54% | 20 000 017 0000 | 22.49% | 3227 | Y |
| 21 002 004 0000 | 137 | 20.44 | 20 000 081 0000 | 18.46% | 20 000 017 0000 | 22.49% | 3885 | Y |
| 21 002 012 0000 | 153 | 11.76 | 20 000 099 0000 | 11.76% | 20 000 020 0000 | 19.33% | NA | N |
| 21 002 014 0000 | 96 | 20.83 | 20 000 098 0000 | 12.96% | 20 000 020 0000 | 19.33% | 3728 | Y |
| 21 002 028 0000 | 123 | 16.26 | 20 000 081 0000 | 18.46% | 20 000 070 0000 | 19.46% | 4429 | Y |
| 21 002 035 0000 | 163 | 16.56 | 20 000 104 0000 | 16.56% | 20 000 072 0000 | 14.76% | NA | N |
| 21 002 036 0000 | 129 | 12.40 | 20 000 426 0000 | 16.41% | 20 000 072 0000 | 14.76% | 4212 | Y |
| 21 002 038 0000 | 127 | 20.47 | 20 000 426 0000 | 16.41% | 20 000 072 0000 | 14.76% | 3250 | Y |
| 21 002 041 0000 | 99 | 10.10 | 20 000 061 000 | 10.28% | 20 000 072 0000 | 14.76% | NA | N |
| 21 002 046 0000 | 115 | 1.74 | 20 000 006 0000 | 1.74% | 20 000 083 0000 | 10.44% | NA | N |

The inventors observed the emergence of hermaphroditic inflorescence in an outdoor field trial of each of the 10 F2 populations described in Table 2. In order to identify genetic regions associated with hermaphroditism in cannabis flowers they scored these 10 F2 populations for the presence or absence of hermaphroditic inflorescence by visually inspecting female flowers for the appearance of stamens or the growth of staminate flowers alongside pistillate flowers. This was used to calculate the percent of the population in which hermaphroditic inflorescence emerged. The inventors note that the evaluation of the trait expression and segregation patterns is complicated by the influence of environmental factors on hermaphroditic flowering. By conducting these experiments in a randomized field trial, they sought to minimize positional effects in the field.

DNA was extracted from about 70 mg of leaf discs from all the plants evaluated in these 10 F2 populations. Using an adapted kit with “sbeadex” magnetic beads by LGC Genomics, which was automated on a KingFisher Flex with 96 Deep-Well Head by Thermo Fisher Scientific.

The extracted DNA served as a template for the subsequent library preparation for sequencing. The library pools were prepared according to the manufacturer's instructions (AgriSeq™ HTS Library Kit-96 sample procedure from Thermo Fisher Scientific). Targeted sequencing of a custom SNP marker panel based on the Cannabis Sativa CS10 reference genome was carried out on the Ion Torrent system by Thermo Fisher Scientific. The library pool was loaded onto Ion 550 chips with Ion Chef and sequenced with Ion GeneStudio S5 Plus according to the manufacturer's instructions (Ion 550™ Kit from Thermo Fisher Scientific).

Targeted DNA sequenced from all 10 F2 populations segregating for the hermaphroditic trait, a population of 1267 individual, was used in a genome-wide association analysis (GWAS) to detect significant associations between genotypic information derived from targeted resequencing of the custom SNP marker panel described above and the appearance of hermaphroditic flowers, individual plants that were scored hermaphroditic were assigned as 1, while those that were not hermaphroditic were assigned as 0.

The genotypic matrix was filtered for SNPs having more than 30% missing values within the population and a minor allele frequency lower than 5%. This resulted in 3858 SNP markers after filtering. The GWAS was performed using GAPIT version 3 (J. Wang & Zhang, 2021) with four statistical models: General Linear Model (GLM), Mixed Linear Model (MLM), FarmCPU and Blink (model=c (“GLM”, “MLM”, “FarmCPU”, “Blink”). A quantile-quantile plot (QQ plot) was used to evaluate the statistical models. The MLM model performed the best by the inventors' evaluation as it best accounted for population structure and was used for the analysis. SNPs surpassing a Bonferroni-corrected LOD (−log 10 (0.05/number of markers)) were considered to have a significant association with trait variation.

SNPs showing a significant association with hermaphroditism, with an LOD value greater than 5, were found only on chromosome NC_044370.1 with reference to the Cannabis Sativa CS10 genome and are listed in Table 3. The homozygous allele of the SNPs in Table 1 that can distinguish the likelihood that a hermaphroditic inflorescence will emerge during growth are listed along with their position and reference sequence. The alternative allele will in this case indicate plants that on average have 0 or close to 0 likelihood of producing hermaphroditic inflorescence.

From the results of the GWA, the inventors identified three QTLs based on the SNPs identified as associated with hermaphroditic inflorescence in the mixed F2 population listed in Table 3. The QTLs are defined by the SNPs on chromosome NC_044370.1. The first QTL is defined by the SNP “GBScompat_common_56” at position 35677966, the second can be defined by the SNP “rare_50” at position 79534090, and the third is at position 94129798-101726389 demarcated by SNPs “common_491” and “common_546”.

The QTL on NC_044372.1 identified in Example 1 was not detected here, nor was the QTL defined by SNP “common_10” on chromosome NC_044370.1. However, the possibility cannot be ruled out that these QTLs and the SNPs that define them are associated with the hermaphroditic inflorescence phenotype. It may be the case that the source of these QTLs is not present in the targeted F2 populations designed in Example 2. The mixed F2 population is confirmation of the other QTLs identified in the 2020 field experiment from Example 1.

TABLE 3
SNPs associated with hermaphroditic inflorescence in Cannabis from a mixed F2 population
on Chromosome NC_044370.1. The presence of the hermaphroditic inflorescence is predicted
by the occurrence of the indicative allele (marked with *). The positions of the SNPs are
provided with reference to the CS10 reference genome as described herein. “Homo_1”
denotes the average phenotypic value associated with homozygous allele 1 based on a score
from 0-1, as described above, where 1 indicates a plant with hermaphroditic inflorescence
and 0 indicates one without, “Homo_2” denotes the average phenotypic value associated
with homozygous allele 2 based on a score from 0-1, as described above, where 1 indicates
a plant with hermaphroditic inflorescence and 0 indicates one without, and “Hetero”
denotes the average phenotypic value associated with the heterozygous allele state based
on a score from 0-1, as described above, where 1 indicates a plant with hermaphroditic
inflorescence and 0 indicates one without. BP refers to the nucleotide position of the SNP.

| SNP Marker | BP | LOD | Allele1 | Allele2 | Homo_1 | Hetero | Homo_2 |
|---|---|---|---|---|---|---|---|
| common_491 | 94129798 | 9.130452 | A | G * | 0.01 | 0.08 | 0.50 |
| common_517 | 99121582 | 7.410864 | A | G * | 0.02 | 0.07 | 0.32 |
| common_518 | 99186615 | 7.227338 | A | G * | 0.01 | 0.04 | 0.26 |
| rare_50 | 79534090 | 6.695617 | A | G * | 0.01 | 0.09 | 0.48 |
| common_534 | 100736341 | 5.744335 | A * | G | 0.25 | 0.06 | 0.01 |
| common_525 | 99830512 | 5.541966 | C * | G | 0.31 | 0.08 | 0.01 |
| common_511 | 97379141 | 5.357322 | A | G * | 0.01 | 0.08 | 0.26 |
| GBScompat_{—} | 35677966 | 5.241665 | A | G * | 0.05 | 0.12 | 0.23 |
| common_56 | | | | | | | |
| common_546 | 101726389 | 5.172549 | A * | C | 0.21 | 0.12 | 0.03 |

### Example 3

### Genome-Wide Association Studies (GWAS) of Hermaphroditic Inflorescence in Individual F2 Population in Cannabis

The inventors then looked at each of the F2 populations from Example 2 individually to assess if associations were masked by looking at the whole population together.

Targeted DNA sequenced from all 10 F2 populations segregating for the hermaphroditic trait, were evaluated separately in a genome-wide association analysis (GWAS) to detect significant associations between genotypic information derived from targeted resequencing of the custom SNP marker panel described in Example 2 above and the appearance of hermaphroditic flowers, evaluated as the percent hermaphroditic (plants with hermaphroditic flowers/total plants).

The experimental design followed the same methodology as conducted in the mixed F2 populations. The number or identity of individuals in each individual population is listed in Table 2 above.

The genotypic matrix was filtered for SNPs having more than 30% missing values within the population and a minor allele frequency lower than 5% for each population individually the results of the SNP filtering is listed in Table 2 for each individual F2 population. The GWAS was performed using GAPIT version 3 (J. Wang & Zhang, 2021) with four statistical models: General Linear Model (GLM), Mixed Linear Model (MLM), FarmCPU and Blink (model=c (“GLM”, “MLM”, “FarmCPU”, “Blink”). A quantile-quantile plot (QQ plot) was used to evaluate the statistical models. The MLM model performed the best by evaluation in all individual F2 populations as it best accounted for population structure and was used for the analysis. SNPs surpassing a Bonferroni-corrected LOD (−log 10 (0.05/number of markers)) were considered to have a significant association with trait variation.

In 6 out of 10 of the F2 populations, the inventors detected significant associations between genotypic information and the presence of hermaphroditic inflorescences Table 2. Surprisingly, even though all F2 populations showed the occurrence of hermaphroditic inflorescence, four of the populations, those with the lowest percent hermaphroditic levels did not show significant association from the GWA experiment. This shows that the population design was important in the identification of SNPs associated with hermaphroditic inflorescence that may otherwise not have been identified. Indeed, conducting this trial in any environment may also not have brought out the expression of the hermaphroditic trait strongly. Conducting this experiment in the field in non-ideal growth conditions may have also strongly contributed to the experiment identifying molecular markers for this trait.

Looking individually at each of F2 population (Tables 5, 6, 7, 8 and 9) did not identify additional QTLs, rather it identified additional associated SNP markers for, as well as supporting, each of the three QTLs defined in Example 2, that are useful in predicting the likelihood that a plant will display hermaphroditic inflorescence.

The inventors detected 1 SNP, termed “common_491”, found in 5 of the 6 individual F2 populations where a significant association was found. The inventors detected 5 additional SNPs, termed “common_512”, “common_517”, “common_518”, “common_525”, and “rare_57”, found in 4 of the 6 individual F2 populations where a significant association was found. Together these 6 SNPs represent ideal markers that are able to identify both the homozygous allele state that increases the likelihood that a particular plant will display the hermaphroditic phenotype. These 6 markers are equally useful in selecting plants that contain the homozygous allele state with no or a very low chance of the occurrence of hermaphroditic inflorescence. The significance of a SNP marker in the association studies does not indicate the presence or absence of that SNP in the population. In all populations the SNP markers or closely linked markers are present.

For F2 population GID 21 002 003, the SNP markers showing a significant association with hermaphroditism, with an LOD value greater than 5, found on chromosome NC_044370.1 with reference to the Cannabis Sativa CS10 genome are listed in Table 4.

For F2 population GID 21 002 004, the SNP markers showing a significant association with hermaphroditism, with an LOD value greater than 5, found on chromosome NC_044370.1 with reference to the Cannabis Sativa CS10 genome are listed in Table 5. Here while only four SNPs were found associated, they define two QTLs.

For F2 population GID 21 002 014, the SNP markers showing a significant association with hermaphroditism, with an LOD value greater than 5, found on chromosome NC_044370.1 with reference to the Cannabis Sativa CS10 genome are listed in Table 6.

For F2 population GID 21 002 028, the SNP markers showing a significant association with hermaphroditism, with an LOD value greater than 5, found on chromosome NC_044370.1 with reference to the Cannabis Sativa CS10 genome are listed in Table 7.

For F2 population GID 21 002 035, the SNP markers showing a significant association with hermaphroditism, with an LOD value greater than 5, found on chromosome NC_044370.1 with reference to the Cannabis Sativa CS10 genome are listed in Table 8.

For F2 population GID 21 002 038, the SNP markers showing a significant association with hermaphroditism, with an LOD value greater than 5, found on chromosome NC_044370.1 with reference to the Cannabis Sativa CS10 genome are listed in Table 9.

The homozygous allele of the SNPs in Tables 5, 6, 7, 8, and 9 that can distinguish the likelihood that a hermaphroditic inflorescence will emerge during growth are listed along with their position.

In each of Tables 4 to 9 presented in this Example 3 below, the presence of the hermaphroditic inflorescence is predicted by the occurrence of the indicative allele (marked with *). The positions of the SNPs are provided with reference to the CS10 reference genome as described herein. “Homo_1” denotes the average phenotypic value associated with homozygous allele 1 based on a score from 0-1, as described above, where 1 indicates a plant with hermaphroditic inflorescence and 0 indicates one without, “Homo_2” denotes the average phenotypic value associated with homozygous allele 2 based on a score from 0-1, as described above, where 1 indicates a plant with hermaphroditic inflorescence and 0 indicates one without and “Hetero” denotes the average phenotypic value associated with the heterozygous allele state based on a score from 0-1, as described above, where 1 indicates a plant with hermaphroditic inflorescence and 0 indicates one without. BP refers to the nucleotide position of the SNP.

TABLE 4
SNPs associated with hermaphroditic inflorescence in Cannabis
from F2 population GID: 21 002 003 on Chromosome NC_044370.1.

| SNP Marker | BP | LOD | Allele1 | Allele2 | Homo_1 | Hetero | Homo_2 |
|---|---|---|---|---|---|---|---|
| common_525 | 99830512 | 6.315994077 | C * | G | 0.67 | 0.12 | 0.10 |
| common_517 | 99121582 | 6.220406615 | A | G * | 0.12 | 0.12 | 0.66 |
| common_511 | 97379141 | 5.93898384 | A | G * | 0.05 | 0.15 | 0.62 |
| common_512 | 97440573 | 5.891622544 | A | T * | 0.05 | 0.17 | 0.63 |
| common_523 | 99748182 | 5.792407444 | A * | G | 0.60 | 0.12 | 0.11 |
| GBScompat_{—} | 89565596 | 5.405502008 | A * | G | 0.56 | 0.18 | 0.05 |
| common_91 | | | | | | | |
| common_531 | 100304872 | 5.208939775 | A | G * | 0.10 | 0.13 | 0.56 |
| common_500 | 95278820 | 5.170970412 | A | G * | 0.04 | 0.18 | 0.59 |
| common_494 | 94521889 | 5.133641208 | A * | G | 0.57 | 0.19 | 0.04 |
| common_484 | 91453227 | 4.82881167 | A | G * | 0.06 | 0.20 | 0.55 |

TABLE 5
SNPs associated with hermaphroditic inflorescence in Cannabis
from F2 population GID: 21 002 004 on Chromosome NC_044370.1.

| SNP Marker | BP | LOD | Allele1 | Allele2 | Homo_1 | Hetero | Homo_2 |
|---|---|---|---|---|---|---|---|
| common_491 | 94129798 | 6.349096748 | A | G * | 0 | 0.03 | 0.66 |
| rare_50 | 79534090 | 5.767453912 | A | G * | 0 | 0.07 | 0.67 |
| common_497 | 95062684 | 5.676689017 | A | G * | 0 | 0.08 | 0.66 |
| common_532 | 100388338 | 5.021729572 | C * | G | 0.68 | 0.11 | 0 |

TABLE 6
SNPs associated with hermaphroditic inflorescence in Cannabis
from F2 population GID: 21 002 014 on Chromosome NC_044370.1.

| SNP Marker | BP | LOD | Allele1 | Allele2 | Homo_1 | Hetero | Homo_2 |
|---|---|---|---|---|---|---|---|
| rare_57 | 98263682 | 6.63 | C * | G | 0.79 | 0.08 | 0.00 |
| common_497 | 95062684 | 6.50 | A | G * | 0.00 | 0.04 | 0.71 |
| common_491 | 94129798 | 6.18 | A | G * | 0.11 | 0.04 | 0.71 |
| common_532 | 100388338 | 5.82 | C * | G | 0.74 | 0.11 | 0.00 |
| common_518 | 99186615 | 5.76 | A | G * | 0.00 | 0.05 | 0.78 |
| common_531 | 100304872 | 4.88 | A | G * | 0.00 | 0.11 | 0.63 |

TABLE 7
SNPs associated with hermaphroditic inflorescence in Cannabis
from F2 population GID: 21 002 028 on Chromosome NC_044370.1.

| SNP Marker | BP | LOD | Allele1 | Allele2 | Homo_1 | Hetero | Homo_2 |
|---|---|---|---|---|---|---|---|
| common_512 | 97440573 | 10.17989 | A | T * | 0.00 | 0.01 | 0.82 |
| common_517 | 99121582 | 9.9980648 | A | G * | 0.00 | 0.01 | 0.78 |
| common_511 | 97379141 | 9.9831618 | A | G * | 0.00 | 0.01 | 0.78 |
| common_525 | 99830512 | 9.4120983 | C * | G | 0.77 | 0.01 | 0.00 |
| common_518 | 99186615 | 9.1416083 | A | G * | 0.00 | 0.01 | 0.74 |
| common_528 | 100088557 | 8.8877226 | A * | G * | 0.00 | 0.50 | 0.01 |
| common_522 | 99561159 | 8.5214968 | A | G * | 0.00 | 0.01 | 0.73 |
| common_521 | 99499651 | 8.5022921 | C * | G | 0.73 | 0.01 | 0.00 |
| common_526 | 99965933 | 8.3072658 | A | C * | 0.00 | 0.01 | 0.73 |
| rare_63 | 101144766 | 8.198918 | A | T * | 0.00 | 0.03 | 0.68 |
| rare_57 | 98263682 | 8.1612221 | C * | G | 0.67 | 0.04 | 0.00 |
| common_488 | 92041720 | 7.9867277 | A | G * | 0.00 | 0.04 | 0.62 |
| common_491 | 94129798 | 7.9038878 | A | G * | 0.00 | 0.04 | 0.67 |
| common_496 | 94684180 | 7.8273302 | A | G * | 0.00 | 0.03 | 0.64 |
| common_479 | 90357946 | 7.825793 | A | G * | 0.00 | 0.04 | 0.62 |
| common_473 | 89460138 | 7.7791312 | A * | C | 0.62 | 0.04 | 0.00 |
| common_533 | 100626851 | 7.7177988 | A * | C | 0.63 | 0.02 | 0.00 |
| GBScompat_{—} | 89565596 | 7.7084491 | A * | G | 0.62 | 0.04 | 0.00 |
| common_91 | | | | | | | |
| rare_50 | 79534090 | 7.680025 | A | G * | 0.00 | 0.04 | 0.62 |
| common_472 | 89337710 | 7.5776055 | A | G * | 0.00 | 0.04 | 0.62 |
| common_470 | 89227662 | 7.495949 | A * | G | 0.59 | 0.04 | 0.00 |
| common_474 | 89539255 | 7.4869673 | A * | G | 0.62 | 0.04 | 0.00 |
| common_484 | 91453227 | 7.4869673 | A | G * | 0.00 | 0.04 | 0.62 |
| common_492 | 94257304 | 7.4338084 | A * | G | 0.65 | 0.04 | 0.00 |
| common_534 | 100736341 | 7.3968511 | A * | G | 0.59 | 0.02 | 0.00 |
| common_520 | 99379219 | 7.2076383 | A * | T | 0.76 | 0.08 | 0.00 |
| common_545 | 101637128 | 7.0443121 | A | G * | 0.00 | 0.05 | 0.59 |
| common_487 | 91953671 | 7.0214515 | A * | G | 0.58 | 0.04 | 0.00 |
| common_527 | 99976278 | 6.9930683 | A | G * | 0.00 | 0.01 | 0.63 |
| common_476 | 89639165 | 6.9753622 | A | C * | 0.00 | 0.04 | 0.58 |
| GBScompat_{—} | 79818534 | 6.974447 | A * | G | 0.60 | 0.06 | 0.00 |
| common_84 | | | | | | | |
| common_483 | 90999267 | 6.9736607 | A | G * | 0.00 | 0.04 | 0.58 |
| GBScompat_{—} | 101069452 | 6.9590037 | A | G * | 0.00 | 0.03 | 0.63 |
| common_98 | | | | | | | |
| common_489 | 92434073 | 6.9175851 | A * | C | 0.58 | 0.05 | 0.00 |
| common_478 | 89807220 | 6.8841185 | A | C * | 0.00 | 0.04 | 0.61 |
| GBScompat_{—} | 92378793 | 6.8553686 | A * | G | 0.58 | 0.05 | 0.00 |
| common_94 | | | | | | | |
| common_544 | 101523781 | 6.6533006 | A * | G | 0.58 | 0.05 | 0.00 |
| common_452 | 81689735 | 6.6344269 | A | G * | 0.00 | 0.06 | 0.56 |
| GBScompat_{—} | 101752789 | 6.5396364 | A | G * | 0.00 | 0.07 | 0.58 |
| common_99 | | | | | | | |
| common_552 | 102179472 | 6.5090881 | A | T * | 0.00 | 0.06 | 0.58 |
| common_445 | 79634333 | 6.4699565 | A * | G | 0.56 | 0.04 | 0.00 |
| common_539 | 101113276 | 6.4224429 | A * | G | 0.61 | 0.03 | 0.00 |
| GBScompat_{—} | 98559392 | 6.422416 | A | C * | 0.00 | 0.04 | 0.62 |
| common_96 | | | | | | | |
| common_486 | 91877425 | 6.3471412 | A * | G | 0.56 | 0.04 | 0.00 |
| common_546 | 101726389 | 6.3370943 | A * | C | 0.56 | 0.07 | 0.00 |
| common_432 | 70956672 | 6.1907734 | A | C | 0.04 | 0.40 | 0.00 |
| common_449 | 79968518 | 6.1808976 | A * | G | 0.56 | 0.06 | 0.00 |
| common_425 | 70407835 | 5.9928012 | A * | C | 0.54 | 0.06 | 0.00 |
| common_453 | 81762017 | 5.881149 | A * | G | 0.58 | 0.04 | 0.05 |
| common_541 | 101321815 | 5.8621967 | A * | G | 0.59 | 0.05 | 0.00 |
| rare_66 | 102037098 | 5.7869904 | A * | T | 0.60 | 0.11 | 0.00 |
| GBScompat_{—} | 100988215 | 5.7831367 | A | G * | 0.00 | 0.03 | 0.57 |
| common_97 | | | | | | | |
| common_455 | 81901321 | 5.760267 | C * | G | 0.56 | 0.04 | 0.05 |
| common_428 | 70624527 | 5.6943233 | A * | G | 0.56 | 0.04 | 0.05 |
| common_426 | 70445826 | 5.6341286 | C * | G | 0.56 | 0.03 | 0.07 |
| common_553 | 102227038 | 5.6149247 | A * | G | 0.54 | 0.07 | 0.00 |
| GBScompat_{—} | 75985317 | 5.5856371 | A | T * | 0.00 | 0.06 | 0.50 |
| rare_10 | | | | | | | |
| GBScompat_{—} | 70516405 | 5.5418631 | A | C * | 0.06 | 0.04 | 0.56 |
| common_79 | | | | | | | |
| common_431 | 70913971 | 5.5281841 | C * | G | 0.54 | 0.04 | 0.05 |
| common_439 | 72435815 | 5.4974102 | C * | G | 0.52 | 0.04 | 0.00 |
| common_437 | 71800647 | 5.419316 | A | C * | 0.00 | 0.04 | 0.52 |
| common_313 | 34512948 | 5.3609989 | A | G * | 0.00 | 0.06 | 0.48 |
| common_375 | 57469730 | 5.2550177 | A * | G | 0.54 | 0.04 | 0.05 |
| common_454 | 81831758 | 5.2448814 | C | G * | 0.05 | 0.04 | 0.56 |
| common_436 | 71705041 | 5.1968557 | A * | G | 0.54 | 0.04 | 0.05 |
| common_288 | 30449533 | 5.1792437 | A * | G | 0.52 | 0.03 | 0.00 |
| common_448 | 79909671 | 5.0247419 | C * | G | 0.52 | 0.06 | 0.00 |
| common_424 | 70255045 | 4.9725568 | A | G * | 0.05 | 0.04 | 0.52 |
| common_434 | 71490671 | 4.9725568 | A | C * | 0.05 | 0.04 | 0.52 |
| common_514 | 98401235 | 4.9531923 | A * | G | 0.55 | 0.03 | 0.00 |
| common_459 | 82560029 | 4.9431435 | C | G * | 0.05 | 0.04 | 0.52 |

TABLE 8
SNPs associated with hermaphroditic inflorescence in Cannabis
from F2 population GID: 21 002 035 on Chromosome NC_044370.1.

| SNP Marker | BP | LOD | Allele1 | Allele2 | Homo_1 | Hetero | Homo_2 |
|---|---|---|---|---|---|---|---|
| common_522 | 99561159 | 7.078061636 | A | G * | 0.00 | 0.07 | 0.56 |
| common_517 | 99121582 | 6.585512305 | A | G * | 0.00 | 0.09 | 0.56 |
| common_525 | 99830512 | 6.199958929 | C * | G | 0.57 | 0.10 | 0.00 |
| common_491 | 94129798 | 6.106741417 | A | G * | 0.00 | 0.10 | 0.57 |
| common_544 | 101523781 | 5.877345351 | A * | G | 0.52 | 0.09 | 0.00 |
| common_510 | 97283326 | 5.8642263 | C * | G | 0.51 | 0.08 | 0.00 |
| common_512 | 97440573 | 5.77051714 | A | T * | 0.00 | 0.10 | 0.53 |
| common_518 | 99186615 | 5.506996042 | A | G * | 0.00 | 0.07 | 0.53 |
| rare_57 | 98263682 | 5.415374722 | C * | G | 0.56 | 0.12 | 0.00 |
| common_550 | 102006836 | 5.41426113 | A * | C | 0.50 | 0.10 | 0.00 |
| common_545 | 101637128 | 5.338539557 | A | G * | 0.02 | 0.09 | 0.53 |
| common_483 | 90999267 | 5.277138081 | A | G * | 0.00 | 0.09 | 0.52 |
| common_539 | 101113276 | 5.213997161 | A * | G | 0.52 | 0.11 | 0.00 |
| common_534 | 100736341 | 5.17925627 | A * | G | 0.50 | 0.11 | 0.00 |
| common_549 | 101942035 | 5.088653633 | A | T * | 0.02 | 0.11 | 0.50 |
| common_515 | 98481067 | 4.993112835 | A | G * | 0.00 | 0.09 | 0.56 |
| common_533 | 100626851 | 4.948369261 | A * | C | 0.50 | 0.12 | 0.00 |
| common_546 | 101726389 | 4.943779589 | A * | C | 0.52 | 0.09 | 0.02 |

TABLE 9
SNPs associated with hermaphroditic inflorescence in Cannabis
from F2 population GID: 21 002 038 on Chromosome NC_044370.1.

| SNP Marker | Position | LOD | Allele1 | Allele2 | Homo_1 | Hetero | Homo_2 |
|---|---|---|---|---|---|---|---|
| common_533 | 100626851 | 8.509493763 | A * | C | 0.72 | 0.07 | 0.00 |
| common_517 | 99121582 | 8.026552008 | A | G * | 0.00 | 0.05 | 0.65 |
| common_532 | 100388338 | 7.455291164 | C * | G | 0.73 | 0.10 | 0.00 |
| common_511 | 97379141 | 7.398320886 | A | G * | 0.00 | 0.09 | 0.63 |
| common_534 | 100736341 | 6.926696529 | A * | G | 0.72 | 0.05 | 0.00 |
| common_545 | 101637128 | 6.815330759 | A | G * | 0.00 | 0.07 | 0.65 |
| common_527 | 99976278 | 6.738436035 | A | G * | 0.00 | 0.04 | 0.68 |
| common_512 | 97440573 | 6.589382242 | A | T * | 0.00 | 0.08 | 0.59 |
| common_525 | 99830512 | 6.386528883 | C * | G | 0.67 | 0.10 | 0.00 |
| common_521 | 99499651 | 6.376695383 | C * | G | 0.62 | 0.02 | 0.00 |
| GBScompat_{—} | 101752789 | 6.235536438 | A | G * | 0.00 | 0.07 | 0.63 |
| common_99 | | | | | | | |
| common_539 | 101113276 | 6.172028988 | A * | G | 0.67 | 0.07 | 0.00 |
| common_491 | 94129798 | 6.156854894 | A | G * | 0.00 | 0.10 | 0.63 |
| common_546 | 101726389 | 5.978964179 | A * | C | 0.59 | 0.08 | 0.00 |
| common_518 | 99186615 | 5.828160881 | A | G * | 0.00 | 0.04 | 0.67 |
| common_522 | 99561159 | 5.612304618 | A | G * | 0.00 | 0.02 | 0.62 |
| rare_57 | 98263682 | 5.566979184 | C * | G | 0.65 | 0.15 | 0.00 |
| common_553 | 102227038 | 5.518969152 | A * | G | 0.60 | 0.09 | 0.00 |
| common_486 | 91877425 | 5.416506194 | A * | G | 0.64 | 0.11 | 0.00 |
| common_474 | 89539255 | 5.299968402 | A * | G | 0.61 | 0.12 | 0.05 |
| common_544 | 101523781 | 5.293858536 | A * | G | 0.61 | 0.09 | 0.00 |
| GBScompat_{—} | 75985317 | 5.158044141 | A | T * | 0.05 | 0.11 | 0.59 |
| rare_10 | | | | | | | |
| rare_50 | 79534090 | 5.12903219 | A | G * | 0.04 | 0.13 | 0.59 |
| common_514 | 98401235 | 5.119430229 | A * | G | 0.67 | 0.11 | 0.00 |
| common_472 | 89337710 | 4.989467159 | A | G * | 0.05 | 0.10 | 0.59 |
| common_203 | 20652741 | 4.976037505 | A | T * | 0.06 | 0.11 | 0.59 |
| common_552 | 102179472 | 4.915029864 | A | T * | 0.00 | 0.13 | 0.59 |
| common_300 | 32721609 | 4.902771546 | A | C * | 0.05 | 0.13 | 0.59 |

### Example 4

### Validation of SNP Markers for Hermaphroditic Inflorescence in Cannabis

The inventors sought to validate the QTLs identified and the broad use of the SNP markers found to distinguish allele states in Cannabis that determine the likelihood that a plant will produce hermaphroditic inflorescences. Representative markers of the four QTLs identified were selected based on the strength of their association with hermaphroditic inflorescence and their commonality in multiple individual F2 populations, Table 10.

The mixed F2 population was used, plants were filtered for those having a genotype call at the markers tested. After filtering, the average phenotypic value associated with each allele state based on a score from 0-1, where 1 indicates a plant with hermaphroditic inflorescence and 0 indicates one without, was calculated (Table 10).

The findings in Table 10, support the broad applicability of these markers, and those in linkage disequilibrium to them, and support the QTLs identified. Specifically, the inventors have provided the allelic state for each SNP that can be used to distinguish plants with a high likelihood of being hermaphroditic. Even though in some of the individual F2 populations a marker in Table 10 was not found, based on this experiment all of the markers show the ability to distinguish the allelic states for the hermaphroditic phenotype in the mixed population. SNP marker “common_491” performed the best at predicting the presence of hermaphroditic plants based on the allelic states in Table 10. On average only 1% of plants in the mixed population of F2 plants examined were found to be hermaphroditic when the homozygous allele state of SNP marker “common_491” is AA. This contrasts with the homozygous allele state for this SNP of GG where on average half of the plants having this marker state are hermaphroditic. There may be other genetic and environmental factors influencing hermaphroditism that are not under the control of the QTLs found here which can explain some of the variation.

The inventors also looked at pairwise combinations of the SNPs that comprise independent QTLs to determine if combinatorial allelic states had an effect on the expected phenotypic outcome. They found no SNP combinations that altered the expected phenotypic outcome as compared with a single SNP alone. There may be combinatorial effects related to these loci however they may be masked by the variable stress induced nature of the hermaphroditic phenotype.

The reference sequences for each of the markers identified in Tables 1 and 3-10 are provided in Table 11 below. The reference or context sequence is based on the CS10 reference genome. The alternative allele “Alt” in Table 11 indicates plants that have 0 or close to 0 likelihood of producing hermaphroditic inflorescence.

Targeted sequencing primers for each of the markers identified in Tables 1 and 3-10 are provided in Table 12. The context sequence and locations are provided with reference to the CS10 reference genome for cannabis.

TABLE 10
Validation of SNPs associated with hermaphroditic inflorescence in Cannabis from
all F2 populations on Chromosome NC_044370.1. The presence of the hermaphroditic
inflorescence is predicted by the occurrence of the indicative allele (marked with *).
The positions of the SNPs are provided with reference to the CS10 reference genome
as described herein. “Homo_1” denotes the average phenotypic value associated
with homozygous allele 1 based on a score from 0-1, as described above, where 1
indicates a plant with hermaphroditic inflorescence and 0 indicates one without,
“Homo_2” denotes the average phenotypic value associated with homozygous allele
2 based on a score from 0-1, as described above, where 1 indicates a plant with
hermaphroditic inflorescence and 0 indicates one without and “Hetero” denotes
the average phenotypic value associated with the heterozygous allele state based on
a score from 0-1, as described above, where 1 indicates a plant with hermaphroditic
inflorescence and 0 indicates one without. BP refers to the nucleotide position of the SNP.

| SNP Marker | BP | LOD | Allele1 | Allele2 | Homo_1 | Hetero | Homo_2 |
|---|---|---|---|---|---|---|---|
| common_491 | 94129798 | 9.130452 | A | G * | 0.01 | 0.08 | 0.5 |
| common_517 | 99121582 | 7.410864 | A | G * | 0.02 | 0.07 | 0.32 |
| common_518 | 99186615 | 7.227338 | A | G * | 0.01 | 0.04 | 0.26 |
| rare_50 | 79534090 | 6.695617 | A | G * | 0.01 | 0.09 | 0.48 |
| common_534 | 100736341 | 5.744335 | A * | G | 0.25 | 0.06 | 0.01 |
| common_525 | 99830512 | 5.541966 | C * | G | 0.31 | 0.08 | 0.01 |
| common_511 | 97379141 | 5.357322 | A | G * | 0.01 | 0.08 | 0.26 |
| GBScompat_{—} | 35677966 | 5.241665 | A | G * | 0.05 | 0.12 | 0.23 |
| common_56 | | | | | | | |
| common_546 | 101726389 | 5.172549 | A * | C | 0.21 | 0.12 | 0.03 |

TABLE 11
Detailed information of each of the SNPs associated with hermaphroditic inflorescence
in Cannabis as provided in Tables 1 and 3 to 10. The reference allele “Ref” is the allele that is
associated with hermaphroditism, while the alternative allele “Alt” indicates the allele that when
homozygous for that allele results in plants that have 0 or close to 0 likelihood of producing
hermaphroditic inflorescence. The positions of the SNPs as well as context sequences are
provided with reference to the CS10 reference genome as described herein. BP refers to the
nucleotide position of the SNP. All of the sequences and alleles are provided with reference to
the plus strand.

| SNP Marker | Chromosome | BP | Ref | Alt | Context sequence |
|---|---|---|---|---|---|
| common_491 | NC_044370.1 | 94129798 | T | C | TTGATTGGAGAAAGGTCATAAAAGAAAAATACACCAGGAAGAAACTGAAGATTCCCAAA |
| | | | | | TTCTGAAGTCCTAAAATGCTCAGTTACAGAAAACTGCAATATGCAATTGGCTAAAGTCAT |
| | | | | | GCTCCATGTTTCATAAATGAAATAAACCAGATAGTAAATATATGCATACGTAAAGAGATA |
| | | | | | ACCACCTGATTAGACTGAATAG[T/C]GTGTCCAGTCACGTCTGTGTAAACAGTAGGTACC |
| | | | | | ACCTAAAACATAAACAGCAACCCTTGATCTGAGAGCTAATAGATTTCCCTTATATCGTCT |
| | | | | | AGTATTAGGTATCAATTTGTGCGTGTGTTTTCAAAGAATGAATCCAAAGGACTTACCTTG |
| | | | | | ATAAAATACTGATACATCCCACTAGGTGTGGCCTGCGACCATTGCAC (SEQ ID NO: 1) |
| common_512 | NC_044370.1 | 97440573 | T | A | TTGGGGAAAAGAGAAAACTGGTAATTTTCATCAACAAATCTCTGAATGTAAGAGCTTGAT |
| | | | | | TCGGAGACTTAAAGGGAAGAATGATGAAGTGTCTGTCGAGGCCCATCTCAAAGCGGAA |
| | | | | | AGAGATCTTTTTGAAGTGCTTACTAAAAGGGAAGTGTTTGGGAAACAACGCTCCAAGCA |
| | | | | | ACTTTGGCTCAAAGAAGGAGATAG[T/A]AATAGTAAGTATTTCCATGTTTTTGCGAGTGC |
| | | | | | TCGTAAACGCCAAAATACGATTCATAAGCTTCAAGATTACAATGGGAGTTGGCTTGATT |
| | | | | | GGGAGCTCCGCTCATGATCATATGGTCGCTTCTAATCGAAGTTATTTTCACTTCTGCAC |
| | | | | | TTCATACCGATATTCTTGATGGTATTGCCTCGCTATTACTTCTTGAAATGA (SEQ ID |
| | | | | | NO: 2) |
| common_517 | NC_044370.1 | 99121582 | T | C | TGTGTGTTAGATCACAAAGACCAACCGTACGAGCTGTTACAGATTTGCATTGAAGATCA |
| | | | | | ATGCCTTATCTACAAATTAGATAGCATTGGCCAAAAATTCAACCCAAAAGTGCTGAAGG |
| | | | | | ATTTGCTTCACGACAGCCGCGTGACGGTGGTCGGAGTGGGTATAGAGAATGTGGCGA |
| | | | | | GGCGGTTGGAGGAGGATCACGGTTGG[T/C]TCATGCCTAAAGTGGTTGAACTGCGAGA |
| | | | | | TTTGGCGGCCCAGGCCCAGGCCCAAACCAAGGCCCAAAAGGCAGATATTCAGGGTAG |
| | | | | | TACTAGCGGTCATGATAATGGAAAGAAGATCATGACGAAGAAGGTGATGATGATGAAAA |
| | | | | | GAAACTTTAGCCGATTTGGTATAGCGAAGCTGGCGAAAGTGGTTTTGGGAAAAGAGA |
| | | | | | (SEQ ID NO: 3) |
| common_525 | NC_044370.1 | 99830512 | C | G | TCTGCCGTGATATTTTTGAAATTTTAGGTTTTGTTTCCGTAAATTTGATCGTTGTACATAA |
| | | | | | TTTGTTAGCTTAAAGTTTATGATTTTTTGCTGAACAAGCTTGGGTGATACAGCTGCTGCA |
| | | | | | GCGAATATGGATGAGTTTGGTGTATTGACGGAGAGGTTTGGATTGAAGCCTCAAGGTA |
| | | | | | AATCGGCTCCAATGGCGGCTTC[C/G]AAGAGATCTGTCAATAGTAACGATGGCCAGAAT |
| | | | | | TGGAATTCGGGGTTTGATTCGGGTGTAAACCCCAACTTTTCATCTTTTTCAGACGATCG |
| | | | | | GAACGGTTTTTATCAATCCGGCAAAGATAAGGTAGCTCAGAACAGCGGAAGCTTGAATA |
| | | | | | TTATGATGATATATTTGGTGGACCTAGTAAGCAATCGGGTAGTCATGGT (SEQ ID NO: 4) |
| common_511 | NC_044370.1 | 97379141 | A | G | ATTTGTTACTTCTTCGTGTCTTGATGCCAAGATAGCGAAAAAGTACATAGATCATTCGTT |
| | | | | | CATTTTGAGATTGCTAGATTTGCTTGATTCCGAGGATCCTAGAGAAAGAGACTGCTTAA |
| | | | | | AGACTATTCTTCATAGGACCTATGGGAAATTTATGGTTCATAGGCCATTTATAAGGAAGT |
| | | | | | CTATAAACAATGTATTTTACCG[A/G]TTTGTGTTTGAGACTGAGAAACATAATGGGATTGC |
| | | | | | TGAGGTATTGGAGATCTATGGGAGTATCATTAGTGGATTTGCATTACCTTTAAAGGAGG |
| | | | | | AGCATAAGATATTCTTGCGGAGGGTTTTGATTCCTCTGCATAGGCCAAAGTCTTTGGGG |
| | | | | | GTTTACTTCCAACAACTCTCCTACTGTGTTACACAATTTATAGAGAAG (SEQ ID NO: 5) |
| common_518 | NC_044370.1 | 99186615 | C | T | CCCATATGATACATATGAATGTAAAACACTTACCCGCTCTTCAGGAAAGGAACTTGAGA |
| | | | | | ATCCGAATATTTTACCAGAAAATACATTTGAAGTTTTTCCGTTTTGAGTGCTTGAACCAA |
| | | | | | GTTGCACTCTCTTTGGAGCTTTATTCTTATCACTTACTCGACCCTTACTTTTTTTATTGAC |
| | | | | | AGCAGTAACGTTACTCTGCTG[C/T]GATGATTTTGCTGCAGTTGCCATAATGTTGACATC |
| | | | | | AGTCGTGTTGATTTCTGGTTTTTTTCCTTTGCTTCCGCCCAATGGTAATGGCATTCCAAT |
| | | | | | TCCACCTCTTATGCTACCAGATAAAGGATTAGATGGCATGATCGGATGAACAGCAGAGA |
| | | | | | CTCTAGCTTGATTTAGACTGGTCAATCCTGTAATTGCTCCTTCAAGA (SEQ ID NO: 6) |
| common_522 | NC_044370.1 | 99561159 | C | T | TCGCGATTGTTATTCCCACCTCGTCTTTTACTTCGAATCTCAGTGTCTCGTAGAAGTTTA |
| | | | | | TTAGAGCTGCCTTCGCCGCCTACACATTAGTGTCTCGTAAATATCAAGTGATGATATGTT |
| | | | | | TTATTGGCTTAAGCAACACATATGTTTTAATCAATTACTCACAGCATATAAGCTCATTCTA |
| | | | | | GGCAGAGGTAACCAGTTCTC[C/T]ACTGAGGCATTAACTATGACTCTACCATTGCTTTGG |
| | | | | | TATAAGTAAGGAAGAGCCACAAATGTTGGATAAACATTTCCCCAAAAGTTAATGTCCTGA |
| | | | | | AAACCCAAAAAGATAAATTACAAAAATTAAGAGTGTTCACGGATTGGTTTAGTCTAGATT |
| | | | | | TTTAATTTTTTCAAAATAAACCACTATTACGGTTTTTTAAAAATT (SEQ ID NO: 7) |
| common_533 | NC_044370.1 | 100626851 | C | A | TTTCCAAGTGTATACGAAACTTCGCACTCTTATCAATGTTGTGGTCCATTGTTATCTAAA |
| | | | | | CGTTTATCATGTTGTGCTTTTGCAGTACTATTCTTTGGATATTACATATTTTAAGTCCTCT |
| | | | | | CTTGATAGCCATCTACTGGATCTGCTATGGAACAAGTACTGGGTGAATACACTTTCTTCT |
| | | | | | TCACCCCTACTGGGTAATGG[C/A]GATTATGTTGCAGGACAAATTTCAGACTTAGGTACC |
| | | | | | TAAATTGTTATGATACCAGATTTTTTTTTTAAATGCTCGTCTAGCTTTCTGTAATGTTTATC |
| | | | | | ATGGTAATAAAATCATTAACTGTGTTGTACTTTTTATCATTTATGCAGCTGAAAAGTTGGA |
| | | | | | GCAAGCAGAGAACCAATTGTCACATTCCCGCTTTGGGCCATT (SEQ ID NO: 8) |
| common_534 | NC_044370.1 | 100736341 | A | G | TTGACTTCATAACTTATTAGTAACTGATGTTATTTCTTCACTTCCAACATCTTGTGGTTGC |
| | | | | | TTATTATGATGGTGTGGTTTCTTTCTGTCCTAACAGGAGAGCAAGATTCTGAAGTTTTTA |
| | | | | | GGCTTTATGTGGAATCCTTTGTCCTGGGTCATGGAAGCTGCTGCTATAATGGCTATTGC |
| | | | | | CTTGGCAAATGGTGGTGGAAG[A/G]CCTCCGGATTGGCAGGACTTTGTTGGTATTATTG |
| | | | | | TCCTGCTGATCATCAACTCAACTATCAGTTTTATTGAAGAAAACAATGCTGGTAATGCAG |
| | | | | | CAGCTGCTCTCATGGCTGGTCTTGCTCCAAAGACTAAGGTAAAACAACAGCCTCACCAT |
| | | | | | CACCATCAGACTTGTTTGGTTTTTAGCTTTTATGGTATTTTTTTTTAA (SEQ ID NO: 9) |
| common_539 | NC_044370.1 | 101113276 | A | G | CTACTCTCTCTTCTCCATTTCCATCTCCAGCTCCAGTTCTAGTTCCATCTCTTTCTAGATA |
| | | | | | CTCAACTGTATACATATATCATCACAAATTTATATCTTAAATCAAAACCATGAAACTTTAA |
| | | | | | TGGCTATGATTCAGCTTCAGAGGGGTTTGTGTTCGGCCACCTCAACCACAACCACAAC |
| | | | | | CACAGTTTCTGCTTCTTTCTC[A/G]AACCCTTCTACTTCATCAGCTCTGCAACTCCGCCC |
| | | | | | TTTCATGGCTAAGGTTCTTAATCTCCATTTCATTATCTTCTTCCTGCTTTOTTAAATTTATT |
| | | | | | ATGGTTTTCATTATTATATTAAACTTCTTCTCTTCAATACTTAGCTATAACAATTTT TTTT |
| | | | | | CGGCTGCTGTCGTAACACTCCTCTAATGAGGAACTTTACAT (SEQ ID NO: 10) |
| common_544 | NC_044370.1 | 101523781 | A | G | TACAATTACATTTACAAATCCCATAAAGGCCTAAACAAGGCCATGATGAAACGACGTCG |
| | | | | | TCGTCGTTGATGGGTAAAACGCTTCTCGAAGCTTGTTGTACTTTTGTATATCAAAGCTAT |
| | | | | | GAATCTCCATGAGCCGTTTTTGCCTGGAGAGAAAGCGGCGTTGGAAGTAGCCTTGGAA |
| | | | | | AGTTGGCTCCGTCGAGGTACGGAG[A/G]AAGAAGCCGTAGCCCAAAGCGAAATGGATC |
| | | | | | GATGTCACAAAGAAGCATATGGGCTCCATCACGTCCCAACTCAGCTCCCAAAAGGTTA |
| | | | | | GTCTCATGGCCGCAAGTGTTTGGGCCAGTACGAAGCCCAGCCCAATATAAAGCTCCCC |
| | | | | | ACGGACCAATTCTCTTGCTTTCTGGTCGATCATCGATTTTTGTTTCTCCATTTCC (SEQ |
| | | | | | ID NO: 11) |
| common_545 | NC_044370.1 | 101637128 | A | G | TCTTCAAACAGTTTCCTGCCATAAATGAAAAAATGTTTGAAAAGAGAAAAACTATGTTTA |
| | | | | | ATTTTGTATCTTTACCAAAACAAATGAGTAAATAGATACCTGAACAAGTCTCTCTTTTTTA |
| | | | | | AAATTGCTGGCCAAGTGAGTTCTGCTAAAGCTTGTGAAAAGACAAGCAATTCAAACAAC |
| | | | | | TTGTTATCCTCGTGGACTGGA[A/G]CTCCCCATTCTTTATCGTGGAATAAAGTATAAATTG |
| | | | | | GGTCTGTGATTCATTACAAAAAGAAAAGGAAAAAAAAGATCGATCGTGTAAGCATTATAA |
| | | | | | ATTATAATTGAACCACCCCGACAAGGTCAAACGTGCCATTTTTGGACAAACATAAGACA |
| | | | | | TGAGCCACGAATATCTCATTACAATTATCTATTCAACAAGTTACAT (SEQ ID NO: 12) |
| common_546 | NC_044370.1 | 101726389 | C | A | ATCCAATTAGTAATCCGTGCCCTTGGTAAAGAGAAGTTTCAGTAACGCCAAAGATCCTC |
| | | | | | AAAGTATATACATCAGGTACAGAGAGGAACAAAAGAGTTGTCATTTGTCACTAGAACCA |
| | | | | | GCCACATACGAAACAACGACTGTGATATCATCAAGCTTTCCCCCATAGTAGCGGAAACC |
| | | | | | AGCATCTTGAGCAGCTGTGGAAAA[C/A]GGTGTCTGCCTGTCCTTGTCCTGTGCTCGTT |
| | | | | | GACGCGCCAATGCTGCTATTTTTTGAGCTGTTGCCTGAGGCCCTAAGCCAGCTCGCAT |
| | | | | | GGCATGCACTACTACTGCCGTTATCTCATTGTTGTACAAGTTGTCAAAGAGTCCATCAG |
| | | | | | TCCCAGCAATGATGACATCCCCGGGAGCTACAGGTACTGTAAAAACCTGAATC (SEQ ID |
| | | | | | NO: 13) |
| rare_50 | NC_044370.1 | 79534090 | A | G | CATCCACTTCGAACCGTACTTTGCCACACAGTAAGCGTCCGTACACCCTTTACCGTCGA |
| | | | | | TGGTCTTGACCGGAACAAGATTCTTTGCCCGAATGATACCGAGTTCCACTGTGCCAACA |
| | | | | | GGAGGCTTCCAGAGTTGCCTAGCCGTGGGGCGGTAGTCGCTGCTCATGTGTGCAACC |
| | | | | | TCATCCATCACGTGATATCCGCCATC[A/G]AAACAGAGCCTTAGGTGAACACGTCCGTTA |
| | | | | | TAAGTTCTTCTTTTCTCATCCTGAATATTTTCAAGGCTGAGCCACCGTGACGCTACTTTT |
| | | | | | CTGTCATCCACTCTCCTCTCTACAACAGTAAGAGGAATTATTGCCGTTCCCAATGTAGTT |
| | | | | | TGTCCCTTTTGATGTCGTATCTCAAGAGTGATTACAATGTGTTCAGAGAAA (SEQ ID |
| | | | | | NO: 14) |
| rare_57 | NC_044370.1 | 98263682 | G | C | AAATGGAGGGCTCTTTTAGCAGGGGCTATAGCTGGACCTTCCATGCTTCTTACTGGAGT |
| | | | | | TAATACGCAGCACACTAGTTTGGCTATTTACATACTTATGCGAGCTGCGGTTTTAGCGT |
| | | | | | CGCGTTGTGGGATTAAAAGCAAACGGTTTGGGAGATATTGTAAGCCACTCACATGGGC |
| | | | | | TCATGGCGACATTTTCCTTATGTGT[G/C]TCTCTTCTTCCCAAATTCTGTACGTATATCTT |
| | | | | | TTCAAATTGGATTGTTATGTTTTCCTACATCTAACTTTGTTTTGTTTTTATTTCTCTCTTAG |
| | | | | | TTTCGTTTTGTATTCTTTCTAATTGGGGATTCGCAAAATGTATTTGGTTTATGGGCATTGT |
| | | | | | ATTGAATTTTCGAGTTTGGCTTGTTCCCCTGTCTTTATGGGCGTGT (SEQ ID NO: 15) |
| common_472 | NC_044370.1 | 89337710 | T | C | ACATTGTAAATCCAAAAATTCAATTGCAACCCTGTTTAAGCAAGTAAATTTCAACTGATTT |
| | | | | | AATCAAGATTTTCTGAGCTATAAACGCAGTTCCCTTATTCAAAAAGTCAAATTTCCTCTTA |
| | | | | | ACATTTTAATTTGTCATAAATCCACAGTTTCTGAATTGGGTCCGGAAGATACAAGCAAGA |
| | | | | | ACATAGTAGAGATCATATT[T/C]CAATCAAGCTGGCTCAAAAAGGAAGCCCCAATATGCA |
| | | | | | AAATCGACCGAATACTGAAAGTTCACAATACCCAGAAAACCATATCCAAGTTCGAGGAG |
| | | | | | TATCGAGAAGCCATTAAAGCCAAGGCCACCAAGCTCCCAAAGAAGCACCCTCGATGCA |
| | | | | | TCGCCGACGGGAACGAGCTCCTCCGCTTCCATTGCGCCACCTTCATG (SEQ ID NO: 16) |
| common_474 | NC_044370.1 | 89539255 | C | T | TATATATACATATATATAATCATTTCTATCTTTCTTCATCATCATCGTCATCAGTTTCTCTG |
| | | | | | TCTGCCGAAAAATCCGTTTGTTTTTTCTGTAAATTGATTAGATGGGAAAGGACGACTACA |
| | | | | | ACAATTACGATGAGAGAGACAGAGGGCTCTTCTCTCATCTTGGTAGTGCATACTATTCC |
| | | | | | CAGCAGCACCACTCTGGATC[C/T]TACCCTCCGCCCCCGCCGTCTGCATATCCACCGCC |
| | | | | | GCCTCATCATCATCAAGCCTACCCTCCTCCACCCGGCTACCCATCTGCTGGATACCCTC |
| | | | | | CTCCGTCGCCTCACGCCGCATACCCACCGCCCTATACTCCTCCCGGTGGATACCCTCC |
| | | | | | TTCCGCTTATCCTCATCCTCCTCCTCCTGCTCCATATCCATATCCTGCA (SEQ ID NO: 17) |
| common_483 | NC_044370.1 | 90999267 | G | A | TTTCACCATTCCTGTCCTCTTGCTCAATTTCCACTACAACCATCCTCCCTCCATTCCGAT |
| | | | | | CATAATAACACCCACATCCTCATTCGTCGTAAAGCAGCCTCATCCAACGGTCGACATTT |
| | | | | | CACAGCTCGCTCTTCTAGCCCGGACTCAATCCCTCCTTCTCCTTCTCCTCCTCCTTCTT |
| | | | | | CCTCCATTACTCAACAGGTCATT[G/A]TCAGCTCAGCGTTGACCATCGCATTCGCCGTTG |
| | | | | | CCAACCGCGTTCTTTATAAGCTAGCTCTCGTTCCCATGAAAGAATATCCTTTCTTTTTAG |
| | | | | | CTCAGTTGGCCACTTTTGGGTGAGTAAGTAGTGTTCATTAATTTATAAAGAAAATAAAAA |
| | | | | | ATAAAAATTTCGGACTCAATGATGATGATTAATAAATAAAATCTCCCT (SEQ ID NO: 18) |
| common_484 | NC_044370.1 | 91453227 | T | C | AATACGTCAACTTGCATAACACGATAATACAAATTGCCACCTTTATGAGAGGTGATCCAC |
| | | | | | AGGCAGCACGTTTCTATGTCCTTTAAGGGCTACAAAATTATCATTTTGATTCAATAGTGT |
| | | | | | TTTCAAAGTTGAAAGACATTGATATTGTTTACCCCCCCATGGTTTTGTGCAGTTGTTGGA |
| | | | | | ATTTGACGAGTCAAAAGCCTC[T/C]CAAGAAGATGAAGATGACAAAGTTAGCAGCAAGA |
| | | | | | AGGAGGAATAGTTTGAGAAGCTCATAACCTAAAACTTCCCTTTTTAGATGAATACTTTTT |
| | | | | | TTTTTTTATTATCAACTTTCTTCTTTTTCTTATTTCCAAGGTCCCAAAACGGCCTTAGAGA |
| | | | | | ACGCATTTTGGCTAATTCTTATGCTGCTCCACACCCCATGTAACAG (SEQ ID NO: 19) |
| common_486 | NC_044370.1 | 91877425 | T | C | CTGTCTAGACCTGACATCACCGTTGCTGTCAACTGTCTCAGCCAGTTTATGGCCAATCC |
| | | | | | ATAGACAACTCATCTCCAAGCAGTTCATCATCTACTACACTACCTTAAAGAAAATCCTGG |
| | | | | | TCAAGGGCTCTTCTATTCACCTACTTCAGCCTTTACACTACGCGGTTTTTCTGACTTTGA |
| | | | | | TTGGGCATCTTGCCCTGTTACT[T/C]GATGTTCAACAACAGGGTATTGTATCTTCCTCGG |
| | | | | | AGACAGTCTCATTTCTTGGAGAGCAAAGAAGCAGTCAATAATATCCAAAAGCTCTGCAG |
| | | | | | AAGTCGAGTATAGAGCCTTGGATGCAACAACAAGTAAAATGACATGGCTGCAATATCTT |
| | | | | | CTCCATGACTTCCAGATACCACAGCCACATCTTGCCTTTATTTACTGTG (SEQ ID NO: 20) |
| common_514 | NC_044370.1 | 98401235 | A | G | GAAGACAGATTTTCAATTTGCAAGCTCAGAGGGCGGTTAGTGGTGGATTAAGGAGATG |
| | | | | | TCAGGAGGTGGATGTAGCATAGTATGGTTCAGGAGAGATCTAAGGGTAGAAGATAATC |
| | | | | | CAGCTTTAGCAGCTGGTGTTAGAGCAGGTGCTGTGATTGCTGTCTTTGTCTGGGCACC |
| | | | | | CGAGGAAGAAGGTCATTACTATCCTGG[A/G]AGGGTATCAAGATGGTGGCTTAAGCAAA |
| | | | | | GTTTGGCTCATCTTGATTTCTCTTTGCGAAGTTTTGGCACTTCTCTCATCACCAAAAGAT |
| | | | | | CTGCTGATAGTGTTTCTTCTCTTCTTGAGGTTGTCAAGTCCACTGGTGCCAAACAAATAC |
| | | | | | TCTTCAACCACTTATATGGTCAGTATTTTCTTTCGTTATATATTTTTCACAGC (SEQ ID |
| | | | | | NO: 21) |
| common_521 | NC_044370.1 | 99499651 | C | G | AACAAAAAAAGTTATCTCTAGTCAACATAAGAAAGAGCAACGACAACAGCTCTTATCTTA |
| | | | | | GCATAAAAAGAACATAAGCCACTACCCTACTTCCTTGGGCGAGTGTCCTCAGACTCGG |
| | | | | | CCATGCCGGCAGCGACCTTGGCTATATCTTTGGCAGTGGCATCAGGCTTTTTGGTAGT |
| | | | | | GTATAGTGTGAAATATCCAATGGTA[C/G]CGATGATGAGTAAGCCACCAATGGCCATGAT |
| | | | | | GGCTGGGCTGTAGGGAAGCTTCCTTCTCTGATGGAGGACACCCCCAGGATTGTGGCCT |
| | | | | | GGTGGCCTCTGAGACCCTTCTACTCCAGCTGCTTTTATTTCCTCTTGACTCATTTTGTGG |
| | | | | | GTGTCTGCCATTTTCCTCCACTCTTCGTTCTTTTCCATTTTCCTTTCCTAGA (SEQ ID |
| | | | | | NO: 22) |
| common_527 | NC_044370.1 | 99976278 | G | A | GAAATACAAGCCAAACCCCATAAAATATTCTCAATCAAATAAGTAGTTTCATCTTCATTG |
| | | | | | GGAGGCAGCAGCAGCAGCAACAGCAGAATCGTTGGGTTTGGGCAAAGGGGTATTAAC |
| | | | | | TTGGATTCTACCATCAATACAGGAAGAGCACTTGGTTTCAGCCCATGTCTCCAAGTCAA |
| | | | | | AATTTCCATAGCCCATGATTCTCTT[G/A]CCTGAGCACAACCGACCCACCATTACTGCCA |
| | | | | | TTGCTCCTAAGATGAGAATAGCAACTAAGACACCAACAACAGGACCAACAGAAGATGAA |
| | | | | | CCATGGCTCACTCTCTGCTGCTGCGGCGACACTACTACTACACTCAGTGGTGGAGCCT |
| | | | | | GTGATGACATTTCGTTAAAAAAAGAGATTAGTTAACACAAGCTGACCACTACA (SEQ ID |
| | | | | | NO: 23) |
| common_532 | NC_044370.1 | 100388338 | C | G | AGAAACCTTAATCTTCAATTTAAGACCATTGTTCTTATTATTCTGCTTCTTCGTCAGCCCA |
| | | | | | TCCCCCTCCTGAACACCGAATCTCCGAATACCCTCCACGTACTTCATGTACATGTCGTT |
| | | | | | GTACGGAAGCTTCATGCCGCCGCCACCCCCACCGTCGTCACGGGGGTTCGGAGGGTT |
| | | | | | CGGCGAAACACCAAAACCCATTCC[C/G]ACCTGACCAACGCTGGCGAATAAGCCACCG |
| | | | | | GGGTGATAATACCCCTCTTGGGAATTCCATTGAGAAGACCCTAAACCGCTGACGGAGT |
| | | | | | AAAACCCATCTTTCGTCTCATCAAATAGTTGAACTCCTCTCTTACCCATTAATCTCAAAAA |
| | | | | | ATAAAAGACACAAACTTTCGGACACGCTCTCTTTTATTTGCTTCTTCTTCTT (SEQ ID |
| | | | | | NO: 24) |
| common_552 | NC_044370.1 | 102179472 | A | T | TGTCCAATCAATCTCTTCCCAACTCCTCCTGGTTCCATTACAAAGAGGCGAAACCGTCG |
| | | | | | TCGTTTTATGCTCCCCAGGGCTGGAACTCGTGGAAATCATATTCGGTTGCCAAAGAGCT |
| | | | | | GGCCTTTTGACCGTACCCATTTCTCCACCAGACCCAACTTCAGACAACTGCCACCATCT |
| | | | | | TATAAGAGTCTTATCCCAAACCAA[A/T]CCCAAAGCCGCAATAGCTCACCAAACTTACAT |
| | | | | | AACCAAAGTTCACCAATTCATGACCTCTTCCTCCTCGGCCACTTCAAAACTTGCAAGCTT |
| | | | | | GTTAAAAAAACTAAAGTGGATCTCAACTCAACATGTCAAGCAGTACTCCAAAACGACAT |
| | | | | | CTTTGAAGTTATTCGATTCTGGTTTGTCGTATAAAGGGTGTAATGCAGAA (SEQ ID |
| | | | | | NO: 25) |
| common_553 | NC_044370.1 | 102227038 | A | G | ATATCGTGCTTAATATATAAGAACAATAAATAACATGTTATTATAGCTAATTAATATAGCA |
| | | | | | CTTAATAAATTCTTATACTATAATGGTACGTACATAATGTGGGAAGCTAGCAATTAAGTG |
| | | | | | ATGATGGATGAGTTATTAATTACCTGTGTTGATCATTATTATGATCATTAGGAAATCCTCT |
| | | | | | AAGGTGGTTGCAGGCAGAG[A/G]CAGAGCCTCTGTAATTGGGTGTAGGGAATTGGTCA |
| | | | | | GATTCAATTTTGAGGAGGTCCTGCACAAGGATTTCGTAGTGTCTCTTGACTTCGTCTGG |
| | | | | | AGTCTTCCCACCCACTGCTTTGGCCACATTTTGCCATCGGTCAGGGGTGTCCTTGTCAT |
| | | | | | ACACAGCTAAAGCCTTTTCAAATTGCTTGTTTTGCTTAGACGTCCAAG (SEQ ID NO: 26) |
| GBScompat_ | NC_044370.1 | 89565596 | C | T | GGTGAAGATACATGATCTTTCTGGCTCAGCTGTTGCCGCAGCGTTCATGACAACTCCGT |
| common_91 | | | | | TTGTGCCTTCCACAGGTTGTGATTGGGTTGCCAAGTCCAACCCAGGAGCTTGGTTGATT |
| | | | | | GTTCGGCCTGATGCTTGCAGGCCAGAGAGCTGGCAGCCATGGGGTAAACTCGAAGCT |
| | | | | | TGGCGTGAACGTGGTATGAGGGACTC[C/T]GTTTGCTGTAGATTTCGTCTCATGTCTGAA |
| | | | | | GGTCATCAGGAGGCAGGGGAGCTTCTCATGTCTGAGATTTATATCAATGCCGAAAGGG |
| | | | | | GAGGGGAGTTTTTCATAGACACGGATAGGCAGGCGCTTGCAACGGCAGCAACTCCAAT |
| | | | | | CCCAAGCCCGCAAAGCAGCGGAGACTTTGCAGGACTCAGCCCAGTTGTTGGAGGC |
| | | | | | (SEQ ID NO: 27) |
| GBScompat_ | NC_044370.1 | 101752789 | A | G | AGCCCCAACACCACCCCCACCACCAACACCACTGCCTCCACCTATGCTCAGACCAAGA |
| common_99 | | | | | AGATCACGCGTCATGGGCGGACTCGCGAACGTCAACGATGGGCCCATGACTAGATCT |
| | | | | | GTCAAGCCAGAATTTCCACCAGGCTGAAGCCCAAGCCCAAGCCCAGCTGCCAAAGTCG |
| | | | | | ACGAACCACTTTCCGGCTTGATTTGAGT[A/G]CTGTTCCACTGCGTGCCAAGACTGTCTT |
| | | | | | TAACGGAGGCTGAAGATGTTGATGTTGTCAAGCCAAAACTGCACAACAGCGAGGAATTT |
| | | | | | GAAGCCGAAGCACCCATTTGGGCTGCTTTTTGAAGCAATGCCGTTGCAGACATTGCTG |
| | | | | | GAGGCTGCAGTGGTGATGGGCTGAAGTTGCGGTGCTCGGCCTGGTCTTGTGTTTGA |
| | | | | | (SEQ ID NO: 28) |
| GBScompat_ | NC_044370.1 | 75985317 | A | T | CAAGAACTTTTGCAGCGATCGATTCCGAAGGCTCTTCGTCAACCTTGAAATCGAAAACT |
| rare_10 | | | | | TCCATAGCTAGACGAGGAGAGGTGTTTGTTCCCGACTCCAGAAATGAACTGGCTGGAG |
| | | | | | TCAAAAGTCAACTGGGAATCGTCAACTAGTTTGCATATGGATCGGGGTGAGGAGTACTT |
| | | | | | TAGAGAGAAGAACTCGGGAGTGTGC[A/T]GCGGAAGGAGGAATTTGGTGGTGTAGAAT |
| | | | | | CGTGCCCTAATTTGTACTGTACTAATCCAGTTATAGAAGAGATCAGCTGACTTTACTTGT |
| | | | | | ACTTGTACTTTGAGAAGTAAAGTGATGATAATGAAACAGAGCAAATATGATCATTATTTT |
| | | | | | TTTGTGTGTGTATGATTAGCGTGTTTGGTAAATCAGATTTTCGTGTTTGCAT (SEQ ID |
| | | | | | NO: 29) |
| common_203 | NC_044370.1 | 20652741 | A | T | AGAATTTTAGAGACGGTGGAGTTTGGTTGTGTGAAAATGCGCCAGGCTTGCTTAGCAA |
| | | | | | GCAACGCCTGGTTGAAGTGAACAAGAGATCGGAACCCAAGCCCGCCTTCTCTTTTAGA |
| | | | | | CCTACAAAGTTTTTTCCAACTTTGCCAATGAATCTTGGGGCAATTGTTGTCCTTGAATCC |
| | | | | | CCACCAGAAATTAGCCATAATGGAC[A/T]CCAGAGAGTGGCAAGTGGCCACAGGGAGA |
| | | | | | CGAAAGCAGGCCATAGTATAGGTGGGAATTGCCTGAATGACAGACTTGAGAAGGATTT |
| | | | | | CCTTACCTCCCTTAGAGAAGACTTTATTTTTCCAATTATGAAGGTGGGCCCAAACTTTGT |
| | | | | | CATGAAGATAGTGAAAAAAATTATTTTTTTCGTTCGGCCAATATATTGAGGGAG (SEQ ID |
| | | | | | NO: 30) |
| common_288 | NC_044370.1 | 30449533 | G | A | CAACCCAAAGAAAGAAAAAAAAAAAATCTAACAACGCATTATATGAGTTGTATTGCTAAA |
| | | | | | ATAACTCCCAATCCCAAATCCAAAACCATCAATAGAAGAAGAAGGAGAAGGAGATGATG |
| | | | | | ATAATGAAGATGAAGATCCAATTCTCAAAACCCTAAGCAAATCCAAACACATACTCCTCA |
| | | | | | CTCTGTGACCCTCACAGTACCC[G/A]CTCTGCATCACCATCAGAATCTTCGCAATTCCAT |
| | | | | | TGCTGTTCACCACCGCCTCCTTCGTCTTCTTCTCCCCCAATAAGCAGCACACGCTCCAA |
| | | | | | ACCACCGCCACCGCGTCCTCTGTCGCCGACCTCGACACCTTCATCATTCTCTCCACGA |
| | | | | | TTGCCACCGCACACCTACAGTCTACTCCGATTGCTGATCTTCCCTCCGCT (SEQ ID |
| | | | | | NO: 31) |
| common_300 | NC_044370.1 | 32721609 | A | c | TGAAACTTCAAAGAATGCATCTTGGTATCCTTGAAATGATCCGGAAAAGAATTTTTTTGA |
| | | | | | AGGAGCCAAAAAGTACTAATCATTTCATTGGTTACCCTGTAATTGGTTGAGAACCATTTC |
| | | | | | AGACTCATTATCTTTGTCATCATTGTCATCGGTATTGAAACTACCTTCCTCAAATGCTAC |
| | | | | | TTCATTGTCATTGTTAGCGTC[A/C]AGTGATAAGCCGGCAATCGATGCATACCACTCACC |
| | | | | | TCCTAAATAGCCTGAATCCCTCATCAATCTAACCAAAACTTCTTTGCTTTCGAGATCATT |
| | | | | | TTCTTGCTCACATAGATCGAGAAAAGCAGATACGTAGACAGGTTTGGGCCTCCAGTTGT |
| | | | | | TCGAACCATCGGCTGCAAAAGCTTTTTTCCAGTAAGTCAAAGCCTCG (SEQ ID NO: 32) |
| common_313 | NC_044370.1 | 34512948 | A | G | GGATAGAAAGACCACGTTATCATGAGGGCCACAACTTAGTAGTGTACAAAATTCAGGGA |
| | | | | | AAAAGTTAATTGTTCTTTAGAAAAAGCAATAATGGGAGCCTAAATTTTGCAAATTAGAATT |
| | | | | | CAATTTAGCATTTTTCTCACCATGAGAAGATTCCACGTCCAAAATCAGATCAAGTGCATA |
| | | | | | GTCATAATATGGGACTTGACT[A/G]CTCAAACCACAAAGGTTGAAATCATCTTGAATGTA |
| | | | | | TTCATCATCGACTTCGCAAAAGAACTCATTTCCTCGCAAATTGCAAAACCATGAAATCCA |
| | | | | | AGAAGTGTCATCTCCATCAGAGCCACTTACATCGGATTCTTCACTATCCGTCTCAGATT |
| | | | | | CCTCTGCAAACAACGACACAACACGTCAATAAGCAAGCTTAGAGAAT (SEQ ID NO: 33) |
| common_375 | NC_044370.1 | 57469730 | A | G | TTTGGGACAGATAAAACTATTTGGGTACCAATCATGTCAAGTCCAAAAGAACTTGGTTCT |
| | | | | | ACTAGGGACCCGTCACCAGGCTTTGTCCAGATGTGCCTTGAAAGTATCAATGTCACAAT |
| | | | | | CTGGTGGACCAGAAAATGTTAATATGAAAATCAATGACGTCAAGGAGAAATTACAGAAA |
| | | | | | (GCCATTCCTGACTCAGTTAAGGA[A/G]CTTGAATGGGAGAAAGGGGCTGATATACTGGT |
| | | | | | GCAGCAATTGCTTTCTCTTGGACAAAAGGCATTCAAATGGCTTACTGTTGTTTTGATTGC |
| | | | | | TGTGAGCTTTTTATCAGATGTTATATTCACTATCTCTAGGAACCAGGAATTAGTCATGCC |
| | | | | | ATTTGGTCTCTTGGCTGGTTGTTTATTGGCTGATTTCTTTAAAGAAACA (SEQ ID NO: 34) |
| common_424 | NC_044370.1 | 70255045 | C | T | TTGTCCGCCTACAAACAAATTATGCAATATCAGAACCTTATCACCCACTACAACACCATG |
| | | | | | TTCTATTCAAATCAACATATCTTAGATAAACAAAAGCTGTAGACATGGACTGACCTGAGA |
| | | | | | CCAGTATGAGATCCAAATGCCGCGGATCTCATGGGTGACACGTGGGTCAGATAACGCA |
| | | | | | TCAATCCATCGTTTGATGAATCG[C/T]TCTTGCCTGAAAAAATCAAGGTGTGAAAGCCAA |
| | | | | | GTCAAGATTAGTGCCTGCAATAAGATTGAGAAATTTTGATTTTACTTTGCAGAAAATAGA |
| | | | | | GGAGGATAAGTACCTGTCTGGTGTAAAGGATCGGTATCTCTCTCCAGGTTGCTTGAAGT |
| | | | | | TGTTCTCCTTCTCGATAATGCACTACACTCACCAAATAAATACACACAT (SEQ ID NO: 35) |
| common_425 | NC_044370.1 | 70407835 | C | A | AAGCAATTACAGAAGCAGCAGAAGGTGGTGCAAATGCAGGTGGATATGATGCCCCATG |
| | | | | | ATCCGGTTTCTTGTTCGGCTGCTCAGGTAGGTTTGGTTGCTTTTGTAAATACCGTTTCAA |
| | | | | | ATTAACAGAAACAGCATTATCTTCTGGAATGTTCTGATTTCTTCGTTTACCTAGTTCCAAA |
| | | | | | TCAAGGCTTATTCCAGCATCCG[C/A]TTCGAGTTCATTTAAACCATTGCCCTCCAAGGAC |
| | | | | | TGCTTGCGCGGAGGAGGGCTACAAGGGCATTCGGAGGTGAGTATCTTAGTTCCAAAAA |
| | | | | | CCTTGATACTATTACTGTCCATCACTGTTCCTCCTGTTATACATGAACTCTCCGGACCAT |
| | | | | | AAGATATGAACCTTCTGTTATCATTTTGCAAGTCCCTTAATGCCACCTT (SEQ ID NO: 36) |
| common_426 | NC_044370.1 | 70445826 | G | C | GAGCTTCTTGTTAGATTCATCAATCGATAAATTCAACAAAGCTGTTACTGCATGCTCTTG |
| | | | | | AATCTTTGAATCTGGATAGGATAGGAGCTGTACTAATGGTGGGATTCCTCCATTGTGGG |
| | | | | | CAATCAAAATTCTGTTCTCAGGGTTCTCTTTGGAGAGCAACCGAACCTTCTTAACCGCC |
| | | | | | TTTCGCTGCGTTTCTAAATGGCT[G/C]GAAGATAGTCCTTCAACAAGGGACAAGATCTCC |
| | | | | | TCTTTGAGATCAGCTGAGGAGCTTTCATGGTCTCCTGGATTATCCTTTGAGGGAAGTTT |
| | | | | | AAAATTGTTCTTCTCACTCCACTGCAAGATTATGTTTTTAAGAGCATAATTTGACGCAAG |
| | | | | | TGTCAGATGAGGAAGAGTTTGCCTGGTTTTGGGACAAGTCCTGTGATTA (SEQ ID |
| | | | | | NO: 37) |
| common_428 | NC_044370.1 | 70624527 | G | A | AGGAAGACAAAAGATCCAAATAAAGAAATTGGAAAACAAGAGTAACAAACATGTCACTT |
| | | | | | TCTCTAAACGGCGCTCTGGTCTCTTCAAGAAAGCTGGCGAGCTCAGCCTTCTTTGTGG |
| | | | | | GTCCAAGGTGGCAGTCATCGTCTTCTCTCCCACTGGCAAACTCTTCTGCTTCGGTCATC |
| | | | | | CATCTGTGGACGACGTCGTTCGCTC[G/A]TATCTCGATTATAATAACCAAACAGTAGTAG |
| | | | | | TATTACCTGGTAGTAATGATAACATTACTGCTACTACTACTACTAGTACTAGTACTTTTGT |
| | | | | | AAAAGCTATGACTGAGTTGGAAGAGGCCGAGAAAAAGAAGAAGCAATTGGTGAGACAA |
| | | | | | GCGAGATTGGCCTCGATTTTAGATAATAATAGTTGCGCGTGGTGGGAGGAA (SEQ ID |
| | | | | | NO: 38) |
| common_431 | NC_044370.1 | 70913971 | G | C | ATCCCCAGCAGAACACCATCAAAGCCGTCAATATCTCATCACATCAGATCCATCAGTCT |
| | | | | | TCCATGCAGATCTCATCCGTTGATTTCCCAGCTCAGGGACGGCGTTGCTGAGCTTCACT |
| | | | | | GCTGGGTAACCCAACCGGAATGTCGGACCTCCGTTTGGCTGACCAACGGGTTGACCC |
| | | | | | GACTGAGAGACCTCCACGACTGTCTC[G/C]ACGATATTCTTCAGCTTCCACAGACCCAA |
| | | | | | GAATCTCTCCGCCGATTACCCAGCTCCGTCGTAGAGAATCTTCTAGAAGATTTCCTCCG |
| | | | | | CTTCGTAGACGTGTACGGTATTTTCCAGACCTCGATTCTGGTAATCAAGGAAGAACAAT |
| | | | | | CAGCGGCGCAAGTGGGTTTACGAAAGAGAGACGAGTTAAAGGTTTCTCTTTACG (SEQ |
| | | | | | ID NO: 39) |
| common_432 | NC_044370.1 | 70956672 | C | A | TCAGTAACAGTCTAAGTCTCTCCATTATTATGGGCTGCGCCAACTCGAAGCTCAACGAT |
| | | | | | CTTCCGGCGGTGGCCTTGTGCCGGGACCGATGTAAATACCTAGACGAAGCTCTCCGCC |
| | | | | | ATAGCCAAGACTTCGCTGAAGCTCACGCCGCTTACCTCGACTCCCTCAAGGCATTGGG |
| | | | | | TCCCGCTTTTGATCGTTTCTTCAGTA[C/A]AAAAGTACCAGTCAACAATGAAACTTTAAAA |
| | | | | | AAGTCTTCTTCTTTATCTCCGGCGGCCGTGGCTTCTTCTTCCCCGCTGCATAAATCAAA |
| | | | | | CTCAGCAGACTCGCACCTTCGATTCCCTTCCGATTCTTCTGAGGACGAGGATAGTGATA |
| | | | | | ATCATAATAAACTCGGGCATAATTGCCTCAACGAAGAGGAATCCCAAGGCTT (SEQ ID |
| | | | | | NO: 40) |
| common_434 | NC_044370.1 | 71490671 | C | A | TCATGACCTGAACTTGTACTTTTTTAATTTAAAAGTCGTTTTGGAAGAAGTTAGGATTTGT |
| | | | | | ATATTAATCTTTTTCGGCTTCCTATGTTTGGACCAAACCATCTGCTGCAGGGTTGTGGA |
| | | | | | GAGTGTTGGGGAAGATGTGAATGAAGTAGTTGAAGGAGATAGAGTGATCCCAACATTT |
| | | | | | CTTTCAGATTGTGGGGAGTGTTT[C/A]GACTGCAAATCAAAGAAGAGTAACCTCTGTTCG |
| | | | | | AATTTCCCCTTCAAGGTCTCACCTTGGATGCCCAGATATGAGTCCACCAGATTCACAGA |
| | | | | | CCTCAAAGGAGACCCTTTGTACCATTTTCTGTTTGTTTCAAGTTTTAGTGAGTATACTGT |
| | | | | | GGTTGATATCTCCAATCTTACAAAAATTAGCTCTGAAATCCCTCCAAAC (SEQ ID NO: 41) |
| common_436 | NC_044370.1 | 71705041 | T | C | CTTCTCTAATATAGTATAGCTATGCTTATTGGATAATCCTAAAGCTCCATTTTAATTATGC |
| | | | | | TATAGCTAAGTTCACTTTTGCCACACTGCATATTTATTGCCAGAAGTTGCTAGTGTCCAC |
| | | | | | TCCCTTCCAGCAGGGCACCAAGAGCCCTCCCCAATCTTCATACAAACATTCTCACCAAT |
| | | | | | TACTGCAGAATAGAGGTTAGG[T/C]TGAGCTTCCAGGATTCTAATGGATGATCGGCTGT |
| | | | | | GAATGTCCTGACGCTTTCGAATTTCAATCTGTGGAGTAGAAATAAAACATAAAGTTTCAC |
| | | | | | CAAAGCATGAATAATTTACTTGGTACACTATCAAAACAAAAACTAGTGTCCTACCAGCTT |
| | | | | | CACAATTTGATCATGAATAGAGCTTCCCCAATCGTAAAAATGGTCAT (SEQ ID NO: 42) |
| common_437 | NC_044370.1 | 71800647 | G | 1 | GATATCTACTTGCAGATGTACGCTAAAGATTTGTTTCAGAAAGACTTTCTTTAGAGGAAG |
| | | | | | AAGAAAATGAAGATGAAAGAGACAAGAGTGAGTGATTATTAAGAGAACCAAACTCAAAA |
| | | | | | CTCAAGAGATTACAAAAAAGAAAAGAAAAGTTGTCAGTGTAACACGCCAAAGTAAAAAA |
| | | | | | AGAGTGTAGAGATAGAGTGTGTT[G/T]GGGTGTATAATTAATTACTATGAAGACGACTTC |
| | | | | | TGATTATTTATCAGTATATAGATTTAAAAGTTTTGGCAGAAAAAGGTAATTATGTATTGAC |
| | | | | | CGTTACATCCCACGCGTCCAATCCCTATTTCTTTTGTTCTCAGCTTTGGTCAGACTAAAA |
| | | | | | TTTACTCCCCTTCTTTAATTACTGTCCTATCTTTGGAACTAGCCCAT (SEQ ID NO: 43) |
| common_439 | NC_044370.1 | 72435815 | G | C | GGCAGACAACATAACCCTTCTATTTCATTTTCCTTTCTTCTTGTTTTTGGTCTCTCTCTCT |
| | | | | | CTTCCACATACAATCCAAGTTTCGTGAAAGTACACAAATAAGAAAATAACAAAGATTTAT |
| | | | | | AATAACGTACTAGATAGGTTTAATATATAGTTATAATCTGATGATGATGCGGAAAGTGAT |
| | | | | | GGCTTCATCTTCTTCTTCTT[G/C]TTGTTTTTATTATCGTCACATAATAATAACAACAATAG |
| | | | | | TATTGTTATTTTTTATTAATCCATCGTGGGGTTTGATAAAGTTACCGCCAAATCAGACAG |
| | | | | | TTCCGGCAGTGATCGTGTTCGGAGACTCGATCATGGACACAGGGAACAACAACGCTCT |
| | | | | | TAAGACTCTGGTCAAGTGTGATTTCGCTCCTTATGGTGAAAATTT (SEQ ID NO: 44) |
| common_445 | NC_044370.1 | 79634333 | T | C | TTTGATATTTTAGGTACGGATCGGGGAAGGAGAGGAAGTTGTTGGAGATGGCGCCGAC |
| | | | | | TACGATTCGGAAAGCGATTGGGGCGGTGAAGGACCAGACTAGTATTGGAATAGCTAAG |
| | | | | | GTGGCTAGCAACATGGCGCCGGAGCTTGAGGTTGCGATTGTCAAGGCGACTAGTCAC |
| | | | | | GACGATGATCCGGCTAGCGAGAAGTACA[T/C]AAGAGAGATCCTGAATCTCACATCTTA |
| | | | | | CTCTCGAGGTTACGTTCATGCCTGTGTTACGGCGATTTCGAAGCGTTTGGGGAAGACG |
| | | | | | CGCGACTGGATTGTGGCACTCAAGGCTTTGATGCTGATCCATCGTTTGCTTAATGAAGG |
| | | | | | GGATCCTTTGTTTCAGGAAGAGATCTTGTTCGCTACCAGAAGAGGGACCAGGCTTCT |
| | | | | | (SEQ ID NO: 45) |
| common_448 | NC_044370.1 | 79909671 | C | G | ATCTCAAATGAATTGGATTGACAATGATGGGTTATTTCTCTTGTCCAACAAGTCTCAATT |
| | | | | | CGCTTTCGGTTTCACCACCACTAACCAAGATGTCACAAAATTTCTGCTAGTAATCGTCCA |
| | | | | | CATGGGAAGCCAACGAGTTATTTGGACAGCGAATATAGACACCCCAGTTGCCAATTCC |
| | | | | | GATAAGTTTGTGTTCGATGAAAA[C/G]GGTAGAGTTTTCTTGCAAAAAGGAGCAAGTGTG |
| | | | | | GTTTGGTCCATTGATACTGGTGGCAAAGGAGCTTCTGCTATGGAGTTGATGGATTCAGG |
| | | | | | TAATTTGGTTCTGTTTGGAGAGGACGAGAATAGTAAAATATGGGAGAGTTTTGACCATC |
| | | | | | CAACCGATACCCTTTTATGGGGACAGGAATTTGTTGAAGGGATGAGACTT (SEQ ID |
| | | | | | NO: 46) |
| common_449 | NC_044370.1 | 79968518 | T | C | AGTGGATCATCTACTGAACAGAAGGTTCTGTTATCCATAGTAAAATTTGTTATGAGAGCA |
| | | | | | CTGAATTTTTCTTCGTTTCTTTCATTTTGTTGATGTGGCGGTGACAAACATTTATTTAATG |
| | | | | | GGTACTCTGTCGTATCTGATGCTGTTGACATTTTTTTTATGCTTTCATCAGCTATCTGGA |
| | | | | | AAGTCTGAGGTAGCTATGCC[T/C]GAGGACCTTACGTGAGTAGTGATTTGTCCTTTCCAA |
| | | | | | TTGTTCTTATCTTGAGCTTATTGATATTGATAATTTGTTCTTTTTCTCTTTACTATAATACA |
| | | | | | ATCTTTTTCTTCAAATCTTTTTATAGTTAGTGGTCTAGCTATATAATCTTTCTTTATTCCCC |
| | | | | | TTCCCTATTATGGTTTGGTGGCAAATTTGACAGAATCGATT (SEQ ID NO: 47) |
| common_452 | NC_044370.1 | 81689735 | C | T | AATTAAAAATGGCATCTTTCGCCTCAGAAACTCTCACTTTCATCTTCTTCATCTCTCTACT |
| | | | | | CCATTTGGGTTCATCGGGGAGGATTCTTTCCGACGAGTCTGACCAAACCCAACAGCCT |
| | | | | | CTTCCCTTTCAATACCATAATGGCCCTCTTCTGTTTGGAAAAATCTCCATTAACTTAATAT |
| | | | | | GGTACGGAAACTTCAAACCAA[C/T]CCAACGAGCCATTGTCTCCGACTTCATTACCTCCC |
| | | | | | TCACTTCATCTTCTAAAACCAACACAGACCAACCATCAGTCAACACGTGGTGGAAGACC |
| | | | | | ATCGAAAGCTACCAATACCACCACAAGCTGAGCAACTCCGTCTCGTTAGGATCCCAGTT |
| | | | | | CATCGACGAGAACTACTCCCTGGGGAAATCGTTGACCAGTCAACAAAT (SEQ ID NO: 48) |
| common_453 | NC_044370.1 | 81762017 | T | C | GAGTATTTTCAAGTTTAAGATCTCTATGACATATTTGCTGCCAGAAGTCATTTATTAAATA |
| | | | | | ATTAGACAATGACAAAGTTGATAATCAATGCAGTTCTTTAGAGATATGTATGTATATCTAT |
| | | | | | CACAAAAGGATAGTTCAAACAAAATTCTTACCATTGAATGACAGTAACTGACTCCTGATA |
| | | | | | TTAGTTGTTGAAAGAAAAA[T/C]CTTGCCTGATTCAATAGAACAAAAAGATGCTTAAGTGC |
| | | | | | TTACATTCATTAAGATTTCATTTGGTGTAAAAACATAAGTTTAGTTAGAAATTTCCTCCAT |
| | | | | | TACCTCATCTTCACTAAATCTACCAGCAGTGCATATTCTTTCAAAGAGCTCTCCACCACC |
| | | | | | AGCATACTCCATCACAATGGCCAGATGAGTTGGAGTTAAAAG (SEQ ID NO: 49) |
| common_454 | NC_044370.1 | 81831758 | G | C | CTTTTTTATTATCTGAGATTTAGTAAGTGCCCATATGTTTGTAGATCGTGGGATTTAGTC |
| | | | | | GATTAGGGTACTAGTTTGGAGGTTTTTGTTTTATCAATTAGTTGCTTCAAGAAACTCACT |
| | | | | | TTTCTTTTTCGTTTTTATGCTAGTAGAAATGGACTTGAACTAGGATCTCCAAGCAGGCGT |
| | | | | | TGTAGGAATCACTGAGAGTGT[G/C]CATGTCGCTCTTGTTGAGGAAGCTCCAACGGAAG |
| | | | | | GTAGCCAGGCTATGGTTCTCGTTGTAATGCCTCAGCAAGGGGCAACTCTAGAAATTGTC |
| | | | | | GTAGAGGATGAACCTACTACAGAGGAAGGACCCTCCAAAAAGGACAATGGAAAAAAGA |
| | | | | | GGGCCAAGACACCCCCAGCCGCACAGGCTTACAATGACTCCATTTGGGAG (SEQ ID |
| | | | | | NO: 50) |
| common_455 | NC_044370.1 | 81901321 | C | G | GTGGTTTTACATACCCCAGCACGCATCTGATTGAGCCCACCATTGGTATGAACTAGAAG |
| | | | | | GTAACCTCGAGACCCGGCAGGGGCTGATACAACATCAAAGATAATTAATACAATTCACA |
| | | | | | TATCTGAATAGAAAAATAATACCATTATGTTCCTTACGTGTATAATTAAGACTTGGGTTTG |
| | | | | | CACATGGCACGAAATCTCGATT[C/G]GATGGAGGTTTCCATAGCTTTTCCATTTCCAAAA |
| | | | | | TTCCACTCGCTTCATCCAACTGTCACGTCTAAAGGAGTGAAAATGGGAAAAAAATGCAT |
| | | | | | GATTTGTTCGCATTATTACAAAAGAAGGGTAGTTATAAATACGAATACCCTTCTCTTCTTT |
| | | | | | TTAATCAAAATTTTCCCTTCTTATTATGGAAGTCTCTCCTACTAAAG (SEQ ID NO: 51) |
| common_459 | NC_044370.1 | 82560029 | C | G | AGCTCACAACAAAGTGAAGAATGCGGGGACCACCTTGGATTCCACGTCGACAATATTC |
| | | | | | GACAATGCCTACTACAAACTACTTCTGCAAGGCAAGAGTATTTTCTCTTCAGACCAATCT |
| | | | | | CTACTCACCACCCCACCAACTAAGGCCTTAGTCTCCAAATTCGCTGCTTCTAAACAAGA |
| | | | | | CTTCGATAAAGCCTTCGTTGAGTC[C/G]ATGATCAAGATGAGTAGCATCCATGGTAGTG |
| | | | | | GCCAAGAAATTAGGCTCAACTGCAAGGTCGTTAATTAATTAATTAATTAGTTTTTTTTCTT |
| | | | | | TCAATATATTATTATAAATTTCTCCATCAATCGATCGTTTAATAATTGAAGAAGAGGAGTG |
| | | | | | ATAAGATGAACCAAACCAATTAAGATGCATGTAATGATTAGTAAGTTT (SEQ ID NO: 52) |
| common_470 | NC_044370.1 | 89227662 | T | C | GAACCTTGTCTGAAAAATAGTTCACATGTAATTAGAGATAGGAAAACAAAGTTCCAGCA |
| | | | | | GTGAATAAAGAGGAAAATTACAAACTCAAATTGATCTCTTGAATACCATACCTTCTGACC |
| | | | | | ACCAACAGGCTTCACACAATGCACACCGAAGAATTTCGTCACAAGGGAATTTTCATACC |
| | | | | | GACAGACATGTTGATGATAACTA[T/C]AAAGCATCCGTAATAGCACCTGAAATAATATTAC |
| | | | | | AATTAAGTTATTTTACCTGACCCTTCAAAACCGCAAATTATGCTACTCAAATGGCTTTTG |
| | | | | | AGACAGCATAAGCTCCATAATAAAGCAAAAGTCAAAGAAGTAGGCAACAAACCTTGACT |
| | | | | | TCTGATTTCTTCACCGTCTTTATCATAAACCTATCATCTTGTGTCAAG (SEQ ID NO: 53) |
| common_473 | NC_044370.1 | 89460138 | T | G | CTATAAATATACTTTCCCAAAAGGGACTATTCGCTTTAATCTAATTGTAATAGTACAAATA |
| | | | | | TGTTTGTTTCACTTTTTTTCTTTATTGTAGAAAAAAGTACCCCTTTTATCCGTTGAAGCAT |
| | | | | | TGCACCATTTGCATATCTTTATCTTCGTCCTAGCCATTGTCCATGTCACTTTCTGTGTTC |
| | | | | | TCACTGTTGTGTTTGGAGG[T/G]GTAAAGGTAAGTATATCTTCCAAATTCATATGCTTCTC |
| | | | | | TCTCTTTTATACTCTACTACTTTGTTTTTGTTCGTTTATGAGAAATATTTCTACACAACTAG |
| | | | | | ATACGTCAATGGAAACGTTGGGAGGATTCTATTGCAAATGAGAGTTATGACACTGAACA |
| | | | | | AGGTAAGTATTCTCAGTACCACTTTTGACAATATTATAGAAA (SEQ ID NO: 54) |
| common_476 | NC_044370.1 | 89639165 | C | A | AATGTCAATCTCTATCGGAACGCCGCCGTTTGATATCTTAGCCATTGCTGACACAGGCA |
| | | | | | GCGATCTGACGTGGACTCAGTGCAGCCCTTGCAAAAAATGTTACAAGCAAGTGGCTCC |
| | | | | | TCTCTTCAAACCCAACTCTTCAAAAACATACAGAGATGCTACCTGTGATTCCTCTGTTTG |
| | | | | | TAAGTCCGCCACCGGAGCTAAAAC[C/A]TCTTGCTCCTCCCTCGACGATTCATGCCAATA |
| | | | | | CTCCGTATCTTACGGCGACCAATCTTTCTCCAACGGTAACATTGCTACTGACGTTCTCA |
| | | | | | CCCTCTCTTCCACCTCTGGAAGACCCGTCACCTTCCCCAATTTCATCATCGGTTGCAGC |
| | | | | | CACAATAGTGATGGTATATATATAATATTAATTATGGCGTGATAACCTTAT (SEQ ID |
| | | | | | NO: 55) |
| common_478 | NC_044370.1 | 89807220 | G | T | CCCTTGGTTGAACCGCTGAGCAAGCTCTACAGTTGCACTTTTGTCACCATCAGTAGGCC |
| | | | | | AGCCAATCTCACCGATGATTATGGACAAGTTTCCAAATCCATTTTTCTGCAGAGCCCATA |
| | | | | | CCAGTGTGTCATGGTTTGCATCCAAGACATTCTGGTAGATTTTTCCATTGTCGTTTATGG |
| | | | | | CAGCGGAATAGCCATCAAAGAA[G/T]GCGAAATCAACAGGGAAATTGGGATCGTTGTGG |
| | | | | | AGGCTGATAAAAGGATAGATGTTTACAGTAAAGGAACCTCCATTGTCGCTTAAGAACTT |
| | | | | | AACGATGGCCACCATGAGATCTTTTATGTCTGTTCTGAAGTCACCGTCGGAAGGTTTCT |
| | | | | | CACTCGAGCTGCCATATACATCAGCGTTTAAAGGGATAGTGACTTTAACT (SEQ ID |
| | | | | | NO: 56) |
| common_479 | NC_044370.1 | 90357946 | T | C | AGTAATTTTAAAAGTTCTGGTTTGTTTCAGGAAGAAAATTGGTGTAATAAAAGTAGGCAA |
| | | | | | TGAGAGTTACAGAAGAGAATCAGAGTCAGATGAGTTAAATACAAAAAAAGCAAATCTTT |
| | | | | | CAGCTTCAAGGAAGGAAAGGGTTCAGCTACCAATAATCCCAAATTATGAGGGTAAGAAA |
| | | | | | TTCCCTATTGGTGAATTTTTAAG[T/C]CAACCTTGTGCAATTGAAGCCCTCCTCAATACCA |
| | | | | | ATGCCTTAAAAAGTTTCCAATGTCTTTCTCCTAACACTTACAGGTAGGCTACCTTTCAAC |
| | | | | | TTCAATTCAACCCTTTTTCTTTGTAAAATCTAACTTAGTATATCAAAGTAGTTTAAGCTAG |
| | | | | | AATATAATAGAACTATTTTCACCATTATTAGAAGAAGTTGGGATTG (SEQ ID NO: 57) |
| common_487 | NC_044370.1 | 91953671 | T | C | GTTTGAATATAAAAGCTAGCAGCACCTTCAATGATTATTATGTTCTAACCAGAAAGAATA |
| | | | | | GCTACAAACTCTGAACTTTTCAAGTACAATGTATGTGTATGTTTGACTAATCAATCTTAC |
| | | | | | CAAAATATTTTGTGGCAATGTGGAACGAATTCCTAGTTGACTGCTCTTTCCCCAAGTGTC |
| | | | | | ACGGTTGAGTTTTCCTTTCAT[T/C]AATGATTCAGGAGTGTCATTTCCTAAAACCTCCTGA |
| | | | | | GCATTGGCGTAAGCAGTTGCGGTATGCTCACTTACTTGTTTCATCAATCCAATCGCATC |
| | | | | | ATAGCTCCTCTCTACAGGGCTCACGTAATACCCCATATCCTGTAGAAGAAGTTTCCACA |
| | | | | | GAATTAGCGAAAAAATTATTTCCCATCAAAGCCAGAAAGTTTAGGTG (SEQ ID NO: 58) |
| common_488 | NC_044370.1 | 92041720 | G | A | TAAGCTTTCTTACAATGTGCTGTCCAATGAAAGCCGCAACAGTAGCCACAGCAACAAAG |
| | | | | | TAAGCAGCTGCAAATAAATCACATCACGTTTTAGTAAAGTACCATATTCGGTTCTGCTGT |
| | | | | | AAAGGTGTTCAACAGAACAAATGAAAATTAGATAATGATCAAAGAAGAGTATATGTAAAG |
| | | | | | GACACTAACCATAGGGAACTGG[G/A]AAACGTTTTAGAAGGTAATATTCTACAACAGACA |
| | | | | | TAGAAGAGGAGAACGTCATTGCAAATGTGGCTGTTGCACTCGAGACCTACACAGAAGT |
| | | | | | AACCAACTAAATTATAACTGATTCAATTGCATAATTATCAAATAATTAACTCTTTAAGGTT |
| | | | | | GAATGCTATTTTGCTTTTACTATGCTAGGGAAAAACTCAATCCCCTTA (SEQ ID NO: 59) |
| common_489 | NC_044370.1 | 92434073 | A | C | ATTCAAGAACTTGGGATGAAGATATTATTCGAGACCTTTTTGAGGATAGAGATCAGACC |
| | | | | | CTCATTTTTTCTATTCAGCTAAGTGAAAATGCCTTGGAGGATCATTGGTGCTAGAAGTTT |
| | | | | | GAGAACGATGGAGGTTACATGGTCAACAATGCGTACAGACATCTTCAGGTGCTCAAGG |
| | | | | | GAGCTTGGCCTGCTGCACAACCGA[A/C]AAATCTTTGGCATACTCTCTGGAGTCTTAAAG |
| | | | | | TGCCCCCAAAGGTCTGCAATTTTTTGTGGAGGGCAGCGTCAGGTTGTTTGCCTACTTGT |
| | | | | | GTACAATTGCAGAAGAGACATGTACCAGTGAGTATAATCTGCCCTGTTTGCAATGTGGA |
| | | | | | TGATGAGACTATTTTCCATGCTCTTGTTGATTGTCCTGTCGCGAGATCGTG (SEQ ID |
| | | | | | NO: 60) |
| common_492 | NC_044370.1 | 94257304 | G | A | CCGCACAAAAGAAAATTATACTCATAAAGTTGATGAAAAAGAAAGGGTCCAAAGCAGTA |
| | | | | | TAATTCATGTTAATTAAGTTGTGATATTATTGTTTTACATTCTCATCCACACAGCGAAAGT |
| | | | | | GTACTGATAAGCAGAACTTGTTATGCTCCACTCTGACTCCAGGGATGGATTTTGTTTCTT |
| | | | | | CCACCAAAATAGTGTACACCT[G/A]TATGCATGCACATCTTTATAATTATTTATGTCTATAT |
| | | | | | ATATATAGGAGAGAAAATTAAATTATAAACTAATTAATTAATTATAATTACCTCATCAATCA |
| | | | | | TTGGTGAAAATTCTGCCGCAGGTTGATACATAACTGCATTCTTAATCTGAGTATCAATTA |
| | | | | | AAAATATAAAATAAACATTAAAATTTTAATGTTAAGGTCAAC (SEQ ID NO: 61) |
| common_494 | NC_044370.1 | 94521889 | C | T | ACCAACAAAGAGTGCCTCAGCATCGGGGCTAAAGGATATTCCAGCAATCTCGCCAAAT |
| | | | | | ATGTCTATCTCTTGCCCTTTAGAGTACCCTGACTGTGTATCGATGATGTGAACAAAGTCT |
| | | | | | GCAGGTTCAGCCATGGCCAGAAACCGGCCATCGTTAGTGAACCTAACAGCTCTTATTG |
| | | | | | CCCCCATTCTTCCCTTCAGGACGGC[C/T]AAGGACTCTGACAGGTTCCTTATGTCCCAC |
| | | | | | AATCTGCAGGTGGTGTCTTGGTTCCCGGTAGCCAAAATACGTCCATCTGGATGCCAAG |
| | | | | | CAGAGGCAAAAGAGTAGTCCAAGTGCCCTTTGAGGCTCCCAGTGATCTGAGAGGAAGG |
| | | | | | AAATTAGAATCAGGAACAGAGAGGAATGGAGACCAAACAAAATCAAGGATAAAGT (SEQ |
| | | | | | ID NO: 62) |
| common_496 | NC_044370.1 | 94684180 | C | T | GCCAAAAACTCGCAACAAAGTCAAGTTTGTGTACTCGGATGACATTAACACGAAGAAAA |
| | | | | | TAATGGAGGATCACTTTGATATGGATCAACTGGAGTCTGCATTTGGTGGAAATGATACT |
| | | | | | ACAGGTTTCGATATTAATAAATATGCAGAGAGAATGAAAGAGGATGACAAAAAGATGCC |
| | | | | | TGCTTTCTGGACTAAAGGAAATCC[C/T]CCAGCTCCAGCAACCTCAGAACCTGTCCCGA |
| | | | | | ACAATGACACTCCATCTTCGGACTCTACCATCAAGTTAGAAGATGATGCAGCTGATTCC |
| | | | | | ATCGAGAAGAGAAATGGATCGGAAGGGGTGCTCCCAGCCCCCAATCACACTATGCTCA |
| | | | | | CTGTTGACAGTAGCAGAAATCCTACTAAAGAGGTTCAGTAATTAAGGATCCTT (SEQ ID |
| | | | | | NO: 63) |
| common_497 | NC_044370.1 | 95062684 | T | C | AGTTTCCGCCTCCAGGGTAGAGTAGGCAGTGGTGAATTCTGATCTCATAGCCCATCTAC |
| | | | | | CCGTATCACCATTAATTACCACCGCAGCAAGACCAGCTGCTCCTTCCTTCCAAGAGGCA |
| | | | | | TCAGTCATCAAGACAATCGAGGCGTTGCTGACTCTCCGACAAGGATTAAGGACAGTCG |
| | | | | | AACCAGGAGGATCTGCAGCAGAGGC[T/C]GAAATACAGAAGTCCTTCCATTGAGCAGCA |
| | | | | | ACCTGTTGGAGGAGCAAAGGGAGGAATACTGAACTTCCTTTGTATAACAACTTGTTCCT |
| | | | | | TTCAGCCCAAACTGCACTAAAAAGATAGCCCATGTAGTTGATGACAGAGCTTCGGTCAG |
| | | | | | GAGTGGGAAAGATGGATACTAAATTCTCCACATACTCCTGCATTGTCTCACCA (SEQ ID |
| | | | | | NO: 64) |
| common_500 | NC_044370.1 | 95278820 | A | G | TCCTTGCCCTGTGGGTAATGCCTCAATCAGATCAATCTCCCATATTATAAATGGGTATG |
| | | | | | GGGAGGTCATCATTCTCAACTCGGTTGGTGGTATGCAAGGTATATGTGAAAACTTTTGG |
| | | | | | CATATCGCATTTCTCAACAAATTCGTGTGTGTCTTTGTTGATTATTGGCTAGTAATATCCT |
| | | | | | TAACGGATAATCTTCTTCCATA[A/G]ACTCTGCCAACCTACATGATCCCCACAGAAGTCT |
| | | | | | TCATGAATTTCCTTGATTATTTCATTTGCTTCTACTGGTAACACACATCGTAAGAGAGGC |
| | | | | | ATGGAATAACGTCTTCTGTATAACTTCCCCTCGACCATTATATAGCGAGGTATTGAGTAT |
| | | | | | AAGAGTTTGTGTGCCTCGGTTTTGTCTGTAGGAAGTTCTCCACTAAC (SEQ ID NO: 65) |
| common_510 | NC_044370.1 | 97283326 | C | G | ATTAGGAAGTCTCCCAAGCTGCAGAACAGCATGTTAGTGAAATTGGGAAATTCTCTCTC |
| | | | | | CAATACTTCGGTACTACCCATCGTCGTCTGAGTTCTCAGAACATTCTTCCATCGCTTCA |
| | | | | | GCAGTCATAAGAGCAGCCACAGCCTTGTGAATTGCTACATCTGCTCTGTAAACCATTCT |
| | | | | | CTGAGCTCTTTCTCTCTTAAGCTT[C/G]GCAATGTTAATAGCATGTTCAGCAGCGCAAGA |
| | | | | | AGCATCACGCAACCTGAACTCATCAAGATCTGGACCATCAAATTTCTCAATGATATGCC |
| | | | | | TCTGAGATCTCTCTAGGTGGAAATGCTGTGGACTCTGCCACTCAGAAGAACCAATATTC |
| | | | | | CACTGATGATGCTCATGTCTTTTTTCTAACATTCTACGATCATATAAAGCC (SEQ ID |
| | | | | | NO: 66) |
| common_515 | NC_044370.1 | 98481067 | C | T | TTTCCCGTTTCTTAATAACTATGAATCTGCCTAAAATTTGTCTTTGATTGCTCACCGAGAA |
| | | | | | TCATCACAAACCCGACAAGAGAAGTATAGTTGTGTAACATGTGAAAAGTAGAAATAGAA |
| | | | | | TTGTTTGTTTAGTATGTAGAATCGCAAAAGGATCTCTGAGAGCTTACCTATCATTCACAT |
| | | | | | GGACTCTTCTCGTGTCAAGAA[C/T]TTCTAGTAAAGGAAGCAATGGCATCTAGATTTTCC |
| | | | | | TTCATGAACCATTCATGAGGATAGACAGAGTTAGCTGATTCAAGATGAAGATGCCAAAC |
| | | | | | CATGATTCTAGAGCAATCTTGCTCCAGTCCTTCCATTTGGCTCTCATCTTCCACAATTTG |
| | | | | | TTCAATAAATGTGCTTACCTGAAAATGCAAATCAACTAGATTAGTCC (SEQ ID NO: 67) |
| common_520 | NC_044370.1 | 99379219 | A | T | TGACACAACCTGCATTGTGATTGATATATTACCTCAAGAGAAGCCACCTGCTCCGCTGC |
| | | | | | CCCCACCTAAGAAGCAGGGAAAAGGAGTGTTTAAGTCCATGTTTCGTAAAAAGTCAACT |
| | | | | | GAATCAACTTCTTATGTTGACAAAGAGTACATAGAGCCAGACGTGGTGGAGGAATTATT |
| | | | | | TGAAGAGGGCTCTGCTATGCTTTC[A/T]GAAAGGTTTGTGCTTGAAAAAGTCACTATCAA |
| | | | | | CTTAACATGATTGCCATGAGAAAGTTCTCTCTCTCTCTCTCTCTCTCTCTCTCTCTCTCT |
| | | | | | CTCTCTCTCTCTCTCTTTTCCTTTTCTTTTTAGTGTCACCTTTCTTTCTTTATTCTTGTTCT |
| | | | | | GTTTTCGTTCTTCCTGTTTAACTTTTTGGGGGTTGACCTATGAACAG (SEQ ID NO: 68) |
| common_523 | NC_044370.1 | 99748182 | G | A | GTCGCGTCTAAGCAAAAACTACCCACCTGATCTTTTCTTTCATGGGTTTCTTCACGAAAT |
| | | | | | CAACAAAAACCCATCTCTCCAATCTTCCATTTTATTCATAGCATGCGCGCAACGAGCCC |
| | | | | | ACGCGCCACCTTGCCTGCCCCTTCAACACAAACTCGTAAGTAAAGAGTTGGCCTGGCG |
| | | | | | TTAGCGTCTTCCATGGCTATTGTC[G/A]ACTCGGAGCGGTTGTTTCTTCTTCTTCTCGTG |
| | | | | | GTTGTTTCGGATATGTTGGTGCTGAGTATGGGGGTTTACGTGTCCCCGCTGAGCTCAG |
| | | | | | AGAAATCTTACGTGTCAGCCGTTGGAGACCCAGGAATGAAGAGCCCAAATGTCAGAGT |
| | | | | | TGGTTTAGAAGCTTGGAACTTCTGCAATGAAGTTGGAGCTGAAGCTCCTGGTA (SEQ ID |
| | | | | | NO: 69) |
| common_526 | NC_044370.1 | 99965933 | C | A | CAATAATTTTGACACAAAACACATTTTCTTTTTGTTGTTGTTTTTTAGGCGGAGAATCTTA |
| | | | | | TGCTTGAGAGGGGAGAAAACAAAGAGGTAAAATCCAAGTAGTCGAAATGTTTGTTGACT |
| | | | | | TGAATTCTATCCCTCTTGATGATAGTTACTAAATGTGATTTGTTTACGTATATAGTACCTT |
| | | | | | CCAATTGAAGGTTTGGCCGC[C/A]TTCAATAAGGCAACGGCAGAATTATTGTTTGGAGCA |
| | | | | | GATAACCCAGTAATCAAAGAACAAAGAGTAAGCTTGAATTTCGAATTTGAGTCAAGAAA |
| | | | | | AATTAATTCTTTTCATGGTACCTTGATTATTTTTGTCATATTTTCTCTGCTTTATATCAGGT |
| | | | | | TGCAACCGTTCAAGGACTTTCAGGAACAGGTTCTCTCCGTCTCG (SEQ ID NO: 70) |
| common_528 | NC_044370.1 | 100088557 | T | C | ATGAGATAATTAAGCAATGAGCATGCATGTTAAATCAATTTTATTAATTATACATACTGAA |
| | | | | | TTTGTTGTTATCATCGTAATATAATAGATATTAATTAATTTAAAAATAAAATTATAATAATA |
| | | | | | ATAATAATATTTACAGGGATATACACTTTTACTGGTGCAAGCCCATCGAAGTGAACTAAG |
| | | | | | GCCCCCAAAATGCGTAGA[T/C]AATATTAATCACCAATGCGTAGAAGCAACCACAGGAG |
| | | | | | AGGCCTCAATCCTATACGTGGGGCTCTACCTTGTTGCTCTGGGGACTGGAGGAATCAA |
| | | | | | AGCAGCACTCCCATCATTGGGTGCTGACCAGTTCGATGAGAGGGATCCAAAGGAGTCG |
| | | | | | AGTCAATTGTCGAGTTTCTTCAACTGGTTCTTGTTCAGCCTCACCATT (SEQ ID NO: 71) |
| common_531 | NC_044370.1 | 100304872 | A | G | AATTACCCTAAAAACTATATGTTTGAGAAAGAAAAAAATAAACAATAAGGTGTGTGAGTG |
| | | | | | TGACAAGACTTATTCAAATGTGCAGGCTCTTATTCTAGGGGGCACTGATACAACAACAG |
| | | | | | TAACAATGACATGGGCATTGGCTCTACTTGTCAACAACCAAGACACGTTGAGAAAAGCA |
| | | | | | CAAGAAGAATTAGACCAAGTGGT[A/G]GGGAGAGAAAGACAAGTAAAGCAATCGGACAT |
| | | | | | AAACAAGCTGGTTTATCTCCAAGCTGTTATCAAAGAAACAATGCGCTTATACCCAGCCG |
| | | | | | CACCACTCGCACTCCCCCACCAATCGGTTGAAGACTGTACTGTGAGTGGGTACCACGT |
| | | | | | TCCAGCTGGCACACGCCTCCTCCTCAACCTTTCAAAGCTACAACGAGATCCA (SEQ ID |
| | | | | | NO: 72) |
| common_541 | NC_044370.1 | 101321815 | C | T | TTTATTATATGTATCACTGTAATGCCATGAGTTTAATATGTAAATTTTTCCAGGCATTATA |
| | | | | | CATGCTTAAGAAATTTAAACAAGGAGTCCCCTTATTTTATCTTTTTTCAGTAGGATTTGTG |
| | | | | | CTGGTTGCCAAAGTGAGATTGGTTTTGGACGGTATCTGAACTGTTTAAATGCATTTTGG |
| | | | | | CATCCAGAATGCTTCCGTTG[C/T]CGTGCTTGCCATCTACCCATTTCTGATTATGAGGTA |
| | | | | | TTTTTAAACCGTTATAATATGAGCTGTCTAAGCAATGGTGCATTGGTATAAGACTTCTAG |
| | | | | | TCATTTAACTGGTGGTTATTTTCCCTTTTGCAGTTTTCTACATCTGGGAACTATCCTTACC |
| | | | | | ATAAAACCTGTTATAAAGAGAACTATCATCCGAAATGTGATGTC (SEQ ID NO: 73) |
| common_549 | NC_044370.1 | 101942035 | T | A | ATATATATATAGATAAGAAGACAGCTAATGGTTTTTTTTTCTTCTTCTTCTTCTTCTTTCTT |
| | | | | | CACCCCGATAAGTTGTTTCGATTGGTTAAAAAGATAAAGGCAAAAGTAGTGATAAAAGTT |
| | | | | | TGATGATATGGGTAAGAAGAAGAAGCAGAATCAGAAAACAAAGGAAATTTCAGTAGCCA |
| | | | | | TAGCTGAAGCAGCTTCATCT[T/A]TAAAAGGAGAACCTCATCATCATCATCATCATCATCA |
| | | | | | TCATCAGTCGACGCCGAGAAAGAGAGGTAGGCCTCGGAAAATTAGCATTGAAAAAAGT |
| | | | | | CTACAGAAAAAAGAAGAAGAAAAAGAAATAGTAGTTGGTGATGATGATGGTATCATTATT |
| | | | | | ATTAGCCAGAGTCAATCCAAGAAAGCTAAAATCGATGAACTTGAAG (SEQ ID NO: 74) |
| common_550 | NC_044370.1 | 102006836 | A | C | TCCTGAATTGTTGTCCACTGAAACCGTCCGGCAACTGCATGCTACCATCGAAAAAGAAT |
| | | | | | GGGATGCCCTTCGAAGGTCAGCATGTCAAACGGCTGCTGGACGAGCACTGTGGAAAC |
| | | | | | ATGTCACTCATGACCCTTTGGCAGCCTTACTTGCTGGAGAGACTTACCTAAGAAGCCTT |
| | | | | | CATGAGAAGATAAAGAAAGACTGTGC[A/C]AACAATGCAAGTGAAATCTCTGGAGTTATT |
| | | | | | CTTGCAGTTAGAACTCTCTGGTTCGATTCAAAACTTGAAGCAGCCCTTCATTCCTTACAT |
| | | | | | GGCACAGAAACACAAGTTGTTCTCCTTGGTGCAGGTAAATGATAAACACCAAATTTTAC |
| | | | | | ATATATTAACAGATAGCATAATTGTACTTTTACATATCTTAATTAGTAGTAC (SEQ ID |
| | | | | | NO: 75) |
| GBScompat_ | NC_044370.1 | 70516405 | T | G | ATATCATAATGCAACATTACTAATTATTTTGATGACTAATTTATAGATACAAGTAACTGAA |
| common_79 | | | | | ATTGATTTATTTGATTCTCAGAATTCTAGCTGTGTACCAGTAAACTATATGGAAAATCAAC |
| | | | | | CGGATATAAATGAAAATATGAGAGGGGTTCTGATTGATTGGCTTGTAGAGGTACTTAAA |
| | | | | | TTGCTGCTTTCCTGTTGTTA[T/G]TGTACTCAATGAATTCCTCTTTAATGGAATTCTCTGA |
| | | | | | CCATTTAGTGCAAATTACAGGTTCACTACAAGTTTGAATTAATGGATGAGACGTTGTATC |
| | | | | | TCATGGTCAACTTGATTGATAGATTCTTAGCTGTTCAATCTGTGCCAAGGAGGAAACTTC |
| | | | | | AGTTGGTTGGGGTTGCTGCCCTACTTCTAGCCTGCAAATACGAA (SEQ ID NO: 76) |
| GBScompat_ | NC_044370.1 | 71626242 | A | T | TAGACGAAAGGAAAGTAGTAGGTACAAATTTATAGAACAAAAAAAAAGAACATAATGAT |
| common_81 | | | | | GCGAATGTATGGTTATAATGTTTTCAAATAACTAGAAAATACAATACTCACTTGATAAGC |
| | | | | | CAGTGAAACTCTTCCTCCTTGTCAGAACAACCATAATCTGAAGTCCACGCGTGGCCTGA |
| | | | | | ATTTTTTTTTTTTTTGGACAAAT[A/T]ATTATAGCAAACAATCAGCTGCAGTTTGAACTTAT |
| | | | | | GCACACATTTAATTAGTTGTGTTAAAAGGTGTATATAGACTAACCTATGGTAAACTTGTG |
| | | | | | GAATCGCAGCATGTCCATTACACCAACATGAGCTAGTGCACAGCCAAAAAGATCAGGT |
| | | | | | CTCTGTAACAAAATGAAAAAATTTGTTTTGTAATCAACCACGCATAAG (SEQ ID NO: 77) |
| GBScompat_ | NC_044370.1 | 79818534 | C | T | CAATATTGGGTGTGATCTATCTTTGCGATTGGGTCCTATGAGTGTTGAGAACAAGCAAC |
| common_84 | | | | | CCCAGGCTATTGCAGACGTTAATCCCCGAGAGGGAGGCAAGTTTTGTGACCAATTACC |
| | | | | | ATTATTGGATAAAGGGTGCTCCATGTTTCCTCCTAGAGGCAATGCTTGCCAGGCATTAA |
| | | | | | GTTCATACAAAGGTGTAGAAGCAGC[C/T]GTGCGAAAACGAAAGCCATCTTTTGATAATG |
| | | | | | GAATCGACGATCAGCAGTTTTGTAATTTGAAGCCAAGTCCTCCTTTTCTCCACTTAAATG |
| | | | | | GAAAAATGAGAAATGCTGGATCATAAGTTCTGATTTTTTCTTACTTGGTTAACTTTGTGG |
| | | | | | TGCAATTTTGTTGTACTCCCGGATGTGTTATGCACCCAAGAGCTACCAGA (SEQ ID |
| | | | | | NO: 78) |
| GBScompat_ | NC_044370.1 | 92378793 | A | G | GCTACATAAAGACAGAAACACCTTTTTTTTTTTTGAAAACTACACAAGTAAAAAAAAATTG |
| common_94 | | | | | TAGAAAAACAAAATCCATAAAAAGAAAAAAAATATATATATTTATAAGAAAAAATACAATT |
| | | | | | TTAAGTAGTGTAAAAGATATTTATAAGTAACCTGGGTTTTGAAATTATGCAATCGCAATTA |
| | | | | | AATGAGATATGGCAGCAG[A/G]GCAGATTGATTGACTCAGGTATGTCATCTTCTAAAATT |
| | | | | | TCAATAACTACTGATTTCTTTTAAATGAAACCACACAATCCAACTCAAATAAACAAATAAA |
| | | | | | AAATAACATATAATGTAATGGTCAACTACTCCTACAGCTATCTAGAAAATGATACATCAA |
| | | | | | ACATGGCTGCATCTCCAGATCAAGAGTGGTCATCCTCCCAGC (SEQ ID NO: 79) |
| GBScompat_ | NC_044370.1 | 98559392 | C | A | CGAATCCTCTTCTTCCTCAACGTCTCAGAGCTTCACACAATGGCGTTTCCCACTACCAA |
| common_96 | | | | | ACTGTCCAGTTCGTAATATCAATATACGATCCGAATCCGATTTAACACCGGAGGAAAGT |
| | | | | | GATCAGACTGGTCACCATTACGGCCCGCCACCTATTTCTCCCACGAACCTCCAAGAAG |
| | | | | | TGTTCCACGCAGCTGAGCTCCAACT[C/AJAGCGTCGGGTCGGATTCGGATCAGTTATCT |
| | | | | | GCCCTACAACTTCTAGAGAGATCTCTGGTTCCCAACCCGCCGACCGATCCTGAATGCC |
| | | | | | CGCCGGAGTTGATGCGCGGTGTAGTGGGGAGTTTAATTTCTCAGGTTGGGGCGAAACC |
| | | | | | TGCGTCGAAGATTTTGCTAGCTCTCTGCTTGGCGGAGCGAAACCGGAGAGTTGCC |
| | | | | | (SEQ ID NO: 80) |
| GBScompat_ | NC_044370.1 | 100988215 | G | A | TGATCTTCAGAAGCAGGTGGCCGAGATGGCTGGAAATGAGAAACAGATTCTTAGTATTG |
| common_97 | | | | | AAGAAGAAATAAGAAGATTACAGAATTTGGTTGGTGATGTGCTACAAGATCCTGGACTA |
| | | | | | GAAGATCAAATTTCTTCTGGCAGTAACATTGAGTGCTTAGAAATGTTGCTGAGGAAGCT |
| | | | | | TCTAGAAAATTATGCAAAATTTTC[G/A]ACCATAAGACCTGTACTTGGTGGTGGAATTGAT |
| | | | | | GAGCTGCAGACTGACGTGATGACTGTAGAGGCAGCTAAGAACTTAAGCAAAACCCATG |
| | | | | | CTGGGGAGTCCGATGAAGTTATCATGAAGAAAGAGCTTGAGGAAGCTTTGCACGAGTT |
| | | | | | GATTCTTGTGAAGGAGGAGAGAGGTGTATTTGTTGAGAAGCAACAATCTTTG (SEQ ID |
| | | | | | NO: 81) |
| GBScompat_ | NC_044370.1 | 101069452 | G | A | TGTGCACCAAACCAAGGAAATGGAGTTGAAGAAAGACAAGTTTGTCAGGTGAACTCCTT |
| common_98 | | | | | TACTTCATTATCTTTCTTTTACAATCATGCATGCTTAACTGGGGTTTTTCATTTTCTCCAA |
| | | | | | TTGTAGATTTTACCATGAAGAAAACAATAAAGATAGTAACTTAGAGAAGCCGTTACCAGT |
| | | | | | GTTCAAAGTAGCAGCAGCAGA[G/A]CCTTTGTTTGAGGCAGAAGGAGGTAGAAGTAGTA |
| | | | | | GTAGTAGAAACAGAATTTATATTCCAAAATTTGGAAGGTTTAAAGTTTTCCCAGAAAATG |
| | | | | | AGAATCCATGGAGGAAGAAGATTCTTGACCCTGGAAGTGATATTTTCCTGCAATGGAAC |
| | | | | | AGAGTTTTTCTCTTCTTTTGCTTGGTAGCACTTTTTGTTGATCCACTC (SEQ ID NO: 82) |
| rare_63 | NC_044370.1 | 101144766 | A | T | AAAGCTCAAGAATTTCCAATCCTGGCGTGGAAACTCTGGTATCCTCCCTACAAACCGGT |
| | | | | | TACTCTCAACGTTTAACTCCACAAGCTTAGTCAACCTCACAATGGAGTTGGGCAATCTC |
| | | | | | CCATCAAACTCATTTCTGTCCAAGTAAACTTTCTTCAAGTAAACCATTCCTTCAAAACTAT |
| | | | | | CGTCCGGTATGTCCCCTGAAAA[A/T]TCATTGTACGACAAGTACAAGGCTCTCAACCCTC |
| | | | | | GAAGCACTTTGAATTCAGGTATTCCACCCCCAAAGTGGTTGCCTATCACGCTGAGGCTG |
| | | | | | CGCAGAGTGGTTGGGAGCTTGGACAATGTGTAGACATCGATGGTGCCACCTAGTOCCA |
| | | | | | TGTTTTCAAGTCTTAATCCGTAAAGGCTTCCATTCACGCATGTCAACCCT (SEQ ID |
| | | | | | NO: 83) |
| rare_66 | NC_044370.1 | 102037098 | A | T | ATCAAGTAACTGCAAGTTGGGAATAGACCAGAGTGAACCAGGAAGAGCACCATCGAGT |
| | | | | | TTATTGTCACTCAGAACCACAACCCGCAGCTGAGAAAGGCTTGAAAACAAGCCCTTTGG |
| | | | | | CAATGGACCCTCTAGACCATTAGCACTAATATCAATAACCCTCAAGCTCCTCAAACCCG |
| | | | | | TGAATTCTTTAGGAAATGACCCAGT[A/T]AAAAAATTCTTGCTACAATTTAGCTCAACAAG |
| | | | | | TCGAGAAAGTTTATTCAACTGAACAGGTATAGAAGCGGTAAGACTATTATCAGAAGTAC |
| | | | | | TTAAAAACTCAAGTCTCGAGAGGTTACCTAAACCTGATGGAATCGACCCAGAAAAGAAA |
| | | | | | TTTGAGGAGAGGTCAAGAGTAGTAAGATTTCGAAGTGAGGTAAACTCGAAC (SEQ ID |
| | | | | | NO: 84) |
| common_1527 | NC_044372.1 | 61687722 | G | A | TGAAAGATAAAACTAGCATTAAAAAAATAAAAATTAAAAATATTCTTATGATATCGAACTG |
| | | | | | CAGGCAGAATGTCTGTTGGTGAAGATCGATATCCATTGCCGTATTCAAGGGGATGTTGT |
| | | | | | TCTTGAGTGTATCCACTTAGACGAAGATCTTGTACGTGAGGAGATGATGTTTAGAGTTA |
| | | | | | TGTTCCATACAGCATTTGTGCG[G/A]TCAAATATTTTGTTGCTAAATCGTGATGAAATTGA |
| | | | | | TGTTTTGTGGGATGCCAAAGACCAGTTTCCCAGGGACTTTAAGGCAGAGGTAGGTATTA |
| | | | | | CTATTCTTCTGTGGTTTCTTTTTGTGAAGCTGTTGGAGTTGAATCATACTCATATCTGTAT |
| | | | | | CAGGTACTTTTTTTGGATGCTGATGCCGTTGTCCCTGATATCACCA (SEQ ID NO: 85) |
| common_524 | NC_044370.1 | 99761993 | T | C | GGTACGTGCAATAAAATATTTAAACTTATTTTCAGCACGATAAATTATTTAAAATTTTATA |
| | | | | | AAATTTAACAATAAATTCAAACAAAATGATAAATAACAGTGTATAATATTTTGATACATAAT |
| | | | | | TTTAGAACACACTTTATTTGAAAGCTGAAAATTAATTATTAATCAGTACTTAAGATAGTTA |
| | | | | | GAGAACCAATCCAACAT[T/C]TTGGAATGGGCCTCATTTGCAGATTTTGATGCTGCTTCA |
| | | | | | TCATCCAAGTTGTACCTCACTGTCCAACCATGTTCAACCTTTGGGATAATCTCAACATGG |
| | | | | | CTCTCAACCTATACCATTGCCAACCATTCATTAATTTATGCATACAATTAATTAATATAAT |
| | | | | | TAAAACTTATTCATTTATACAATTCATATCAATTATTATCA (SEQ ID NO: 86) |
| rare_30 | NC_044370.1 | 28544332 | G | T | AAAATCATCTTCTGCTTCTTCTTCAGCTGTTAGAAGACCCAAATTATCGATCTCTGATCA |
| | | | | | ACAATCTAAAATTAACGGTGGTGATCGCACTGTTAAATCCTCTTTGGACTCGATGACCG |
| | | | | | TCGATGGCTACCTTCGCACTGAAGCCTCAGCTATCACTGCTGAGTCTACTCTTTTAGAT |
| | | | | | GCACAAATTACTCTGATCGACCC[G/T]ACTCCGACAACCTCCATTTCTGCTGCTGCGGT |
| | | | | | GGCAGTGGCTCCTGGAGATTTGAATTCCGGCAGCATTGGCTCATCTAGTGCTCCTAAG |
| | | | | | ACTGTTGATGAAGTGTGGCGGGAGATCGTGTCTGGAGATAGGAAGGAGTGTAAAGAGG |
| | | | | | AGGAGCAGGATATGGTGATGACGCTTGAAGACTTTTTACTTGCCAAAACTGGG (SEQ ID |
| | | | | | NO: 87) |
| common_10 | NC_044370.1 | 889040 | T | G | GAGCACACTATTGTTATGTTACTATTTTATGACATTTTTAATGAATGTGATAAGTATTATT |
| | | | | | CAAATCAATTGCCATGAATAGCTTCTGAATCTCCATGCCGATGATTTTCATCGAGGTTAG |
| | | | | | GATTATTTGTCTGTATTTGTGGCGGGGTTGCAGGTAGTTGATCCATCTGAGATGTTTCT |
| | | | | | ACGGAAGCCTGCTGTTGGTTT[T/G]CCTGAGAGCGTCGTTGTCTTCTCCATTTTGAAATC |
| | | | | | TCGACAAGTATAGAATTCCCACACATAGTAACCCCGAATCCGGTAAATGTAGCTAACAG |
| | | | | | AACAGATAGAACTCCTTGCATATGAAGCTGTAGATTACATGATAGCAACATTTTAAACAG |
| | | | | | TCTATACTGAAGATCTTAATTGAAAATAGTTTTCTTTTGTAAACCAA (SEQ ID NO: 88) |
| GBScompat_ | NC_044370.1 | 32763867 | T | G | CAAAGTCTAGCTGTAAGATAGAACTACTTCTGAATTTTGATTCAACTTCTGTAGTTCTAA |
| common_54 | | | | | CTATATCTTCTAACACTGGCACTGAACGTTCAGAGTGGTATAGCAAACAGAGCTGTGGG |
| | | | | | AGAGACCCGTATCCACAATTAACTTTTACAATATAATGACAGTAGATTCCTCTGTATTCT |
| | | | | | CTTTGGGGTACTCTAGATTTAT[T/G]TGAAGACCATATTTTCCTTGTTTCTGACCTTATTAT |
| | | | | | AAATAGAAATGAACATTACCAGCAGCAGAAGTCATTGTGTTTACATATTCACTCTCCAGC |
| | | | | | AAGAATTGAAGAAAGATAATCGGTACAACATCTATTTGTGTTTGTATCAGGTTTTTACAT |
| | | | | | AAACTAATATTGAACTTTGAAAATCTACTGACAGGTCGAGAACTA (SEQ ID NO: 89) |
| GBScompat_ | NC_044370.1 | 35677966 | A | G | GTTGTATCGAAACCTATTCGCCCAGCTTTGGCAAATCTCAAGGGAAACTTTTGTTTGTA |
| common_56 | | | | | GCAACCATTTCTGTTAGAGAAAGCAGCTTGGCAATTACACTCATTCAAACAGCTTTGTTT |
| | | | | | ACAAGTATCCTTGTCAACCAGTGAGAGGAATTCGTAAGTGTCCGGTTCCCATCTCAAAC |
| | | | | | CGTCTAATTCAGCTATAGAAACC[A/G]CCTTGTCACAGCCTTCTATGGTTGAATTTCTGTT |
| | | | | | GCAGCCTAAAGTCTTCTGGTTTTTATCAATGTAATCTAAACCAGGAAGACAAAGACAAAC |
| | | | | | AGGTTCTTGATTTATAAGCTGACAATATGAATTGATGCCACATAATCCAATTGGAGCACA |
| | | | | | AAGATTGGTAGTTGAAGACCACTCAACAAACCAGCTGCTGTTCTGAA (SEQ ID NO: 90) |

TABLE 12
Targeted sequencing primers (5′ to 3′) for the SNPs identified in Tables 1
and 3-10, as described in Examples 1 to 4.

| SNP Name | Forward Primer | Reverse Primer |
|---|---|---|
| common_491 | TGGCTAAAGTCATGCTCCATGT | CACACCTAGTGGGATGTATCAGT |
| | (SEQ ID NO: 91) | (SEQ ID NO: 92) |
| common_512 | CTGTCGAGGCCCATCTCAAA | AGCGAGGCAATACCATCAAGA |
| | (SEQ ID NO: 93) | (SEQ ID NO: 94) |
| common_517 | GGATTTGCTTCACGACAGCC | CCCAAAACCACTTTCGCCAG |
| | (SEQ ID NO: 95) | (SEQ ID NO: 96) |
| common_525 | AGCTTGGGTGATACAGCTGC | ACCCGATTGCTTACTAGGTCC |
| | (SEQ ID NO: 97) | (SEQ ID NO: 98) |
| common_511 | TGCTTGATTCCGAGGATCCT | GACTTTGGCCTATGCAGAGGA |
| | (SEQ ID NO: 99) | (SEQ ID NO: 100) |
| common_518 | ACACTTACCCGCTCTTCAGG | AGAGGTGGAATTGGAATGCCA |
| | (SEQ ID NO: 101) | (SEQ ID NO: 102) |
| common_522 | TATTAGAGCTGCCTTCGCCG | AACCAATCCGTGAACACTCT |
| | (SEQ ID NO: 103) | (SEQ ID NO: 104) |
| common_533 | AGTCCTCTCTTGATAGCCATCT | CCAAAGCGGGAATGTGACAA |
| | (SEQ ID NO: 105) | (SEQ ID NO: 106) |
| common_534 | TGGTGTGGTTTCTTTCTGTCCT | TGATGGTGATGGTGAGGCTG |
| | (SEQ ID NO: 107) | (SEQ ID NO: 108) |
| common_539 | AGCTTCAGAGGGGTTTGTGT | AGGAGTGTTACGACAGCAGC |
| | (SEQ ID NO: 109) | (SEQ ID NO: 110) |
| common_544 | AGGCCATGATGAAACGACGT | TATTGGGCTGGGCTTCGTAC |
| | (SEQ ID NO: 111) | (SEQ ID NO: 112) |
| common_545 | TTGCTGGCCAAGTGAGTTCT | CCTTGTCGGGGTGGTTCAAT |
| | (SEQ ID NO: 113) | (SEQ ID NO: 114) |
| common_546 | TCCGTGCCCTTGGTAAAGAG | ACGGCAGTAGTAGTGCATGC |
| | (SEQ ID NO: 115) | (SEQ ID NO: 116) |
| rare_50 | TCGAACCGTACTTTGCCACA | GAAAAGTAGCGTCACGGTGG |
| | (SEQ ID NO: 117) | (SEQ ID NO: 118) |
| rare_57 | GGTTTTAGCGTCGCGTTGTG | ACACGCCCATAAAGACAGGG |
| | (SEQ ID NO: 119) | (SEQ ID NO: 120) |
| common_472 | TGGGTCCGGAAGATACAAGC | ATGAAGGTGGCGCAATGGAA |
| | (SEQ ID NO: 121) | (SEQ ID NO: 122) |
| common_474 | TCAGTTTCTCTGTCTGCCGA | GGAGTATAGGGCGGTGGGTA |
| | (SEQ ID NO: 123) | (SEQ ID NO: 124) |
| common_483 | TCCTCCCTCCATTCCGATCA | ACTCACCCAAAAGTGGCCAA |
| | (SEQ ID NO: 125) | (SEQ ID NO: 126) |
| common_484 | CCACAGGCAGCACGTTTCTA | CTAAGGCCGTTTTGGGACCT |
| | (SEQ ID NO: 127) | (SEQ ID NO: 128) |
| common_486 | GACCTGACATCACCGTTGCT | ACTCGACTTCTGCAGAGCTT |
| | (SEQ ID NO: 129) | (SEQ ID NO: 130) |
| common_514 | AGGAGATGTCAGGAGGTGGA | TTGTTTGGCACCAGTGGACT |
| | (SEQ ID NO: 131) | (SEQ ID NO: 132) |
| common_521 | TACCCTACTTCCTTGGGCGA | AGGAAAATGGCAGACACCCA |
| | (SEQ ID NO: 133) | (SEQ ID NO: 134) |
| common_527 | TCTTCATTGGGAGGCAGCAG | AGGCTCCACCACTGAGTGTA |
| | (SEQ ID NO: 135) | (SEQ ID NO: 136) |
| common_532 | TCTCCGAATACCCTCCACGT | GCGTGTCCGAAAGTTTGTGT |
| | (SEQ ID NO: 137) | (SEQ ID NO: 138) |
| common_552 | TCTTCCCAACTCCTCCTGGT | TGCAAGTTTTGAAGTGGCCG |
| | (SEQ ID NO: 139) | (SEQ ID NO: 140) |
| common_553 | TCTAAGGTGGTTGCAGGCAG | TGGACGTCTAAGCAAAACAAG |
| | (SEQ ID NO: 141) | (SEQ ID NO: 142) |
| GBScompat_ | CAAGTCCAACCCAGGAGCTT | GGCTGAGTCCTGCAAAGTCT |
| common_91 | (SEQ ID NO: 143) | (SEQ ID NO: 144) |
| GBScompat_ | CACCACTGCCTCCACCTATG | CAAATGGGTGCTTCGGCTTC |
| common_99 | (SEQ ID NO: 145) | (SEQ ID NO: 146) |
| GBScompat_ | TTTTGCAGCGATCGATTCCG | GGCACGATTCTACACCACCA |
| rare_10 | (SEQ ID NO: 147) | (SEQ ID NO: 148) |
| common_203 | ACGCCTGGTTGAAGTGAACA | ACAAAGTTTGGGCCCACCTT |
| | (SEQ ID NO: 149) | (SEQ ID NO: 150) |
| common_288 | CCCAATCCCAAATCCAAAACCA | GGTGGCAATCGTGGAGAGAA |
| | (SEQ ID NO: 151) | (SEQ ID NO: 152) |
| common_300 | TGGTTACCCTGTAATTGGTTGAGA | CTTTTGCAGCCGATGGTTCG |
| | (SEQ ID NO: 153) | (SEQ ID NO: 154) |
| common_313 | TGAGGGCCACAACTTAGTAGTG | TGGCTCTGATGGAGATGACAC |
| | (SEQ ID NO: 155) | (SEQ ID NO: 156) |
| common_375 | CAGGCTTTGTCCAGATGTGC | ACAACCAGCCAAGAGACCAA |
| | (SEQ ID NO: 157) | (SEQ ID NO: 158) |
| common_424 | TGGACTGACCTGAGACCAGT | GCAACCTGGAGAGAGATACCG |
| | (SEQ ID NO: 159) | (SEQ ID NO: 160) |
| common_425 | ATCCGGTTTCTTGTTCGGCT | ATGGTCCGGAGAGTTCATGT |
| | (SEQ ID NO: 161) | (SEQ ID NO: 162) |
| common_426 | GGATTCCTCCATTGTGGGCA | ACTTGTCCCAAAACCAGGCA |
| | (SEQ ID NO: 163) | (SEQ ID NO: 164) |
| common_428 | GCGCTCTGGTCTCTTCAAGA | AGGCCAATCTCGCTTGTCTC |
| | (SEQ ID NO: 165) | (SEQ ID NO: 166) |
| common_431 | GCAGAACACCATCAAAGCCG | CGTCTACGAAGCGGAGGAAA |
| | (SEQ ID NO: 167) | (SEQ ID NO: 168) |
| common_432 | CTCTCCGCCATAGCCAAGAC | GCCTTGGGATTCCTCTTCGT |
| | (SEQ ID NO: 169) | (SEQ ID NO: 170) |
| common_434 | CTGCAGGGTTGTGGAGAGTG | GTTTGGAGGGATTTCAGAGCT |
| | (SEQ ID NO: 171) | (SEQ ID NO: 172) |
| common_436 | GTTCACTTTTGCCACACTGCA | TGTGAAGCTGGTAGGACACT |
| | (SEQ ID NO: 173) | (SEQ ID NO: 174) |
| common_437 | GCAGATGTACGCTAAAGATTTGTT | GCGTGGGATGTAACGGTCAA |
| | (SEQ ID NO: 175) | (SEQ ID NO: 176) |
| common_439 | TGGTCTCTCTCTCTCTTCCACA | TGATCGAGTCTCCGAACACG |
| | (SEQ ID NO: 177) | (SEQ ID NO: 178) |
| common_445 | CGGGGAAGGAGAGGAAGTTG | GCATCAAAGCCTTGAGTGCC |
| | (SEQ ID NO: 179) | (SEQ ID NO: 180) |
| common_448 | CGGTTTCACCACCACTAACC | GGGTATCGGTTGGATGGTCA |
| | (SEQ ID NO: 181) | (SEQ ID NO: 182) |
| common_449 | TGTTGATGTGGCGGTGACAA | TGCCACCAAACCATAATAGGGA |
| | (SEQ ID NO: 183) | (SEQ ID NO: 184) |
| common_452 | TCCGACGAGTCTGACCAAAC | CAACGATTTCCCCAGGGAGT |
| | (SEQ ID NO: 185) | (SEQ ID NO: 186) |
| common_453 | TGACAAAGTTGATAATCAATGCAGT | TATGCTGGTGGTGGAGAGCT |
| | (SEQ ID NO: 187) | (SEQ ID NO: 188) |
| common_454 | CTAGGATCTCCAAGCAGGCG | GTCATTGTAAGCCTGTGCGG |
| | (SEQ ID NO: 189) | (SEQ ID NO: 190) |
| common_455 | TTTACATACCCCAGCACGCA | CGTGACAGTTGGATGAAGCG |
| | (SEQ ID NO: 191) | (SEQ ID NO: 192) |
| common_459 | ACCACCTTGGATTCCACGTC | CGACCTTGCAGTTGAGCCTA |
| | (SEQ ID NO: 193) | (SEQ ID NO: 194) |
| common_470 | CTTCTGACCACCAACAGGCT | CAGAAGTCAAGGTTTGTTGCCT |
| | (SEQ ID NO: 195) | (SEQ ID NO: 196) |
| common_473 | CCGTTGAAGCATTGCACCAT | TCCCAACGTTTCCATTGACG |
| | (SEQ ID NO: 197) | (SEQ ID NO: 198) |
| common_476 | ATCTGACGTGGACTCAGTGC | CACTATTGTGGCTGCAACCG |
| | (SEQ ID NO: 199) | (SEQ ID NO: 200) |
| common_478 | CCAGCCAATCTCACCGATGA | GTGAGAAACCTTCCGACGGT |
| | (SEQ ID NO: 201) | (SEQ ID NO: 202) |
| common_479 | AGTTCTGGTTTGTTTCAGGAAGA | TGAGGAGGGCTTCAATTGCA |
| | (SEQ ID NO: 203) | (SEQ ID NO: 204) |
| common_487 | AGCTAGCAGCACCTTCAATGA | AGGAGCTATGATGCGATTGGA |
| | (SEQ ID NO: 205) | (SEQ ID NO: 206) |
| common_488 | GTCCAATGAAAGCCGCAACA | TAGGTCTCGAGTGCAACAGC |
| | (SEQ ID NO: 207) | (SEQ ID NO: 208) |
| common_489 | CCTTGGAGGATCATTGGTGCT | CCACATTGCAAACAGGGCAG |
| | (SEQ ID NO: 209) | (SEQ ID NO: 210) |
| common_492 | AAGAAAGGGTCCAAAGCAGT | ATGTATCAACCTGCGGCAGA |
| | (SEQ ID NO: 211) | (SEQ ID NO: 212) |
| common_494 | CCAACAAAGAGTGCCTCAGC | TCTGCTTGGCATCCAGATGG |
| | (SEQ ID NO: 213) | (SEQ ID NO: 214) |
| common_496 | TGGAGTCTGCATTTGGTGGA | GCATAGTGTGATTGGGGGCT |
| | (SEQ ID NO: 215) | (SEQ ID NO: 216) |
| common_497 | CCTCCAGGGTAGAGTAGGCA | TGCAGTTTGGGCTGAAAGGA |
| | (SEQ ID NO: 217) | (SEQ ID NO: 218) |
| common_500 | AACTCGGTTGGTGGTATGCA | ACCGAGGCACACAAACTCTT |
| | (SEQ ID NO: 219) | (SEQ ID NO: 220) |
| common_510 | CGGTACTACCCATCGTCGTC | TGGCAGAGTCCACAGCATTT |
| | (SEQ ID NO: 221) | (SEQ ID NO: 222) |
| common_515 | TGCTCACCGAGAATCATCACA | AGAGCCAAATGGAAGGACTGG |
| | (SEQ ID NO: 223) | (SEQ ID NO: 224) |
| common_520 | ATAGAGCCAGACGTGGTGGA | CTGTTCATAGGTCAACCCCCA |
| | (SEQ ID NO: 225) | (SEQ ID NO: 226) |
| common_523 | ATTCATAGCATGCGCGCAAC | TCAGCTCCAACTTCATTGCAG |
| | (SEQ ID NO: 227) | (SEQ ID NO: 228) |
| common_526 | AGGTTTGGCCGCCTTCAATA | CGAGACGGAGAGAACCTGTT |
| | (SEQ ID NO: 229) | (SEQ ID NO: 230) |
| common_528 | TGCAAGCCCATCGAAGTGAA | GGTGAGGCTGAACAAGAACC |
| | (SEQ ID NO: 231) | (SEQ ID NO: 232) |
| common_531 | GGCTCTTATTCTAGGGGGCAC | GAAAGGTTGAGGAGGAGGCG |
| | (SEQ ID NO: 233) | (SEQ ID NO: 234) |
| common_541 | GTGCTGGTTGCCAAAGTGAG | ACATCACATTTCGGATGATAGTTCT |
| | (SEQ ID NO: 235) | (SEQ ID NO: 236) |
| common_549 | TCTTCTTCTTTCTTCACCCCGA | GCCTACCTCTCTTTCTCGGC |
| | (SEQ ID NO: 237) | (SEQ ID NO: 238) |
| common_550 | GGCAACTGCATGCTACCATC | ACCTGCACCAAGGAGAACAA |
| | (SEQ ID NO: 239) | (SEQ ID NO: 240) |
| GBScompat_ | AGGGGTTCTGATTGATTGGCT | GCAGGCTAGAAGTAGGGCAG |
| common_79 | (SEQ ID NO: 241) | (SEQ ID NO: 242) |
| GBScompat_ | CACGCGTGGCCTGAATTTTT | TTTGGCTGTGCACTAGCTCA |
| common_81 | (SEQ ID NO: 243) | (SEQ ID NO: 244) |
| GBScompat_ | GGGAGGCAAGTTTTGTGACC | TGCATAACACATCCGGGAGT |
| common_84 | (SEQ ID NO: 245) | (SEQ ID NO: 246) |
| GBScompat_ | ATGGCAGCAGAGCAGATTGA | GCTGGGAGGATGACCACTCT |
| common_94 | (SEQ ID NO: 247) | (SEQ ID NO: 248) |
| GBScompat_ | AGAGCTTCACACAATGGCGT | TAAACTCCCCACTACACCGC |
| common_96 | (SEQ ID NO: 249) | (SEQ ID NO: 250) |
| GBScompat_ | CCGAGATGGCTGGAAATGAGA | AACTTCATCGGACTCCCCAG |
| common_97 | (SEQ ID NO: 251) | (SEQ ID NO: 252) |
| GBScompat_ | TCATGCATGCTTAACTGGGGT | ACTCTGTTCCATTGCAGGAA |
| common_98 | (SEQ ID NO: 253) | (SEQ ID NO: 254) |
| rare_63 | ATCCTGGCGTGGAAACTCTG | CCAAGCTCCCAACCACTCTG |
| | (SEQ ID NO: 255) | (SEQ ID NO: 256) |
| rare_66 | TCACTCAGAACCACAACCCG | CTGGGTCGATTCCATCAGGT |
| | (SEQ ID NO: 257) | (SEQ ID NO: 258) |
| common_1527 | TGCCGTATTCAAGGGGATGT | ATCAGGGACAACGGCATCAG |
| | (SEQ ID NO: 259) | (SEQ ID NO: 260) |
| common_524 | TGGAATGGGCCTCATTTGCA | AGGTTGAGAGCCATGTTGAGA |
| | (SEQ ID NO: 261) | (SEQ ID NO: 262) |
| rare_30 | CGGTGGTGATCGCACTGTTA | GCGTCATCACCATATCCTGC |
| | (SEQ ID NO: 263) | (SEQ ID NO: 264) |
| common_10 | GCTTCTGAATCTCCATGCCG | ACAGCTTCATATGCAAGGAGT |
| | (SEQ ID NO: 265) | (SEQ ID NO: 266) |
| GBScompat_ | GCAAACAGAGCTGTGGGAGA | AGTTCTCGACCTGTCAGTAGA |
| common_54 | (SEQ ID NO: 267) | (SEQ ID NO: 268) |
| GBScompat_ | TTCGTAAGTGTCCGGTTCCC | GCAGCTGGTTTGTTGAGTGG |
| common_56 | (SEQ ID NO: 269) | (SEQ ID NO: 270) |

### Example 5

### Gene Identification

There are presently no known genes identified in Cannabis that have been shown to regulate hermaphroditism or sex determination in Cannabis. Several molecular markers have been proposed for sex determination, but these regions are distinct from the QTLs identified herein. Genes that control sex determination have been described and characterized in several plant species, however the multitude of different genes involved in this process does not easily allow the identification of sex determination genes in Cannabis. The inventors considered genes that may stimulate homeotic transformation or those that are involved in the interplay between flowering and the stress response to be potentially involved in enhancing or eliminating the likelihood of the emergence of hermaphroditic inflorescence in Cannabis. They next sought to identify putative genes that could encode proteins that may be responsible for both the emergence of anthers in pistillate flowers or those that could stimulate male flower development on female plants. Using the findings of the association studies they identified candidate genes at the QTLs identified.

Based on the results of the association studies for hermaphroditic inflorescence from a QTL found on NC_044370.1 based on the SNP marker “common_10” at position 889040 in Example 1, the inventors searched for genes that may encode proteins involved in the stress response or those that could play a role in floral development from an annotated gene list for this region of NC_044370.1 from the Cannabis sativa CS10 genome. Upon inspection of this genomic region and BLAST analysis of putative candidates they identified a single candidate gene LOC115715793, Table 13. LOC115715793 encodes a protein with homology to Arabidopsis thaliana DELLA protein RGL2. DELLA proteins are repressors of Gibberellin signalling. This candidate makes sense in light of Gibberellin being able to induce hermaphroditic flowering.

Based on the results of the association studies for hermaphroditic inflorescence, a QTL demarcated by position 28544332 to 35677966 on chromosome NC_044370.1 was identified from Examples 1, 2 and 3. The inventors searched for genes that may encode proteins involved in the stress response or those that could play a role in floral development from an annotated gene list for this region of NC_044370.1 from the Cannabis sativa CS10 genome. Upon inspection of this genomic region and BLAST analysis of putative candidates they identified a single candidate gene LOC115702418 (Table 13). LOC115702418 encodes a protein with homology to nodulation-signaling pathway 2 protein in Arabidopsis. This protein contains a GRAS domain and is likely part of the GRAS domain family, member of this family play roles in Gibberellin signalling. This candidate makes sense in light of Gibberellin being able to induce hermaphroditic flowering.

Based on the results of the association studies for hermaphroditic inflorescence a QTL demarcated by position 70255045 to 79818534 on chromosome NC_044370.1 was identified from Examples 1, 2 and 3. The inventors searched for genes that may encode proteins involved in the stress response or those that could play a role in floral development from an annotated gene list for this region of NC_044370.1 from the Cannabis sativa CS10 genome. Upon inspection of this genomic region and BLAST analysis of putative candidates they identified a single candidate gene LOC115719981 (Table 13). LOC115719981 encodes a protein with homology to FT-interacting protein 7 in Arabidopsis. This protein regulates the movement of FT a major regulator of flowering in plants. Mis regulation of FT-interacting protein 7 could potentially lead to the aberrant induction of flowering and could potentially explain parts of the emergence of hermaphroditic inflorescence.

Based on the results of the association studies for hermaphroditic inflorescence from the QTL found on NC_044370.1 approximately between positions 94,000,000 and 102,000,000, the inventors searched for genes that may encode proteins involved in the stress response or those that could play a role in floral development from an annotated gene list for this region of NC_044370.1 from the Cannabis sativa CS10 genome. Upon inspection of this genomic region and BLAST analysis of putative candidates they identified ten candidate genes LOC15698113, LOC115698183, LOC115698538, LOC115695629, | LOC115699142, LOC115699627, LOC115699728, LOC115703125, LOC115700107, LOC115700622 listed in Table 13.

The gene IDs LOC115698113, LOC115698183, LOC115699627, LOC115703125 encode putative ethylene responsive transcription factors. From research on likely homologs in Arabidopsis, these proteins may have a role in mediating ethylene signaling and may be involved in the regulation of gene expression by stress factors and by components of stress signal transduction pathways. Ethylene signalling has been shown to be involved in some cases in plant floral development, the stress response is a known trigger of hermaphroditism in many plant species.

The inventors further identified LOC115698538 that encodes for a Clavata1-like receptor kinase. Clavata1-like receptor kinases have been implicated in playing central roles in meristem and anther development in Arabidopsis. LOC115695629 is a gene that may encode a protein with a Myb/SANT-like DNA-binding domain. In Arabidopsis proteins containing similar domains play roles in organ morphogenesis and floral development. LOC115699728 is a gene that encodes a protein with homology to Arabidopsis agamous-like MADS-box protein AGL12. In Arabidopsis, MADS=box proteins have been shown to be involved in the flowering transition. LOC115700107 is a gene that encodes a protein with homology to Arabidopsis E3 ubiquitin-protein ligase MBR2. MBR2 in Arabidopsis encodes an E3 ubiquitin-protein ligase that functions as a positive regulator of FLOWERING LOCUS T (FT) and is important to induce the expression of FT and consequently to promote flowering.

The inventors also identified LOC115700622, a gene that encodes a protein with homology to Arabidopsis transcription repressor OFP7. In Arabidopsis OFP7 acts in the regulation of homeodomain transcription factors that could play roles in floral development.

Finally, the inventors identified LOC115699142, a gene that encodes a protein with homology to Arabidopsis transcription repressor TIFY8. In Arabidopsis likely acts as a negative regulator of jasmonate signalling. Jasmonate signalling has been connected to stress induced flowering in a number of species including Arabidopsis and Tomato.

TABLE 13
Gene list of candidate genes identified. The gene ID is provided with reference to the
publicly available CS10 genome as updated in Apr. 2020 and accessed in Feb. 2022.

| Start | End | | | |
|---|---|---|---|---|
| Position | Position | Gene ID | Protein ID | Description |
| 9247972 | 9249855 | LOC115715793 | XP_030500325.1 | DELLA protein RGL2 |
| 31248309 | 31250365 | LOC115702418 | XP_030485743.1 | nodulation-signaling |
| | | | | pathway 2 protein |
| 79532538 | 79536136 | LOC115719981 | XP_030505012.1 | FT-interacting protein 7 |
| 94086108 | 94088167 | LOC115698113 | XP_030481151.1 | ethylene-responsive transcription |
| | | | | factor RAP2-4-like |
| 94441292 | 94442472 | LOC115698183 | XP_030481233.1 | ethylene-responsive transcription |
| | | | | factor RAP2-4 |
| 95149366 | 95152936 | LOC115698538 | XP_030481483.1 | Clavata 1 Receptor like kinase |
| 95437048 | 95447718 | LOC115695629 | XP_030478545.1 | Myb/SANT-like DNA-binding domain |
| 97492172 | 97496609 | LOC115699142 | XP_030482249.1 | TIFY domain |
| 99557456 | 99558278 | LOC115699627 | XP_030482993.1 | AP2 domain |
| 99684325 | 99689041 | LOC115699728 | XP_030483127.1 | MADS box AGL12 |
| 99933474 | 99934499 | LOC115703125 | XP_030486484.1 | AP2 domain |
| 100782096 | 100786765 | LOC115700107 | XP_030483528.1 | E3 ubiquitin-protein ligase MBR2 |
| 101822631 | 101823768 | LOC115700622 | XP_030484093.1 | Ovate-Transcriptional repressor |

### Example 6

### Genome-Wide Association Studies (GWAS) of an Expanded F2 Population

The inventors engaged in speculation regarding the validity of certain QTLs identified in the GWA of mixed populations conducted in 2020 and from a corresponding GWA conducted on a mixed F2 population. To address this disparity the inventors expanded the diversity of the mixed F2 population experiment by including additional F2 populations, including several populations that were crossed with high THC varieties.

The inventors sought to understand the genetic basis for hermaphroditism by analysing the hermaphroditism trait in 24 designed F2 populations, totaling 2758 individuals. This was accomplished by visually examining female plants for the presence of stamens or the growth of staminate flowers alongside pistillate flowers until the time of harvest during the 2021 field trial season. Hermaphroditic plants were scored as 1, while non-hermaphroditic plants were scored as 0. The percentage of hermaphroditic flowers was calculated for each of the populations. Table 14 presents an overview of the distribution of hermaphroditism in the 24 F2 populations.

TABLE 14
An overview of the F2 populations used in the GWAS study described
in Example 6, including the sample size denoted by the number
of plants, and the percentage of hermaphroditic flowers present.

| F2 Populations | Sample Size | Percent presence |
|---|---|---|
| 21 002 001 | 229 | 37 |
| 21 002 002 | 144 | 21 |
| 21 002 003 | 145 | 28 |
| 21 002 004 | 133 | 20 |
| 21 002 007 | 79 | 18 |
| 21 002 012 | 153 | 12 |
| 21 002 014 | 96 | 21 |
| 21 002 016 | 75 | 15 |
| 21 002 025 | 120 | 34 |
| 21 002 026 | 108 | 24 |
| 21 002 027 | 112 | 24 |
| 21 002 028 | 123 | 16 |
| 21 002 029 | 89 | 12 |
| 21 002 031 | 87 | 13 |
| 21 002 032 | 81 | 10 |
| 21 002 035 | 158 | 16 |
| 21 002 036 | 128 | 12 |
| 21 002 037 | 80 | 0 |
| 21 002 038 | 116 | 21 |
| 21 002 039 | 117 | 9 |
| 21 002 040 | 82 | 11 |
| 21 002 041 | 99 | 10 |
| 21 002 046 | 113 | 0 |
| 21 002 057 | 90 | 0 |

Following hermaphroditism scoring detailed in Table 14, DNA was extracted from each of the 2758 F2 plants using approximately 70 mg leaf discs using an adapted kit with “sbeadex” magnetic beads by LGC Genomics, which was automated on a KingFisher Flex with 96 Deep-Well Head by Thermo Fisher Scientific. The extracted DNA served as a template for the library preparation for sequencing with the library pools prepared according to the manufacturer's instructions for the AgriSeq™ HTS Library Kit-96 sample procedure from Thermo Fisher Scientific. The subsequent targeted sequencing was conducted using a custom SNP marker panel based on the Cannabis Sativa CS10 reference genome on the Ion Torrent system by Thermo Fisher Scientific. The primers for the identified SNPs are provided in Table 17. The library pool was loaded onto Ion 550 chips with Ion Chef and sequenced with Ion GeneStudio S5 Plus according to the manufacturer's instructions for the Ion 550™ Kit from Thermo Fisher Scientific.

A genome-wide association study (GWAS) was performed using the data from the 24 F2 populations, totaling 2758 individuals. The study aimed to detect significant associations between genotypic information derived from targeted resequencing of the custom SNP marker panel described above and the binary trait of hermaphroditism—with 1 indicating hermaphroditism and 0 indicating the lack of hermaphroditism.

The genotypic matrix was filtered for SNPs with more than 30% missing values within the population and a minor allele frequency below 5%. This resulted in 3627 SNP markers after filtering.

To improve the estimation of population structure and kinship among individuals the inventors recognised that a high rate of missing values may be impacting the modeling. To address this, they incorporated an additional step. The GWA was performed again on the combined F2 populations using instead a SNP matrix that underwent a round of imputation to reduce the number of missing values. The imputation was performed using the HapMap_imputation software (GitHub-mwylerCH/HapMap_Imputation). Briefly, the genotype file was converted to a hapmap format (comma separated, http://augustogarcia.me/statgen-esalg/Hapmap-and-VCF-formats-and-its-integration-with-onemap/#hapmap).

In a first step, HapMap_Imputation counts the occurrence of each nucleotide at every genotyped position. The most common nucleotide is defined as major allele, the second is defined as minor allele. Missing genotyping information is excluded. In the case major and minor alleles occur at the same number, the nucleotide of the reference cs10 genome (available as GCF_900626175.1 on NCBI) is chosen as major allele. In a second step, HapMap_Imputation sorts markers by position and parses the hapmap into the required fastPHASE (Scheet & Stephens, 2006) input format. Briefly, HapMap_Imputation splits the haplotypes into two separate rows, converts major and minor alleles into 0 and 1, respectively, and produces temporary files for each chromosome.

During the third step, HapMap_Imputation downloads the latest fastPHASE version and runs the imputation using 8 cores in parallel. fastPHASE is run with ten random starts of the imputation algorithm. After imputation, HapMap_Imputation reverses the 0 and 1 coding into the major and minor nucleotide, respectively. Subsequently, the two haplotypes are combined, and the separate chromosomes are merged into a single file.

The imputed genotypic matrix was filtered for SNPs with more than 30% missing values within the population and a minor allele frequency below 5%, resulting in 5077 SNP markers for association analysis, a significant increase compared to the non-imputed dataset. The GWAS was performed using GAPIT version 3 (Wang & Zhang, 2021) with four statistical models: General Linear Model (GLM), Mixed Linear Model (MLM), FarmCPU and Blink. A quantile-quantile plot (QQ plot) was used to assess the performance of the models. The Blink model was chosen by the inventors and was used for the analysis. SNPs with a LOD (−log 10 (p-value)) value greater than 5 were considered to be significantly associated with trait variation.

The inventors identified two major QTLs from the GWA on Chromosome NC_044370.1, based on the SNPs found to be significantly associated with the presence or absence of hermaphroditic flowers. The first QTL is found in the region of 18371013-35677966, where the most significantly associated SNP, “common_294” is at position 30974345, with reference to the CS10 genome. This QTL was identified by the inventors in the 2020 mixed population experiment of Example 1, where it was based on SNPs “rare_30” and “GBScompat_common_54” from position 28544332-32763867, respectively. The QTL was further validated in the expanded mixed F2 population as a determining factor of hermaphroditism. The SNPs identified in this experiment, as listed in Table 15, can be used as markers for the selection for or against the hermaphroditism trait.

The second QTL is found in the region of 90451268-102978171, where the most significantly associated SNP, “common_517”, is at position 99121582, with reference to the CS10 genome. This QTL was also identified in the 2020 mixed population experiment described in Example 1, where it was defined by the SNP “common_524” at position 99761993. The QTL was further validated in the expanded mixed F2 population described in Example 2, where it was defined by SNPs “common_491” and “common_546” in the region of 94129798-101726389, respectively. The additional SNPs identified in this experiment can be used as markers for the selection for or against hermaphroditism.

The inventors demonstrated the use of a genomic selection model and tested this on a training population of mixed lineage diverse cannabis plants where ˜36% of the plants displayed hermaphroditism. Twenty-five markers were selected in the region of the QTL at position 90451268-102978171 to test the prediction power for the selection of hermaphroditic plants compared to 25 randomly selected markers. The inventors performed a multiple regression analysis with the allele as variable and hermaphroditism as target using the random forest algorithm implemented in the ranger package (v 0.12.1, Wright and Zieger 2017). The resulting R squares are derived from the comparison of the predictions from the developed model with the measured phenotype of the training population.

The approach tested 100 permutations for the specific markers and 100 permutations of the 25 random markers resampled for each permutation. The results of the genomic selection model demonstrate that the specific markers in this region greatly improve the accuracy of selecting for hermaphroditism, with R-squared of ˜0.25, in comparison with the use of random markers, with R-squared of ˜0.0125.

Table 15 lists additional SNPs significantly associated with variations in the hermaphroditism trait. These SNPs, representing QTLs, can serve as markers in marker-based selection or to improve genomic selection models for the hermaphroditism trait.

TABLE 15
Additional SNPs associated with variations in hermaphroditism in the F2 populations.
The table presents the positions and chromosomes of the SNPs with reference to the CS10
reference genome. The BLINK model's LOD score is included as LOD_BLINK, and
the mean phenotypic values for Allele 1, 2, and 3, are presented as “Mean 1”, “Mean
2”, and “Mean 3” respectively. The number of plants having Allele 1,
2, and 3 are presented as “Count 1”, “Count 2”, and “Count 3”, respectively.

| SNP | Chromosome | Position | Alleles_1 | Alleles_2 | Alleles_3 | Mean_1 |
|---|---|---|---|---|---|---|
| common_517 | NC_044370.1 | 99121582 | AA | AG | GG | 0.06871609 |
| common_507 | NC_044370.1 | 97056996 | AA | AT | TT | 0.25238898 |
| common_294 | NC_044370.1 | 30974345 | AA | AT | TT | 0.93333333 |
| GBScompat_{—} | NC_044370.1 | 35677966 | AA | AG | GG | 0.08256881 |
| common_56 | | | | | | |
| GBScompat_{—} | NC_044370.1 | 18371013 | AA | AG | GG | 0.18352223 |
| common_36 | | | | | | |
| common_480 | NC_044370.1 | 90451268 | CC | CG | GG | 0.24893617 |
| common_561 | NC_044370.1 | 102978171 | AA | AG | GG | 0.06020558 |
| common_685 | NC_044371.1 | 14838226 | AA | AG | GG | 0.14431984 |
| common_1025 | NC_044371.1 | 86862040 | AA | AT | TT | 0.19169719 |
| common_2949 | NC_044375.1 | 13190709 | AA | AG | GG | 0.16041979 |
| rare_7 | NC_044370.1 | 6234467 | AA | AC | CC | 0.36030534 |
| common_119 | NC_044370.1 | 11475121 | AA | AG | GG | 0.17193897 |
| common_528 | NC_044370.1 | 100088557 | AA | AG | GG | 0.20042531 |
| GBScompat_{—} | NC_044371.1 | 25913418 | AA | AT | TT | 0.13581184 |
| common_139 | | | | | | |
| GBScompat_{—} | NC_044378.1 | 37840619 | AA | AG | GG | 0.29737609 |
| rare_186 | | | | | | |
| common_489 | NC_044370.1 | 92434073 | AA | AC | CC | 0.28432732 |
| GBScompat_{—} | NC_044378.1 | 100163 | CC | CG | GG | 0.1152815 |
| common_883 | | | | | | |
| common_1524 | NC_044372.1 | 60699277 | AA | AG | GG | 0.10067114 |
| GBScompat_{—} | NC_044374.1 | 44536110 | AA | AG | GG | 0.15967197 |
| common_504 | | | | | | |

| SNP | Mean_2 | Mean_3 | Count_1 | Count_2 | Count_3 | LOD_BLINK |
|---|---|---|---|---|---|---|
| common_517 | 0.09589041 | 0.30748422 | 553 | 1095 | 1109 | 37.53816716 |
| common_507 | 0.04481793 | 0.01136364 | 1779 | 714 | 264 | 27.53549948 |
| common_294 | 0.45652174 | 0.16654303 | 15 | 46 | 2696 | 17.0615317 |
| GBScompat_{—} | 0.13923077 | 0.23002421 | 218 | 1300 | 1239 | 13.47064117 |
| common_56 | | | | | | |
| GBScompat_{—} | 0.12112676 | 0.18518519 | 2294 | 355 | 108 | 10.96401867 |
| common_36 | | | | | | |
| common_480 | 0.11869436 | 0.17792932 | 470 | 674 | 1613 | 9.684858537 |
| common_561 | 0.15433213 | 0.28099174 | 681 | 1108 | 968 | 9.440837483 |
| common_685 | 0.25813449 | 0.28163265 | 2051 | 461 | 245 | 8.498430839 |
| common_1025 | 0.19555556 | 0.13161132 | 819 | 1125 | 813 | 8.328623727 |
| common_2949 | 0.238921 | 0.16455696 | 2001 | 519 | 237 | 8.14447613 |
| rare_7 | 0.15095677 | 0.05065123 | 655 | 1411 | 691 | 7.662803717 |
| common_119 | 0.35849057 | 0.17647059 | 2687 | 53 | 17 | 7.510824509 |
| common_528 | 0.22461538 | 0.06170599 | 1881 | 325 | 551 | 7.494616121 |
| GBScompat_{—} | 0.20283976 | 0.23178808 | 1318 | 986 | 453 | 6.182667096 |
| common_139 | | | | | | |
| GBScompat_{—} | 0.23658537 | 0.11794228 | 343 | 820 | 1594 | 5.880347694 |
| rare_186 | | | | | | |
| common_489 | 0.06272401 | 0.0201005 | 1442 | 1116 | 199 | 5.521817722 |
| GBScompat_{—} | 0.15242019 | 0.20736023 | 373 | 971 | 1413 | 5.299066177 |
| common_883 | | | | | | |
| common_1524 | 0.19734904 | 0.18700184 | 447 | 679 | 1631 | 5.1934005 |
| GBScompat_{—} | 0.19607843 | 0.25382263 | 2073 | 357 | 327 | 5.053464113 |
| common_504 | | | | | | |

The underlying mechanism and gene(s) involved in the initiation of hermaphroditism in cannabis are unknown to date. Based on a candidate gene approach the inventors were unable to identify the causative SNP responsible for the trait under investigation. Therefore, the inventors used the results of the association studies to identify candidate genes at the QTLs identified by comparing a collection of cannabis genomic sequences to identify putative causative SNPs that appeared in a similar pattern as one of the most significantly associated polymorphisms listed in Table 3, “common_517”.

The inventors used SNP differences between cannabis genomes to identify candidate genes associated with the trait of interest. To achieve this, short reads from sequenced lines were dereplicated with NGSReads Treatment (version 1.3, Gaia et al. (2019)) and pre-processed with fastp (version 0.23.2, S. Chen et al. (2018)) before being aligned to the CS10 reference genome with Bowtie2 (version 2.3.5.1, with options --rg and --rg-id to add read-group identifiers, Langmead and Salzberg (2012)). Only unique alignments with a mapping quality of at least 10 were kept. SNPs were called with freebayes and filtered for a minimal quality of 20 (version v1.3.2-40-gcce27fc, parameters -p 2 --min-coverage 20-g 30000 --min-alternate-count 4 --min-alternate-fraction 0.1 --min-mapping-quality 10 --max-complex-gap-1, Garrison and Marth (2012)). SNPs were finally filtered for a coverage between 5 and 10,000 within each line and annotated with snpEff (version 4_3t, Cingolani et al. (2012)).

For each line, the inventors constructed a pseudogenome by incorporating its variants into the CS10 reference genome with vcf-consensus (Danecek et al. (2011)). To align genes from a reference genome to a target genome, CS10 annotation was lifted over with liftoff (version 1.6.3, Shumate and Salzberg (2021)). Protein and cDNA sequences were extracted with custom scripts and aligned with muscle (v3.8.31, Edgar (2004)).

The inventors extracted proteins located between 90451268-102978171 bps on chromosome NC_044370.1 and performed multiple sequence alignments which to generate tables representing the variant positions and protein variants. These were tested for correlation with the significant SNPs from the GWAS marker panel, “common_517”. Proteins with significant associations were kept and used to extract the SNPs within the associated genes. SNPs were tested for association with the significant SNP “common_517” from the GWAS marker panel and only significant SNPs were used as candidates to identify genes potentially associated with hermaphroditism. The remaining 117 SNPs were further filtered by examining the gene expression in a published study of masculinization in cannabis where female flowers were treated with colloidal silver to induce male flower appearance (Adal A M, Doshi K, Holbrook L, Mahmoud S S. Comparative RNA-Seq analysis reveals genes associated with masculinization in female Cannabis sativa. Planta. 2021 Jan. 4; 253 (1): 17). Using transcript data from this study the inventors identified a candidate gene, LOC115696400, that is strongly regulated in response to colloidal silver and shows differential expression between male and female flowers. This gene encodes the protein, XP_030479162.1 predicted to be a 1-aminocyclopropane-1-carboxylate synthase (ACC synthase), which produces ACC, a precursor to the gaseous hormone ethylene, known to be involved in sex determination and flowering in many plant species. The inventors identified polymorphisms present in their collection of sequence cannabis lines. A number of SNP differences were identified in LOC115696400 (NCBI cs10 reference genome gene ID) based on the genome collection, some of which impacted the amino acid sequence; 99811627 (A>G, loss of start), 99811644 (G>A, synonymous), 99811652 (C>A, Pro>His), 99811653 (C>T, synonymous), 99811703 (T>A, Val>Glu), 99811709 (C>T, Thr>Ile), 99811742 (A>G, Lys>Arg), 99814345 (A>G, Gln>Arg), 99814346 (A>T, Gln>His), 99814409 (G>A, synonymous), and 99814578 (G>C, Arg>Thr) (where the first allele given is the reference allele based on the CS10 reference genome, and the effect of the SNP on the amino acid sequence follows).

Of most interest to the inventors was the surprising discovery of a SNP in LOC115696400 at 99811627 that changed the start codon to Val. Based on the alignments from the genome collection, the inventors identified several genomes where LOC115696400 lacked a start codon as a result of a polymorphism A>G at position 99811627 on NC_044370.1 (position 1 of SEQ ID NO: 343).

The inventors found that the polymorphism leading to the stop codon was associated with an increased propensity for hermaphroditism. It was present in 16 genotypes that were all predicted to have an increased likelihood of hermaphroditism. The inventors believe that the loss of a functional ACC synthase in the specific tissue type where it is expressed results in an increased propensity to hermaphroditism while a functional ACC synthase can prevent the emergence of hermaphroditic flowers. The inventors recognize that the polymorphism that confers the loss of the start codon in LOC115696400 at position 99811627 (A/G) can be used as a diagnostic tool to identify plants with or without the propensity for hermaphroditism. Based on the findings from Examples 1-3, the inventors point out that the heterozygous allele state for this polymorphism likely results in an intermediate propensity for hermaphroditism compared to the homozygous case. The identification of this polymorphism as a causative factor in hermaphroditism in cannabis can be used to manipulate this trait through a number of genetic modification techniques known in the art, including targeted mutagenesis, gene silencing, genetic modification, etc. The inventors note that in monecious cannabis the ratio of male to female flowers can determine seed and grain yield. The number of female to male flowers could be influenced by controlling the dose of the ACC synthase XP_030479162.1 at the specific tissue type where it is expressed, for example through providing one copy of the gene or two.

Context sequences for each of the markers identified in Table 15, as well as for the polymorphism at position 99811627 are provided in Table 16 below. The context sequence is based on the CS10 reference genome. The reference (context) sequence can be used as the basis of a KASP assay to genotype at this position. Targeted sequencing primers for each of the markers identified in Table 15 and 16 are provided in Table 17. The nucleic acid sequence from position 99811627 to 99817925 on chromosome NC_044370.1 is also provided below as SEQ ID NO: 343 with the polymorphism at position 99811627 in square brackets.

TABLE 16
Detailed information of each of the additional SNPs associated with hermaphroditism
in Cannabis as provided in Table 15, and for the SNP at position 99811627. The “context
sequence” is provided with the SNP given in brackets. All of the sequences and alleles
are provided with reference to the plus strand.

| SNP | Context sequence |
|---|---|
| common_507 | TTAATCTGAACACACTCATTTCTTCAGGAGCAGATTCTCAGGTTAACCTTTTCAAACTTTCC |
| | TTGTTCAATTTCAACATCTTAACTAGCATAGTTAACATAATATACATGCATCTCTTAATTATC |
| | TTGAGGATGTGACATATTCTAGTATTCCATTTTCTCAGATTATCGCATGGAGCTCTGATAAT |
| | TGGGAAAGACAAAA[A/T]AGCACGTTCTTGCAGACTCCAGCTGGAAGGACAGGAATGTCA |
| | GACACACAAGTGCAGTTTCATCAGGACCAGACACACTTCCTCGTCGTACATGAGACTCAG |
| | CTATCTATATACGAAGCCTCGAAACTCGAACGCATATTGCAGGTAGAACAATCAAACATTG |
| | CTTCAAAAACAGTCATGAATCTAAAAATTTATTACAAT (SEQ ID NO: 271) |
| common_294 | GGCTACTTACGGTGAGAAAGAGTGGTATTTCTTTTCGCCGCGTGATCGGAAATACCCGAA |
| | CGGTTCGAGACCGAATCGGGCAGCTGGAACCGGGTACTGGAAGGCAACCGGAGCGGAT |
| | AAACCGATCGGGAATCCGAAACCGGTTGGGATTAAGAAGGCTCTGGTTTTTTACGCCGGA |
| | AAAGCTCCGAAAGGAGAGAAAAC[A/T]AATTGGATTATGCATGAGTACCGGCTTGCTGACG |
| | TGGACAGATCAGCTCGTAAAAAAAACAGCTTAAGGGTACATTTTCTAATTAAACTCCATGA |
| | TATTAGCAGGGGTAGTTTCGTCATTTCACAGCATGTCCAAGCTGTGGTGGTAGTAAATGG |
| | GTCCCACGTGTGCTATCTGACACGTGGCGGCTTATAGCGCTAGATT (SEQ ID NO: 272) |
| GBScompat_ | GGTAGAGTTAAGATAATTAATTTAGTTATACAGAACTGCATACCGACGTATTTTATAGTGTA |
| common_36 | TACCTTTCCTCGTACGCTTTAGAAGTTCTCCTCCTCTGCAAACAAAGTTCAATGCTGTGAA |
| | AGCTTTCGATTCATGGCCTTCGCCTTTTTCGTCAGCCAATGCAAATGTAGTACTTCCACAG |
| | CCCCTTTGAAGCCTTGT[T/C]CTTAGAATAGCAGCTCCTCTTAAGGCTGTAGTATACACAAA |
| | GAAAACTCTCCCTCATAATTTTATTCAAAGAAGCTATTGTTTAATTAGTTTTTTACATGCTAA |
| | TCTATTGTTATGAGTAAAAATAAATAGGATTGTCCTCTTTAAACTATGACTATTGTAGTAAAT |
| | AATATTTTGATGTGGCACTAACGTGACGTTAATC (SEQ ID NO: 273) |
| common_480 | CAAGGAGTTATCTCACTAGAGCTACATCACTAACAAAATTGATTTCCCACTCTCATAAATAA |
| | AACTAAAAGACTGTAACCAACACATATTATTGTACTCTTGTTTAGTTTTGGTGAGAATAAAG |
| | AGATGAGAATGAACTACATCCTTATTGCCACTTGATCTGCAGAACTTTTCTTCACAACACCT |
| | CTAGTGCATAACTCA[C/G]TTAACAACTCCAAAGCCTCAGTTGCAAATCCTTCATAAGCTAG |
| | ACCTTCAATGAGAATGGTATATGTCACTTCAGTGGGTTTACATCCTTTGGATACCATATGAT |
| | CCAGAAAATCGATGGCACGACTGGTTTGCCTAGTCTTACAAAGTCCCAACATGATAGAGTT |
| | GAAGGTGATTGCATTAGGCTTTATTCCAAGTCCTT (SEQ ID NO: 274) |
| common_561 | ATGGTTCGTACATTGTTGCTATATTTATTCTCTTTTTAACTTAAATTCTTTCTTGTGAAAACT |
| | TTCCGATTCCGGTTTGGGTCGGGTGAGTCTTGTCTTGTGACATGTTTGAGAGTTAGTATTT |
| | GTGTTTGTGCGTCCGATTACGGTTGTTTATGATCTAATGGCGTCTAGTAGTAGAACTATGG |
| | AAGATTTGGGACATCA[A/G]TGGGCTGATCTACAAGTGGAAGATGAGAATGAAGTGGGGC |
| | TTTTATTTGATCAGGAGGAGGTGTTGGTTGATGAGTTTGATGGGAGGTGGTGTTTAGTCG |
| | GGAAATTACTATCTGATAGGCCGGCTGATTTTGACTCTCTAAGGAATGTAATGGCGTCTTT |
| | GTGGCGACCGGGGAAAGGGATGTACGTCAAAGAGTTGGAG (SEQ ID NO: 275) |
| common_685 | TACACCAGAAGCAAACTTCCCAACAAGATCATCTTCAGTCATATCAAGAACCTCAGGGCTG |
| | AAGACTGAGCCATTGTCATAAACAGATTGGACAACAAGACCATATGAAAATGGCCTGATG |
| | CCAAGCTTGGCAAGAAGGGCAGCCTCAGAGGAACCAACCTTGTCACCCTTCTTGATGAGC |
| | TCAACTGGGGTGATAATTTC[G/A]ACAGTACCCTTGTTAATCTTAGTTGGGATATTAAGCAC |
| | CTGTTGAGCCCAACGGTGTTAGTCATAAATTCTAAGGAAATACAATGAATGAAAAAATATA |
| | GGTTAGGTAAGTGTGCAGACCTGGAAGAAAGAAGTCTGAGATGGATCGAGACCAGTGTT |
| | GCCAGGAGGAACAACCACGTCAATCGGGGCAATCAAACCCACA (SEQ ID NO: 276) |
| common_1025 | TGTAGTTTCCGAGTGGATGCATGTGCTTTTACCTATAGCTATGGAAAGAGGAGTTTGCATA |
| | ATCACAAACATGGGTGCAAGTAAGATTACTAAATAAAATTTTATGCAATCAACAAACTTTTT |
| | TTTTTCTTAAAAGAAATAGAGTTTAGATAAATGTTCTTATTATTGTTTCTTTTTCTGGGCAGT |
| | GGATCCATGTGGTGC[T/A]CAGGAGAAAGTTGTAGAAATAGCCAGCAGCCTGGGACTGAA |
| | CGTATCTGTGGGTGTTGCTTATGAGGTTTTTGTCAGCAAAACAGGTATTTGTAGTCACTTT |
| | TAGGAGGATGAATTGGAGATGATACGTTTGAGATTTTATAAACACTAAATGCTTCACAACA |
| | TTCAATTAAATCAAGGATTGTTTTATGATTTAATCATG (SEQ ID NO: 277) |
| common_2949 | GAACTCTTCTTCAAATCATCATATTGCTTCTTCTATACTACTAGTACTACTACTTGTACTTGT |
| | AATGAGTTTTGGGCAACAACAAGCTGAGGCAAGAGCTTTCTTTGTGTTTGGAGATTCTCTT |
| | GTGGACAATGGCAATAACAATTATTTAGCCACCACAGCAAGAGCTGACTCTCCTCCTTATG |
| | GGATTGACTACCCTAC[T/C]CATAGACCCACTGGCCGTTTCTCTAATGGCCTCAACATGCC |
| | TGACCTTATCAGTAAATCTCTCATTAATTTTCTCTTTAATTATCTAAGTATACATATATATGTA |
| | TGTATCTCTGATTTGTGTATATATCTATTAACTTGAATAAATAGGTGAGCAAATTGGGTCAG |
| | AGCCCACATTGCCATACTTGAGTCCGGAGCTCAC (SEQ ID NO: 278) |
| rare_7 | TAGTCAACTTGGATGGCCAAGAGTGTGTATTTGTATGGAGTTAGAAGATTATGCATTAAGT |
| | GTATTGAACTTAAATAGCTGACTGGTTTTCAATCTATATAATCAACATAGAACTCTCACTAT |
| | CATGCGGGGATCTCTGATCACTGCTTATGATTTATACTCTCAGATGTTTTAATCCATTATTG |
| | ACTTAAATGGGTTTTT[G/T]GATATAGGTATTCTTGAAGGGCCCATCCTTGTATGCCTTTAG |
| | GGGCCTGGCTGGCCGGTTTGCTCCTATTGGAGTTCATATAGCAATGCTACTAATAATGGC |
| | AGGTGGAACTCTAAGTGCAGCTGGGAGCTTCAAAGGAACAGTCACAGTGCCGCAGGGGC |
| | TGAATTTTGTGGTCGGAGATGTACTAGGCCCAACTGGGTT (SEQ ID NO: 279) |
| common_119 | AAAAAGCACAGGAAGAGATTTTGAGATCCATGTTTGGACCAAAGGAAAGGAGTACTCTCT |
| | CTCTCTCTCTCTGAACTGTCTGTTTGAGCTTTGGTGAGCTAAACCAACGAACGAGCGAGC |
| | TTCAAGCTCAACAGGCCTTCTTTGAGTATGTATGTAATGTACCCGTCTTCACCTTCTTCTCA |
| | TGAAACGCCTTATGAAATG[T/C]CATCCAACTCTACTACCACTGTCATTACCCATAGGATAT |
| | CAACCTCTTTCCCGCTCTCTCACTTCCTCAACCTCCACATATCAACGTAAATGGAAACCAA |
| | AGACCACTCTCATCATTACCAACCCAACTTTATTACTCATGGAGTCTTGCTCCACCATGCT |
| | CCAGATGAAGCAAATTCAAGCTCAAATGACTCTCACTGGT (SEQ ID NO: 280) |
| GBScompat_ | TCATCTGTTTGTGACATTATTCTTTGCTTATTTTTGCTTAAGAAAACTTCGTTCGATTTATTA |
| common_139 | AATTATATGAAAGTTCGGTCCTTTATGTGGAATGTATATTTTCCTTTCCTGCAGACAAATTT |
| | GCAATTGTTAGCCAGCTGCTACTTGCGGAACAGTCAGGCATACTCTGCGTACCATATTTTG |
| | AAAGGTACATATGTC[A/T]TAAAATATATATCTTTAGCTTACTATGGTTACGGTTTACTATTTT |
| | TTCATTTTGCTTGAGTTTGATTATTTTTTTCCTTTACACTATTATCTGAAGGAACACAATTGG |
| | CTCAATCCCGCTACTTGTTTGCAATGTCTTGCTTTCGAATGGATCTTCTTAATGAAGCTGAA |
| | ACAGCATTGTGTCCTCCTAATGAACCCAGTG (SEQ ID NO: 281) |
| GBScompat_ | TCGATCTGCTACCCTAGTATGATGAGTGTATAAAAGTACAGTATAGAGATTTTCATACGTAT |
| rare_186 | AAATAATATCGTGGTCTGACAGTACATAATATATTTTTTTATATATATACAAATACAATTGAG |
| | AGTACATAATATTTAACGTATGTCCAAGTTAGTTTGATCGAGCCCTTTCGCAGCTTCGAAG |
| | CGCGTGACGCGTGTT[G/A]TGTCAAGTCTTGCTAGCCGACAAAGGTCTGCTTTGTCAGTTT |
| | ATGAGGACTTTACTTGGCAGTTGCCACCTTCATCTACGGACTCAATAAATCTTACTTTGCA |
| | CGCGCTTCAACGTGGTGCGTGAAATCTTTTCTTATTTTGCCAAGTGGACTTAGTCAAAGCT |
| | GCCTCACCTTCGTTTATTTATTTAGAGAAATTTACAT (SEQ ID NO: 282) |
| GBScompat_ | AAATGAGGTTAACCCCAGCACTAAACAGTCTAACTTAAAAATAAGAACTACCTATCACCAT |
| common_883 | CCCCTACAAACTTACAAGTTTAATTATGCCACTCTAGTATAAAACGAAAAAGGCAATCATTG |
| | TCATAGACAAAACACAAGGCATTTAAATTTAATAAAGTAGCAACTGCAGCACCAGGACAAT |
| | ACAAGTTTCAGTCAATA[C/G]CCAATTTACACATAAACCTATTTCAATATCTGCTGTTGTCTC |
| | AACAGAAGAGCACTAAGCACCCTCAATATTTAGAACAAACATATATACATTATATAATTACA |
| | TTTATCATCCACAAAGTACCACAACCAAATAGACCCTATTGAAAGTTGAAACTGTTTACCTT |
| | AGCAGCAAAGTGAAAATTAGTTCAGCAAAAGAGTT (SEQ ID NO: 283) |
| common_1524 | ACACTAAGGGACACCAAAAATAATAAATTTCCCCTTCATTATTTTCAGATACAATGGGGATT |
| | TAGTCCCCCCCTCCCACACACATATACCCAATCACTTGAGGGCATGCAGTGCCCCGTCAA |
| | TGATAATTGTTCAGAAGACTAATGGAATAATAACTGCCATTCTTACCCACATCTGTATAGGT |
| | TGATTAAAATGAATTTC[G/A]GCAAGGTACTCCATTTCTGGATCTGCTCGCATCCATAACAA |
| | GGGCGAGTCCATACTGTGAAAAGAAAAGTTTGACACACGCATGCAAAGACACAAGAAAAT |
| | AATAATTAGATCCTCCTATCCAAATAAACTTTAACAGAGCTTAAAGAAATGAACCTACCTGG |
| | AGCGCATATCTAGAGGAAGAGTACCATCTCCATTGTCA (SEQ ID NO: 284) |
| GBScompat_ | AAATCAAACAGATAAACCTACAAGCATACTAGTCAAACTCCTTTCAGAAACAACCAACCAC |
| common_504 | CATTTTTGCTTTATAAATATACAATGACCCCCAATGCACGAAAAACCACCTCGGTTTAGGA |
| | GGAAAGAATATCAACAAATAGTCAAATAGATAAAGATTAGAACGATATTCATCAGAAGGTA |
| | AGGAAACAGAGCTGTGAA[T/C]GCACCGCACCTTATTCTAAAAATCGTGGAATTGCTGCTT |
| | CTATGGATTCTCAATGGTGGCATTCATAATCCGCAGTGATCTTGATCTTTCCTGCTGCCCA |
| | CTGCGTCTCCAAAAAGCCCTCCTCCACCAAACTTCGAACCCTCTTTGTATGTTCGGGCAAT |
| | CTTCAGCAGAGTTCAAGAATCGGTTAAACCAAGCAAGCAA (SEQ ID NO: 285) |
| SNP at position | ATGAATCATGAGAGAATAGTTCTGATGATTTTGAAGATGTTCATCCAGAC[A/G]TGCCTAAA |
| 99811627 on | GATGATCAGGACATGCCCGCCGATGGTGCTGCTAGAAAGAAA |
| NC_044370.1 | (SEQ ID NO: 286) |

TABLE 17
Targeted sequencing primers (5′ to 3′) for the SNPs identified in Table 15, as described
in this Example 6.

| SNP | Forward Primer 1 | Reverse Primer 1 | Forward Primer 2 | Reverse Primer 2 |
|---|---|---|---|---|
| common_517 | GGATTTGCTTCACG | CCCAAAACCACTTT | GGATTTGCTTCACG | TCCCAAAACCACTT |
| | ACAGCC | CGCCAG | ACAGCC | TCGCCA |
| | (SEQ ID NO: 95) | (SEQ ID NO: 96) | (SEQ ID NO: 95) | (SEQ ID NO: 287) |
| common_507 | TCAGGAGCAGATTC | CGAGGAAGTGTGT | TCAGGAGCAGATTC | ACGACGAGGAAGT |
| | TCAGGT | CTGGTCC | TCAGGT | GTGTCTG |
| | (SEQ ID NO: 288) | (SEQ ID NO: 289) | (SEQ ID NO: 288) | (SEQ ID NO: 290) |
| common_294 | GTACTGGAAGGCA | TGTCAGATAGCACA | GTACTGGAAGGCA | TATAAGCCGCCAC |
| | ACCGGAG | CGTGGG | ACCGGAG | GTGTCAG |
| | (SEQ ID NO: 291) | (SEQ ID NO: 292) | (SEQ ID NO: 291) | (SEQ ID NO: 293) |
| GBScompat_ | CGTAAGTGTCCGG | GCAGCTGGTTTGTT | TTCGTAAGTGTCCG | GCAGCTGGTTTGTT |
| common_56 | TTCCCAT | GAGTGG | GTTCCC | GAGTGG |
| | (SEQ ID NO: 294) | (SEQ ID NO: 270) | (SEQ ID NO: 269) | (SEQ ID NO: 270) |
| GBScompat_ | GCCTTCGCCTTTTT | CACGTTAGTGCCAC | TTTCGATTCATGGC | CACGTTAGTGCCAC |
| common_36 | CGTCAG | ATCAAAA | CTTCGC | ATCAAAA |
| | (SEQ ID NO: 295) | (SEQ ID NO: 296) | (SEQ ID NO: 297) | (SEQ ID NO: 296) |
| common_480 | TGCCACTTGATCTG | AAACCAGTCGTGC | TGCCACTTGATCTG | AACCAGTCGTGCC |
| | CAGAACT | CATCGAT | CAGAACT | ATCGATT |
| | (SEQ ID NO: 298) | (SEQ ID NO: 299) | (SEQ ID NO: 298) | (SEQ ID NO: 300) |
| common_561 | TTTGGGTCGGGTG | CGCCACAAAGACG | GTTTGGGTCGGGT | TCGCCACAAAGAC |
| | AGTCTTG | CCATTAC | GAGTCTT | GCCATTA |
| | (SEQ ID NO: 301) | (SEQ ID NO: 302) | (SEQ ID NO: 303) | (SEQ ID NO: 304) |
| common_685 | ACCTCAGGGCTGA | TCCAGGTCTGCACA | ACCTCAGGGCTGA | CCAGGTCTGCACA |
| | AGACTGA | CTTACC | AGACTGA | CTTACCT |
| | (SEQ ID NO: 305) | (SEQ ID NO: 306) | (SEQ ID NO: 305) | (SEQ ID NO: 307) |
| common_1025 | TTCCGAGTGGATG | AGCAACACCCACA | CGAGTGGATGCAT | AGCAACACCCACA |
| | CATGTGC | GATACGT | GTGCTTT | GATACGT |
| | (SEQ ID NO: 308) | (SEQ ID NO: 309) | (SEQ ID NO: 310) | (SEQ ID NO: 309) |
| common_2949 | AGCTGAGGCAAGA | ATGGCAATGTGGG | GCAACAACAAGCT | GGCTCTGACCCAAT |
| | GCTTTCT | CTCTGAC | GAGGCAA | TTGCTC |
| | (SEQ ID NO: 311) | (SEQ ID NO: 312) | (SEQ ID NO: 313) | (SEQ ID NO: 314) |
| rare_7 | GCGGGGATCTCTG | TGGGCCTAGTACAT | CGGGGATCTCTGA | TGGGCCTAGTACAT |
| | ATCACTG | CTCCGA | TCACTGC | CTCCGA |
| | (SEQ ID NO: 315) | (SEQ ID NO: 316) | (SEQ ID NO: 317) | (SEQ ID NO: 316) |
| common_119 | GCTAAACCAACGAA | TGCTTCATCTGGAG | GCTAAACCAACGAA | GAGCATGGTGGAG |
| | CGAGCG | CATGGT | CGAGCG | CAAGACT |
| | (SEQ ID NO: 318) | (SEQ ID NO: 319) | (SEQ ID NO: 318) | (SEQ ID NO: 320) |
| common_528 | TTACTGGTGCAAGC | GGTGAGGCTGAAC | TGCAAGCCCATCG | GGTGAGGCTGAAC |
| | CCATCG | AAGAACC | AAGTGAA | AAGAACC |
| | (SEQ ID NO: 321) | (SEQ ID NO: 232) | (SEQ ID NO: 231) | (SEQ ID NO: 232) |
| GBScompat_ | TGCAATTGTTAGCC | CACTGGGTTCATTA | TGCAATTGTTAGCC | ACTGGGTTCATTAG |
| common_139 | AGCTGC | GGAGGACA | AGCTGC | GAGGACAC |
| | (SEQ ID NO: 322) | (SEQ ID NO: 323) | (SEQ ID NO: 322) | SEQ ID NO: 324) |
| GBScompat_ | GTTTGATCGAGCCC | TAAACGAAGGTGA | TTTGATCGAGCCCT | TAAACGAAGGTGA |
| rare_186 | TTTCGC | GGCAGCT | TTCGCA | GGCAGCT |
| | (SEQ ID NO: 325) | (SEQ ID NO: 326) | (SEQ ID NO: 327) | (SEQ ID NO: 326) |
| common_489 | TGCCTTGGAGGAT | ATCCACATTGCAAA | CCTTGGAGGATCAT | CCACATTGCAAACA |
| | CATTGGT | CAGGGC | TGGTGCT | GGGCAG |
| | (SEQ ID NO: 328) | (SEQ ID NO: 329) | (SEQ ID NO: 209) | (SEQ ID NO: 210) |
| GBScompat_ | ATGAGGTTAACCCC | GGGTGCTTAGTGC | ATGAGGTTAACCCC | TGAGGGTGCTTAGT |
| common_883 | AGCACT | TCTTCTGT | AGCACT | GCTCTTC |
| | (SEQ ID NO: 330) | (SEQ ID NO: 331) | (SEQ ID NO: 330) | (SEQ ID NO: 332) |
| common_1524 | CCAATCACTTGAGG | GCGCTCCAGGTAG | CCCTCCCACACACA | GCGCTCCAGGTAG |
| | GCATGC | GTTCATT | TATACCC | GTTCATT |
| | (SEQ ID NO: 333) | (SEQ ID NO: 334) | (SEQ ID NO: 335) | (SEQ ID NO: 334) |
| GBScompat_ | AATGACCCCCAATG | CTCTGCTGAAGATT | ATGACCCCCAATGC | CTCTGCTGAAGATT |
| | CACGAA | GCCCGA | ACGAAA | GCCCGA |
| common_504 | (SEQ ID NO: 336) | (SEQ ID NO: 337) | (SEQ ID NO: 338) | (SEQ ID NO: 337) |
| SNP at | CCTATGGTCGAAGA | ATTTGGTCAGCATT | GAAGACGATGAATC | TTTATTTATCGCGG |
| position | CGATGAAT | AGCCATTT | ATGAGAG | GTGAAGAT |
| 99811627 on | (SEQ ID NO: 339) | (SEQ ID NO: 340) | (SEQ ID NO: 341) | (SEQ ID NO: 342) |
| NC_044370.1 | | | | |

SEQ ID NO: 343: Nucleic acid sequence from position 99811627 to 99817925 on
chromosome NC_044370.1 (LOC115696400) showing the polymorphism as position
99811627 in square brackets (position 1 of SEQ ID NO: 343).
[A/G]TGCCTAAAGATGATCAGGACATGCCCGCCGATGGTGCTGCTAGAAAGAAAGATAATCTTGATGAG
TCAGATCCTGTGGTCACAATGGCCAAGAAATTACAAATAGCTAAAAATAAAGGTGTCCCCTACCATTTTG
TACCAAGTTAAAAATTACATAAATTGAGTTAGAAAACATAGCCTTTTCCATTAAATTATTCTCTCGGATA
CTATTCTTATACTTTCCAGAAAGTCGTTAACAAAAATGGCTAATGCTGACCAAATATTTTCGGTAATCTT
CACCCGCGATAAATAAATTAATGTTTTTCCAAGTGTGCACCAAACTTAGAAACTCAAAATTCCCAATTAA
TTGTAATGGATTACATGGCATATATTTACAGTTTGTAATATATCGTACGGATTACCAATATTGGAAGTCA
GAGACATGGCTTTTGTTCTTTTACTCTAAACGGAAAATATTGTGTGTATGTGTGTGTGAGCTTGATGCAT
GTGATGTTACCCTTTTCAACAGTGTAAAACTCTTTCCTTCTTCTTCTTCTTCAAACTCAACAAATTAATA
AAATGAAAAGTTAAAGTACTAACTCACGTGTAGCAAAGTTTTAACACATATTGCTCAGCATATTCAAAAT
AATATATATATATATAGAAAGCCGAAAAATTATTTAATGTGATGAATATATGTTGCACGAAAACGTGAAC
TATTTTAATATGTACTCAATATTCACTGTACGTACGTGAGCCATGCATGTGTGATTGCTCTCATTAATTT
TGATGAAAAAAAAAAGGAAACTGATGATATAGCACCCGTAGATGTTAGAAAATAATAGTACTATTAGGTA
AAACTAAGAATGACTATGACTTCACATCAGTGACTTAAGACAAAAAACAAATTGATGTTGATAGCTATAG
CATATACTTTTTTTTTTTTTTTTTTTTTTTATGGGAGTTTAACCTATACCATAGATCTTACATCAGTGAA
ATTAAGGCTTTTACACTGTAGGAACTCATTGACACTGTTTTATTGTTTAAAATCAAATATTATATAGTTT
TCTAAGATGAGAAATTTAGTTGTAATTATAATCTTTTTAGGGGGAAAAAATTAGCGAGTGGATTTTAATT
TATTGAAAGAATTTTTATCTTTTTCAGTAATAAATCAAAAAATTTGATGATGTATACATTTAATATGTAT
TATTTTATTTTTAAAAGAAAGAATTCTTATGTGATATTGGCATATATAGGTGGGCCATGCATAGCAGCCC
ACTCATAGTTTTAATGGTCAGGTTACAAATGAAGAAGGTTAAAACTTAAGTATTAGTATAATTTAATGTC
AAGTTAGTCAGTAGTTTGTTTATCCTTAATTAATTTATTGTGTGAATTTCGAATCTATGATGATTGATGA
ATGTTAAAACTCAAAGTATTAGTATAATTTAATGTCAAGTTTCACCTATTTTATTTAATTTGTAACAGAA
CATGTAATCAAGACTTGTTACTCAATTTTACCAATTAAGATGTGATGCAATTGAGTACTGTTAGAGTATA
GTACAAGTGTAAGAATATTGAACAATTTATAAGAGTGTGACTAACTCTACTCATCACTATAGTAGGTTAG
ATATAGCCTCATAATTCTTGTGATAATATTGTATTCAATCACTAACCAAATTGTGTGCACGAAAAAAAAT
AGTAGAAAGTTGCACGAGATCTCTCATAGTGCCTAGATCAAAACAAAATAAGCGAGCAAGTGTCTCACAT
GTAACCGCTATAAATATATATATATAAAACAAGCAATAATATAGTTCCATTTATTAGAGTATGAGATAGA
ATCTCATAATTATTGTCAAGCACCATAAGTGACTTATTGCAGTGCTCTATTTAGGATTGTATACTGCTAT
ATATAAGTAATGTATACTACACATTACTTAATATTAATATGGTTTTACACAATAATAATATTTATTTAAT
TTTAGTAAATAAGGAATATAATTATAAACCAATTTATAAATTGCGTTTCAAATGGATTATTCATGTGGTA
TATATACTTTCATAATCACATTATAATTTTTTAAAAAATTAATACAATTATTATATGCGTGCGTGTATAA
ATAATAATAATAATCTGAACCTCTATACGTACTTCTAATTAATTGTAATTTTCTTATAAAGCCACTTTAT
TATTTCCCATTTGTGTCGTTATCTTATTTCTTTGAACTATTTTAATATTAATTAAAAAACTTATATATTT
ATATATTTAGTTCTGTTCTACCTAGCTAATATATATATACGTAGATCTCTCACAAACCATCCTCACTACT
ACATATACTACTACTCTTTCTCTCTTCTATTCTTCTACATTTCTCTCACATTTATTTATATATATACATC
GATCTTTTATTTATATATATACATCGATCTATTTGAAAAGAAAAAAATGAGTAGTTTATTATCTAAGAAA
GCAGCATGCAATGCTCATGGTCAAGATTCTTCCTACTTCTTAGGATGGGAAGAATACGAGAGAAATTCTT
ATGATAGAATTACCAACCCGGAAGGGATTATCCAAATGGGTCTTGCAGAAAACCAGGTTAATTATTATAT
ATATAGATTATAGAAAAATGTATTATATATGAATTAAAATTTTCTTACAATTCTTTGTTTTTGGACAGCT
TTGTTACCACCTTCTAGAGTCATGGCTTGCTAATAATCCACATGCACTTGGGTTTAAAAGTCAAGGTCAA
TACATTTTCAGAAAACTTGCTCTGTTCCAAGATTATCATGGACTCCCCGAATTCAAGAAGGTAATTATCA
TTAGATATTCTATAAAAATTAATATATTAAATTATAGGAAGAATACTAAACATTATTATTTTTCTTTTCC
AAATTCAATGTGCATATATATAATAAAATTTGGTTACAGCAGGCAATGGTTGAATTCATGTCCGAAATAA
GTGGAAACAAAGTGAGATTTGAGCCGCAAAGTCTAGTGCTCATCGCAGGTGCAACCTCAGCCAACGAGGC
CCTCATATTTTGCCTAGCTGATCCCAACGATGCCTTCCTTCTCCCAACTCCATACTACCCTGGGTACGTA
ACCTAATTAATTAACTTTAGTTACCCTTATTATTAATTTCATTAATTATTCTATCACTACTTTTCACTTT
AATTAGCTAAACGTACGTACGTTAGTCGTAGTAAAGTCACTAATAATGCCACAGCTAGCTTTCACTGGGT
CCCACTTCCTTCGTGCATACGTGATTCGGGTTCTACATCAATCATCATAGGATATTATAATATCTTGTTT
CTTATTCATTTTAGTATAGATAAAAATTGTTTAGCCTATTTTTTTTTAAAAGATGGTATACCTTATATGA
CTTAGTTATTTTTAAAATTATTTATAAATTTTTTAAAAAAAATTAATTTTTTTAAATTTTAAATATTCTG
AATAATTTATAGTATTGTAAATTATTTAAAATTTTCTAAAAATTTACAAATAATCTTAAATAATTACGGG
ATTATACAAACAAAAATTAAACTAAAAATCTATTCTAAATGTTAAAACAGATAAAGAATTTATTAAAACA
CTATTAGTGGTTTTACCAAAAATCTTCTCAATATTTTAATATTTAAAAACAAATAGATATGATAAAGAAG
AATTGCATATATGTAATGTACTTAGTTTTTGCAGATTTGATAGGGATCTTAAGTGGCGAACTGAAGTGGT
AATAGTGCCTATTCATTGCAAAAGCTCAAATGGCTTTCAAATCACGGAAGAGGGATTGGAACAAGCCTAT
GAAGATGCCACAAACCGTAATCTAAGAGTTAAAGGTGTTTTGATTACCAACCCATCTAATCCATGTGGCA
CCACAATGACAGTGGATGAGTTGAATCTCCTCCTTAACTTCATTGAACTTAAGAAAATCCATCTCATCAG
TGATGAAATCTATTCAGGAACAGTTTTCAACAAACCAGACTTTAAAAGTGTCATAGAAGTTCTCAACGAG
AGAAACAATAACAACAATAATAGTACTAATCAAATCATGATTCGGGAACAAGTTCATGTCGTTTATAGCC
TTTCCAAAGATCTAGGACTCCCTGGATTTCGTGTTGGAGCAATATATTCAAATCACAAGATGGTTTTAGA
TGCAGCCACAAAAATGTCGAGTTTCGGTTTGGTATCTTCTCAAACTCAATTCTTACTCTCGGTTATGTTA
TCAGACAAATATTTTACGAAAACATACATAAAAGAAAACCAAAAGAGACTTAAGAGAAGGCATAAAATGC
TTGTTGGGGGTCTTAAGAAAGCAGGAATAAGTTGTCTTAAGAGTAATGCTGGATTATTTTGTTGGGTTGA
TATGAGACACCTTCTTAAGTCGAATACTTTCAATGCTGAAATTGAGCTATGGAAAAAGATCATTTACGAT
GTGAAACTCAATATATCACCTGGTTCATCGTGTCATTGTAACGAACCAGGTTGGTTTAGAGTTTGTTTTG
CAAACATGTCCGGGAGAACATTGAAGCTCGCAATAAAAAGGTTGAAAGACTTTGTTGCTGATCATGATGA
GTTTAATTACCACACTAAATATCACCAACCTACTACGTAATAGGCTTTTCCACCTATCATTTCACATCCA
CCAATGCTATCAGCTCCCCATATTGTAAGTACAGTTTATATATATATATATATTCTCTTCTCTTATATAT
ACGTATAAATGAATTATTACACATGCATATACATCAATAAACTAGTATCATCATCAATCCAATCGCTCGG
TAGTTTTTTTCAAAAAAAAGAGAAAAAAACGTTAATATGATCATTTTGGACCGGTATTAATTAAAATGCA
TGGTACAACATGTTATACTGAACTCAATTTTCGATAATTTTTTTCTTTTAATACATATATAATCAACTTC
AAAACAATTTCTAACACGAACAAATATAAAAATTGTAACAAGTCTTGTTGTAACACTTTTATATTGGGTT
ATTTTTAAAGTTTATTTTAACTAAAAATCAATTCTAGGCATTACTTTGTACAAATCTAGAAAATACAATG
TCAAATTTATTATTTAATAAACTATTTAATTAGTAATTTTTATAAAATACAGAATTTAAAATAGTATTTA
TTAGATTGTTAATATTATTAAGAAGTTATTTGTGACTAAAAATATTATTAACATAGAAATCTGTAATTAA
ATGTGTAGTTGAGGCAAAACTTAAAACTAAATTAGTAATGAATTGTATCTAAATACTAGCATTTTAAAAA
TAATATTTAGTTAAAAAATTATATTATAATTAAAAATTTGTTACTAAACAAAAACTGTTTTTGCAGTGTG
ATTGAATCGTCTTTGCCTCTGAATTAATGGTTTTTACCTAGCTAGCGAAATTCCCCAATTACAACTAACT
GTTAAAATTATGTTATATCTGTTTATTTTTTAACGATTTGATGGATCAGAACTGTCTAAAAAGTAGGCAG
AAAGCAAACCAACATATATATGTTTTTTAATGGTTGTTATAATTGTTATTAGGAACAATTTTTGTCAGCT
AAAAAAAATAATTTACGGTATAAATATCTAAGTTTAATTTTACGATTGTAAATAAATATTTAAATTTAAT
TTTTAATCGTAATAATACTTAATTTATATTTTTAAAATTTTTGTAAGTACCTAGTCTTTAACTGTTAAAT
TATTATTCAAATGTTATTTTTTTATTAGTATATTTAAAGTAAGTTTTAAATAAAAAATACATATATTTAA
TGACTTAGACCAATTAGAGATTTAACTTGACATTTAAAGGTAAGGTATTTAGAAAAATTTTAAAATTATA
ACTTAATTGCTCTAAAAAATTAAACTTAACTATTCATGTATAAATTACTCTAAAAAAAACTTTACATAAG
CTATAAGAGAAAACAAATCATTGCTTTGTCAAAAACAAAAATTAAAAGGCACTAATAGTTCTAACACTAC
TTAAAATGTTAAATCACTATTAATAATATTAAATGTTAAGTCATATATAATTTAATATAATATTTTTTTT
TTTTTGAAATATGGCTATAAATATTAATTTATTAATCTTCCTTGGTTCTACCATGCATAAGTACTGTATC
TAATTAAAGAAAATAATTATTTTGTGCCCACGTGTTGTATGCATATATAAATTATGGAAGTTTTTTTTTT
TTTTTGAAAAAAGAAAAATTATGTCTTGGCGCAGGTATTAGTTTATGACATCTGTAAGAGAGAGAGGCTG
AGAAGTCGAGGTTAAGTTGCTGTCTCTGATATTTTCTAGGGTTTGTAATATTATATCCTTAATTCATA

1. A method for characterizing a Cannabis spp. plant with respect to a hermaphroditism trait, the method comprising the steps of:
(i) genotyping at least one plant with respect to a hermaphroditism QTL by detecting (a) one or more polymorphisms associated with the hermaphroditism trait as defined in any one of Tables 1, 3 to 11 and 15; and/or (b) a polymorphism causal for the hermaphroditism trait which is an A/G SNP at position 99811627 on chromosome NC_044370.1 with reference to the cs10 reference genome; and
(ii) characterizing the plant as having a hermaphroditism presence QTL or a hermaphroditism absence QTL based on the genotype at the polymorphism.

2. The method of claim 1, wherein the polymorphism is selected from the group consisting of “common_491”, “common_512”, “common_517”, “common_518”, “common_525”, “rare_57”, “rare_50”, “common_534”, “common_511”, “GBScompat_common_56”, “common_54”, “common 294”, as defined in any one of Tables 3 to 11 and 15, the A/G SNP at position 99811627 on chromosome NC 044370.1 with reference to the cs10 reference genome, and combinations thereof.

3. (canceled)

4. The method of claim 1, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

5. The method of claim 4, wherein the molecular markers are for detecting polymorphisms at regular intervals within the hermaphroditism QTL such that recombination can be excluded; or wherein the molecular markers are for detecting polymorphisms at regular intervals within the hermaphroditism QTL such that recombination can be quantified to estimate linkage disequilibrium between a particular polymorphism and a hermaphroditic phenotype; or wherein the molecular markers are designed based on a context sequence for the polymorphism in Tables 1, 3 to 11 or 15 or are selected from the primer pairs as defined in Table 12 or 17.

6. (canceled)

7. (canceled)

8. The method of claim 1, wherein the hermaphroditism QTL is a quantitative trait locus having a sequence that corresponds to nucleotides 28544332 to 35677966 of NC_044370.1 with reference to the CS10 genome and is defined by one or more polymorphisms associated with hermaphroditism as defined in any one of Tables 1, 3 to 11 and 15, or a genetic marker linked to the QTL.

9. A method of producing a Cannabis spp. plant having a hermaphroditism trait of interest, the method comprising the steps of:
(i) providing a donor parent plant having in its genome a hermaphroditism QTL characterized by (a) one or more polymorphisms associated with the hermaphroditism trait of interest as defined in any one of Tables 1, 3 to 11 and 15; and/or (b) a polymorphism causal for the hermaphroditism trait of interest which is an A/G SNP at position 99811627 on chromosome NC 044370.1 with reference to the cs10 reference genome;
(ii) crossing the donor parent plant having the hermaphroditism QTL with at least one recipient parent plant to obtain a progeny population of cannabis plants;
(iii) screening the progeny population of cannabis plants for the presence of the hermaphroditism QTL; and
(iv) selecting one or more progeny plants having the hermaphroditism QTL, wherein the mature plant displays the hermaphroditism trait of interest.

10. The method of claim 9, further comprising:
(v) crossing the one or more progeny plants with the donor recipient plant; or
(vi) selfing the one or more progeny plants.

11. The method of claim 9, wherein the screening comprises genotyping at least one plant from the progeny population with respect to the hermaphroditism QTL by detecting one or more polymorphisms associated with the hermaphroditism trait of interest as defined in any one of Tables 1, 3 to 11 and 15 and/or the polymorphism causal for the hermaphroditism trait of interest, and optionally wherein the method further comprises a step of genotyping the donor parent plant with respect to the hermaphroditism QTL by detecting one or more polymorphisms associated with the hermaphroditism trait of interest as defined in any one of Tables 1, 3 to 11 and 15 and/or the polymorphism causal for the hermaphroditism trait of interest, prior to step (i).

12. (canceled)

13. The method of claim 11, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

14. The method of claim 13, wherein the molecular markers are for detecting polymorphisms at regular intervals within the hermaphroditism QTL such that recombination can be excluded or such that recombination can be quantified to estimate linkage disequilibrium between a particular polymorphism and the hermaphroditism trait of interest, optionally wherein the molecular markers are designed based on a context sequence for the polymorphism in Tables 1, 3 to 11 or 15 or are selected from the primer pairs as defined in Table 12 or 17.

15. (canceled)

16. The method of claim 9, wherein the polymorphism is selected from the group consisting of “common_491”, “common_512”, “common_517”, “common_518”, “common_525”, “rare_57”, “rare_50”, “common_534”, “common_511”, “GBScompat_common_56”, “common_54”, “common_294”, as defined in any one of Tables 3 to 11 and 15, an A/G SNP at position 99811627 on chromosome NC 044370.1 with reference to the cs10 reference genome, and combinations thereof.

17. (canceled)

18. The method of claim 9, wherein the hermaphroditism QTL is a hermaphroditism absence QTL and the hermaphroditism trait of interest is a hermaphroditism absence trait.

19. The method of claim 9, wherein the hermaphroditism QTL is a quantitative trait locus having a sequence that corresponds to nucleotides 28544332 to 35677966 of NC 044370.1 with reference to the CS10 genome and is defined by one or more polymorphisms associated with hermaphroditism as defined in any one of Tables 1, 3 to 11 and 15, or a genetic marker linked to the QTL.

20. A method of producing a Cannabis spp. plant comprising a hermaphroditism trait of interest, the method comprising introducing into a Cannabis spp. plant a hermaphroditism QTL:
(a) characterized by one or more polymorphisms associated with the hermaphroditism trait of interest as defined in any one of Tables 1, 3 to 11 and 15, wherein said hermaphroditism QTL is associated with the hermaphroditism trait of interest in the plant; and/or
(b) comprising a polymorphism causal for the hermaphroditism trait of interest which is an A/G SNP at position 99811627 on chromosome NC_044370.1 with reference to the cs10 reference genome.

21. The method of claim 20, wherein introducing the hermaphroditism QTL comprises crossing a donor parent plant having the hermaphroditism QTL with a recipient parent plant.

22. The method of claim 20, wherein introducing the hermaphroditism QTL comprises genetically modifying the Cannabis spp. plant.

23. The method of claim 22, wherein genetically modifying the Cannabis spp. plant is by targeted mutagenesis of guanine to adenine (G>A) at position 99811627 on chromosome NC_044370.1 with reference to the cs10 reference genome.

24. The method of claim 20, wherein the hermaphroditism QTL is a quantitative trait locus having a sequence that corresponds to nucleotides 28544332 to 35677966 of NC 044370.1 with reference to the CS10 genome and is defined by one or more polymorphisms associated with hermaphroditism as defined in any one of Tables 1, 3 to 11 and 15, or a genetic marker linked to the QTL, optionally wherein the hermaphroditism QTL is a hermaphroditism absence QTL, and the hermaphroditism trait of interest is a hermaphroditism absence trait.

25. (canceled)

26. (canceled)

27. (canceled)

28. (canceled)

29. A Cannabis spp. plant comprising a hermaphroditism QTL:
(a) characterized by one or more polymorphisms associated with the hermaphroditism trait of interest as defined in any one of Tables 1, 3 to 11 and 15, wherein said hermaphroditism QTL is associated with the hermaphroditism trait of interest in the plant; and/or
(b) comprising a polymorphism causal for the hermaphroditism trait of interest which is an A/G SNP at position 99811627 on chromosome NC_044370.1 with reference to the cs10 reference genome, optionally wherein the plant comprises a hermaphroditism absence QTL and displays a hermaphroditism absence trait.

30.-37. (canceled)
